(19) United States
(12) Patent Application Publication
(10) Pub. No.: US 2026/0248783 A1
(43) Pub. Date: Aug. 27, 2026

(54) COMPOUNDS AS IMMUNOMODULATORS OF PD-L1 INTERACTIONS

(71) Applicant: ASCLETIS BIOSCIENCE CO., LTD., Hangzhou, Zhejiang (CN)

(72) Inventors: Jinhua CHEN, Hangzhou, Zhejiang (CN); Bin LIANG, Hangzhou, Zhejiang (CN); Peng WANG, Hangzhou, Zhejiang (CN); Yang LAI, Hangzhou, Zhejiang (CN); Bailing YANG, Hangzhou, Zhejiang (CN); Gudmundsson KRISTJAN, Hangzhou, Zhejiang (CN); Jinzi Jason WU, Hangzhou, Zhejiang (CN)

(21) Appl. No.: 18/290,813

(22) PCT Filed: Aug. 17, 2021
(86) PCT No.: PCT/CN2021/113010

§ 371 Date: Jan. 22, 2024

(51) Int. Cl. A61K 31/4985; A61K 31/4365; A61K 31/437; A61K 31/522; A61K 31/53; A61K 31/5377; A61P 35/00; A61P 37/02; C07D 519/00

(52) U.S. Cl. CPC A61K 31/4985; A61K 31/4365; A61K 31/437; A61K 31/522; A61K 31/53; A61K 31/5377; A61P 35/00; A61P 37/02; C07D 519/00

(57) ABSTRACT

PD-L1 inhibitors of various compound formulas, both generically and specifically are disclosed. Methods of making such PD-L1 inhibitor compounds are disclosed, both generically and specifically. Methods of using such PD-L1 inhibitor compounds singly or in combination with additional agents and compositions of such PD-L1 inhibitor compounds for the treatment of cancer and other conditions are disclosed.

### FIELD OF THE INVENTION

The present application is concerned with the field of pharmaceutically active compounds that modulate PD-L1 protein interactions, in particular, specific compounds, compositions and methods of use.

### BACKGROUND OF THE INVENTION

Programmed death-ligand 1 (“PD-L1”) is a protein that acts as a kind of “brake” to keep the body's immune responses under control. PD-L1 may be found on some normal cells and in higher-than-normal amounts on some types of cancer cells. When PD-L1 binds to another protein called PD-1 (a protein found on T cells), it keeps T cells from killing the PD-L1-containing cells, including the cancer cells. Anticancer drugs called immune checkpoint inhibitors bind to PD-L1 and block its binding to PD-1. This releases the “brakes” on the immune system and leaves T cells free to kill cancer cells. PD-L1 may also play a major role in suppressing the adaptive arm of immune system during particular events such as pregnancy, tissue allografts, autoimmune disease and other disease states such as hepatitis.

Accordingly, there is a need for new compounds that block PD-1/PD-L1 protein/protein interaction.

### SUMMARY OF THE INVENTION

One aspect of the present application relates to a compound of Formula (I):

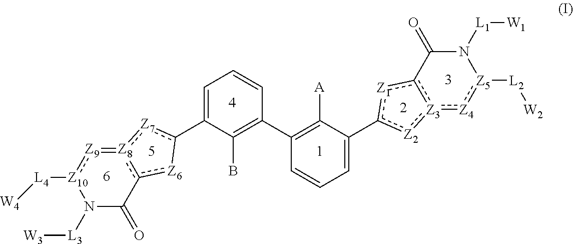

wherein:
each of A and B is independently selected from the group consisting of halogen, cyano, —N₃, alkyl and substituted alkyl, amine, alkylamine, and alkoxy;
Z₁ is ═N—, —N(R¹)—, ═C(R²)—, or —S—;
Z₂ is ═N—, —N(R¹)—, ═C(R²)—, or —S—;
Z₆ is ═N—, —N(R¹)—, ═C(R²)—, or —S—;
Z₇ is ═N—, —N(R¹)—, ═C(R²)—, or —S—;
each R¹ is independently —H, alkyl, cycloalkyl, substituted alkyl, alkenyl, alkynyl, or aryl; each R² is independently halogen, cyano, cycloalkyl, substituted alkyl, alkenyl, alkynyl, or aryl;
Z₃ is —N═, —N(R³)—, ═C(R⁴)—, or —C(R⁵R⁶H
Z₄ is —N═, —N(R³)—, ═C(R⁴)—, or —C(R⁵R⁶;
Z₅ is —N=, —N(R³)—, ═C(R⁴)—, or —C(R⁵R⁶;
Z₈ is —N═, —N(R³)—, ═C(R⁴)—, or —C(R⁵R⁶;
Z₉ is —N═, —N(R³)—, ═C(R⁴)—, or —C(R⁵R⁶H
Z₁₀ is —N═, —N(R³)—, ═C(R⁴)—, or —C(R⁵R₆).
each R³ is independently —H, alkyl, cycloalkyl, substituted alkyl, alkenyl, alkenyl, alkynyl, or aryl;
each of R⁴, R⁵ and R⁶ is independently halogen, cyano, cycloalkyl, substituted alkyl, alkenyl, alkenyl, alkynyl, or aryl;
each of L₁ and L₃ is independently an alkyl, substituted alkyl or hetereoatom chain, containing m atoms between Ring 3 and W₁, and between Ring 6 and W₃, wherein m=0, 1, 2, 3, 4, 5 or 6; when m is 0, W₁ or W₃ is directly linked to the corresponding nitrogen in Ring 3 or Ring 6, respectively;
each of W₁ and W₃ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.
each of L₂ and L₄ is independently an alkyl, substituted alkyl or hetereoatom chain, containing m atoms between Ring 3 and W₂, and between Ring 6 and W₄, wherein m=0, 1, 2, 3, 4, 5 or 6; when m is 0, W₂ or W₄ is directly linked to the corresponding nitrogen in Ring 3 or Ring 6, respectively;
each of W₂ and W₄ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

Another aspect of the present application relates to a method for treating a disease or condition relating to the interaction between PD-L1 and PD-1 in a subject, comprising the step of administering to the subject an effective amount of the compound of Formula (I), or a pharmaceutically acceptable salt, stereoisomer, mixture of stereoisomers, solvate, or tautomer thereof.

Another aspect of the present application relates to a method for making a compound of Formula (I).

### DETAILED DESCRIPTION OF THE INVENTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

At various places in the present specification, certain features of the compounds are disclosed in groups or in ranges. It is specifically intended that such a disclosure include each and every individual subcombination of the members of such groups and ranges.

The compounds described herein can be asymmetric (e.g, having one or more stereocenters). All stereoisomers, such as enantiomers and diastereomers, are intended unless otherwise indicated. Compounds of the present application that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present application. Cis and trans geometric isomers of the compounds of the present application are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as b-camphorsulfonic acid. Other resolving agents suitable for fractional crystallization methods include stereoisomerically pure forms of a-methylbenzylamine (e.g., S and R forms, or diastereomerically pure forms), 2-phenylglycinol, norephedrine, ephedrine, TV-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In compounds with more than one chiral centers, each of the chiral centers in the compound may be independently (R) or (S), unless otherwise indicated.

Compounds of the application also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3/f-imidazole, 1H-, 2H- and 4H-1,2,4-triazole, †H- and 211-isoindole and 1H- and 2//-pyrazole. Tautomeric forms can be in equilibrium or sterically locked into one form by appropriate substitution.

Compounds of the application can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the application can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms.

### I. Definitions

The term, “compound,” as used herein is meant to include all stereoisomers, geometric isomers, tautomers and isotopes of the structures depicted. The term is also meant to refer to compounds of the applications, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the application, or salts thereof, are substantially isolated. By “substantially isolated” is meant that the compound is at least partially or substantially separated from the environment in which it was formed or detected. Partial separation can include, e.g., a composition enriched in the compounds of the application.

Substantial separation can include compositions containing at least about 50%, at least about 60%, at least about 70%, at least about 80%, at least about 90%, at least about 95%, at least about 97%, or at least about 99% by weight of the compounds of the application, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present application also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the non-toxic salts of the parent compound formed, e.g., from non toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17th Ed., (Mack Publishing Company, Easton, 1985), p. 1418, Berge et al., J. Pharm. Sci, 1977, 66(1), 1-19 and in Stahl et al., Handbook of Pharmaceutical Salts: Properties, Selection, and Use, (Wiley, 2002). In some embodiments, the compounds described herein include the N-oxide forms.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

The term “solvate” refers to the compound formed by the interaction of a solvent and an EPI, a metabolite, or salt thereof. Suitable solvates are pharmaceutically acceptable solvates including hydrates.

The term “substituted” or “optionally substituted” as used in the present invention means that one or more hydrogen atoms of the group to which the term “substitute” or “optionally substituted” refers is replaced with one of the substituents, independently selected from lower alkyl, lower aryl, lower aralkyl, lower cyclic alkyl, lower heterocycloalkyl, hydroxy, lower alkoxy, lower aryloxy, perhaloalkoxy, aralkoxy, lower heteroaryl, lower heteroaryloxy, lower heteroarylalkyl, lower heteroaralkoxy, azido, amino, halo, lower alkylthio, oxo, lower acylalkyl, lower carboxy esters, carboxyl, carboxamido, nitro, lower acyloxy, lower aminoalkyl, lower alkylaminoaryl, lower alkylaryl, lower alkylaminoalkyl, lower alkoxyaryl, lower arylamino, lower aralkylamino, sulfonyl, lower carboxamidoalkylaryl, lower carboxamidoaryl, lower hydroxyalkyl, lower haloalkyl, lower alkylaminoalkylcarboxy-, lower aminocarboxamidoalkyl, cyano, lower alkoxyalkyl, lower perhaloalkyl, and lower arylalkyloxyalkyl, provided that the normal valency of the atom on which the substitution is considered is not exceeded and that the substitution results in a stable chemical compound, that is to say a compound that is sufficiently robust to be isolated from a reaction mixture.

The term “alkyl” refers to a straight or branched or cyclic chain hydrocarbon radical with only single carbon-carbon bonds. Representative examples include methyl, ethyl, propyl, isopropyl, cyclopropyl, butyl, isobutyl, tert-butyl, cyclobutyl, pentyl, cyclopentyl, hexyl, and cyclohexyl, all of which may be optionally substituted.

The term “aryl” refers to aromatic groups which have 5-14 ring atoms and at least one ring having a conjugated pi electron system and includes carbocyclic aryl, heterocyclic aryl and biaryl groups, all of which may be optionally substituted

Carbocyclic aryl groups are groups which have 6-14 ring atoms wherein the ring atoms on the aromatic ring are carbon atoms. Carbocyclic aryl groups include monocyclic carbocyclic aryl groups and polycyclic or fused compounds such as optionally substituted naphthyl groups.

Heterocyclic aryl or heteroaryl groups are groups which have 5-14 ring atoms wherein 1 to 4 heteroatoms are ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “biaryl” represents aryl groups which have 5-14 atoms containing more than one aromatic ring including both fused ring systems and aryl groups substituted with other aryl groups. Such groups may be optionally substituted. Suitable biaryl groups include naphthyl and biphenyl.

The term “substituted aryl” and “substituted heteroaryl” refers to aryl and heteroaryl groups substituted with 1-3 substituents. These substituents are selected from the group consisting of lower alkyl, lower alkoxy, lower perhaloalkyl, halo, hydroxy, and amino.

The term “aralkyl” refers to an alkylene group substituted with an aryl group. Suitable aralkyl groups include benzyl, picolyl, and the like, and may be optionally substituted.

The term “heteroarylalkyl” refers to an alkylene group substituted with a heteroaryl group.

The term “alkylaryl” refers to an aryl group substituted with an alkyl group. “Lower alkylaryl” refers to such groups where alkyl is lower alkyl.

The term “lower” referred to herein in connection with organic radicals or compounds respectively refers to 6 carbon atoms or less. Such groups may be straight chain, branched, or cyclic.

The term “higher” referred to herein in connection with organic radicals or compounds respectively refers to 7 or more carbon atoms. Such groups may be straight chain, branched, or cyclic.

The term “cyclic alkyl” or “cycloalkyl” refers to alkyl groups that are cyclic of 3 to 10 carbon atoms, and in one aspect are 3 to 6 carbon atoms Suitable cyclic groups include norbornyl and cyclopropyl. Such groups may be substituted.

The term “heterocyclic,” “heterocyclic alkyl” or “heterocycloalkyl” refer to cyclic groups of 3 to 10 atoms, and in one aspect are 3 to 6 atoms, containing at least one heteroatom, in a further aspect are 1 to 3 heteroatoms. Suitable heteroatoms include oxygen, sulfur, and nitrogen. Heterocyclic groups may be attached through a nitrogen or through a carbon atom in the ring. The heterocyclic alkyl groups include unsaturated cyclic, fused cyclic and spirocyclic groups. Suitable heterocyclic groups include pyrrolidinyl, morpholino, morpholinoethyl, and pyridyl.

The terms “arylamino” (a), and “aralkylamino” (b), respectively, refer to the group —NRR′ wherein respectively, (a) R is aryl and R′ is hydrogen, alkyl, aralkyl, heterocycloalkyl, or aryl, and (b) R′ is aralkyl and R′ is hydrogen, aralkyl, aryl, alkyl or heterocycloalkyl.

The term “acyl” refers to —C(O)—R where R is alkyl, heterocycloalkyl, or aryl.

The term “carboxy esters” refers to —C(O)—OR where R is alkyl, aryl, aralkyl, cyclic alkyl, or heterocycloalkyl, all optionally substituted.

The term “carboxyl” refers to —C(O)—OH.

The term “oxo” refers to ═O in an alkyl or heterocycloalkyl group.

The term “amino” refers to —NRR′ where R and R′ are independently selected from hydrogen, alkyl, aryl, aralkyl and heterocycloalkyl, all except H are optionally substituted; and R and R′ can form a cyclic ring system.

The term “carboxylamido” refers to —C(O)NR₂ where each R is independently hydrogen or alkyl.

The term “sulphonylamido” or “-sulfonylamido” refers to —S(═O)₂R₂ where each R is independently hydrogen or alkyl.

The term “halogen” or “halo” refers to —F, —Cl, —Br and —I.

The term “alkylaminoalkylcarboxy” refers to the group alkyl-NR-alk-C(O)—O— where “alk” is an alkylene group, and R is a H or lower alkyl.

The term “sulphonyl” or “sulfonyl” refers to —SO₂R, where R is H, alkyl, aryl, aralkyl, or heterocycloalkyl.

The term “sulphonate” or “sulfonate” refers to —SO₂—OR, where R is —H, alkyl, aryl, aralkyl, or heterocycloalkyl.

The term “alkenyl” refers to unsaturated groups which have 2 to 12 atoms and contain at least one carbon-carbon double bond and includes straight-chain, branched-chain and cyclic groups. Alkenyl groups may be optionally substituted. Suitable alkenyl groups include allyl. “1-Alkenyl” refers to alkenyl groups where the double bond is between the first and second carbon atom. If the 1-alkenyl group is attached to another group, e.g., it is a W substituent attached to the cyclic phosphonate, it is attached at the first carbon.

The term “alkynyl” refers to unsaturated groups which have 2 to 12 atoms and contain at least one carbon-carbon triple bond and includes straight-chain, branched-chain and cyclic groups. Alkynyl groups may be optionally substituted. Suitable alkynyl groups include ethynyl. “1-alkynyl” refers to alkynyl groups where the triple bond is between the first and second carbon atom. If the 1-alkynyl group is attached to another group, e.g., it is a W substituent attached to the cyclic phosphonate, it is attached at the first carbon.

The term “alkylene” refers to a divalent straight chain, branched chain or cyclic saturated aliphatic group. In one aspect the alkylene group contains up to and including 10 atoms. In another aspect the alkylene group contains up to and including 6 atoms. In a further aspect the alkylene group contains up to and including 4 atoms. The alkylene group can be either straight, branched or cyclic.

The term “acyloxy” refers to the ester group —O—C(O)R, where R is H, alkyl, alkenyl, alkynyl, aryl, aralkyl, or heterocycloalkyl.

The term “aminoalkyl” refers to the group NR₂-alk- wherein“alk” is an alkylene group and R is selected from —H, alkyl, aryl, aralkyl, and heterocycloalkyl.

The term “alkylaminoalkyl” refers to the group alkyl-NR-alk- wherein each“alk” is an independently selected alkylene, and R is H or lower alkyl. “Lower alkylaminoalkyl” refers to groups where the alkyl and the alkylene group is lower alkyl and alkylene, respectively.

The term “arylaminoalkyl” refers to the group aryl-NR-alk- wherein “alk” is an alkylene group and R is —H, alkyl, aryl, aralkyl, or heterocycloalkyl. In“lower arylaminoalkyl,” the alkylene group is lower alkylene.

The term “alkylaminoaryl-” refers to the group alkyl-NR-aryl- wherein “aryl” is a divalent group and R is —H, alkyl, aralkyl, or heterocycloalkyl. In “lower alkylaminoaryl,” the alkyl group is lower alkyl.

The term “alkoxyaryl” refers to an aryl group substituted with an alkyloxy group. In “lower alkyloxyaryl,” the alkyl group is lower alkyl.

The term “aryloxyalkyl” refers to an alkyl group substituted with an aryloxy group.

The term “aralkyloxyalkyl” refers to the group aryl-alk-O-alk- wherein “alk” is an alkylene group. “Lower aralkyloxyalkyl refers to such groups where the alkylene groups are lower alkylene.

The term “alkoxy-” or “alkyloxy-” refers to the group alkyl-O—.

The term “alkoxyalkyl” or “alkyloxyalkyl” refer to the group alkyl-O-alk- wherein “alk” is an alkylene group. In “lower alkoxyalkyl,” each alkyl and alkylene is lower alkyl and alkylene, respectively.

The term “alkylthio-” refers to the group alkyl-S—.

The term “alkylthioalkyl” refers to the group alkyl-5-alk- wherein “alk” is an alkylene group. In “lower alkylthioalkyl,” each alkyl and alkylene is lower alkyl and alkylene, respectively.

The term “alkoxycarbonyloxy-” refers to alkyl-O—C(O)—O—.

The term “aryloxycarbonyloxy-” refers to aryl-O—C(O)—O—.

The term “alkylthiocarbonyloxy” refers to alkyl-S—C(O)—O—.

The term “amido” refers to the NR₂ group next to an acyl or sulfonyl group as in NR₂—C(O)—, RC(O)—NR¹—, NR₂—S(═O)₂— and RS(═O)₂—NR¹—, where R and R¹ include —H, alkyl, aryl, aralkyl, and heterocycloalkyl

The term “carboxamido” refer to NR₂—C(O)— and RC(O)—NR¹—, where R and R¹ include —H, alkyl, aryl, aralkyl, and heterocycloalkyl. The term does not include urea, —NR—C(O)—NR—.

The term “sulphonamido” or “sulfonamido” refer to NR₂—S(═O)₂— and RS(═O)₂—NR¹—, where R and R¹ include —H, alkyl, aryl, aralkyl, and heterocycloalkyl. The term does not include sulfonylurea, —NR—S(═O)₂—NR—.

The term “carboxamidoalkylaryl” or “carboxamidoaryl” refers to an aryl-alk-NR¹—C(O), and ar-NR¹—C(O)-alk-, respectively where “ar” is aryl, “alk” is alkylene, R¹ and R include H, alkyl, aryl, aralkyl, and heterocycloalkyl.

The term “sulfonamidoalkylaryl” or “sulfonamidoaryl” refers to an aryl-alk-NR¹—S(═O)₂—, and ar-NR¹—S(═O)₂—, respectively where “ar” is aryl, “alk” is alkylene, R¹ and R include —H, alkyl, aryl, aralkyl, and heterocycloalkyl.

The term “hydroxyalkyl” refers to an alkyl group substituted with one —OH.

The term “haloalkyl” refers to an alkyl group substituted with halo.

The term “cyano” refers to —CN.

The term “nitro” refers to —NO₂.

The term “acylalkyl” refers to an alkyl-C(O)-alk-, where“alk” is alkylene.

The term “aminocarboxamidoalkyl” refers to the group NR₂—C(O)—N(R)-alk- wherein R is an alkyl group or H and “alk” is an alkylene group. “Lower aminocarboxamidoalkyl” refers to such groups wherein “alk” is lower alkylen

The term “heteroarylalkyl” refers to an alkylene group substituted with a heteroaryl group.

### II. Compounds that interfere with PD-L1 activity

An aspect of the present application relates to compounds that interfere with PD-L1 activity. In some embodiments, the compound has a generic structure as shown in formula (I): 1. A compound of Formula (I):

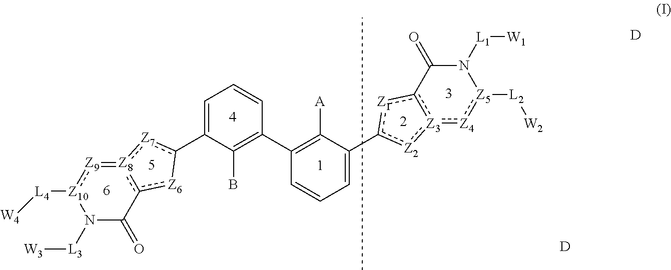

or a pharmaceutically acceptable salt, stereoisomer, mixture of stereoisomers, solvate, or tautomer thereof, wherein:
each of A and B is independently selected from the group consisting of halogen, cyano, —N₃, alkyl and substituted alkyl, amine, alkylamine, and alkoxy;
Z₁ is ═N—, —N(R¹)—, ═C(R²)—, or —S—;
Z₂ is ═N—, —N(R¹)—, ═C(R²)—, or —S—;
Z₆ is ═N—, —N(R¹)—, ═C(R²)—, or —S—;
Z₇ is ═N—, —N(R¹)—, ═C(R²)—, or —S—;
each R¹ is independently —H, alkyl, cycloalkyl, substituted alkyl, alkenyl, alkynyl, or aryl; each R² is independently halogen, cyano, cycloalkyl, substituted alkyl, alkenyl, alkynyl, or aryl;
Z₃ is —N═, —N(R³)—, ═C(R⁴)—, or —C(R⁵R⁶H
Z₄ is —N═, —N(R³)—, ═C(R⁴)—, or —C(R⁵R⁶H
Z₅ is —N═, —N(R³)—, ═C(R⁴)—, or —C(R⁵R⁶H
Z₈ is —N═, —N(R³)—, ═C(R⁴)—, or —C(R⁵R⁶H
Z₉ is —N═, —N(R³)—, ═C(R⁴)—, or —C(R⁵R⁶;
Z₁₀ is —N═, —N(R³)—, ═C(R⁴)—, or —C(R⁵R⁶)—;
each R³ is independently —H, alkyl, cycloalkyl, substituted alkyl, alkenyl, alkenyl, alkynyl, or aryl;
each of R⁴, R⁵ and R⁶ is independently halogen, cyano, cycloalkyl, substituted alkyl, alkenyl, alkenyl, alkynyl, or aryl;
each of L₁ and L₃ is independently an alkyl, substituted alkyl or hetereoatom chain, containing m atoms between Ring 3 and W₁, and between Ring 6 and W₃, wherein m=0, 1, 2, 3, 4, 5 or 6; when m is 0, W₁ or W₃ is directly linked to the corresponding nitrogen in Ring 3 or Ring 6, respectively;
each of W₁ and W₃ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.
each of L₂ and L₄ is independently an alkyl, substituted alkyl or hetereoatom chain, containing m atoms between Ring 3 and W₂, and between Ring 6 and W₄, wherein m=0, 1, 2, 3, 4, 5 or 6; when m is 0, W₂ or W₄ is directly linked to the corresponding nitrogen in Ring 3 or Ring 6, respectively;
each of W₂ and W₄ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

The compounds of formula (I) may be symmetrical (i.e., the left portion of the formula (I) is a mirror image of the right portion of formula (I)) or asymmetrical (i.e., the left portion of the formula (I) is different from the right portion of formula (I)) with respect to axis DD.

The compounds of the present application also include the pharmaceutically acceptable salts, stereoisomers, mixture of stereoisomers, solvates, enantiomers and tautomers of the compound of Formula (I).

In some embodiments, each of A and B is independently —Cl or methyl, wherein
Z₁ is ═N—, —N(R¹)—, ═C(R²)—, or —S—;
Z₂ is ═N—, —N(R¹)—, ═C(R²)—, or —S—;
Z₆ is ═N—, —N(R¹)—, ═C(R²)—, or —S—;
Z₇ is ═N—, —N(R¹)—, ═C(R²)—, or —S—;
each R¹ is independently —H, alkyl, cycloalkyl, or substituted alkyl; each R² is independently —H, —F, or methyl;
Z₃ is —N═, —N(R³)—, ═C(R⁴)—, or —C(R⁵R⁶H
Z₄ is —N═, —N(R³)—, ═C(R⁴)—, or —C(R⁵R⁶H
Z₅ is —N═, —N(R³)—, ═C(R⁴)—, or —C(RsR⁶;
Z₈ is —N═, —N(R³)—, ═C(R⁴)—, or —C(R⁵R⁶H
Z₉ is —N═, —N(R³)—, ═C(R⁴)—, or —C(R⁵R⁶;
Z₁₀ is —N═, —N(R³)—, ═C(R⁴)—, or —C(R⁵R⁶)—;
each R³ is independently —H, alkyl, cycloalkyl, or substituted alkyl;
each of R⁴, R⁵ and R⁶ is independently halogen, cyano, cycloalkyl, or substituted alkyl;
each of L₁ and L₃ is independently an alkyl, substituted alkyl or hetereoatom chain, containing m atoms between Ring 3 and W₁, and between Ring 6 and W₃, wherein m=0, 1, 2, 3, 4, 5 or 6; when m is 0, W₁ or W₃ is directly linked to the corresponding nitrogen in Ring 3 or Ring 6, respectively;
each of W₁ and W₃ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid;
each of L₂ and L₄ is independently an alkyl, substituted alkyl or hetereoatom chain, containing m atoms between Ring 3 and W₂, and between Ring 6 and W₄, wherein m=0, 1, 2, 3, 4, 5 or 6; when m is 0, W₂ or W₄ is directly linked to the corresponding nitrogen in Ring 3 or Ring 6, respectively; and
each of W₂ and W₄ is independently hydrogen, a three member ring, four member ring, five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

In some embodiments, the combination of Ring 2 and Ring 3, and/or the combination of Ring 5 and Ring 6, of the compound of Formula (I) are independently selected from the group consisting of:

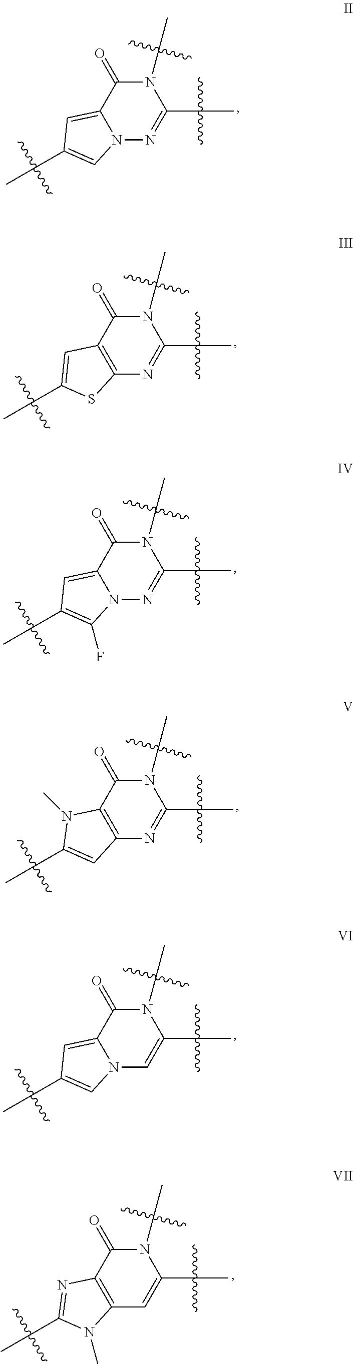 [SMILES: *c1cc2c(=O)n(*)c(*)nn2c1 ; *c1cc2c(=O)n(*)c(*)nc2s1 ; *c1cc2c(=O)n(*)c(*)nn2c1F ; *c1nc2cc(*)n(C)c2c(=O)n1* ; *c1cc2c(=O)n(*)c(*)cn2c1 ; *c1cc2c(nc(*)n2C)c(=O)n1*]

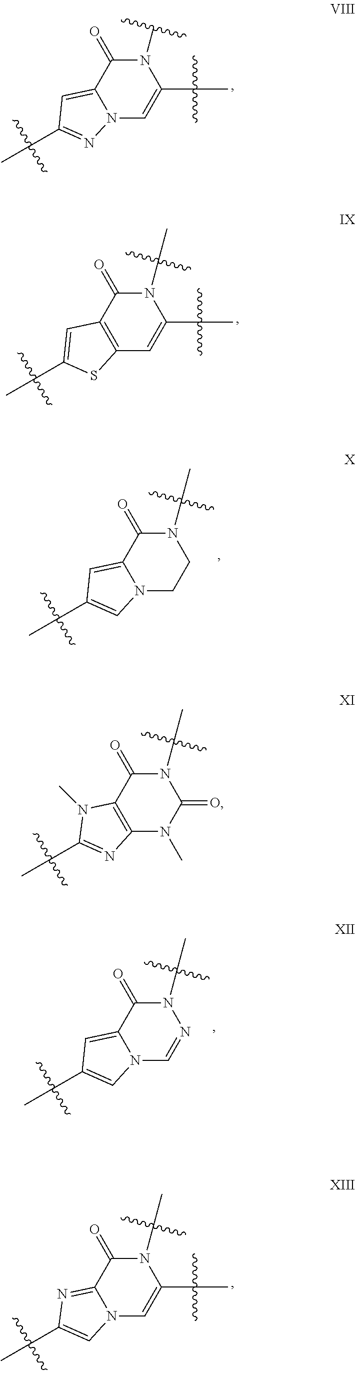 [SMILES: *c1cc2c(=O)n(*)c(*)cn2n1 ; *c1cc2c(=O)n(*)c(*)cc2s1 ; *c1cc2n(c1)CCN(*)C2=O ; *c1nc2c(c(=O)n(*)c(=O)n2C)n1C ; *c1cc2c(=O)n(*)ncn2c1 ; *c1cn2cc(*)n(*)c(=O)c2n1]

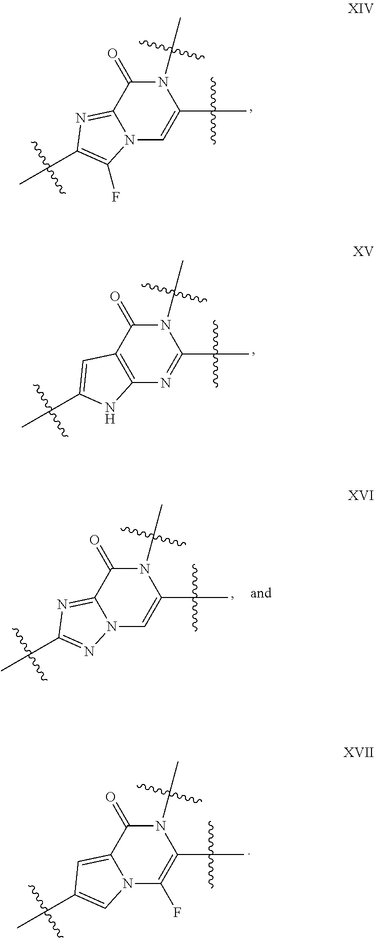 [SMILES: *c1nc2c(=O)n(*)c(*)cn2c1F ; *c1cc2c(=O)n(*)c(*)nc2[nH]1 ; *c1nc2c(=O)n(*)c(*)cn2n1 ; *c1cc2c(=O)n(*)c(*)c(F)n2c1]

In some embodiments, the combination of Ring 2 and Ring 3, and/or the combination of Ring 5 and Ring 6, of the compound of Formula (I) are independently selected from the group consisting of:

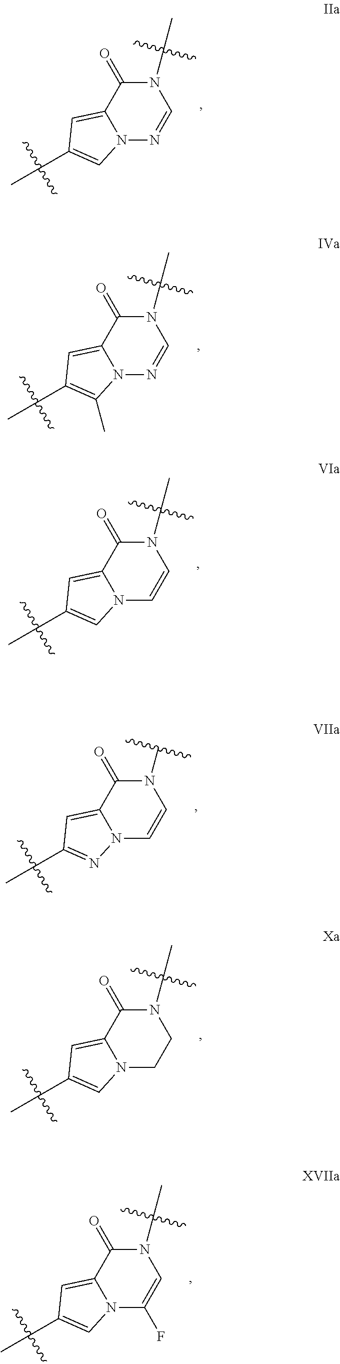 [SMILES: *c1cc2c(=O)n(*)cnn2c1 ; *c1cc2c(=O)n(*)cnn2c1C ; *c1cc2c(=O)n(*)ccn2c1 ; *c1cc2c(=O)n(*)ccn2n1 ; *c1cc2n(c1)CCN(*)C2=O ; *c1cc2c(=O)n(*)cc(F)n2c1]

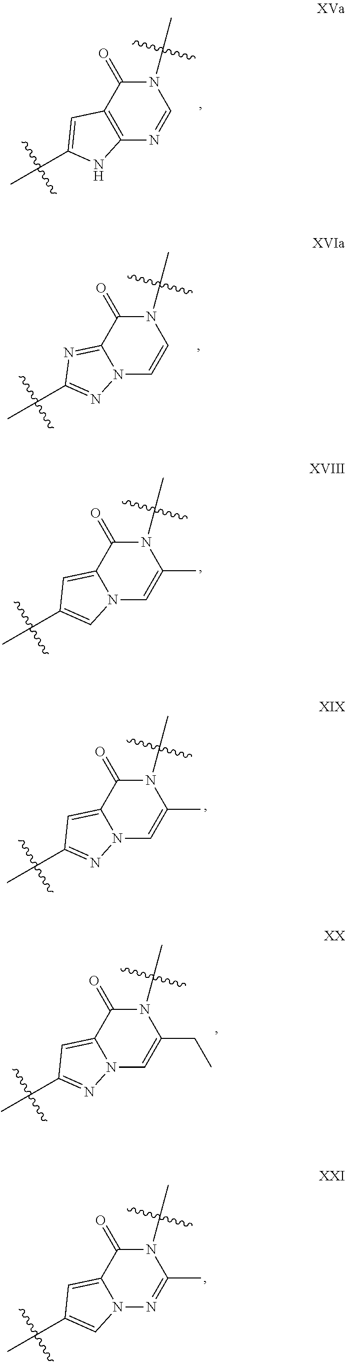 [SMILES: *c1cc2c(=O)n(*)cnc2[nH]1 ; *c1nc2c(=O)n(*)ccn2n1 ; *c1cc2c(=O)n(*)c(C)cn2c1 ; *c1cc2c(=O)n(*)c(C)cn2n1 ; *c1cc2c(=O)n(*)c(CC)cn2n1 ; *c1cc2c(=O)n(*)c(C)nn2c1]

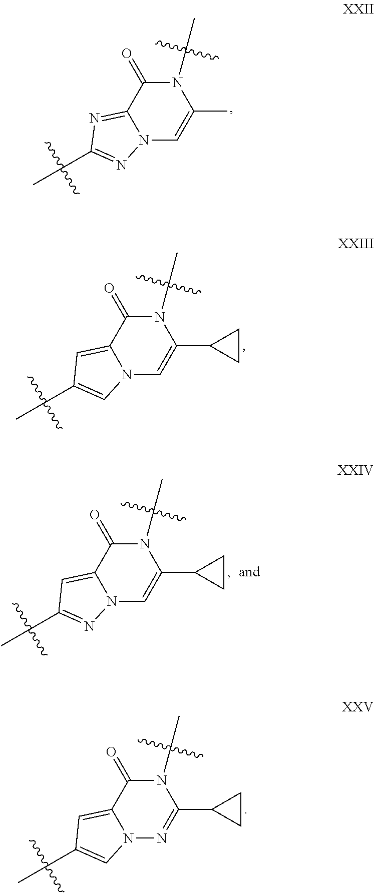 [SMILES: *c1nc2c(=O)n(*)c(C)cn2n1 ; *c1cc2c(=O)n(*)c(C3CC3)cn2c1 ; *c1cc2c(=O)n(*)c(C3CC3)cn2n1 ; *c1cc2c(=O)n(*)c(C3CC3)nn2c1]

In some embodiments, each of the L₁, L₂, L₃ and L₄ is independently a C₁-C₃ alkyl.

In some embodiments, each of W₁, W₂, W₃ and W₄ is independently selected from the group consisting of:

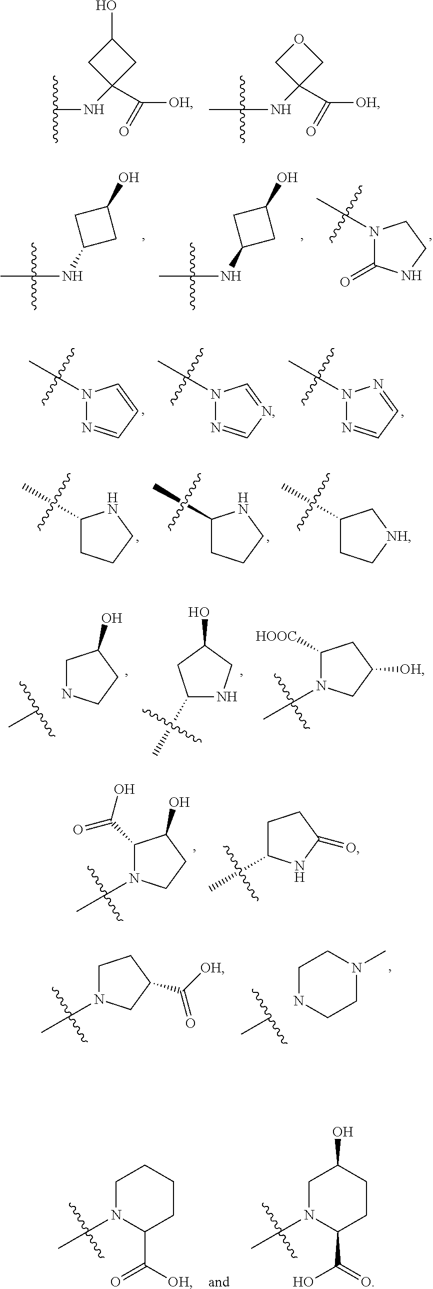

In some embodiments, one or more of W₁, W₂, W₃ and W₄ have a general formula of:

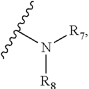

wherein each of R⁷ and R⁸ is independently —H, alkyl, or substituted alkyl. In some related embodiments, R7 is one of

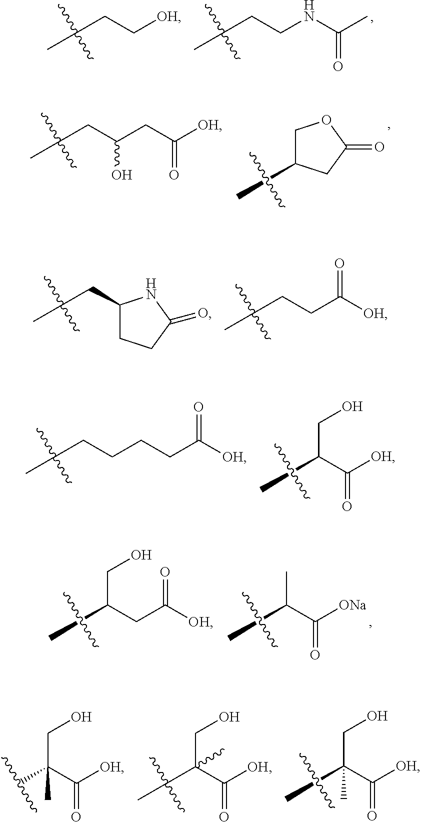

and

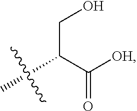

and R⁸ is independently —H, alkyl, or substituted alkyl. Certain embodiments of the compounds disclosed herein are listed, without limitation, in Table 1. The compounds of the present application also include the pharmaceutically acceptable salts, stereoisomers, mixtures of stereoisomers, solvates, or tautomers of the compounds in Table 1.

TABLE 1
Exemplary compounds of formula (I)

| ID | Structure |
|---|---|
| 1 | 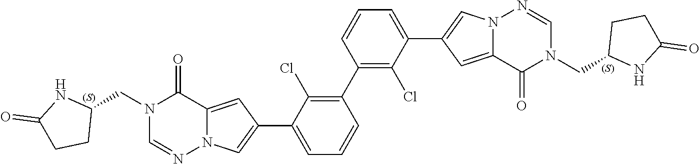 |
| | 1 |
| 2 | 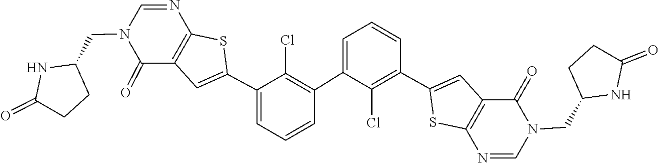 [SMILES: O=C1CC[C@@H](Cn2cnc3sc(-c4cccc(-c5cccc(-c6cc7c(=O)n(C[C@@H]8CCC(=O)N8)cnc7s6)c5Cl)c4Cl)cc3c2=O)N1] |
| | 2 |
| 3 | 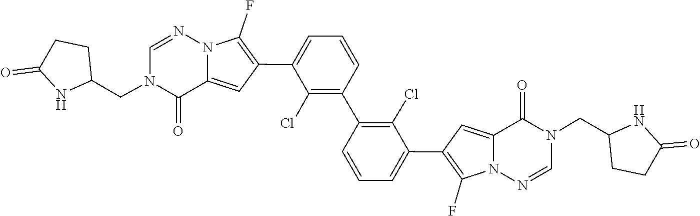 [SMILES: O=C1CCC(Cn2cnn3c(F)c(-c4cccc(-c5cccc(-c6cc7c(=O)n(CC8CCC(=O)N8)cnn7c6F)c5Cl)c4Cl)cc3c2=O)N1] |
| | 3 |
| 4 | 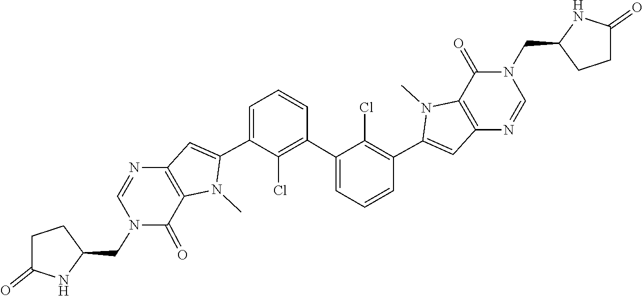 [SMILES: Cn1c(-c2cccc(-c3cccc(-c4cc5ncn(C[C@@H]6CCC(=O)N6)c(=O)c5n4C)c3Cl)c2Cl)cc2ncn(C[C@@H]3CCC(=O)N3)c(=O)c21] |
| | 4 |
| 5 | 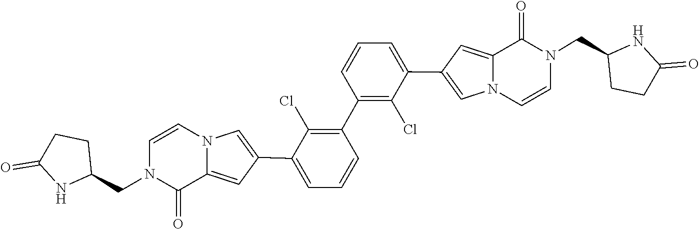 [SMILES: O=C1CC[C@@H](Cn2ccn3cc(-c4cccc(-c5cccc(-c6cc7c(=O)n(C[C@@H]8CCC(=O)N8)ccn7c6)c5Cl)c4Cl)cc3c2=O)N1] |
| | 5 |
| 6 | 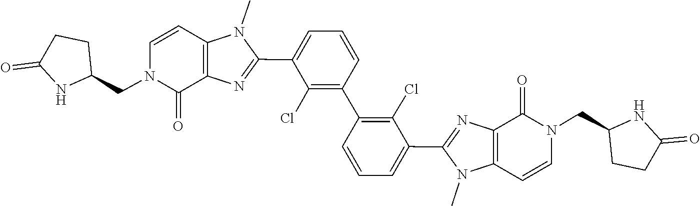 [SMILES: Cn1c(-c2cccc(-c3cccc(-c4nc5c(=O)n(C[C@@H]6CCC(=O)N6)ccc5n4C)c3Cl)c2Cl)nc2c(=O)n(C[C@@H]3CCC(=O)N3)ccc21] |
| | 6 |
| 7 | 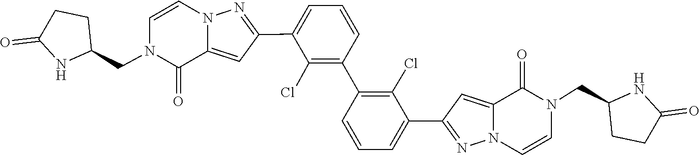 [SMILES: O=C1CC[C@@H](Cn2ccn3nc(-c4cccc(-c5cccc(-c6cc7c(=O)n(C[C@@H]8CCC(=O)N8)ccn7n6)c5Cl)c4Cl)cc3c2=O)N1] |
| | 7 |
| 8 | 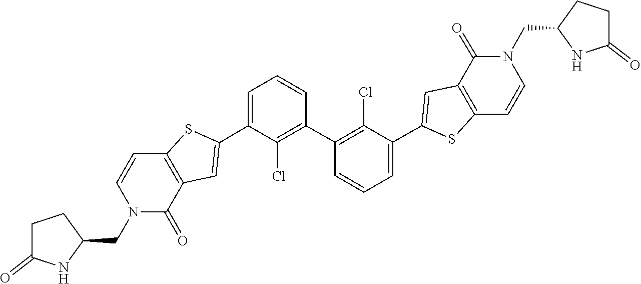 [SMILES: O=C1CC[C@@H](Cn2ccc3sc(-c4cccc(-c5cccc(-c6cc7c(=O)n(C[C@@H]8CCC(=O)N8)ccc7s6)c5Cl)c4Cl)cc3c2=O)N1] |
| | 8 |
| 9 | 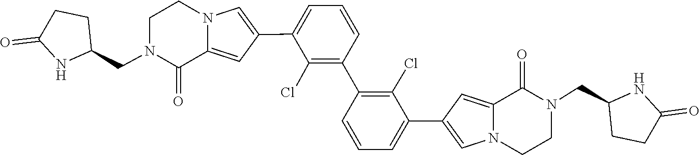 [SMILES: O=C1CC[C@@H](CN2CCn3cc(-c4cccc(-c5cccc(-c6cc7n(c6)CCN(C[C@@H]6CCC(=O)N6)C7=O)c5Cl)c4Cl)cc3C2=O)N1] |
| | 9 |
| 10 | 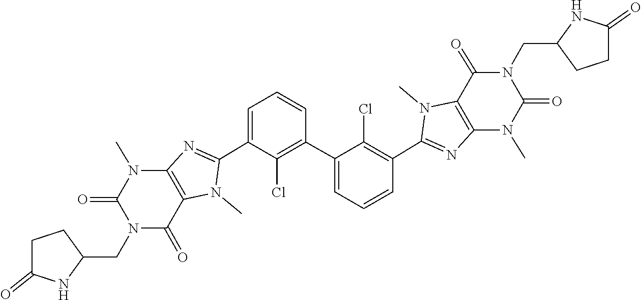 [SMILES: Cn1c(-c2cccc(-c3cccc(-c4nc5c(c(=O)n(CC6CCC(=O)N6)c(=O)n5C)n4C)c3Cl)c2Cl)nc2c1c(=O)n(CC1CCC(=O)N1)c(=O)n2C] |
| | 10 |
| 11 | 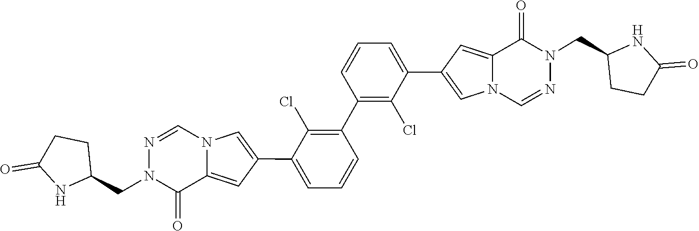 [SMILES: O=C1CC[C@@H](Cn2ncn3cc(-c4cccc(-c5cccc(-c6cc7c(=O)n(C[C@@H]8CCC(=O)N8)ncn7c6)c5Cl)c4Cl)cc3c2=O)N1] |
| | 11 |
| 12 | 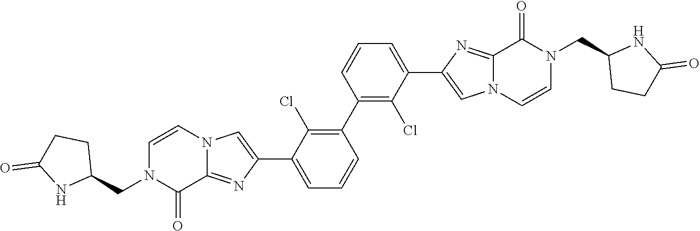 [SMILES: O=C1CC[C@@H](Cn2ccn3cc(-c4cccc(-c5cccc(-c6cn7ccn(C[C@@H]8CCC(=O)N8)c(=O)c7n6)c5Cl)c4Cl)nc3c2=O)N1] |
| | 12 |
| 13 | 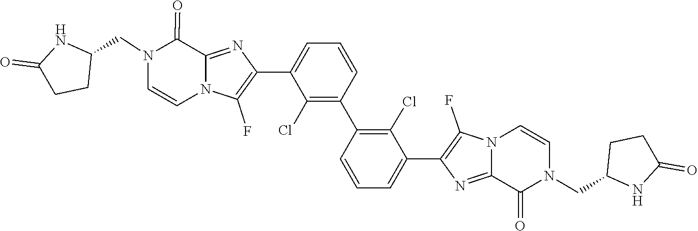 [SMILES: O=C1CC[C@@H](Cn2ccn3c(F)c(-c4cccc(-c5cccc(-c6nc7c(=O)n(C[C@@H]8CCC(=O)N8)ccn7c6F)c5Cl)c4Cl)nc3c2=O)N1] |
| | 13 |
| 14 | 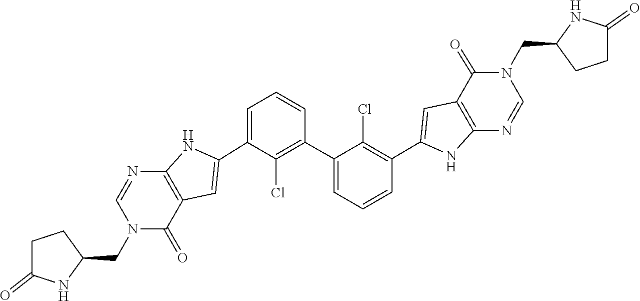 [SMILES: O=C1CC[C@@H](Cn2cnc3[nH]c(-c4cccc(-c5cccc(-c6cc7c(=O)n(C[C@@H]8CCC(=O)N8)cnc7[nH]6)c5Cl)c4Cl)cc3c2=O)N1] |
| | 14 |
| 15 | 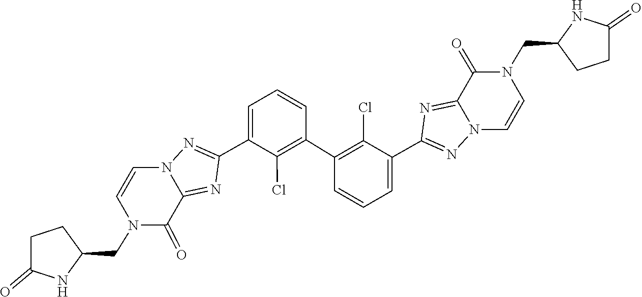 [SMILES: O=C1CC[C@@H](Cn2ccn3nc(-c4cccc(-c5cccc(-c6nc7c(=O)n(C[C@@H]8CCC(=O)N8)ccn7n6)c5Cl)c4Cl)nc3c2=O)N1] |
| | 15 |
| 16 | 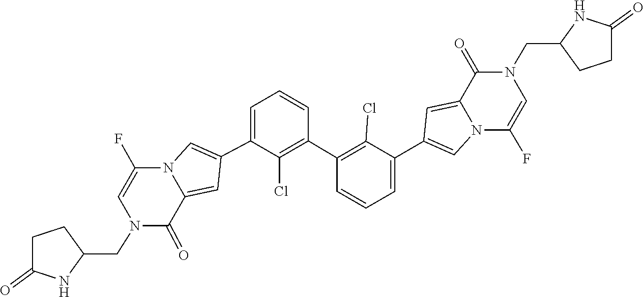 [SMILES: O=C1CCC(Cn2cc(F)n3cc(-c4cccc(-c5cccc(-c6cc7c(=O)n(CC8CCC(=O)N8)cc(F)n7c6)c5Cl)c4Cl)cc3c2=O)N1] |
| | 16 |
| 17 | 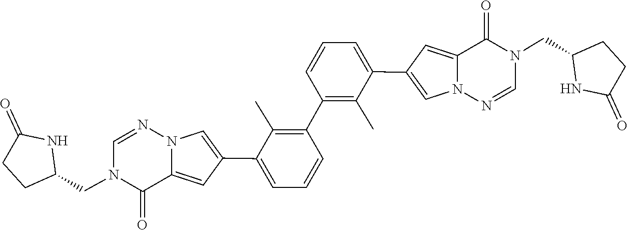 [SMILES: Cc1c(-c2cc3c(=O)n(C[C@@H]4CCC(=O)N4)cnn3c2)cccc1-c1cccc(-c2cc3c(=O)n(C[C@@H]4CCC(=O)N4)cnn3c2)c1C] |
| | 17 |
| 18 | 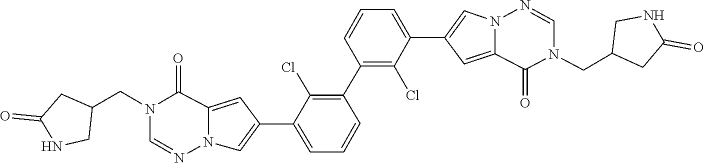 [SMILES: O=C1CC(Cn2cnn3cc(-c4cccc(-c5cccc(-c6cc7c(=O)n(CC8CNC(=O)C8)cnn7c6)c5Cl)c4Cl)cc3c2=O)CN1] |
| | 18 |
| 19 | 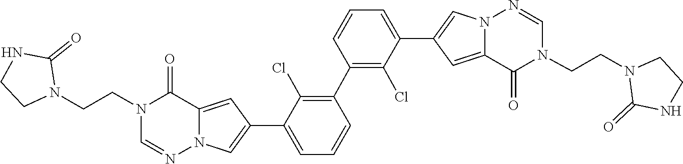 [SMILES: O=C1NCCN1CCn1cnn2cc(-c3cccc(-c4cccc(-c5cc6c(=O)n(CCN7CCNC7=O)cnn6c5)c4Cl)c3Cl)cc2c1=O] |
| | 19 |
| 20 | 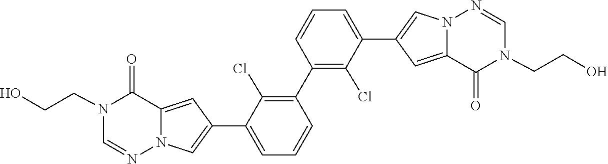 [SMILES: O=c1c2cc(-c3cccc(-c4cccc(-c5cc6c(=O)n(CCO)cnn6c5)c4Cl)c3Cl)cn2ncn1CCO] |
| | 20 |
| 21 | 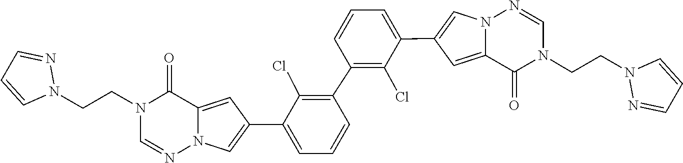 [SMILES: O=c1c2cc(-c3cccc(-c4cccc(-c5cc6c(=O)n(CCn7cccn7)cnn6c5)c4Cl)c3Cl)cn2ncn1CCn1cccn1] |
| | 21 |
| 22 | 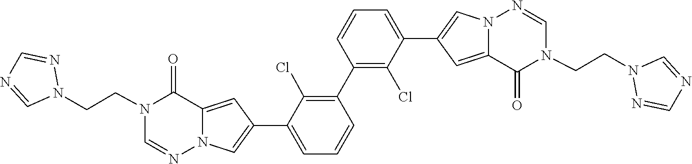 [SMILES: O=c1c2cc(-c3cccc(-c4cccc(-c5cc6c(=O)n(CCn7cncn7)cnn6c5)c4Cl)c3Cl)cn2ncn1CCn1cncn1] |
| | 22 |
| 23 | 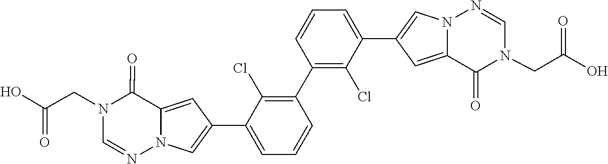 [SMILES: O=C(O)Cn1cnn2cc(-c3cccc(-c4cccc(-c5cc6c(=O)n(CC(=O)O)cnn6c5)c4Cl)c3Cl)cc2c1=O] |
| | 23 |
| 24 | 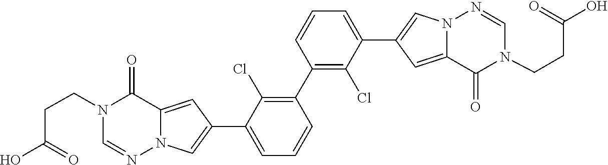 [SMILES: O=C(O)CCn1cnn2cc(-c3cccc(-c4cccc(-c5cc6c(=O)n(CCC(=O)O)cnn6c5)c4Cl)c3Cl)cc2c1=O] |
| | 24 |
| 25 | 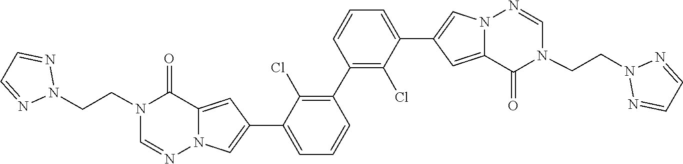 [SMILES: O=c1c2cc(-c3cccc(-c4cccc(-c5cc6c(=O)n(CCn7nccn7)cnn6c5)c4Cl)c3Cl)cn2ncn1CCn1nccn1] |
| | 25 |
| 26 | 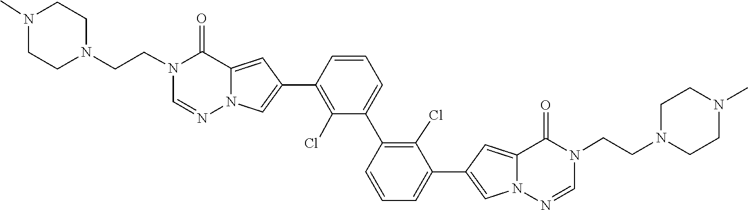 |
| | 26 |
| 27 | 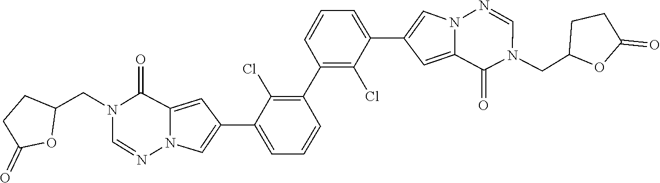 [SMILES: O=C1CCC(Cn2cnn3cc(-c4cccc(-c5cccc(-c6cc7c(=O)n(CC8CCC(=O)O8)cnn7c6)c5Cl)c4Cl)cc3c2=O)O1] |
| | 27 |
| 28 | 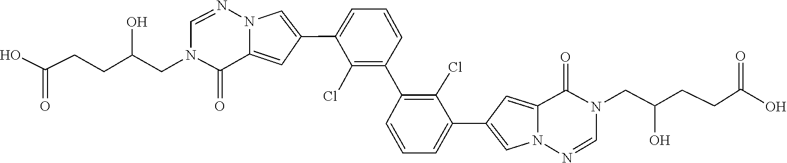 [SMILES: O=C(O)CCC(O)Cn1cnn2cc(-c3cccc(-c4cccc(-c5cc6c(=O)n(CC(O)CCC(=O)O)cnn6c5)c4Cl)c3Cl)cc2c1=O] |
| | 28 |
| 29 | 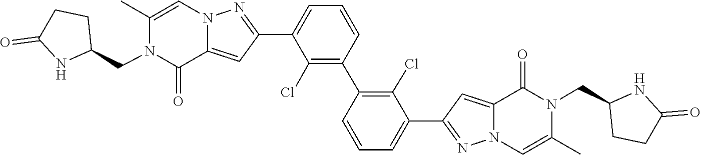 [SMILES: Cc1cn2nc(-c3cccc(-c4cccc(-c5cc6c(=O)n(C[C@@H]7CCC(=O)N7)c(C)cn6n5)c4Cl)c3Cl)cc2c(=O)n1C[C@@H]1CCC(=O)N1] |
| | 29 |
| 30 | 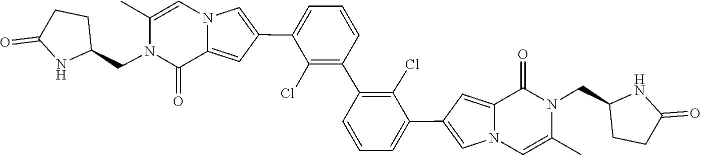 [SMILES: Cc1cn2cc(-c3cccc(-c4cccc(-c5cc6c(=O)n(C[C@@H]7CCC(=O)N7)c(C)cn6c5)c4Cl)c3Cl)cc2c(=O)n1C[C@@H]1CCC(=O)N1] |
| | 30 |
| 31 | 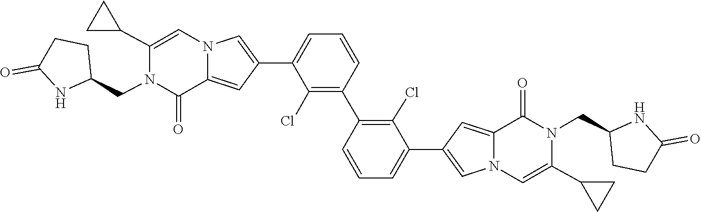 [SMILES: O=C1CC[C@@H](Cn2c(C3CC3)cn3cc(-c4cccc(-c5cccc(-c6cc7c(=O)n(C[C@@H]8CCC(=O)N8)c(C8CC8)cn7c6)c5Cl)c4Cl)cc3c2=O)N1] |
| | 31 |
| 32 | 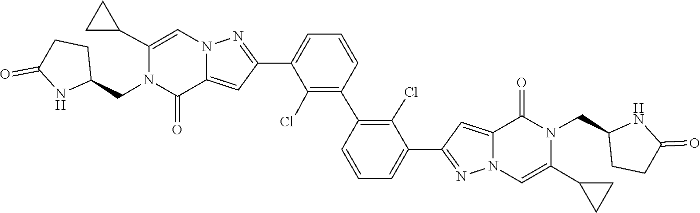 [SMILES: O=C1CC[C@@H](Cn2c(C3CC3)cn3nc(-c4cccc(-c5cccc(-c6cc7c(=O)n(C[C@@H]8CCC(=O)N8)c(C8CC8)cn7n6)c5Cl)c4Cl)cc3c2=O)N1] |
| | 32 |
| 33 | 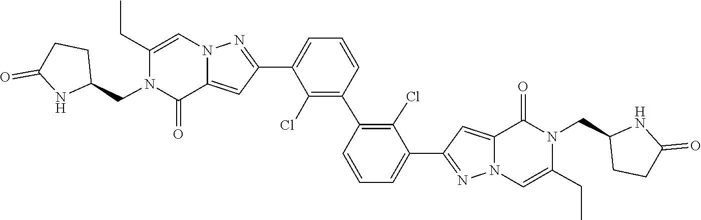 [SMILES: CCc1cn2nc(-c3cccc(-c4cccc(-c5cc6c(=O)n(C[C@@H]7CCC(=O)N7)c(CC)cn6n5)c4Cl)c3Cl)cc2c(=O)n1C[C@@H]1CCC(=O)N1] |
| | 33 |
| 34 | 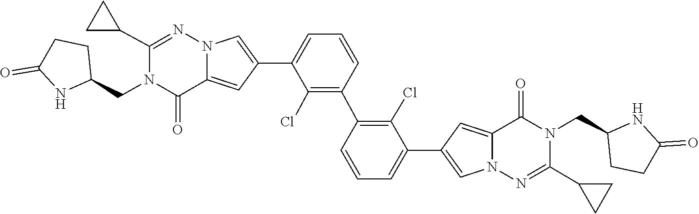 [SMILES: O=C1CC[C@@H](Cn2c(C3CC3)nn3cc(-c4cccc(-c5cccc(-c6cc7c(=O)n(C[C@@H]8CCC(=O)N8)c(C8CC8)nn7c6)c5Cl)c4Cl)cc3c2=O)N1] |
| | 34 |
| 35 | 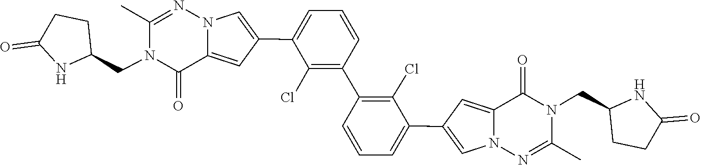 [SMILES: Cc1nn2cc(-c3cccc(-c4cccc(-c5cc6c(=O)n(C[C@@H]7CCC(=O)N7)c(C)nn6c5)c4Cl)c3Cl)cc2c(=O)n1C[C@@H]1CCC(=O)N1] |
| | 35 |
| 36 | 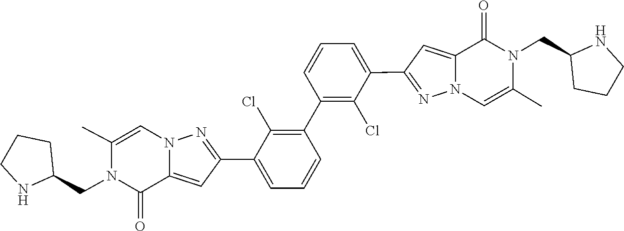 [SMILES: Cc1cn2nc(-c3cccc(-c4cccc(-c5cc6c(=O)n(C[C@@H]7CCCN7)c(C)cn6n5)c4Cl)c3Cl)cc2c(=O)n1C[C@@H]1CCCN1] |
| | 36 |
| 37 | 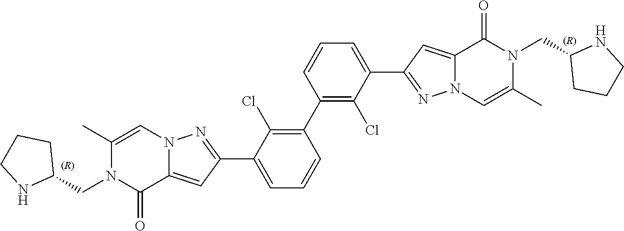 |
| | 37 |
| 38 | 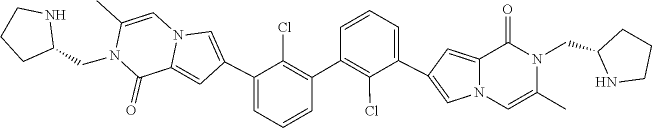 [SMILES: Cc1cn2cc(-c3cccc(-c4cccc(-c5cc6c(=O)n(C[C@@H]7CCCN7)c(C)cn6c5)c4Cl)c3Cl)cc2c(=O)n1C[C@@H]1CCCN1] |
| | 38 |
| 39 | 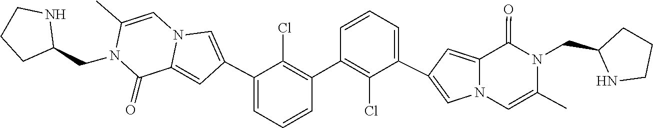 [SMILES: Cc1cn2cc(-c3cccc(-c4cccc(-c5cc6c(=O)n(C[C@H]7CCCN7)c(C)cn6c5)c4Cl)c3Cl)cc2c(=O)n1C[C@H]1CCCN1] |
| | 39 |
| 40 | 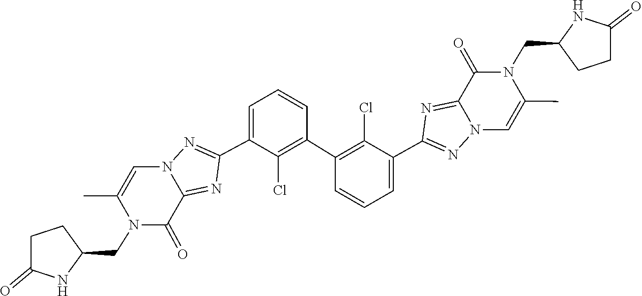 [SMILES: Cc1cn2nc(-c3cccc(-c4cccc(-c5nc6c(=O)n(C[C@@H]7CCC(=O)N7)c(C)cn6n5)c4Cl)c3Cl)nc2c(=O)n1C[C@@H]1CCC(=O)N1] |
| | 40 |
| 41 | 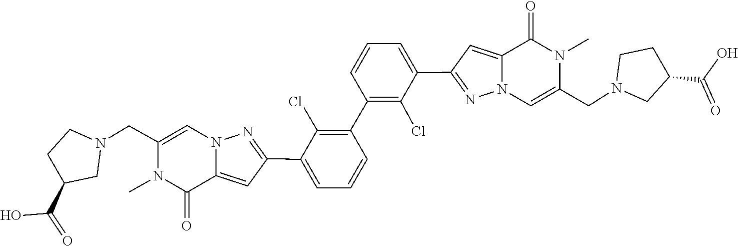 [SMILES: Cn1c(CN2CC[C@H](C(=O)O)C2)cn2nc(-c3cccc(-c4cccc(-c5cc6c(=O)n(C)c(CN7CC[C@H](C(=O)O)C7)cn6n5)c4Cl)c3Cl)cc2c1=O] |
| | 41 |
| 42 | 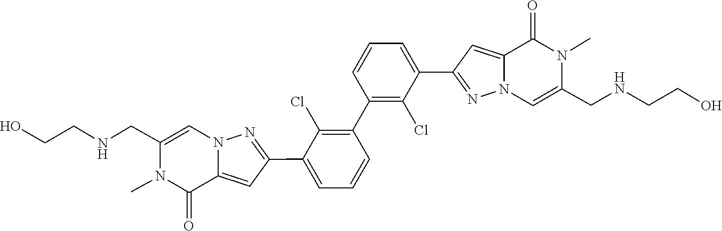 [SMILES: Cn1c(CNCCO)cn2nc(-c3cccc(-c4cccc(-c5cc6c(=O)n(C)c(CNCCO)cn6n5)c4Cl)c3Cl)cc2c1=O] |
| | 42 |
| 43 | 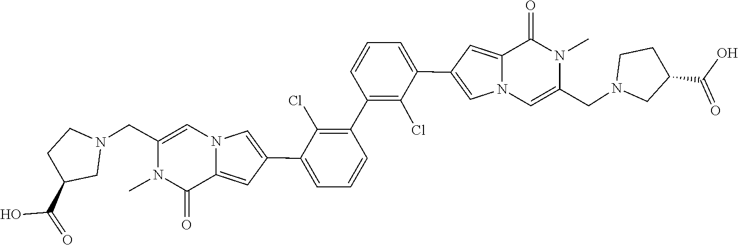 [SMILES: Cn1c(CN2CC[C@H](C(=O)O)C2)cn2cc(-c3cccc(-c4cccc(-c5cc6c(=O)n(C)c(CN7CC[C@H](C(=O)O)C7)cn6c5)c4Cl)c3Cl)cc2c1=O] |
| | 43 |
| 44 | 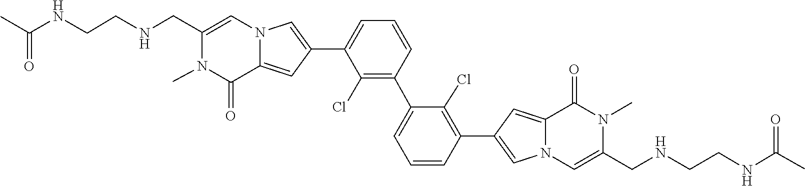 [SMILES: CC(=O)NCCNCc1cn2cc(-c3cccc(-c4cccc(-c5cc6c(=O)n(C)c(CNCCNC(C)=O)cn6c5)c4Cl)c3Cl)cc2c(=O)n1C] |
| | 44 |
| 45 | 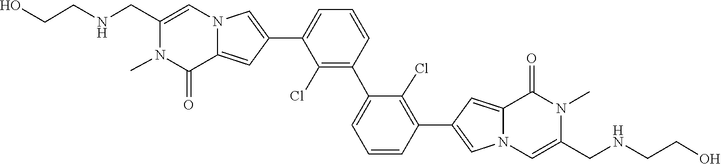 [SMILES: Cn1c(CNCCO)cn2cc(-c3cccc(-c4cccc(-c5cc6c(=O)n(C)c(CNCCO)cn6c5)c4Cl)c3Cl)cc2c1=O] |
| | 45 |
| 46 | 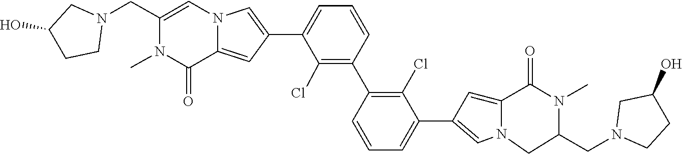 [SMILES: CN1C(=O)c2cc(-c3cccc(-c4cccc(-c5cc6c(=O)n(C)c(CN7CC[C@H](O)C7)cn6c5)c4Cl)c3Cl)cn2CC1CN1CC[C@H](O)C1] |
| | 46 |
| 47 | 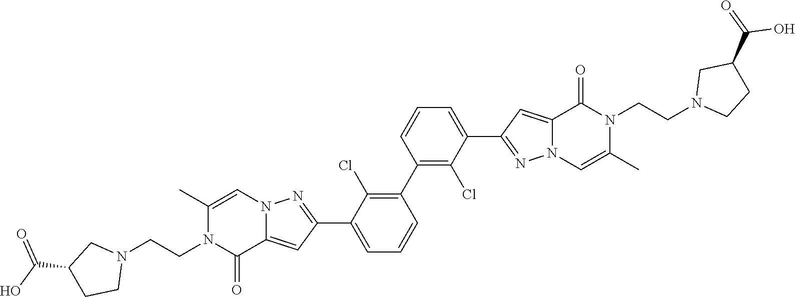 [SMILES: Cc1cn2nc(-c3cccc(-c4cccc(-c5cc6c(=O)n(CCN7CC[C@H](C(=O)O)C7)c(C)cn6n5)c4Cl)c3Cl)cc2c(=O)n1CCN1CC[C@H](C(=O)O)C1] |
| | 47 |
| 48 | 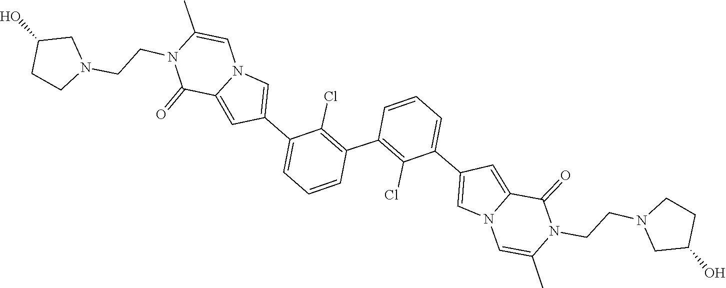 [SMILES: Cc1cn2cc(-c3cccc(-c4cccc(-c5cc6c(=O)n(CCN7CC[C@H](O)C7)c(C)cn6c5)c4Cl)c3Cl)cc2c(=O)n1CCN1CC[C@H](O)C1] |
| | 48 |
| 49 | 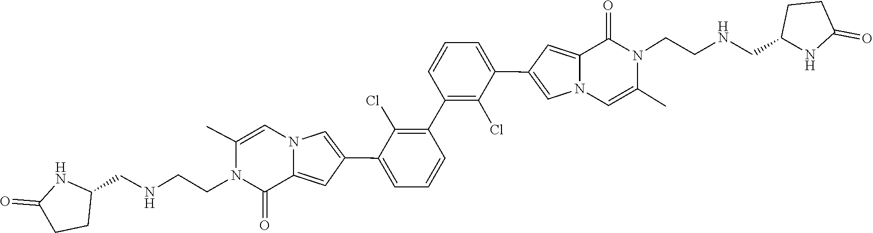 [SMILES: Cc1cn2cc(-c3cccc(-c4cccc(-c5cc6c(=O)n(CCNC[C@@H]7CCC(=O)N7)c(C)cn6c5)c4Cl)c3Cl)cc2c(=O)n1CCNC[C@@H]1CCC(=O)N1] |
| | 49 |
| 50 | 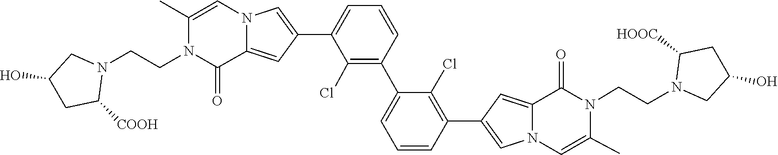 [SMILES: Cc1cn2cc(-c3cccc(-c4cccc(-c5cc6c(=O)n(CCN7C[C@@H](O)C[C@H]7C(=O)O)c(C)cn6c5)c4Cl)c3Cl)cc2c(=O)n1CCN1C[C@@H](O)C[C@H]1C(=O)O] |
| | 50 |
| 51 | 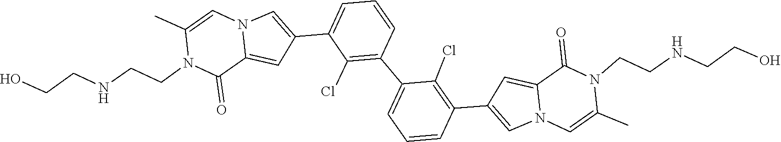 [SMILES: Cc1cn2cc(-c3cccc(-c4cccc(-c5cc6c(=O)n(CCNCCO)c(C)cn6c5)c4Cl)c3Cl)cc2c(=O)n1CCNCCO] |
| | 51 |
| 52 | 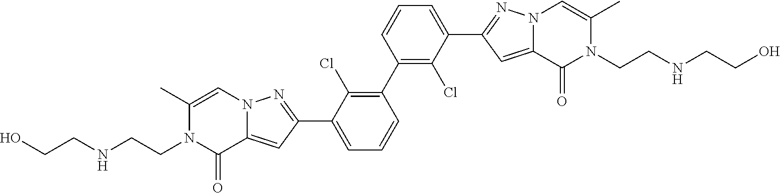 [SMILES: Cc1cn2nc(-c3cccc(-c4cccc(-c5cc6c(=O)n(CCNCCO)c(C)cn6n5)c4Cl)c3Cl)cc2c(=O)n1CCNCCO] |
| | 52 |
| 53 | 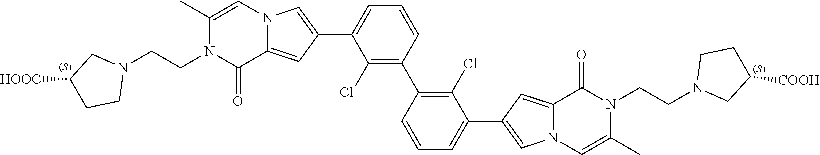 |
| | 53 |
| 54 | 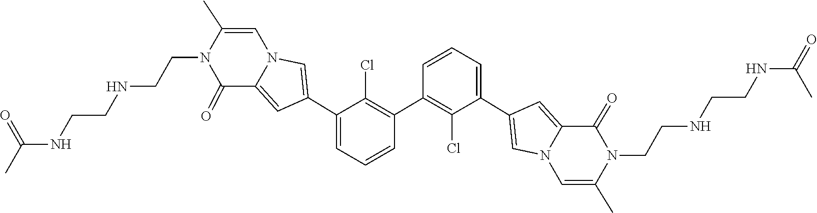 [SMILES: CC(=O)NCCNCCn1c(C)cn2cc(-c3cccc(-c4cccc(-c5cc6c(=O)n(CCNCCNC(C)=O)c(C)cn6c5)c4Cl)c3Cl)cc2c1=O] |
| | 54 |
| 55 | 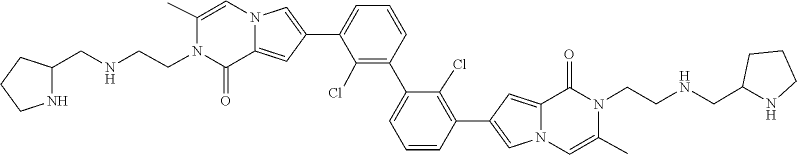 [SMILES: Cc1cn2cc(-c3cccc(-c4cccc(-c5cc6c(=O)n(CCNCC7CCCN7)c(C)cn6c5)c4Cl)c3Cl)cc2c(=O)n1CCNCC1CCCN1] |
| | 55 |
| 56 | 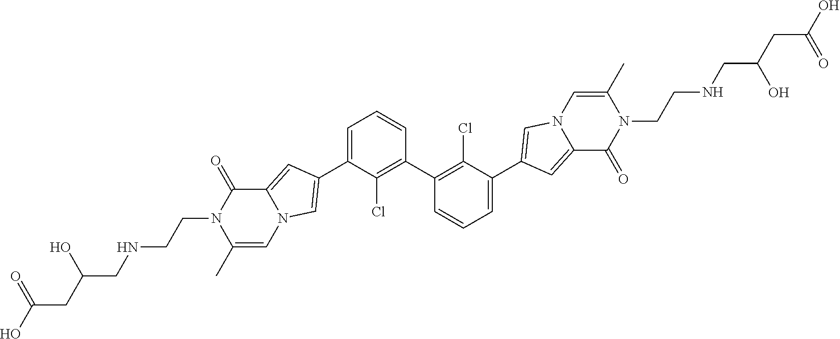 [SMILES: Cc1cn2cc(-c3cccc(-c4cccc(-c5cc6c(=O)n(CCNCC(O)CC(=O)O)c(C)cn6c5)c4Cl)c3Cl)cc2c(=O)n1CCNCC(O)CC(=O)O] |
| | 56 |
| 57 | 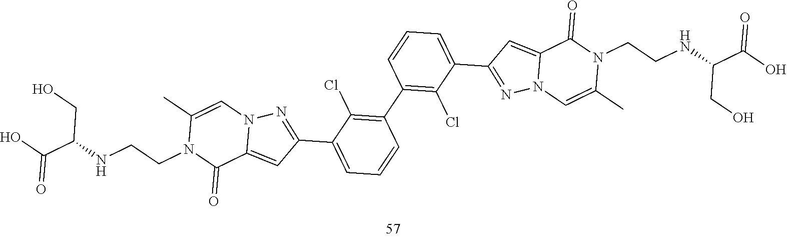 [SMILES: Cc1cn2nc(-c3cccc(-c4cccc(-c5cc6c(=O)n(CCN[C@@H](CO)C(=O)O)c(C)cn6n5)c4Cl)c3Cl)cc2c(=O)n1CCN[C@@H](CO)C(=O)O] |
| 58 | 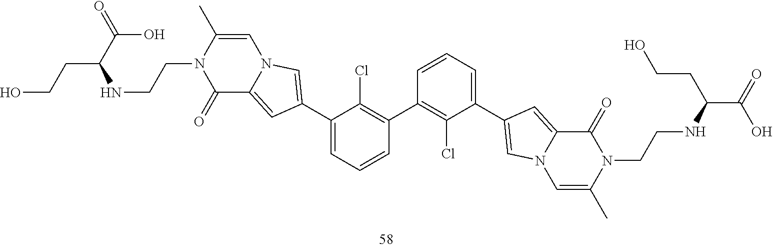 [SMILES: Cc1cn2cc(-c3cccc(-c4cccc(-c5cc6c(=O)n(CCN[C@@H](CCO)C(=O)O)c(C)cn6c5)c4Cl)c3Cl)cc2c(=O)n1CCN[C@@H](CCO)C(=O)O] |
| 59 | 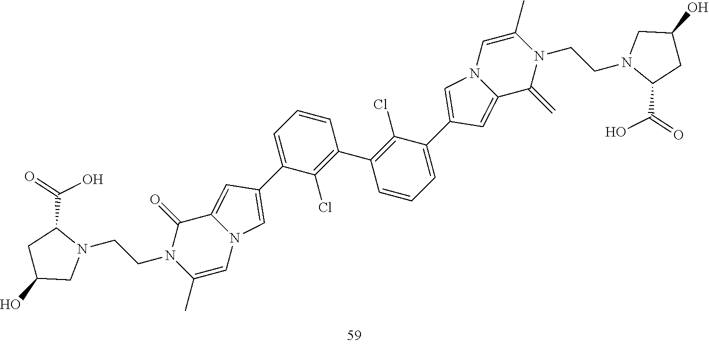 [SMILES: C=C1c2cc(-c3cccc(-c4cccc(-c5cc6c(=O)n(CCN7C[C@@H](O)C[C@@H]7C(=O)O)c(C)cn6c5)c4Cl)c3Cl)cn2C=C(C)N1CCN1C[C@@H](O)C[C@@H]1C(=O)O] |
| 60 | 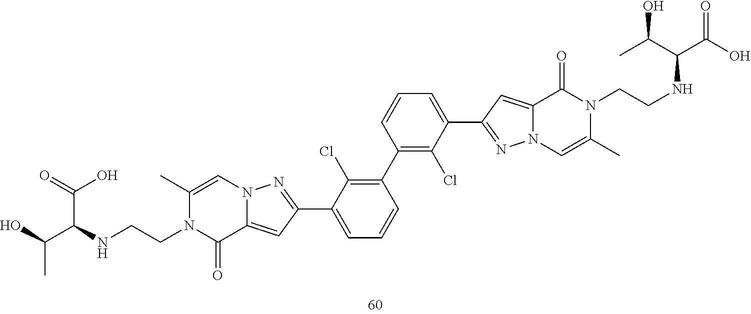 [SMILES: Cc1cn2nc(-c3cccc(-c4cccc(-c5cc6c(=O)n(CCN[C@H](C(=O)O)[C@@H](C)O)c(C)cn6n5)c4Cl)c3Cl)cc2c(=O)n1CCN[C@H](C(=O)O)[C@@H](C)O] |
| 61 | 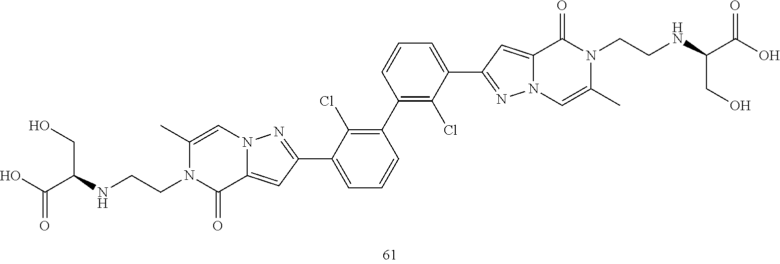 [SMILES: Cc1cn2nc(-c3cccc(-c4cccc(-c5cc6c(=O)n(CCN[C@H](CO)C(=O)O)c(C)cn6n5)c4Cl)c3Cl)cc2c(=O)n1CCN[C@H](CO)C(=O)O] |
| 62 | 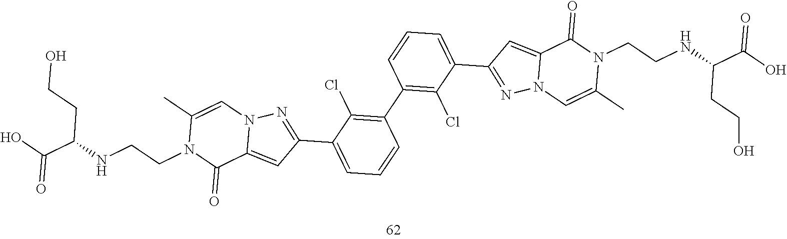 [SMILES: Cc1cn2nc(-c3cccc(-c4cccc(-c5cc6c(=O)n(CCN[C@@H](CCO)C(=O)O)c(C)cn6n5)c4Cl)c3Cl)cc2c(=O)n1CCN[C@@H](CCO)C(=O)O] |
| 63 | 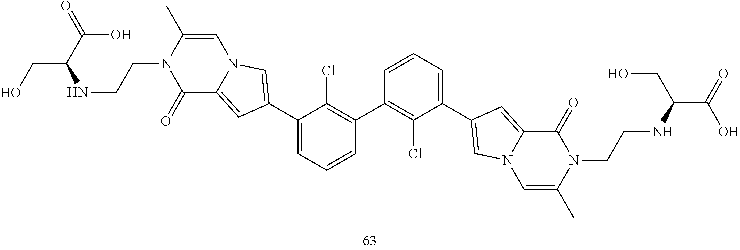 [SMILES: Cc1cn2cc(-c3cccc(-c4cccc(-c5cc6c(=O)n(CCN[C@@H](CO)C(=O)O)c(C)cn6c5)c4Cl)c3Cl)cc2c(=O)n1CCN[C@@H](CO)C(=O)O] |
| 64 | 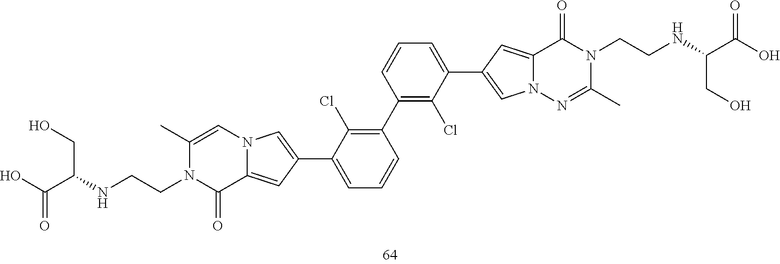 [SMILES: Cc1cn2cc(-c3cccc(-c4cccc(-c5cc6c(=O)n(CCN[C@@H](CO)C(=O)O)c(C)nn6c5)c4Cl)c3Cl)cc2c(=O)n1CCN[C@@H](CO)C(=O)O] |
| 65 | 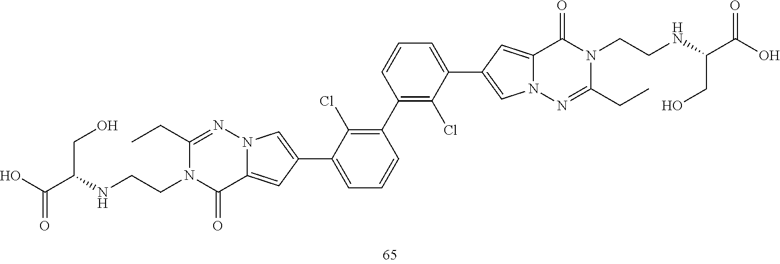 [SMILES: CCc1nn2cc(-c3cccc(-c4cccc(-c5cc6c(=O)n(CCN[C@@H](CO)C(=O)O)c(CC)nn6c5)c4Cl)c3Cl)cc2c(=O)n1CCN[C@@H](CO)C(=O)O] |
| 66 | 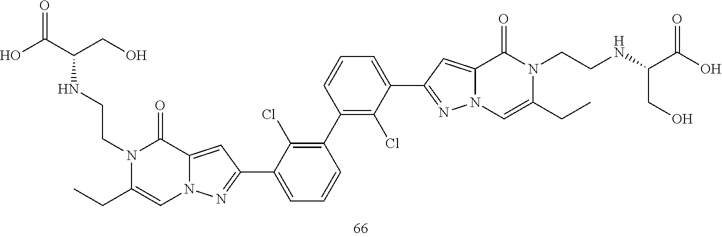 [SMILES: CCc1cn2nc(-c3cccc(-c4cccc(-c5cc6c(=O)n(CCN[C@@H](CO)C(=O)O)c(CC)cn6n5)c4Cl)c3Cl)cc2c(=O)n1CCN[C@@H](CO)C(=O)O] |
| 67 | 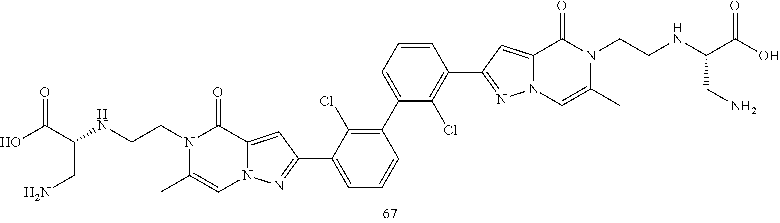 [SMILES: Cc1cn2nc(-c3cccc(-c4cccc(-c5cc6c(=O)n(CCN[C@H](CN)C(=O)O)c(C)cn6n5)c4Cl)c3Cl)cc2c(=O)n1CCN[C@@H](CN)C(=O)O] |
| 68 | 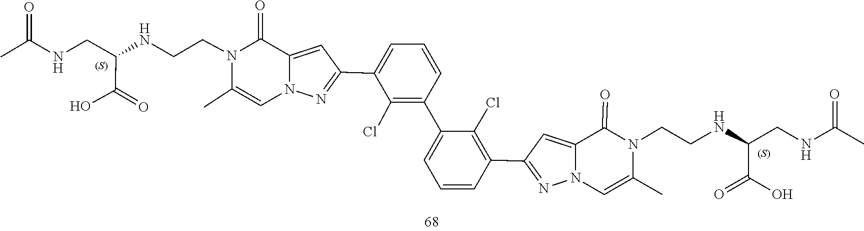 |
| 69 | 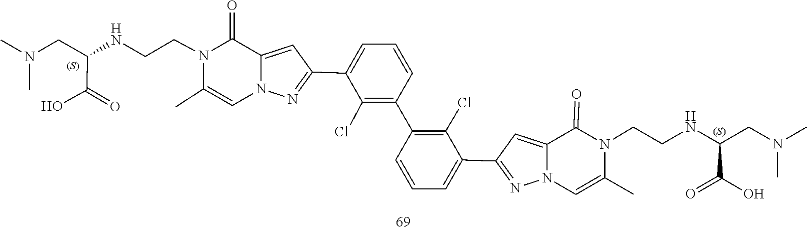 |
| 70 | 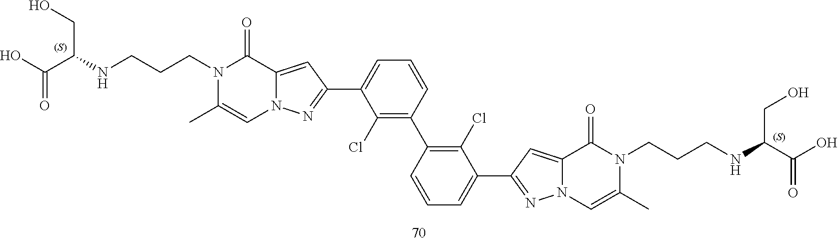 |
| 71 | 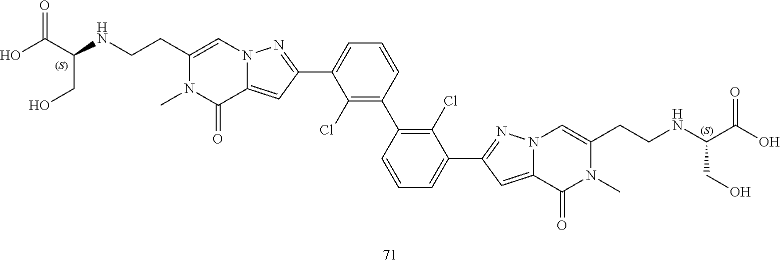 |
| 72 | 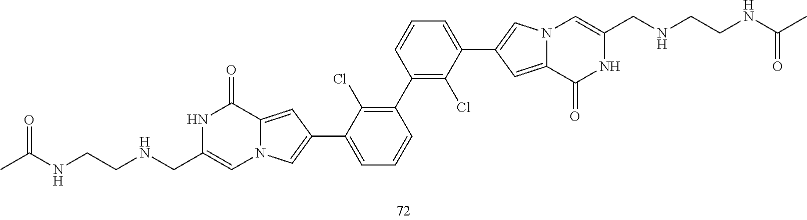 [SMILES: CC(=O)NCCNCc1cn2cc(-c3cccc(-c4cccc(-c5cc6c(=O)[nH]c(CNCCNC(C)=O)cn6c5)c4Cl)c3Cl)cc2c(=O)[nH]1] |
| 73 | 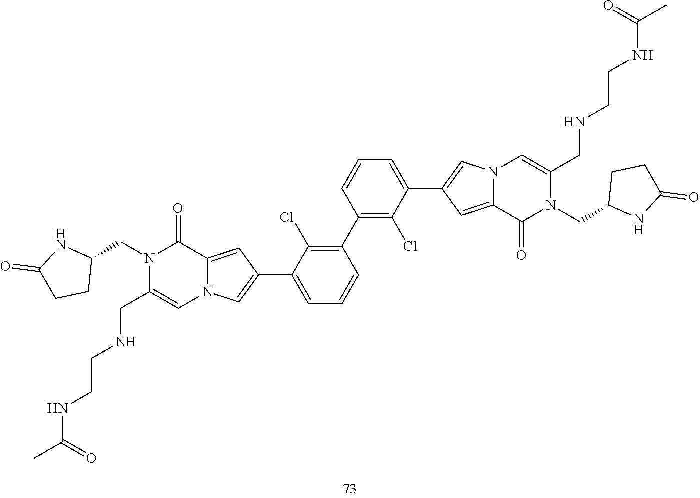 [SMILES: CC(=O)NCCNCc1cn2cc(-c3cccc(-c4cccc(-c5cc6c(=O)n(C[C@@H]7CCC(=O)N7)c(CNCCNC(C)=O)cn6c5)c4Cl)c3Cl)cc2c(=O)n1C[C@@H]1CCC(=O)N1] |
| 74 | 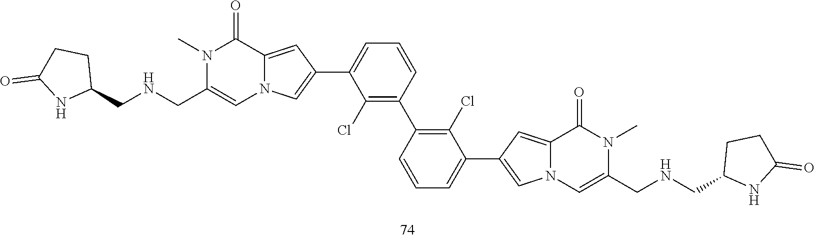 [SMILES: Cn1c(CNC[C@@H]2CCC(=O)N2)cn2cc(-c3cccc(-c4cccc(-c5cc6c(=O)n(C)c(CNC[C@@H]7CCC(=O)N7)cn6c5)c4Cl)c3Cl)cc2c1=O] |
| 75 | 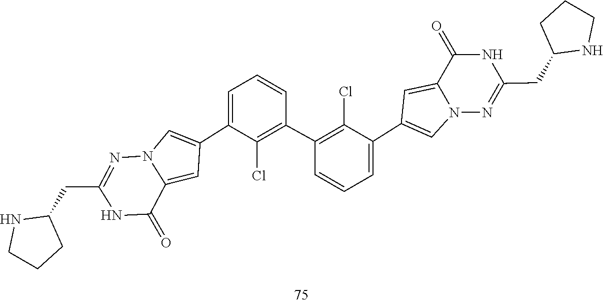 [SMILES: O=c1[nH]c(C[C@@H]2CCCN2)nn2cc(-c3cccc(-c4cccc(-c5cc6c(=O)[nH]c(C[C@@H]7CCCN7)nn6c5)c4Cl)c3Cl)cc12] |
| 76 | 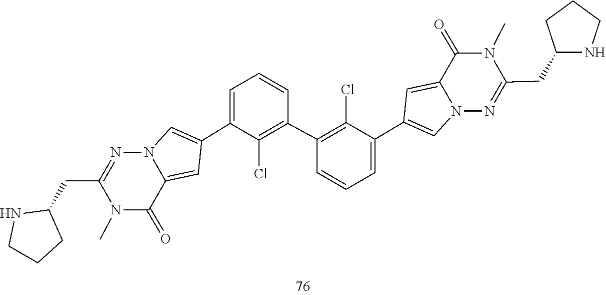 [SMILES: Cn1c(C[C@@H]2CCCN2)nn2cc(-c3cccc(-c4cccc(-c5cc6c(=O)n(C)c(C[C@@H]7CCCN7)nn6c5)c4Cl)c3Cl)cc2c1=O] |
| 77 | 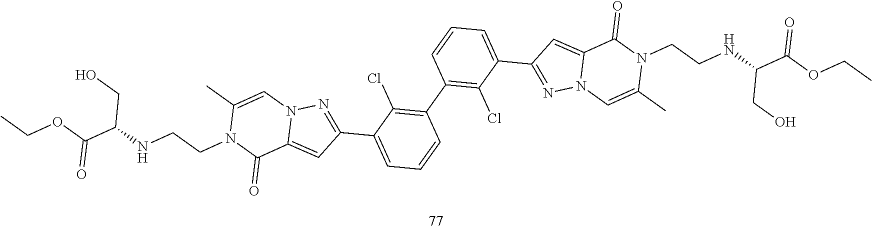 [SMILES: CCOC(=O)[C@H](CO)NCCn1c(C)cn2nc(-c3cccc(-c4cccc(-c5cc6c(=O)n(CCN[C@@H](CO)C(=O)OCC)c(C)cn6n5)c4Cl)c3Cl)cc2c1=O] |
| 78 | 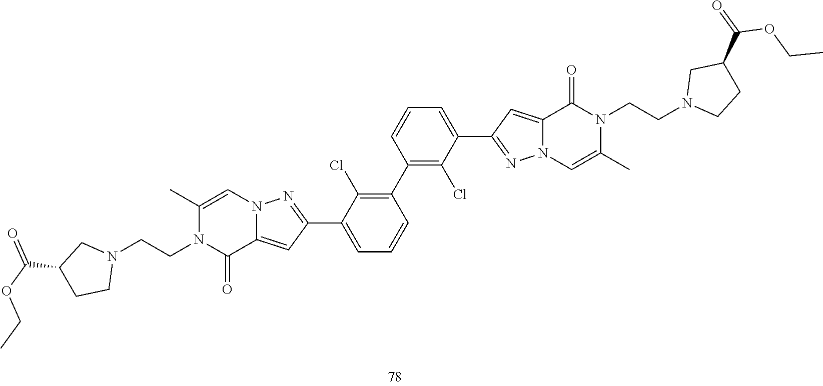 [SMILES: CCOC(=O)[C@H]1CCN(CCn2c(C)cn3nc(-c4cccc(-c5cccc(-c6cc7c(=O)n(CCN8CC[C@H](C(=O)OCC)C8)c(C)cn7n6)c5Cl)c4Cl)cc3c2=O)C1] |
| 79 | 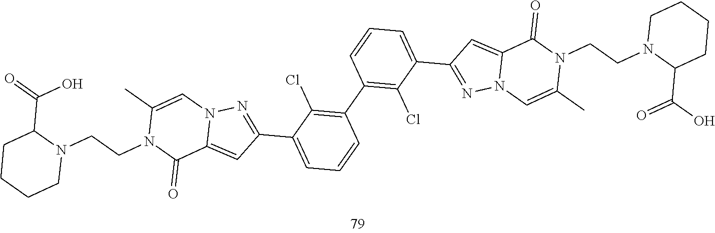 [SMILES: Cc1cn2nc(-c3cccc(-c4cccc(-c5cc6c(=O)n(CCN7CCCCC7C(=O)O)c(C)cn6n5)c4Cl)c3Cl)cc2c(=O)n1CCN1CCCCC1C(=O)O] |
| 80 | 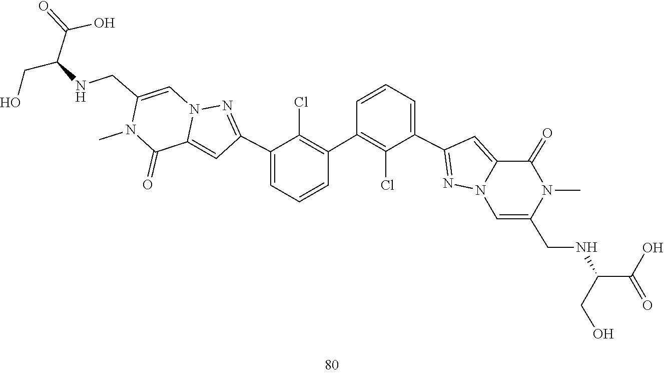 [SMILES: Cn1c(CN[C@@H](CO)C(=O)O)cn2nc(-c3cccc(-c4cccc(-c5cc6c(=O)n(C)c(CN[C@@H](CO)C(=O)O)cn6n5)c4Cl)c3Cl)cc2c1=O] |
| 81 | 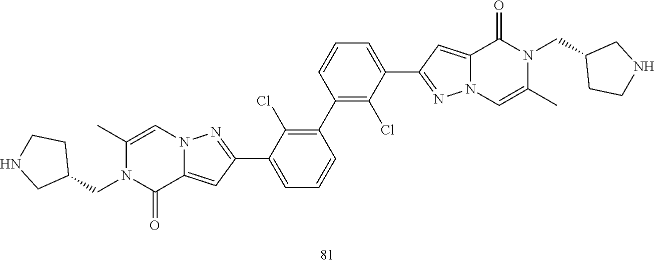 [SMILES: Cc1cn2nc(-c3cccc(-c4cccc(-c5cc6c(=O)n(C[C@H]7CCNC7)c(C)cn6n5)c4Cl)c3Cl)cc2c(=O)n1C[C@H]1CCNC1] |
| 82 | 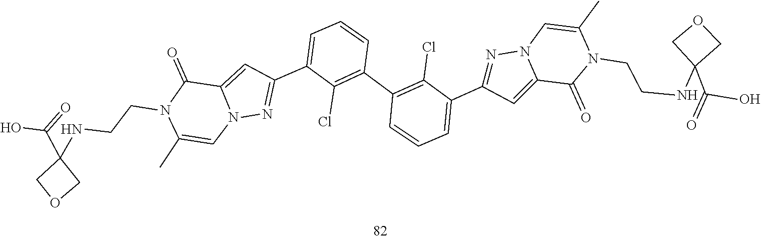 [SMILES: Cc1cn2nc(-c3cccc(-c4cccc(-c5cc6c(=O)n(CCNC7(C(=O)O)COC7)c(C)cn6n5)c4Cl)c3Cl)cc2c(=O)n1CCNC1(C(=O)O)COC1] |
| 83 | 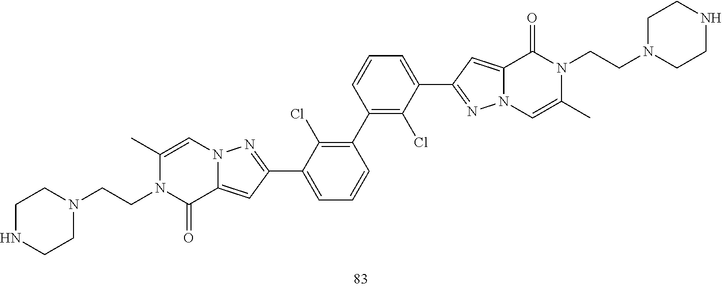 [SMILES: Cc1cn2nc(-c3cccc(-c4cccc(-c5cc6c(=O)n(CCN7CCNCC7)c(C)cn6n5)c4Cl)c3Cl)cc2c(=O)n1CCN1CCNCC1] |
| 84 | 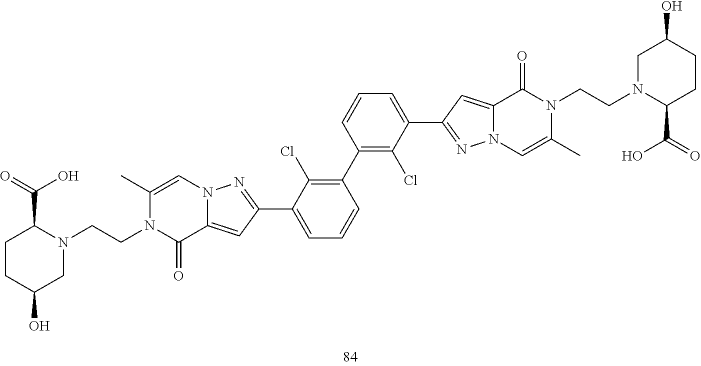 [SMILES: Cc1cn2nc(-c3cccc(-c4cccc(-c5cc6c(=O)n(CCN7C[C@@H](O)CC[C@H]7C(=O)O)c(C)cn6n5)c4Cl)c3Cl)cc2c(=O)n1CCN1C[C@@H](O)CC[C@H]1C(=O)O] |
| 85 | 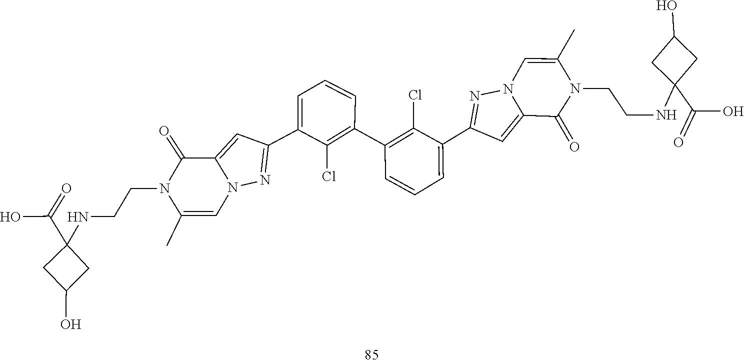 [SMILES: Cc1cn2nc(-c3cccc(-c4cccc(-c5cc6c(=O)n(CCNC7(C(=O)O)CC(O)C7)c(C)cn6n5)c4Cl)c3Cl)cc2c(=O)n1CCNC1(C(=O)O)CC(O)C1] |
| 86 | 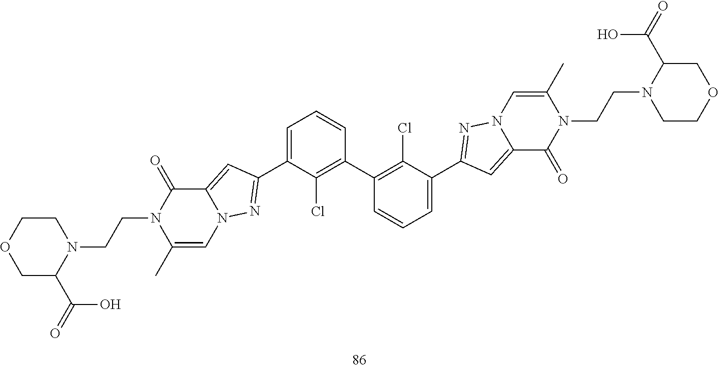 [SMILES: Cc1cn2nc(-c3cccc(-c4cccc(-c5cc6c(=O)n(CCN7CCOCC7C(=O)O)c(C)cn6n5)c4Cl)c3Cl)cc2c(=O)n1CCN1CCOCC1C(=O)O] |
| 87 | 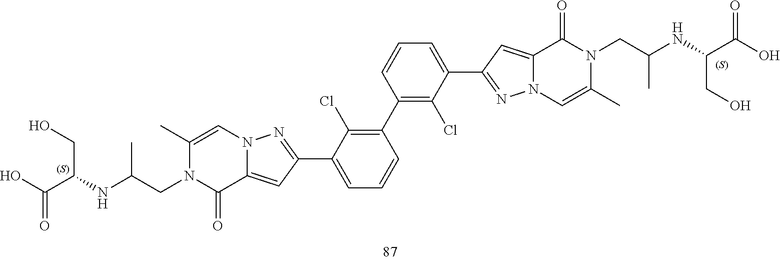 |
| 88 | 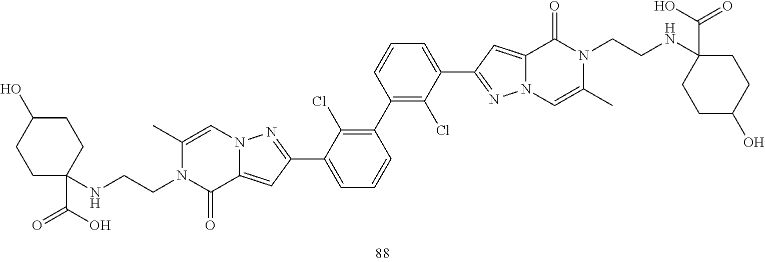 [SMILES: Cc1cn2nc(-c3cccc(-c4cccc(-c5cc6c(=O)n(CCNC7(C(=O)O)CCC(O)CC7)c(C)cn6n5)c4Cl)c3Cl)cc2c(=O)n1CCNC1(C(=O)O)CCC(O)CC1] |
| 89 | 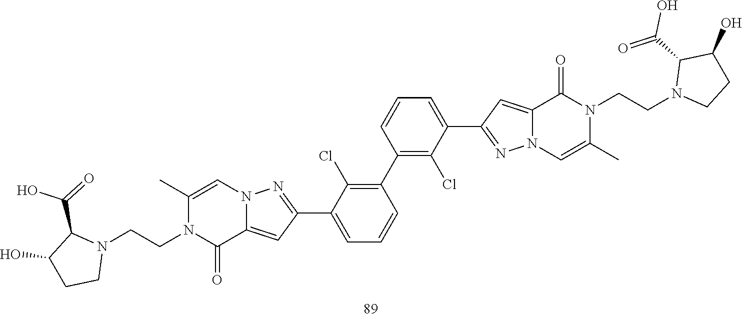 [SMILES: Cc1cn2nc(-c3cccc(-c4cccc(-c5cc6c(=O)n(CCN7CC[C@H](O)[C@H]7C(=O)O)c(C)cn6n5)c4Cl)c3Cl)cc2c(=O)n1CCN1CC[C@H](O)[C@H]1C(=O)O] |
| 90 | 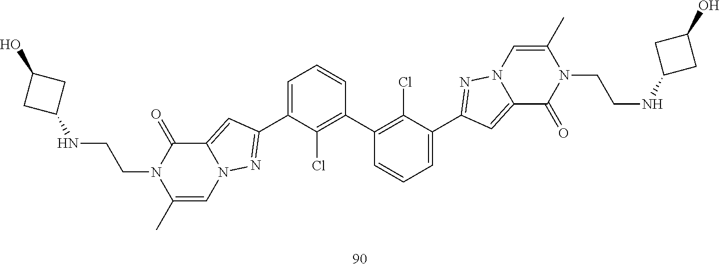 [SMILES: Cc1cn2nc(-c3cccc(-c4cccc(-c5cc6c(=O)n(CCN[C@H]7C[C@H](O)C7)c(C)cn6n5)c4Cl)c3Cl)cc2c(=O)n1CCN[C@H]1C[C@H](O)C1] |
| 91 | 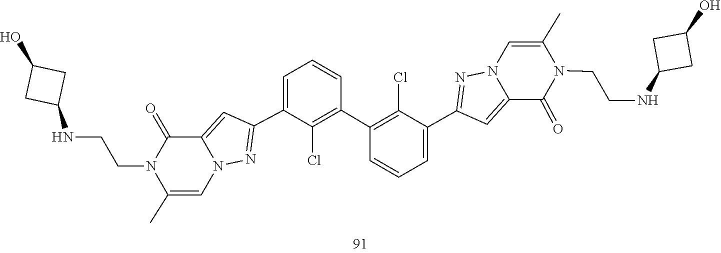 [SMILES: Cc1cn2nc(-c3cccc(-c4cccc(-c5cc6c(=O)n(CCN[C@H]7C[C@@H](O)C7)c(C)cn6n5)c4Cl)c3Cl)cc2c(=O)n1CCN[C@H]1C[C@@H](O)C1] |
| 92 | 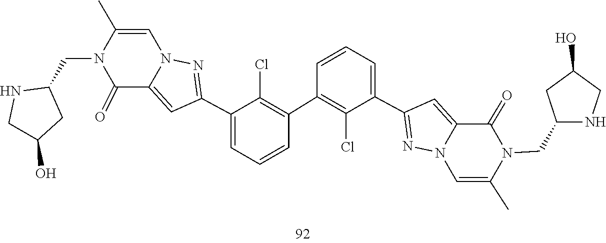 [SMILES: Cc1cn2nc(-c3cccc(-c4cccc(-c5cc6c(=O)n(C[C@@H]7C[C@@H](O)CN7)c(C)cn6n5)c4Cl)c3Cl)cc2c(=O)n1C[C@@H]1C[C@@H](O)CN1] |
| 93 | 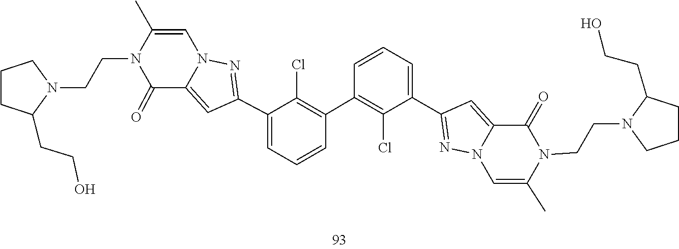 [SMILES: Cc1cn2nc(-c3cccc(-c4cccc(-c5cc6c(=O)n(CCN7CCCC7CCO)c(C)cn6n5)c4Cl)c3Cl)cc2c(=O)n1CCN1CCCC1CCO] |
| 94 | 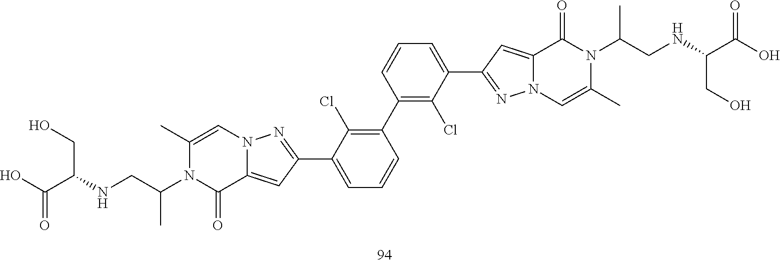 [SMILES: Cc1cn2nc(-c3cccc(-c4cccc(-c5cc6c(=O)n(C(C)CN[C@@H](CO)C(=O)O)c(C)cn6n5)c4Cl)c3Cl)cc2c(=O)n1C(C)CN[C@@H](CO)C(=O)O] |
| 95 | 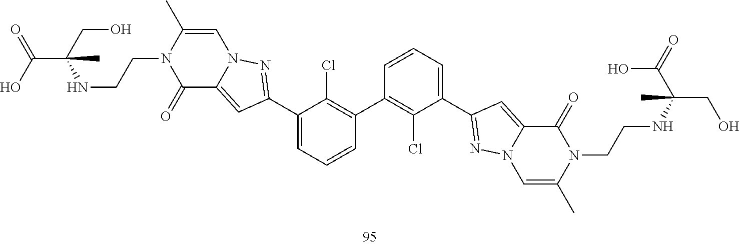 [SMILES: Cc1cn2nc(-c3cccc(-c4cccc(-c5cc6c(=O)n(CCN[C@@](C)(CO)C(=O)O)c(C)cn6n5)c4Cl)c3Cl)cc2c(=O)n1CCN[C@@](C)(CO)C(=O)O] |
| 96 | 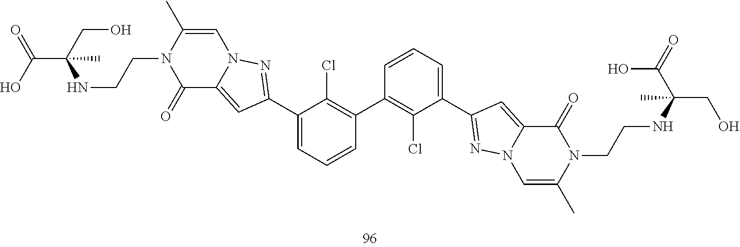 [SMILES: Cc1cn2nc(-c3cccc(-c4cccc(-c5cc6c(=O)n(CCN[C@](C)(CO)C(=O)O)c(C)cn6n5)c4Cl)c3Cl)cc2c(=O)n1CCN[C@](C)(CO)C(=O)O] |
| 97 | 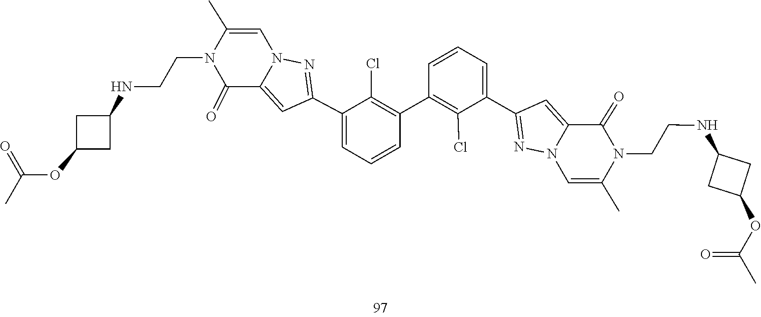 [SMILES: CC(=O)O[C@H]1C[C@@H](NCCn2c(C)cn3nc(-c4cccc(-c5cccc(-c6cc7c(=O)n(CCN[C@H]8C[C@@H](OC(C)=O)C8)c(C)cn7n6)c5Cl)c4Cl)cc3c2=O)C1] |
| 98 | 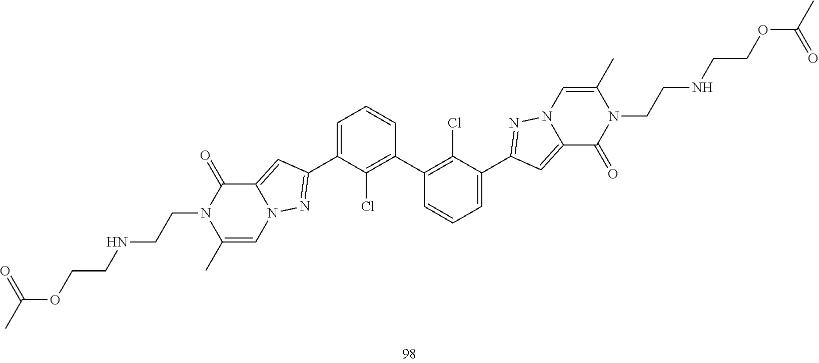 [SMILES: CC(=O)OCCNCCn1c(C)cn2nc(-c3cccc(-c4cccc(-c5cc6c(=O)n(CCNCCOC(C)=O)c(C)cn6n5)c4Cl)c3Cl)cc2c1=O] |
| 99 | 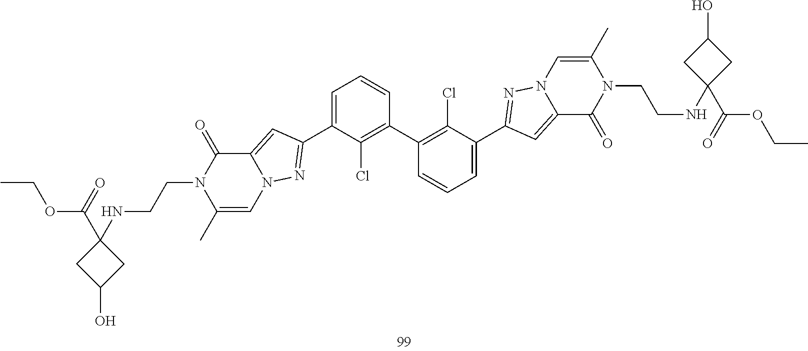 [SMILES: CCOC(=O)C1(NCCn2c(C)cn3nc(-c4cccc(-c5cccc(-c6cc7c(=O)n(CCNC8(C(=O)OCC)CC(O)C8)c(C)cn7n6)c5Cl)c4Cl)cc3c2=O)CC(O)C1] |
| 100 | 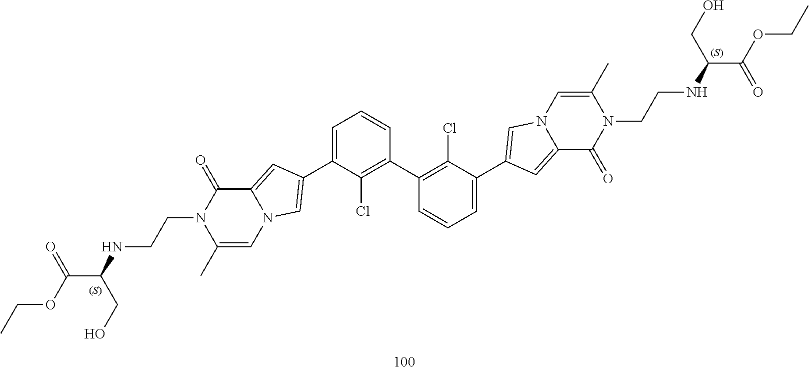 |
| 101 | 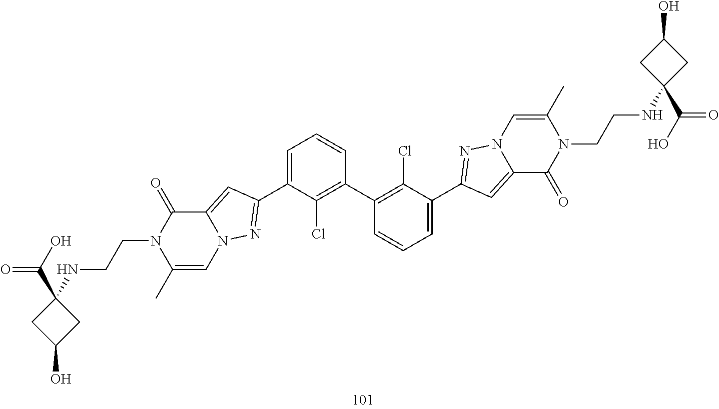 [SMILES: Cc1cn2nc(-c3cccc(-c4cccc(-c5cc6c(=O)n(CCN[C@]7(C(=O)O)C[C@@H](O)C7)c(C)cn6n5)c4Cl)c3Cl)cc2c(=O)n1CCN[C@]1(C(=O)O)C[C@@H](O)C1] |
| 102 | 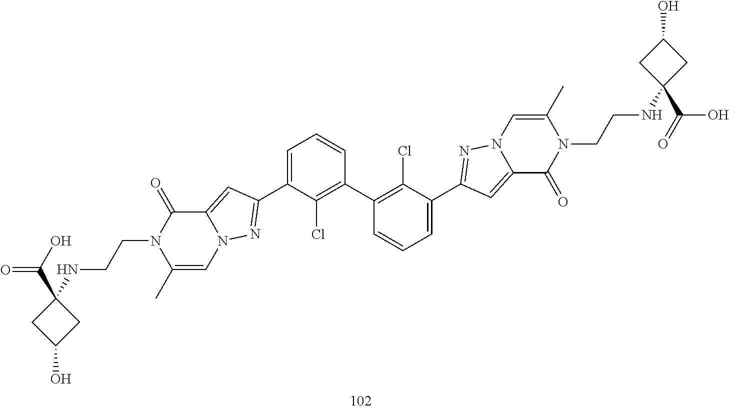 [SMILES: Cc1cn2nc(-c3cccc(-c4cccc(-c5cc6c(=O)n(CCN[C@]7(C(=O)O)C[C@H](O)C7)c(C)cn6n5)c4Cl)c3Cl)cc2c(=O)n1CCN[C@]1(C(=O)O)C[C@H](O)C1] |
| 103 | 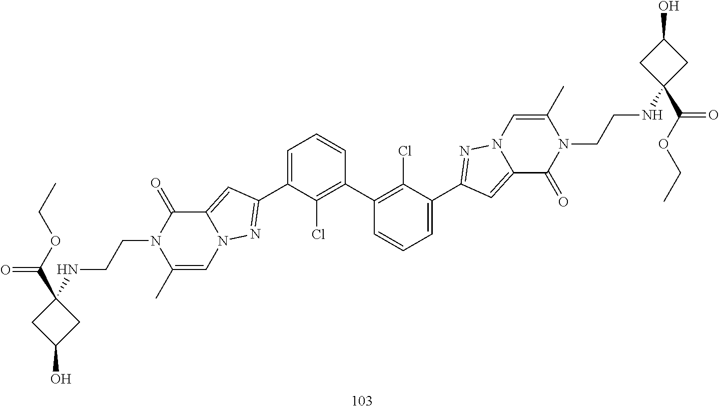 [SMILES: CCOC(=O)[C@]1(NCCn2c(C)cn3nc(-c4cccc(-c5cccc(-c6cc7c(=O)n(CCN[C@]8(C(=O)OCC)C[C@@H](O)C8)c(C)cn7n6)c5Cl)c4Cl)cc3c2=O)C[C@H](O)C1] |
| 104 | 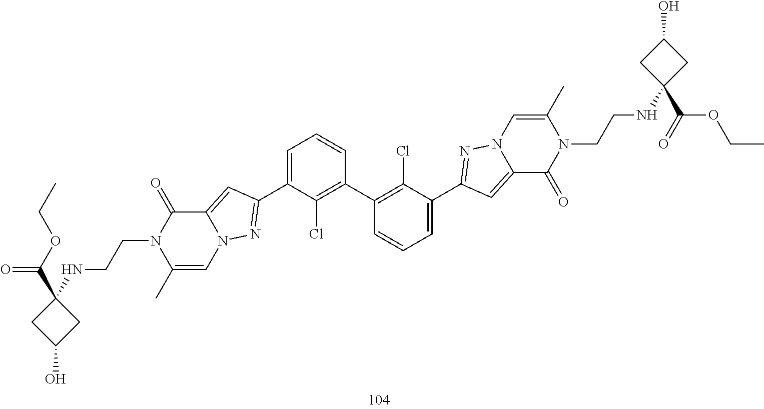 [SMILES: CCOC(=O)[C@]1(NCCn2c(C)cn3nc(-c4cccc(-c5cccc(-c6cc7c(=O)n(CCN[C@]8(C(=O)OCC)C[C@H](O)C8)c(C)cn7n6)c5Cl)c4Cl)cc3c2=O)C[C@@H](O)C1] |
| 105 | 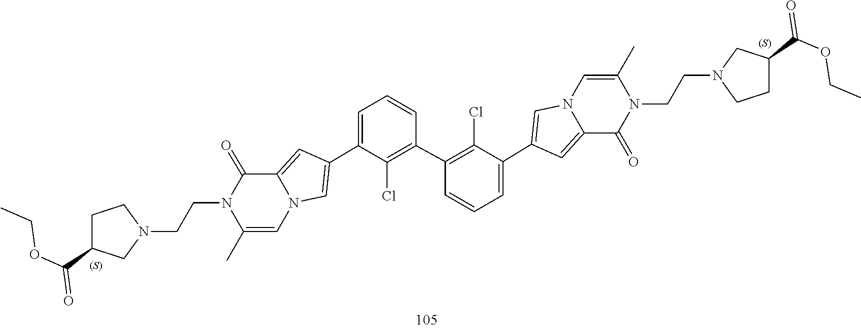 |
| 106 | 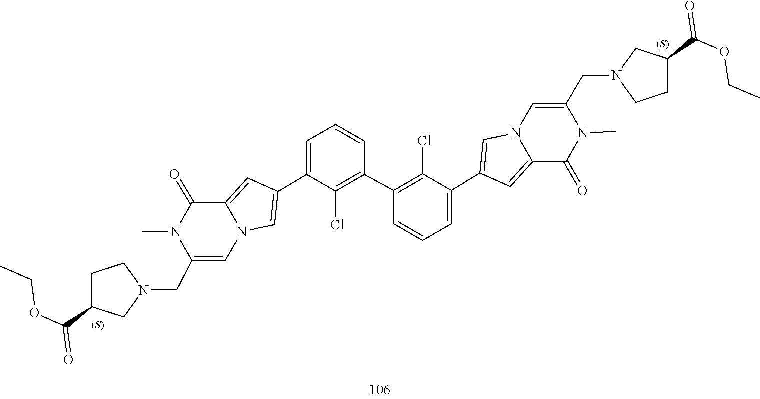 |

The present application further includes isotopically-substituted compounds of the disclosure. An “isotopically-substituted” compound is a compound of the application where one or more atoms are replaced or substituted by an atom having the same atomic number but a different atomic mass or mass number, e.g., a different atomic mass or mass number from the atomic mass or mass number typically found in nature (i.e., naturally occurring). It is to be understood that a “radio-labeled” compound is a compound that has incorporated at least one isotope that is radioactive (e.g., radionuclide).

### III. Uses of the Compound of Formula (I)

Another aspect of the present application relates the use of the compounds of formula (I). The compounds of formula (I) interfere with the interaction between PD-L1 and PD-1 and, thus, are useful in treating diseases and disorders associated with activity of PD-1 and the diseases and disorders associated with PD-L1. In some embodiments, the compounds of formula (I) promote the formation of PD-L1 dimers and therefore, inhibits the interaction between PD-L1 and PD-1.

In certain embodiments, the compounds of the present disclosure, or pharmaceutically acceptable salts or stereoisomers thereof, are useful for therapeutic administration to enhance, stimulate and/or increase immunity in cancer, chronic infection or sepsis, including enhancement of response to vaccination. In some embodiments, the present disclosure provides a method for inhibiting the PD-1/PD-L1 protein/protein interaction. The method includes administering to an individual or a patient a compound of any of the formulas as described herein, or of a compound as recited in any of the claims and described herein, or a pharmaceutically acceptable salt or a stereoisomer thereof. The compounds of the present disclosure can be used alone, in combination with other agents or therapies or as an adjuvant or neoadjuvant for the treatment of diseases or disorders, including cancer or infection diseases. For the uses described herein, any of the compounds of the disclosure, including any of the embodiments thereof, may be used.

The compounds of the present disclosure inhibit the PD-1/PD-L1 protein/protein interaction, resulting in a PD-1 pathway blockade. The blockade of PD-1 can enhance the immune response to cancerous cells and infectious diseases in mammals, including humans. In some embodiments, the present disclosure provides treatment of an individual or a patient in vivo using a compound of any of the formulas herein or a salt or stereoisomer thereof such that growth of cancerous tumors is inhibited. A compound of any of the formulas as described herein, or a compound as recited in any of the claims and described herein, or a salt or stereoisomer thereof, can be used to inhibit the growth of cancerous tumors. Alternatively, a compound of any of the formulas as described herein, or a compound as recited in any of the claims and described herein, or a salt or stereoisomer thereof, can be used in conjunction with other agents or standard cancer treatments, as described below. In one embodiment, the present disclosure provides a method for inhibiting growth of tumor cells in vitro. The method includes contacting the tumor cells in vitro with a compound of any of the formulas as described herein, or of a compound as recited in any of the claims and described herein, or of a salt or stereoisomer thereof. In another embodiment, the present disclosure provides a method for inhibiting growth of tumor cells in an individual or a patient. The method includes administering to the individual or patient in need thereof a therapeutically effective amount of a compound of any of the formulas as described herein, or of a compound as recited in any of the claims and described herein, or a salt or a stereoisomer thereof.

In some embodiments, provided herein is a method for treating cancer. The method includes administering to a patient in need thereof, a therapeutically effective amount of any of the formulas as described herein, a compound as recited in any of the claims and described herein, or a salt thereof. Examples of cancers include those whose growth may be inhibited using compounds of the disclosure and cancers typically responsive to immunotherapy.

In some embodiments, the present disclosure provides a method of enhancing, stimulating and/or increasing the immune response in a patient. The method includes administering to the patient in need thereof a therapeutically effective amount of any of the formulas as described herein, a compound or composition as recited in any of the claims and described herein, or a salt thereof.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers, especially metastatic cancers that express PD-L1.

In some embodiments, cancers treatable with compounds of the present disclosure include melanoma (e.g., metastatic malignant melanoma, cutaneous melanoma), renal cancer (e.g. clear cell carcinoma), prostate cancer (e.g. hormone refractory prostate adenocarcinoma), breast cancer (e.g., breast invasive carcinoma), colon cancer, lung cancer (e.g. non-small cell lung cancer and small cell lung cancer), squamous cell head and neck cancer (e.g., squamous cell carcinoma of the head and neck), urothelial cancer (e.g., bladder cancer, nonmuscle invasive bladder cancer (NMIBC)) and cancers with high microsatellite instability (MSIhlgh).

Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, biliary tract cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC) (e.g., squamous cell NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (carcinoma, squamous cell carcinoma, adenocarcinoma, leiomyosarcoma, lymphoma), stomach (carcinoma, lymphoma, leiomyosarcoma, adenocarcinoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer (e.g., colorectal adenocarcinoma).

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma). In some embodiments, the cancer is a urological cancer (e.g., papilliary kidney carcinoma, testicular germ cell cancer, chromophobe renal cell carcinoma, clear cell renal carcinoma, or prostate adenocarcinoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, serous adenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma (e.g., cutaneous squamous cell carcinoma), Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

PD-1 pathway blockade with compounds of the present disclosure can also be used for treating infections such as viral, bacteria, fungus and parasite infections. The present disclosure provides a method for treating infections such as viral infections. The method includes administering to a patient in need thereof, a therapeutically effective amount of any of the formulas as described herein, a compound as recited in any of the claims and described herein, a salt thereof. Examples of viruses causing infections treatable by methods of the present disclosure include, but are not limit to, human immunodeficiency virus, human papillomavirus, influenza, hepatitis A, B, C or D viruses, adenovirus, poxvirus, herpes simplex viruses, human cytomegalovirus, severe acute respiratory syndrome virus, ebola virus, and measles virus. In some embodiments, viruses causing infections treatable by methods of the present disclosure include, but are not limit to, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus, tuberculosis and arboviral encephalitis virus.

The present disclosure provides a method for treating bacterial infections. The method includes administering to a patient in need thereof, a therapeutically effective amount of a compound of any of the formulas as described herein, a compound as recited in any of the claims and described herein, or a salt thereof.

Non-limiting examples of pathogenic bacteria causing infections treatable by methods of the disclosure include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

The present disclosure provides a method for treating fungus infections. The method includes administering to a patient in need thereof, a therapeutically effective amount of a compound of any of the formulas as described herein, a compound as recited in any of the claims and described herein, or a salt thereof. Non-limiting examples of pathogenic fungi causing infections treatable by methods of the disclosure include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Flistoplasma capsulatum.

The present disclosure provides a method for treating parasite infections. The method includes administering to a patient in need thereof, a therapeutically effective amount of a compound of any of the formulas as described herein, a compound as recited in any of the claims and described herein, or a salt thereof. Non-limiting examples of pathogenic parasites causing infections treatable by methods of the disclosure include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

The present disclosure provides a method for treating neurodegenerative diseases or disorders. The method includes administering to a patient in need thereof, a therapeutically effective amount of a compound of any of the formulas as described herein, a compound as recited in any of the claims and described herein, or a salt thereof. Non-limiting examples of neurodegenerative diseases or disorders include Alzheimer's disease, Parkinson's disease, Huntington's disease, prion disease, Motor neuron diseases, Spinocerebellar ataxia and Spinal muscular atrophy.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the compounds of the application are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

Cancer cell growth and survival can be impacted by dysfunction in multiple biological pathways. Thus, it may be useful to combine inhibitors of different mechanisms, such as enzyme inhibitors, signal transduction inhibitors, inhibitors of chromatin dynamics or modulators of immune responses, to treat such conditions. Targeting more than one signaling pathway (or more than one biological molecule involved in a given signaling pathway) may reduce the likelihood of drug-resistance arising in a cell population, or reduce the toxicity of treatment.

The compounds of the present disclosure can be used in combination with one or more other therapies for the treatment of diseases, such as cancer or infections. Examples of diseases and indications treatable with combination therapies include those as described herein.

Examples of cancers include solid tumors and non-solid tumors, such as liquid tumors, blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK, TGF-PR, PKA, PKG, PKC, CaM-kinase, phosphorylase kinase, MEKK, ERK, MAPK, mTOR, EGFR, HER2, HER3, HER4, INS—R, IDH2, IGF-1R, IR—R, PDGFotR, PDGi′PR, PI3K (alpha, beta, gamma, delta, and multiple or selective), CSF1R, KIT, FLK-1I, KDR/FLK-1, FLK-4, flt-1, FGFR1, FGFR2, FGFR3, FGFR4, c-Met, PARP, Ron, Sea, I RKA, TRKB, TRKC, TAM kinases (Axl, Mer, Tyro3), FLT3, VEGFR/FH2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCY54828), INCB62079), a JAK inhibitor (JAK1 and/or JAK2, e.g., ruxolitinib, baricitinib or itacitinib (INCB39110)), an IDO inhibitor (e.g., epacadostat, NLG919, or BMS-986205, MK7162), an LSD1 inhibitor (e.g., INCB59872 and INCB60003), a TDO inhibitor, a PI3K-delta inhibitor (e.g., Parsaclisib (INCB50465) and INCB50797), a PI3K-gamma inhibitor such as PI3K-gamma selective inhibitor, a Pirn inhibitor (e.g., INCB53914), an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, axitinib, vandetanib, ramucirumab, lenvatinib, ziv-aflibercept), a PARP inhibitor (e.g. olaparib, rucaparib, veliparib, talazoparib, or niraparib), a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), an arginase inhibitor (INCB001158), a PARP inhibitor (such as rucaparib or olaparib), sitravatinib, a B-Raf inhibitor-MEK inhibitor combination (such as encorafenib plus binimetinib, dabrafenib plus trametinib, or cobimetinib plus vemurafenib), and an adenosine receptor antagonist or combinations thereof.

In some embodiments, the compounds of the present disclosure can be combined with a TLR7 agonist (e.g., imiquimod).

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, STING agonists, RIG-I agonists, oncolytic virotherapy and immunomodulating small molecules, including thalidomide or JAK1/2 inhibitor, PI3K6 inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paelitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

Other anti-cancer agent(s) include antibody therapeutics such as trastuzumab (Herceptin), antibodies to costimulatory molecules such as CTLA-4 (e.g., ipilimumab), 4-1BB (e.g. urelumab, utomilumab), antibodies to PD-1 and PD-L1, or antibodies to cytokines (IL-10, TGF-b, etc.). Examples of antibodies to PD-1 and/or PD-L1 that can be combined with compounds of the present disclosure for the treatment of cancer or infections such as viral, bacteria, fungus and parasite infections include, but are not limited to nivolumab, pembrolizumab, atezolizumab, durvalumab, avelumab and SHR-1210.

Compounds of the present disclosure can be used in combination with one or more immune checkpoint inhibitors for the treatment of diseases, such as cancer or infections.

Exemplary immune checkpoint inhibitors include inhibitors against immune checkpoint molecules such as CBL-B, CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD 160 inhibitors, 2B4 inhibitors and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab or pembrolizumab. In some embodiments, the anti-PD1 antibody is pembrolizumab.

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab.

In some embodiments, the inhibitor of an immune checkpoint molecule is an inhibitor of LAG3, e.g., an anti-LAG3 antibody. In some embodiments, the anti-LAG3 antibody is BMS-986016, LAG525 or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an inhibitor of OX40, e.g., an anti-OX40 antibody or OX40L fusion protein. In some embodiments, the anti-0X40 antibody is MEDI0562, MOXR-0916, PF-04518600, GSK3174998, or BMS-986178. In some embodiments, the OX40L fusion protein is MEDI6383.

The compounds of the present disclosure can further be used in combination with one or more anti-inflammatory agents, steroids, immunosuppressants or therapeutic antibodies.

The compounds of any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Vims (KHSV). In some embodiments, the compounds of the present disclosure can be used in combination with tumor specific antigen such as heat shock proteins isolated from tumor tissue itself. In some embodiments, the compounds of any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

The compounds of the present disclosure can be used in combination with bone marrow transplant for the treatment of a variety of tumors of hematopoietic origin.

The compounds of any of the formulas as described herein, a compound as recited in any of the claims and described herein, or salts thereof can be used in combination with vaccines, to stimulate the immune response to pathogens, toxins, and self antigens.

When more than one pharmaceutical agent is administered to a patient, they can be administered simultaneously, separately, sequentially, or in combination (e.g., for more than two agents).

### Formulation, Dosage Forms and Route of Administration

When employed as pharmaceuticals, the compounds of the present disclosure can be administered in the form of pharmaceutical compositions. Thus the present disclosure provides a composition comprising a compound of any of the formulas as described herein, a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary {e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration.

Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This application also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the application, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the application may be milled using known milling procedures such as wet milling to obtain a particle size appropriate for tablet formation and for other formulation types. Finely divided (nanoparticulate) preparations of the compounds of the application can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the application can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g, Poly ox WSR 1105™.

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g, at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present application can vary according to, e.g., the particular use for which the treatment is made, the manner of administration of the compound, the health and condition of the patient, and the judgment of the prescribing physician. The proportion or concentration of a compound of the application in a pharmaceutical composition can vary depending upon a number of factors including dosage, chemical characteristics (e.g., hydrophobicity), and the route of administration. For example, the compounds of the application can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present application. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present application.

The tablets or pills of the present application can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g; glycerinemonostearate, PEG-glycerinemonostearate and cetylstearyl alcohol. Gels can be formulated using isopropyl alcohol and water, suitably in combination with other components such as, e.g., glycerol, hydroxy ethyl cellulose, and the like. In some embodiments, topical formulations contain at least about 0.1, at least about 0.25, at least about 0.5, at least about 1, at least about 2 or at least about 5 wt % of the compound of the application. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present application can vary according to, e.g., the particular use for which the treatment is made, the manner of administration of the compound, the health and condition of the patient, and the judgment of the prescribing physician. The proportion or concentration of a compound of the application in a pharmaceutical composition can vary depending upon a number of factors including dosage, chemical characteristics (e.g., hydrophobicity), and the route of administration. For example, the compounds of the application can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### IV. Methods of Synthesis

Compounds of the application, including salts thereof, can be prepared using known organic synthesis techniques and can be synthesized according to any of numerous possible synthetic routes.

The reactions for preparing compounds of the application can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the application can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith el al., March's Advanced Organic Chemistry: Reactions, Mechanisms, and Structure, 6th Ed. (Wiley, 2007); Peturssion et al., “Protecting Groups in Carbohydrate Chemistry,” J. Chem. Educ., 1997, 77(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., 'H or 13C), infrared spectroscopy, spectrophotometry (e.g., UV-visible), mass spectrometry or by chromatographic methods such as high performance liquid chromatography (HPLC) or thin layer chromatography (TLC).

The present application is further illustrated by the following examples that should not be construed as limiting. The contents of all references, patents, and published patent applications cited throughout this application, as well as the Figures and Tables, are incorporated herein by reference.

### EXAMPLES

### Chemical Synthesis Examples

### Example 1. Preparation of 6,6′-(2,2′-dichloro-[1,1′-biphenyl]-3,3′-diyl)bis(3-((5-oxopyrrolidin-2-yl)meth yl)pyrrolo[2,1-f][1,2,4]triazin-4(3H)-one) (Compound 1)

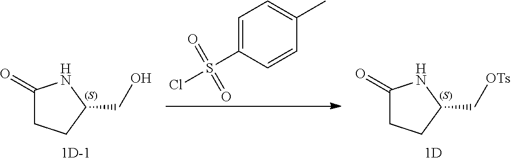

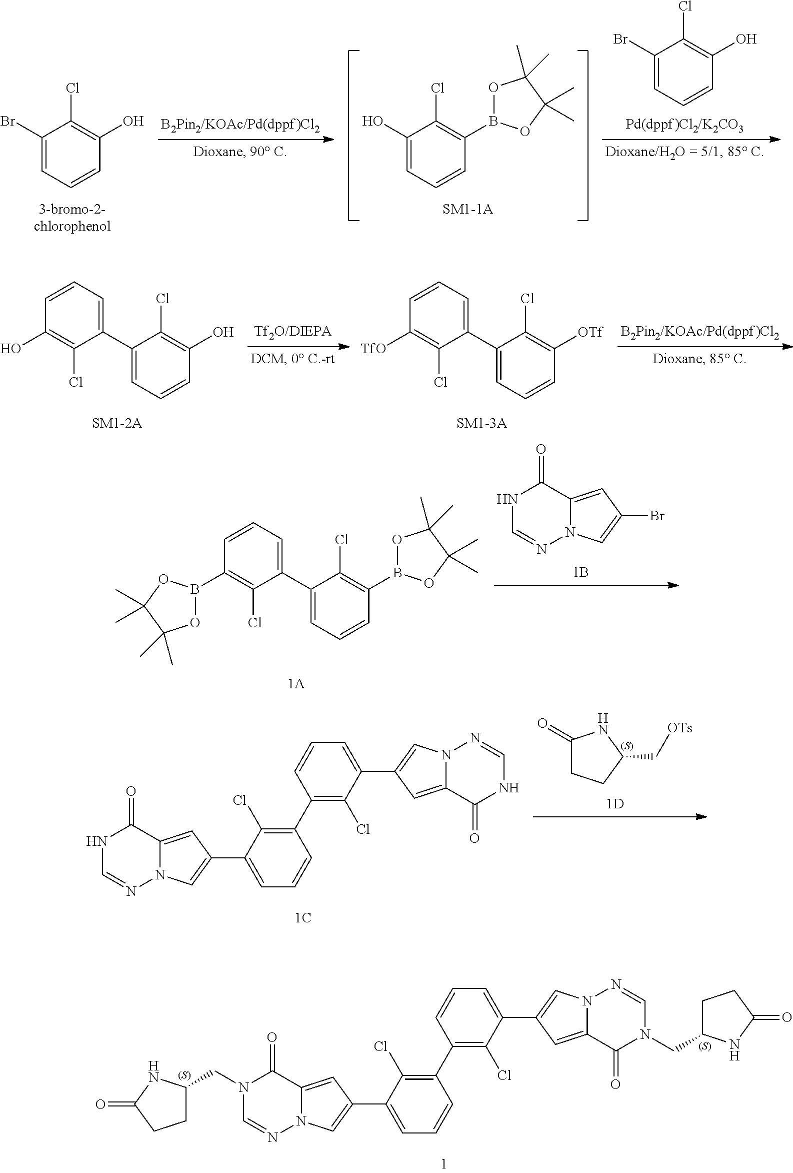

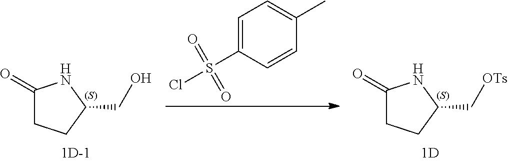

Compound 1D-1 (1.00 g, 8.7 mmol, 1.0 eq.), trimethylamine (1.76 g, 17.37 mmol, 2.0 eq.) were dissolved in DCM (20 mL). At 0° C., 4-methylbenzenesulfonyl chloride (1.99 g, 10.42 mmol, 1.20 eq) was added, and stirred for 1 h. The mixture solution was concentrated and purified by column chromatography to give Compound 1 D (2.1 g, yield: 89.8%).

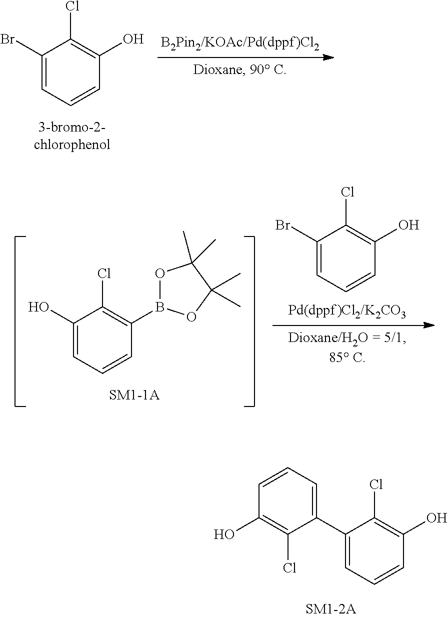

To a 500 mL round-bottom flask were added 3-bromo-2-chlorophenol (12.4 g, 0.060 mol, 1.0 eq), B₂Pin₂ (16.4 g, 0.065 mol, 1.08 eq), KOAc (20.5 g, 0.210 mol, 3.5 eq), Pd(dppf)Cl₂·DCM (4.1 g, 5.1 mmol, 0.085 eq), followed by dioxane (300 mL), the final mixture was charged with N₂ and stirred at 95° C. for 3 h. Then the reaction mixture was cooled down to r.t., filtered, the filter cake was washed with dioxane (100 mL), the filtrate was used directly in the next step.

To the previous filtrate were added 3-bromo-2-chlorophenol (12.0 g, 0.059 mol, 0.99 eq), K₂CO₃ (24.8 g, 0.180 mol, 3.0 eq) and Pd(dppf)Cl₂-DCM (2.1 g, 2.40 mmol, 0.042 eq), followed by H₂O (80 mL), the final mixture was charged with N₂ and stirred at 85° C. for 3.5 h. Then the reaction mixture was cooled down to r.t. and filtered, the filter cake was washed with EtOAc (300 mL). Brine (300 mL) was added to the filtrate and layers were separated. The aqueous phase was extracted with EtOAc (100 mL×2). The combined organic phase was discolored with activated carbon at r.t. overnight. The mixture was filtered through a pad of celite, the filter cake was washed with EtOAc, the combined organic phase was concentrated under vacuum. The residue was purified by recrystallization from DCM/PE=1.5/1 to afford desired product SM1-2A (10.1 g, yield: 66%).

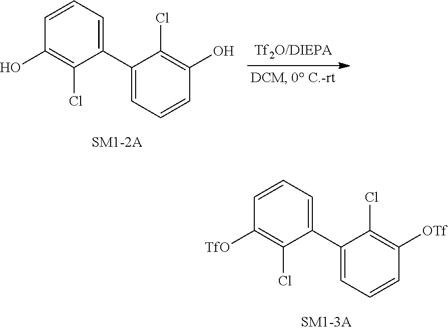 [SMILES: Oc1cccc(-c2cccc(O)c2Cl)c1Cl ; O=S(=O)(Oc1cccc(-c2cccc(OS(=O)(=O)C(F)(F)F)c2Cl)c1Cl)C(F)(F)F]

To a stirred mixture of SM1-2A (10.1 g, 0.039 mol, 1.0 eq) in DCM (200 mL) was added DIPEA (19.4 g, 0.151 mol, 3.8 eq) at 0° C., followed by Tf₂O (26.8 g, 0.095 mol, 2.4 eq) at this temperature, then stirred at r.t. for additional 2 h. Water (100 mL) was added, and layers were separated. The aqueous phase was extracted with DCM (100 mL). The combined organic phase was washed with brine (200 mL), dried over anhydrous sodium sulfate, filtered, the filtrate was concentrated under vacuum. The residue was purified by recrystallized with EtOH/H₂O=1/1 to afford desired product SM1-3A (18.3 g, yield 89%).

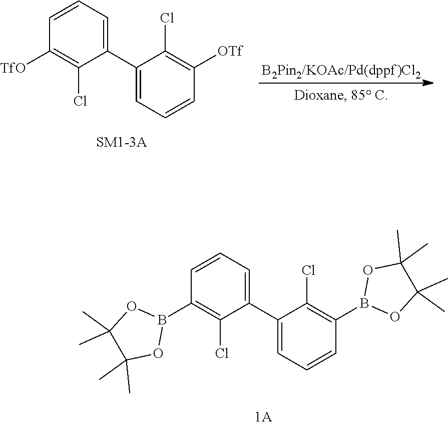

To a stirred solution of SM1-3A (14.2 g, 0.027 mol, 1.0 eq) in dioxane (80 mL) were added B₂Pin₂ (27.8 g, 0.109 mol, 4.0 eq), KOAc (16 g, 0.164 mol, 6.0 eq) and Pd(dppf)Cl₂-DCM (3.3 g, 4.1 mmol, 0.15 eq.) at r.t., then charged with N₂ and stirred at 85° C. for 2 h. After cooled down to r.t., EtOAc (150 mL) and water (150 mL) were added to the mixture, separated, the aqueous phase was extracted with EtOAc (100 mL). The combined organic phase was washed with brine (200 mL), dried over anhydrous sodium sulfate, filtered, the filtrate was concentrated under vacuum. The residue was dissolved with EtOAc (50 mL), then PE (300 mL) was added slowly to the solution to form a black suspension. After stirring for 30 min, it was filtered, the filter cake was washed with 140 mL (PE/EtOAc=6/1). The filtrate was concentrated under vacuum, and the residue was recrystallized with EtOH (150 mL) to afford desired product 1A (9.5 g, yield: 75%) as an off-white solid.

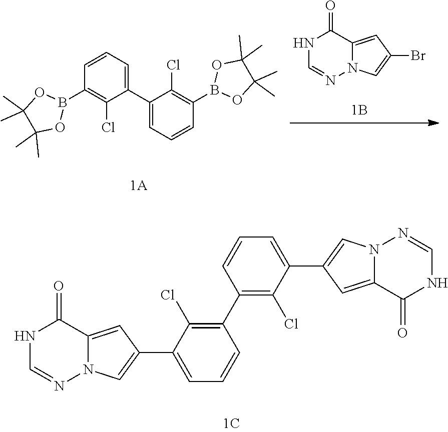

Under N₂ atmosphere, Compound 1A (1.0 g, 2.1 mmol, 1.0 eq.), Compound 1B (1.138 g, 5.32 mmol, 2.5 eq.), K₂CO₃ (1.45 g, 10.5 mmol, 5.0 eq.) and Pd(dppf)Cl₂-DCM (0.52 g, 0.63 mmol, 0.3 eq.) are dissolved in DMF (15 mL) and H₂O (3 mL). The reaction mixture was stirred at 100° C. for 3 h. After cooling, 30 mL water and 30 mL EtOAc were added. The organic phase was washed with water and concentrated to afford residue, which was washed by EtOAc to give compound 1C (0.80 g, yield: 77.8%). LCMS (ESI): calculated for C₂₄H₁₄Cl₂N₆O₂; [M+H]+: 489.1, found: 489.1. ¹H NMR (500 MHz, DMSO-d6) δ 8.11 (s, 2H), 7.97 (d, J=1.9 Hz, 2H), 7.71 (dd, J=7.9, 1.7 Hz, 2H), 7.48 (t, J=7.6 Hz, 2H), 7.32 (dd, J=7.6, 1.7 Hz, 2H), 7.25 (d, J=1.9 Hz, 2H), 6.31 (s, 2H).

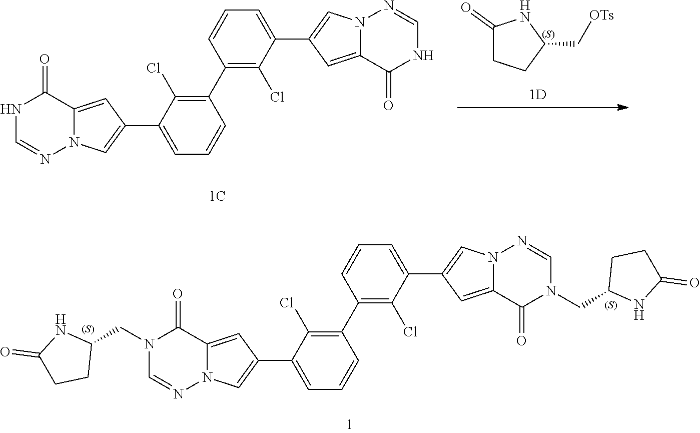

Compound 1C (100 mg, 0.20 mmol, 1.0 eq.), Compound 1D (121 mg, 0.45 mol, 2.2 eq.), Cs₂CO₃ (333 mg, 1.02 mmol, 5.0 eq.) were dissolved in DMF (2.0 mL). The reaction was stirred at 60° C. for 1 h. After cooling, 10 mL water was added, extracted with EtOAc, and concentrated. It was purified by prep-HPLC to give compound 1 (80 mg, yield: 57.3%). LCMS (ESI): calculated for C₃₄H₂₈Cl₂N₈O₄; [M+H]+: 683.2, found: 683.2. ¹H NMR (500 MHz, DMSO-d₆) δ 8.11 (s, 2H), 8.01 (d, J=1.2 Hz, 2H), 7.87 (s, 2H), 7.70 (d, J=6.4 Hz, 2H), 7.49 (t, J=6.4 Hz, 2H), 7.32 (d, J=6 Hz, 2H), 7.26 (d, J=1.6 Hz, 2H), 3.88-3.94 (m, 6H), 2.21-2.27 (m, 2H), 2.05-2.18 (m, 4H), 1.72-1.77 (m, 2H).

### Example 2. Preparation of 6,6′-(2,2′-dichloro-[1,1′-biphenyl]-3,3′-diyl)bis(3-(((S)-5-oxopyrrolidin-2-yl)methyl)thieno[2,3-d]pyrimidin-4(3H)-one) (Compound 2)

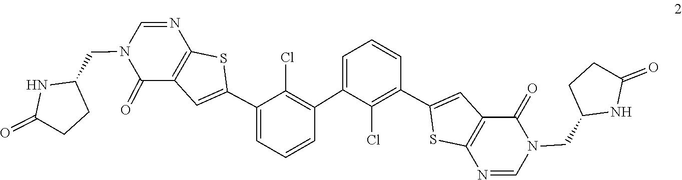 [SMILES: O=C1CC[C@@H](Cn2cnc3sc(-c4cccc(-c5cccc(-c6cc7c(=O)n(C[C@@H]8CCC(=O)N8)cnc7s6)c5Cl)c4Cl)cc3c2=O)N1]

Following the procedure of Compound 1, the title Compound 2 was obtained by substituting 6-bromothieno[2,3-d]pyrimidin-4(3H)-one for 1B. LCMS (ESI): calculated for C₃₄H₂₆Cl₂N₆O4S₂; [M+H]+: 717.1, 719.1, found: 717.1, 719.1. 1H NMR (500 MHz, DMSO-d6): δ 8.43 (s, 2H), 7.85 (s, 2H), 7.80 (d, J=7.7 Hz, 2H), 7.70 (s, 2H), 7.61-7.55 (m, 3H), 7.50 (d, J=7.4 Hz, 2H), 4.06-4.01 (m, 4H), 4.01-3.96 (m, 2H), 2.29-2.22 (m, 2H), 2.18-2.09 (m, 4H), 1.81-1.73 (m, 2H).

### Example 3. Preparation of 6,6′-(2,2′-dichloro-[1,1′-biphenyl]-3,3′-diyl)bis(7-fluoro-3-((5-oxopyrrolidin-2-yl)methyl)pyrrolo[2,1-f][1,2,4]triazin-4(3H)-one) (Compound 3)

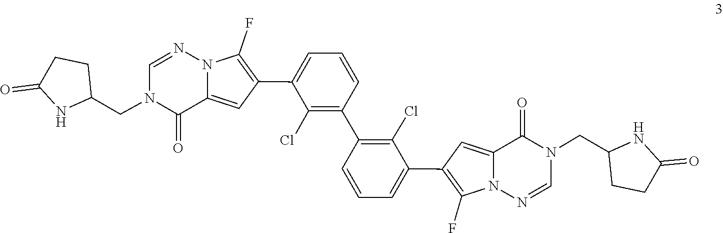 [SMILES: O=C1CCC(Cn2cnn3c(F)c(-c4cccc(-c5cccc(-c6cc7c(=O)n(CC8CCC(=O)N8)cnn7c6F)c5Cl)c4Cl)cc3c2=O)N1]

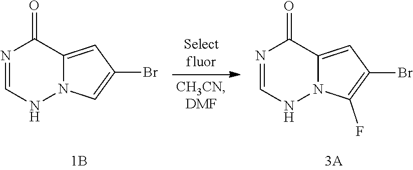 [SMILES: O=c1nc[nH]n2cc(Br)cc12 ; O=c1nc[nH]n2c(F)c(Br)cc12]

Compound 1B (1.0 g, 4.6 mmol, 1.0 eq.), Selectfluor (2.48 g, 7.0 mmol, 1.5 eq) were dissolved into CH₃CN (16 mL) and DMF (4.0 mL). The reaction was carried out at 50° C. for 2 h. 40 mL water was added to precipitate to give compound 3A (0.30 g, yield: 27.7%). LCMS (ESI): calculated for C₆H₃BrFN₃O; [M+H]+: 231.9, found: 231.9.

Following the procedure of Compound 1, the title Compound 3 was obtained by substituting 6-bromo-7-fluoropyrrolo[2,1-f][1,2,4]triazin-4(1H)-one (3A) for 1B. LCMS (ESI): calculated for C₃₄H₂₆Cl₂F₂N₈O₄; [M+H]+: 719.1721.1, found: 719.1, 721.1. ¹H NMR (500 Mhz, DMSO-d6): δ 8.01 (d, J=1.2 Hz, 2H), 7.87 (s, 2H), 7.72 (d, J=6.4 Hz, 2H), 7.49 (t, J=6.4 Hz, 2H), 7.32 (d, J=6 Hz, 2H), 7.26 (d, J=1.6 Hz, 2H), 3.94-3.88 (m, 6H), 2.27-2.22 (m, 2H), 2.18-2.10 (m, 4H), 1.78-1.77 (m, 2H).

### Example 4. Preparation of 6,6′-(2,2′-dichloro-[1,1′-biphenyl]-3,3′-diyl)bis(5-methyl-3-(((S)-5-oxopyrrolidin-2-yl)methyl)-3,5-dihydro-4H-pyrrolo[3,2-d]pyrimidin-4-one) (Compound 4)

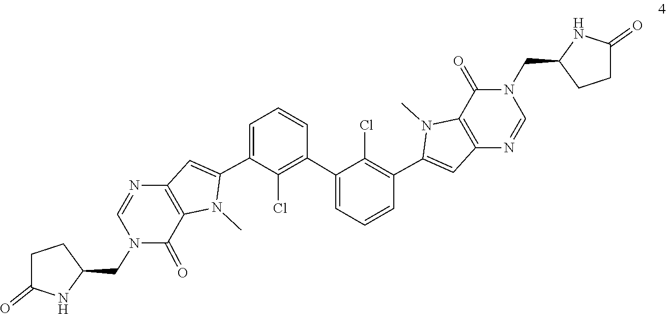 [SMILES: Cn1c(-c2cccc(-c3cccc(-c4cc5ncn(C[C@@H]6CCC(=O)N6)c(=O)c5n4C)c3Cl)c2Cl)cc2ncn(C[C@@H]3CCC(=O)N3)c(=O)c21]

Following the procedure of Compound 1, the title Compound 4 was obtained by substituting 6-bromo-5-methyl-3,5-dihydro-4H-pyrrolo[3,2-d]pyrimidin-4-one for 1B. LCMS (ESI): calculated for C₃₆H₃₂Cl₂N₈O₄; [M+H]+: 711.2, 713.2, found: 711.2, 713.2. ¹H NMR (500 MHz, DMSO-d₆) δ 8.10 (d, J=2.0 Hz, 2H), 7.83-7.79 (m, 4H), 7.48 (t, J=7.5 Hz, 2H), 7.29 (d, J=7.5 Hz, 2H), 4.08 (s, 6H), 3.99-3.95 (m, 6H), 2.23-2.19 (m, 2H), 2.12-2.04 (m, 2H), 2.02-1.96 (m, 2H), 1.79-1.76 (m, 2H).

### Example 5. Preparation of 7,7′-(2,2′-dichloro-[1,1′-biphenyl]-3,3′-diyl)bis(2-(((S)-5-oxopyrrolidin-2-yl)methyl)pyrrolo[1,2-a]pyrazin-1(2H)-one) (Compound 5)

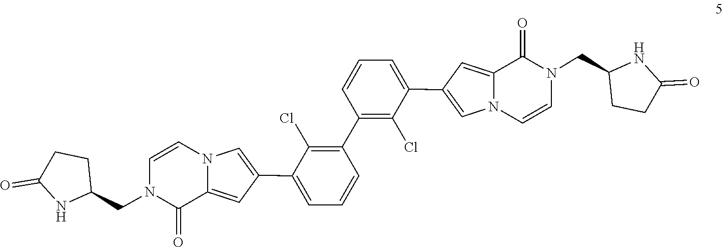 [SMILES: O=C1CC[C@@H](Cn2ccn3cc(-c4cccc(-c5cccc(-c6cc7c(=O)n(C[C@@H]8CCC(=O)N8)ccn7c6)c5Cl)c4Cl)cc3c2=O)N1]

Following the procedure of Compound 1, the title Compound 5 was obtained by substituting 7-bromopyrrolo[1,2-a]pyrazin-1(2H)-one for 1B. LCMS (ESI): calculated for C₃₆H₃₀Cl₂N₆O₄; [M+H]+: 681.2, found: 683.2. ¹H NMR (500 Mhz, DMSO-d₆) δ 7.87 (s, 2H), 7.79 (s, 2H), 7.71-7.65 (m, 2H), 7.51-7.42 (m, 4H), 7.33-7.27 (m, 2H), 7.25 (s, 2H), 6.87 (d, J=5.9 Hz, 2H), 4.02-3.94 (m, 3H), 3.76-3.69 (m, 3H), 2.23-2.14 (m, 2H), 2.13-2.02 (m, 4H), 1.81-1.72 (m, 2H).

### Example 6. Preparation of 2,2′-(2,2′-dichloro-[1,1′-biphenyl]-3,3′-diyl)bis(1-methyl-5-(((S)-5-oxopyrrolidin-2-yl)methyl)-1,5-dihydro-4H-imidazo[4,5-c]pyridin-4-one) (Compound 6)

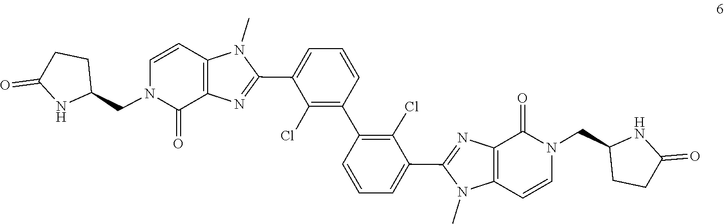 [SMILES: Cn1c(-c2cccc(-c3cccc(-c4nc5c(=O)n(C[C@@H]6CCC(=O)N6)ccc5n4C)c3Cl)c2Cl)nc2c(=O)n(C[C@@H]3CCC(=O)N3)ccc21]

Following the procedure of Compound 1, the title Compound 6 was obtained by substituting 2-bromo-1-methyl-1,5-dihydro-4H-imidazo[4,5-c]pyridin-4-one for 1B. LCMS (ESI): calculated for C₃₆H₃₂Cl₂N₈O₄; [M+H]+: 711.2, 713.2, found: 711.2, 713.2. ¹H NMR (500 Mhz, DMSO-d₆) δ 7.87 (s, 2H), 7.79 (s, 2H), 7.71-7.65 (m, 2H), 7.51-7.42 (m, 4H), 7.33-7.27 (m, 2H), 7.25 (s, 2H), 6.87 (d, J=5.9 Hz, 2H), 4.02-3.94 (m, 3H), 3.76-3.69 (m, 3H), 2.23-2.14 (m, 2H), 2.13-2.02 (m, 4H), 1.81-1.72 (m, 2H).

### Example 7. Preparation of 2,2′-(2,2′-dichloro-[1,1′-biphenyl]-3,3′-diyl)bis(5-(((S)-5-oxopyrrolidin-2-yl)methyl)pyrazolo[1,5-a]pyrazin-4(5H)-one) (Compound 7)

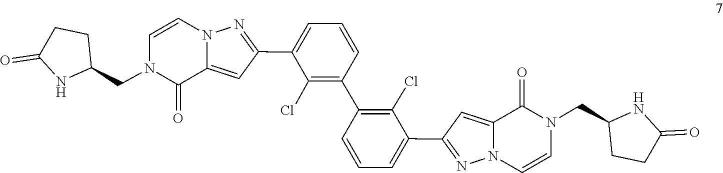 [SMILES: O=C1CC[C@@H](Cn2ccn3nc(-c4cccc(-c5cccc(-c6cc7c(=O)n(C[C@@H]8CCC(=O)N8)ccn7n6)c5Cl)c4Cl)cc3c2=O)N1]

Following the procedure of Compound 1, the title Compound 7 was obtained by substituting 2-bromopyrazolo[1,5-a]pyrazin-4(5H)-one for 1B. LCMS (ESI): calculated for C₃₄H₂₈Cl₂N₈O₄; [M+H]+: 683.2, 685.2, found: 683.2, 685.2. ¹H NMR (500 MHz, DMSO-d₆) δ 7.89-7.87 (m, 4H), 7.80 (s, 2H), 7.57 (t, J=10 Hz, 2H), 7.48-7.46 (s, 2H), 7.40 (s, 2H), 7.20 (t, J=4.8 Hz, 2H), 4.05-3.97 (m, 4H), 3.87-3.83 (m, 2H), 2.26-2.21 (m, 2H), 2.14-2.07 (m, 4H), 1.79-1.74 (m, 2H).

### Example 8. Preparation of 2,2′-(2,2′-dichloro-[1,1′-biphenyl]-3,3′-diyl)bis(5-(((S)-5-oxopyrrolidin-2-yl)methyl)thieno[3,2-c]pyridin-4(5H)-one) (Compound 8)

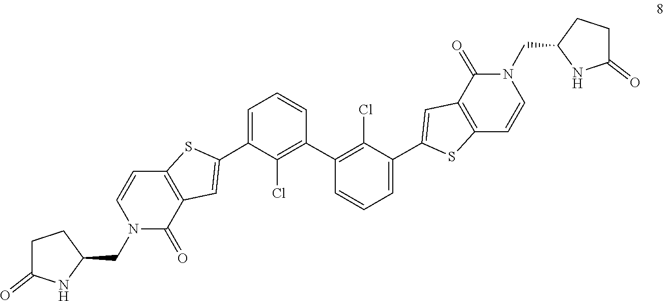 [SMILES: O=C1CC[C@@H](Cn2ccc3sc(-c4cccc(-c5cccc(-c6cc7c(=O)n(C[C@@H]8CCC(=O)N8)ccc7s6)c5Cl)c4Cl)cc3c2=O)N1]

Following the procedure of Compound 1, the title Compound 8 was obtained by substituting 2-bromothieno[3,2-c]pyridin-4(5H)-one for 1B. LCMS (ESI): calculated for C₃₆H₂₈Cl₂N₄O₄S₂; [M+H]+: 715.1, 717.1, found: 715.1, 717.1. ¹H NMR (500 MHz, DMSO-d6) δ 7.80-7.76 (m, 6H), 7.61-7.54 (m, 4H), 7.47 (dd, J=7.6, 1.6 Hz, 2H), 6.97 (d, J=7.2 Hz, 2H), 4.15-4.09 (m, 2H), 4.00-3.87 (m, 4H), 2.26-2.12 (m, 2H), 2.11-1.89 (m, 4H), 1.85-1.70 (m, 2H).

### Example 9. Preparation of 7,7′-(2,2′-dichloro-[1,1′-biphenyl]-3,3′-diyl)bis(2-(((S)-5-oxopyrrolidin-2-yl)methyl)-3,4-dihydropyrrolo[1,2-a]pyrazin-1(2H)-one) (Compound 9)

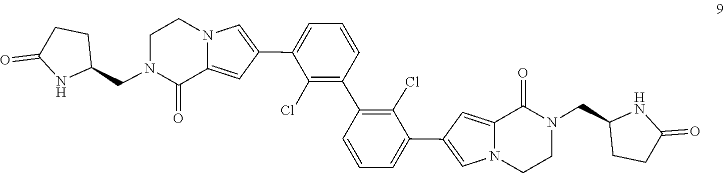 [SMILES: O=C1CC[C@@H](CN2CCn3cc(-c4cccc(-c5cccc(-c6cc7n(c6)CCN(C[C@@H]6CCC(=O)N6)C7=O)c5Cl)c4Cl)cc3C2=O)N1]

Following the procedure of Compound 1, the title Compound 9 was obtained by 7-bromo-3,4-dihydropyrrolo[1,2-a]pyrazin-1(2H)-one for 1B. LCMS (ESI): calculated for C₃₆H₄Cl₂N₆O₄; [M+H]+: 684.2, 686.2, found: 684.2, 686.2.

¹H NMR (500 MHz, DMSO-d₆) δ 7.77 (s, 2H), 7.58 (d, J=6 Hz, 2H), 7.47 (s, 2H), 7.41 (t, J=6 Hz, 2H), 7.20 (d, J=6 Hz, 2H), 7.00 (s, 2H), 4.15 (t, J=4.4 Hz, 4H), 3.53 (s, 4H).

### Example 10. Preparation of 8,8′-(2,2′-dichloro-[1,1′-biphenyl]-3,3′-diyl)bis(3,7-dimethyl-1-(((S)-5-oxopyrrolidin-2-yl)methyl)-3,7-dihydro-1H-purine-2,6-dione) (Compound 10)

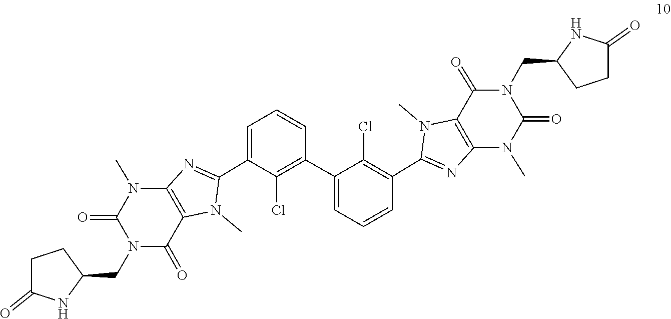 [SMILES: Cn1c(-c2cccc(-c3cccc(-c4nc5c(c(=O)n(C[C@@H]6CCC(=O)N6)c(=O)n5C)n4C)c3Cl)c2Cl)nc2c1c(=O)n(C[C@@H]1CCC(=O)N1)c(=O)n2C]

Following the procedure of Compound 1, the title Compound 10 was obtained by substituting 8-bromo-3,7-dimethyl-3,7-dihydro-1H-purine-2,6-dione for 1B. LCMS (ESI): calculated for C₃₆H₄Cl₂N₁₀O₆; [M+H]+: 772.2, 774.2, found: 772.2, 774.2. 1H NMR (500 MHz, DMSO-d6) δ 7.81 (d, J=5.0 Hz, 2H), 7.68 (d, J=5.0 Hz, 2H), 7.52 (t, J=10.0 Hz, 2H), 7.12 (d, J=5.0 Hz, 2H), 3.98-3.92 (m, 6H), 3.58 (s, 6H), 3.32 (s, 6H), 2.38-2.26 (m, 2H), 2.14-2.09 (m, 4H), 1.83-1.76 (m, 2H).

### Example 11. Preparation of 7,7′-(2,2′-dichloro-[1,1′-biphenyl]-3,3′-diyl)bis(2-(((S)-5-oxopyrrolidin-2-yl)methyl)pyrrolo[1,2-d][1,2,4]triazin-1(2H)-one) (Compound 11)

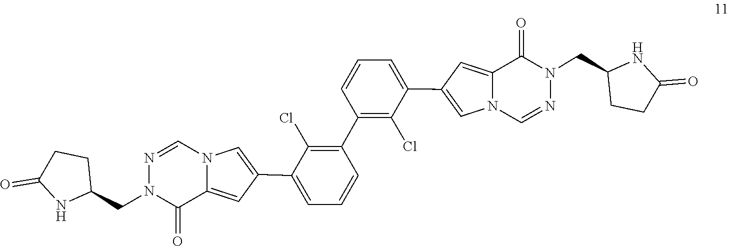 [SMILES: O=C1CC[C@@H](Cn2ncn3cc(-c4cccc(-c5cccc(-c6cc7c(=O)n(C[C@@H]8CCC(=O)N8)ncn7c6)c5Cl)c4Cl)cc3c2=O)N1]

Following the procedure of Compound 1, the title Compound 11 was obtained by substituting 8-bromo-3,7-dimethyl-3,7-dihydro-1H-purine-2,6-dione for 1B. LCMS (ESI): calculated for C₃₄H₂₈C₂N₈O₄; [M+H]+: 682.1, 684.1, found: 682.1, 684.1. ‘H’H NMR (500 MHz, DMSO-d6) δ 8.36 (s, 2H), 7.92 (d, J=1.8 Hz, 2H), 7.71-7.63 (m, 6H), 7.54-7.48 (m, 2H), 7.30-7.24 (m, 2H), 4.25-4.09 (m, 6H), 2.40-2.26 (m, 4H), 1.99-1.77 (m, 4H).

### Example 12. Preparation of 2,2′-(2,2′-dichloro-[1,1′-biphenyl]-3,3′-diyl)bis(7-(((S)-5-oxopyrrolidin-2-yl)methyl)imidazo[1,2-a]pyrazin-8(7H)-one) (Compound 12)

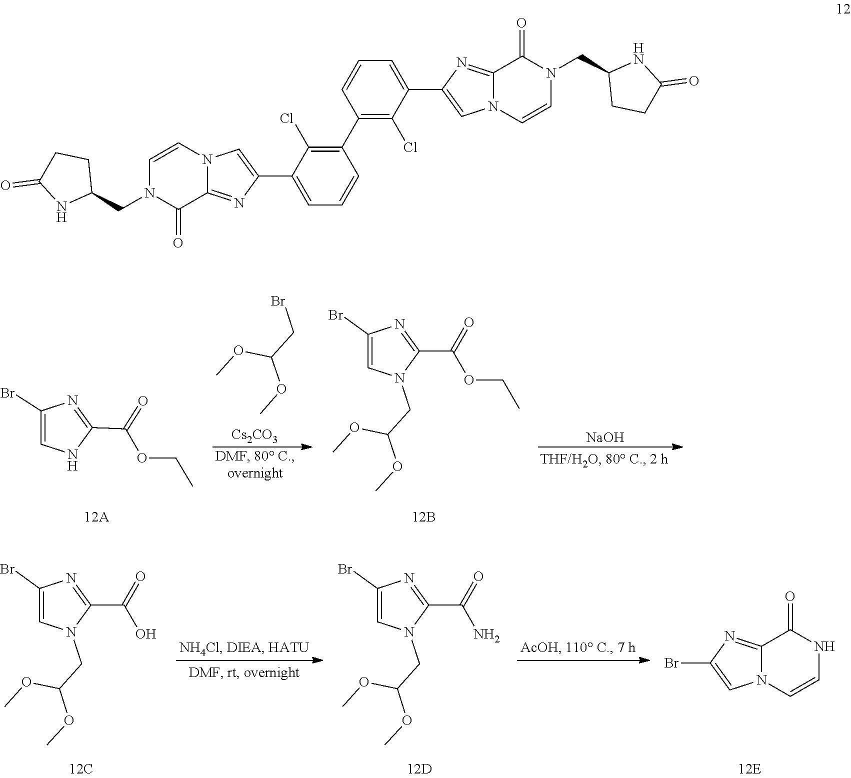

To a stirred solution of Compound 12A (770 mg, 3.53 mmol, 1 eq) in DMF (10 mL) was added Cs₂CO₃ (2302 mg, 7.06 mmol, 2.0 eq.) and 2-bromo-1,1-dimethoxyethane (891 mg, 5.30 mmol, 1.5 eq.) at 25° C. The resulting mixture was stirred for 16 h at 80° C. After cooled to 25° C., the reaction was quenched with water (10 ml) and extracted with EtOAc (30 mL×2). The combined organic layers were washed with brine (20 mL), dried over anhydrous sodium sulfate. After filtration, the filtrate was concentrated under reduced pressure. The residue was purified by silica gel column chromatography, eluted with PE/EtOAC (5:1-3:1) to afford Compound 12B (451 mg. yield: 41.7%).

To a stirred solution of Compound 12B (451 mg, 1.47 mmol, 1.0 eq) in EtOH/H₂O (5 mL/1 mL) was added NaOH (89 mg, 2.22 mmol, 1.5 eq) at 25° C. The resulting mixture was stirred for 2 h at 80° C. After cooled to 25° C., the reaction was quenched with HOAc solution (1M, in water) and adjusted to pH~6.0. It was extracted with EtOAc (30 mL×2), the combined organic layers were washed with brine (20 mL), dried over anhydrous sodium sulfate. After filtration, the filtrate was concentrated under reduced pressure to afford Compound 12C (410 mg. yield: 100%).

To a stirred solution of Compound 12C (410 mg, 1.47 mmol, 1.0 eq) in DMF (5 mL) was added NH₄Cl (786 mg, 14.7 mmol, 10 eq), DIEA (508 mg, 4.41 mmol, 3.0 eq) and HATU (1040 mg, 2.94 mmol, 2 eq), at 25° C. The resulting mixture was stirred for 16 h at 25° C. The reaction was quenched with water (100 mL) and extracted with EtOAc (30 mL×2). The combined organic layers were washed with brine (20 mL), dried over anhydrous sodium sulfate. After filtration, the filtrate was concentrated under reduced pressure. The residue was purified by silica gel column chromatography, eluted with PE/EtOAc (5:1-2:1) to afford Compound 12D (300 mg. yield: 66.8%). LCMS (ESI): calculated for C₈H₁₂BrN₃O₃; [M+H]+: 277.01, 279.00, found: 246.11, 248.09.

Compound 12D (300 mg, 1.08 mmol, 1.0 eq) was dissolved in HOAc (3.0 mL) at 25° C. and the resulting mixture was stirred at for 4 h at 105° C. After the reaction was completed, the mixture was concentrated under reduced pressure to afford Compound 12E (230 mg. yield: 100%). LCMS (ESI): calculated for C₆H₄BrN₃O; [M+H]+: 214.02, 216.02, found: 214.09, 216.07.

Following the procedure of Compound 1, the title Compound 12 was obtained by substituting 12E for lB. LCMS (ESI): calculated for C₃₄H₂₈Cl₂N₈O₄; [M+H]+: 682.2, 684.2, found: 682.2, 684.2. ¹H NMR (500 Mhz, DMSO-d₆) δ ¹H NMR (500 Mhz, DMSO-d6) 8.43 (d, J=2.0 Hz, 2H), 8.23 (d, J=7.9 Hz, 2H), 7.79 (d, J=6.5 Hz, 2H), 7.63 (d, J=5.7 Hz, 2H), 7.57 (t, J=7.7 Hz, 2H), 7.38 (d, J=7.5 Hz, 2H), 7.15 (dd, J=5.9, 1.7 Hz, 2H), 4.04-4.02 (m, 1H), 4.00-3.97 (m, 2H), 3.87-3.84 (m, 2H), 3.84-3.81 (m, 1H), 2.31-2.18 (m, 3H), 2.13-2.08 (m, 3H), 1.81-1.74 (m, 2H).

### Example 13. Preparation of 2,2′-(2,2′-dichloro-[1,1′-biphenyl]-3,3′-diyl)bis(3-fluoro-7-(((S)-5-oxopyrrolidin-2-yl)methyl)imidazo[1,2-a]pyrazin-8(7H)-one) (Compound 13)

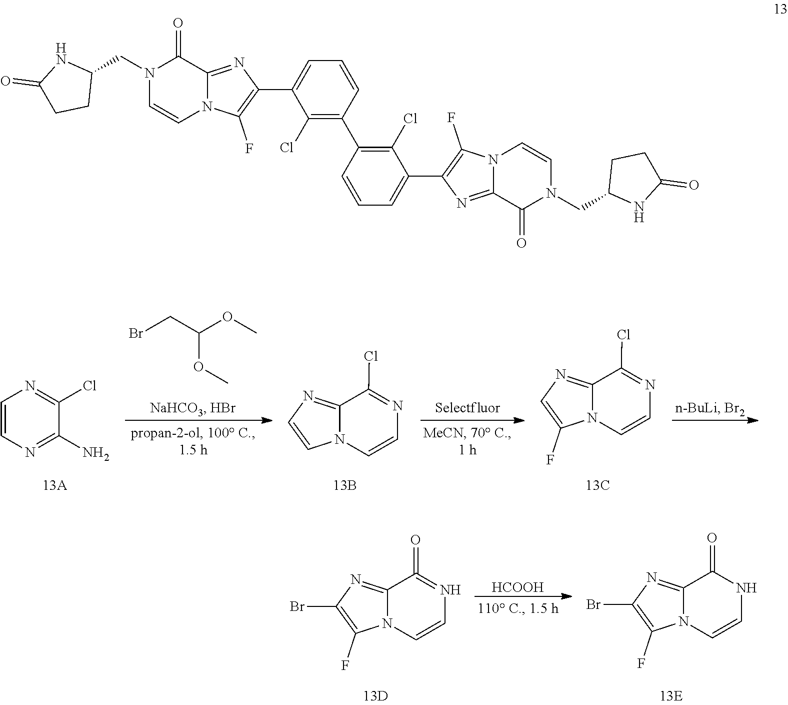

A mixture solution of 2-bromo-1,1-dimethoxyethane (5.8 mL, 49.3 mmol, 4.2 eq) and HBr (1.4 mL, 48%, in water, 12.5 mol, 1.08 eq) were stirred at 100° C. After 2 h, the mixture was cooled to room temperature and added to a stirred solution of NaHCO₃ in propan-2-ol (30 mL) dropwise slowly. The white solid was separated out. After filtration, 13A (1.5 g, 11.6 mmol, 1.0 eq) was added to the filtrate and the reaction was stirred for 2 h at 100° C. After cooling, the mixture was filtered to give the cake, dissolved in DCM (50 mL), washed with saturated sodium bicarbonate solution (50 mL×2), dried over anhydrous magnesium sulphate. After filtration, the filtrate was concentrated under reduced pressure. The mixture was concentrated under reduced pressure to afford Compound 13B (1.5 g. yield: 84%). LCMS (ESI): calculated for C₆H₄ClN₃; [M+H]+: 154.01, found: 154.23

To a solution of Compound 13B (1.5 g, 9.8 mmol, 1.0 eq) in MeCN (20 mL) was added Selectfluor (3.47 g, 9.8 mmol, 1.0 eq) at 25° C. and the reaction was stirred for 1 h at 70° C. The mixture was concentrated under reduced pressure. The residue was purified by silica gel column chromatography, eluted with PE/EtOAc (5:1-3:1) to afford 13C (0.56 g. yield: 33.5%). LCMS (ESI): calculated for C₆H₃ClFN₃; [M+H]+: 171.56, found: 172.10.

To a solution of Compound 13C (363 mg, 2.12 mmol, 1 eq) in THF (10 mL) was added n-BuLi (1.56 mL, 1.6 M in THF, 2.54 mmol, 1.2 eq) at −60° C. and the reaction was stirred for 30 min at −60° C. Then Br₂ (0.16 mL, 3.18 mmol, 1.5 eq) was added to the mixture at −60° C. and stirred for 2 h. The mixture was quenched with saturated NH₄Cl solution (3.0 mL) and concentrated under reduce pressure. The residue was purified by silica gel column chromatography, eluted with PE/EtOAc (5:1-3:1) to afford 13D, 183 mg. yield: 34.6%. LCMS (ESI): calculated for C₆H₂BrClFN₃; [M+H]+: 250.46, found: 250.32.

Compound 13D (83 mg, 0.33 mmol, 1.0 eq) was dissolved in HCOOH (2.0 mL) at 25° C. and the reaction was stirred for 1.5 h at 110° C. After the reaction was completed, the mixture was concentrated under reduce pressure to afford 13E (76 mg. yield: 100%). LCMS (ESI): calculated for C₆H₃BrFN₃O; [M+H]+: 234.9, found: 234.1

Following the procedure of Compound 1, the title Compound 13 was obtained by substituting 13E for 1B. LCMS (ESI): calculated for C₃₄H₂₆Cl₂F₂N₈O₄; [M+H]+: 719.5, 721.5, found: 719.5, 721.5. ¹H NMR (500 MHz, DMSO-d6) δ 8.42 (d, J=2.0 Hz, 2H), 8.22 (d, J=7.9 Hz, 2H), 7.62 (d, J=5.7 Hz, 2H), 7.56 (t, J=7.7 Hz, 2H), 7.37 (d, J=7.5 Hz, 2H), 7.14 (dd, J=5.9, 1.7 Hz, 2H), 4.03-3.81 (m, 6H), 2.32-2.19 (m, 3H), 2.14-2.09 (m, 3H), 1.80-1.73 (m, 2H).

### Example 14. Preparation of 6,6′-(2,2′-dichloro-[1,1′-biphenyl]-3,3′-diyl)bis(3-(((S)-5-oxopyrrolidin-2-yl)methyl)-3,7-dihydro-4H-pyrrolo[2,3-d]pyrimidin-4-one) (Compound 14)

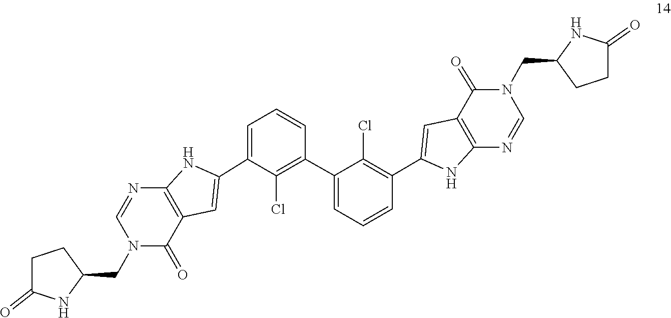 [SMILES: O=C1CC[C@@H](Cn2cnc3[nH]c(-c4cccc(-c5cccc(-c6cc7c(=O)n(C[C@@H]8CCC(=O)N8)cnc7[nH]6)c5Cl)c4Cl)cc3c2=O)N1]

Following the procedure of Compound 1, the title Compound 14 was obtained by substituting 6-bromo-3,7-dihydro-4H-pyrrolo[2,3-d]pyrimidin-4-one for 1B. LCMS (ESI): calculated for C₃₄H₂₈Cl₂N₈O₄; [M+H]+: 683.5, 685.5, found: 683.5, 685.5. ¹H NMR (500 MHz, DMSO-d₆) δ 12.40 (s, 2H), 8.17 (s, 2H), 7.83 (s, 2H), 7.71 (d, J=2.5 Hz, 2H), 7.54 (t, J=5.0 Hz, 2H), 7.38 (d, J=5.0 Hz, 2H), 6.89 (d, J=2.5 Hz, 2H), 4.08-4.01 (m, 2H), 3.98-3.93 (m, 4H), 2.24-2.17 (m, 2H), 2.10-2.03 (m, 4H), 1.79-1.74 (m, 2H).

### Example 15. Preparation of 2,2′-(2,2′-dichloro-[1,1′-biphenyl]-3,3′-diyl)bis(7-(((S)-5-oxopyrrolidin-2-yl)methyl)-[1,24]triazolo[1,5-a]pyrazin-8(7H)-one) (Compound 15)

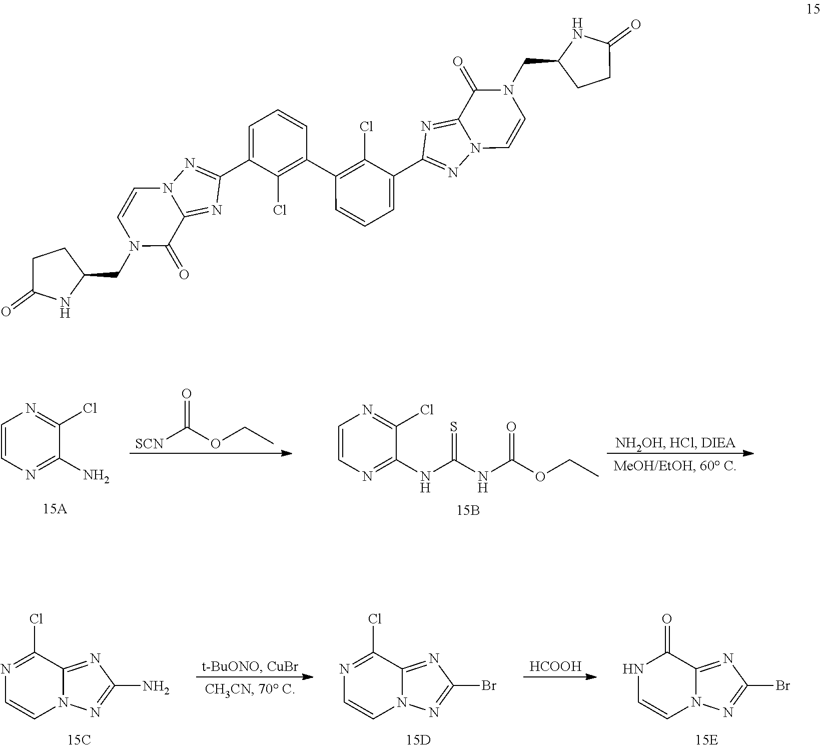

To a solution of 3-chloropyrazine-2-amine (1.0 g, 7.78 mmol, 1.0 eq.) in dioxane (100 mL) was added dropwise isothiocyanate-O-ethyl carbonisothiocyanatidate (1.18 g, 7.9 mmol, 1.02 eq.) and stirred 24 h at r.t. The solvent was evaporated and the residue was purified by flash chromatography on silica gel using a gradient of 0% to 5% MeOH in DCM. The title compound 15B was obtained as a yellow solid (1.13 g, 56%), LCMS (ESI): calculated for C₈H₉ClN₄O₂S; [M+H]+: 261.0, 263.0, found: 261.0, 263.0.

Compound 15B (0.73 g, 2.98 mmol, 1.0 eq.), hydroxylamine hydrochloride (1.03 g, 74.2 mmol, 25 eq.) and diisopropylethylamine (1.15 g, 1.55 mL, 8.9 mmol, 3.0 eq.) were dissolved into MeOH (4.0 mL) and EtOH (4.0 mL). The reaction mixture was heated to 60° C. for 3 h. The solvent was evaporated and the residue was suspended in DCM (30 mL) and water (6.0 mL). The suspension was stirred for 10 min and the solid was filtered off. The aqueous phase was extracted four times with DCM, the combined organic layer was dried with Na₂SO₄ and the solvent was removed in vacuo. The residue was combined with the filtered solid compound 15C (808 mg, yield: 170%) and used in the next step without purification. LCMS (ESI): calculated for C₅H₄ClN₅; [M+H]+: 170.0, 172.0, found: 170.0, 172.0.

Cuprous (I) bromide (1.06 g, 4.8 mmol), tert-butyl nitrite (505 mg, 0.605 mL, 4.8 mmol) and compound 15C were dissolved in acetonitrile (60 mL). The reaction mixture was heated to 75° C. for 35 min. After cooling, saturated NaHCO₃ solution (20 mL) was added and separated and extracted with EtOAc (40 mL). Then combined organic layer was dried with Na₂SO₄ and the solvent was removed in vacuo. The crude material was purified by flash chromatography on silica gel (0% to 50% EtOAc in heptane) to afford an off-white solid 15D (202 mg, 29%). LCMS (ESI): calculated for C₅H₂BrClN₄; [M+H]+: 232.9, 234.9, 236.9, found: 232.9, 234.9, 236.9.

Compound 15D (100 mg, 0.465 mmol, 1.0 eq.) was dissolved in 5.0 mL of formic acid and heated to 100° C. for 8 h. The reaction solution was cooled, concentrated, and a solid precipitated out to give compound 15E (102 mg, 55.9%), LCMS (ESI): calculated for C₅H₃BrN₄O; [M+H]+: 215.0, 217.0, found: 215.0, 217.0.

Following the procedure of Compound 1, the title Compound 15 was obtained by substituting 15E for 1B. LCMS (ESI): calculated for C₃₂H₂₆Cl₂N₁₀O₄; [M+H]+ 685.5, 687.5, found: 685.5, 687.5. ¹H NMR (500 MHz, DMSO-d₆) δ 8.09 (d, J=5.0 Hz, 2H), 7.95 (d, J=5.0 Hz, 2H), 7.75 (s, 2H), 7.63 (t, J=7.5 Hz, 2H), 7.58 (t, J=5.0 Hz, 2H), 7.45 (d, J=10.0 Hz, 2H), 4.13-4.01 (m, 4H), 3.99-3.93 (m, 2H), 2.32-2.26 (m, 2H), 2.19-2.07 (m, 4H), 1.80-1.74 (m, 2H).

### Example 16. Preparation of 7,7′-(2,2′-dichloro-[1,1′-biphenyl]-3,3′-diyl)bis(4-fluoro-2-((5-oxopyrrolidin-2-yl)methyl)pyrrolo[1,2-a]pyrazin-1(2H)-one) (Compound 16)

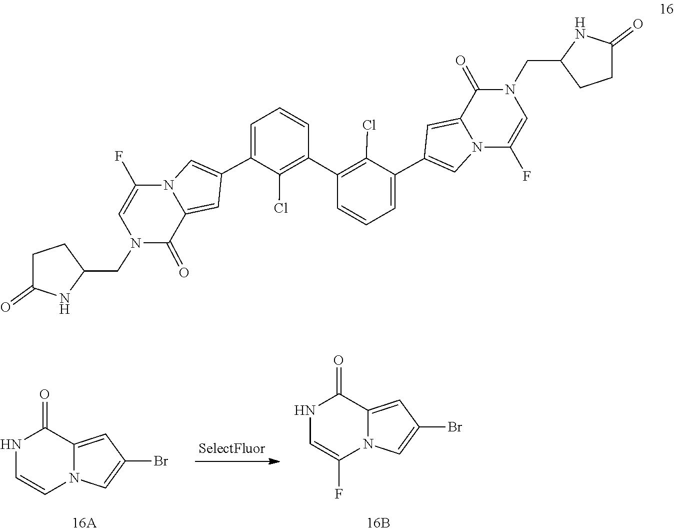 [SMILES: O=C1CCC(Cn2cc(F)n3cc(-c4cccc(-c5cccc(-c6cc7c(=O)n(CC8CCC(=O)N8)cc(F)n7c6)c5Cl)c4Cl)cc3c2=O)N1 ; O=c1[nH]ccn2cc(Br)cc12 ; O=c1[nH]cc(F)n2cc(Br)cc12]

Compound 16A (200 mg, 0.938 mmol, 1.0 eq.) and SelectFluor (300 mg, 0.75 mmol, 0.8 eq.) were dissolved into DCM (4.0 mL) and H₂O (4 mL), and stirred for overnight at 25° C. The mixture reaction was added water (20 mL), and extracted with DCM (20 mL×3) and the combined organics were dried over Na₂SO₄, filtered and concentrated under reduce pressure to give the crude. The residue was purified by silica gel column chromatography, eluted with PE/EtOAc (3:1) to afford Compound 16B (110 mg, yield: 49.4%). LCMS (ESI): calculated for C₇H₄BrFN₂O; [M+H]+: 231.01, 233.01, found: 231.01, 233.01.

Following the procedure of Compound 1, the title Compound 16 was obtained by substituting 16B for 1B. LCMS (ESI): calculated for C₃₆H₂₈Cl₂F₂N₆O₄; [M+H]+: 717.2, 719.2, found: 717.2, 719.2. ¹H NMR (500 MHz, DMSO-d6) δ 8.10 (d, J=2.5 Hz, 2H), 7.87 (d, J=5.0 Hz, 2H), 7.66 (d, J=7.5 Hz, 2H), 7.45 (d, J=5.0 Hz, 2H), 7.40 (d, t=7.5 Hz, 2H), 7.32 (d, J=2.5 Hz, 2H), 6.73 (d, J=2.5 Hz, 2H), 3.93-3.81 (m, 6H), 2.24-2.19 (m, 2H), 2.12-2.04 (m, 4H), 1.77-1.69 (m, 2H).

### Example 17. Preparation of 6,6′-(2,2′-dimethyl-[1,1′-biphenyl]-3,3′-diyl)bis(3-(((S)-5-oxopyrrolidin-2-yl) methyl)pyrrolo[2,1-f][1,2,4]triazin-4(3H)-one) (Compound 17)

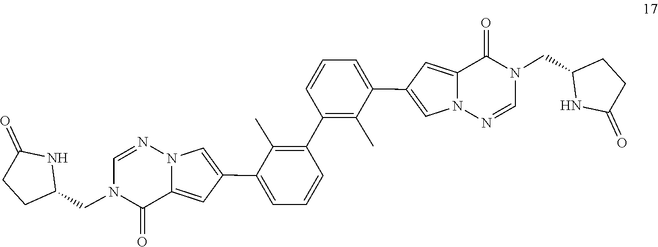 [SMILES: Cc1c(-c2cc3c(=O)n(C[C@@H]4CCC(=O)N4)cnn3c2)cccc1-c1cccc(-c2cc3c(=O)n(C[C@@H]4CCC(=O)N4)cnn3c2)c1C]

Following the procedure of Compound 1, the title Compound 17 was obtained by substituting 3-bromo-2-methylphenol for 3-bromo-2-chlorophenol. LCMS (ESI): calculated for C₃₆H₃₄N₈O₄; [M+H]+: 643.3, 645.3, found: 643.3, 645.3. ¹H NMR (500 Mhz, DMSO-d₆) δ 8.09 (s, 2H), 7.83-7.87 (m, 4H), 7.40-7.42 (m, 2H), 7.31 (t, J=6.4 Hz), 7.09 (s, 2H), 3.89-3.93 (m, 6H), 2.18-2.26 (m, 2H), 2.04-2.11 (m, 10H), 1.73-1.77 (m, 2H).

### Example 18. Preparation of 6,6′-(2,2′-dichloro-[1,1′-biphenyl]-3,3′-diyl)bis(3-((5-oxopyrrolidin-3-yl)meth yl)pyrrolo[2,1-f][1,2,4]triazin-4(3H)-one) (Compound 18)

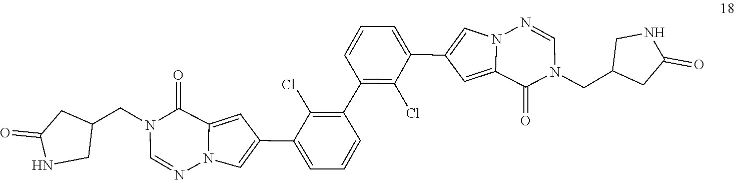 [SMILES: O=C1CC(Cn2cnn3cc(-c4cccc(-c5cccc(-c6cc7c(=O)n(CC8CNC(=O)C8)cnn7c6)c5Cl)c4Cl)cc3c2=O)CN1]

Following the procedure of Compound 1, the title Compound 18 was obtained by substituting 4-(hydroxymethyl)pyrrolidin-2-one for 1D-1. LCMS (ESI): calculated for C₃₄H₂₈Cl₂N₈O₄; [M+H]+: 683.2, 685.2, found: 683.2, 685.2. ¹H NMR (500 Mhz, DMSO-d₆) δ 8.61 (d, J=1.6 Hz, 2H), 8.09 (s, 2H), 7.94 (d, J=1.6 Hz, 2H), 7.68 (m, 4H), 7.52 (t, J=7.6 Hz, 2H), 6.45 (m, 2H), 4.24-3.71 (m, 4H), 3.46 (m, 2H), 3.31 (m, 2H), 2.55-2.47 (m, 2H), 2.45 (m, 2H), 2.36 (m, 2H).

### Example 19. Preparation of 6,6′-(2,2′-dichloro-[1,1′-biphenyl]-3,3′-diyl)bis(3-(2-(2-oxoimidazolidin-1-yl)ethyl)pyrrolo[2,1-f][1,2,4]triazin-4(3H)-one) (Compound 19)

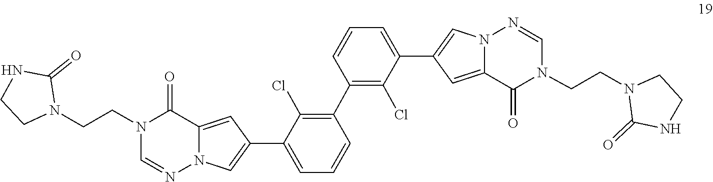 [SMILES: O=C1NCCN1CCn1cnn2cc(-c3cccc(-c4cccc(-c5cc6c(=O)n(CCN7CCNC7=O)cnn6c5)c4Cl)c3Cl)cc2c1=O]

Following the procedure of Compound 1, the title Compound 19 was obtained by substituting 1-(2-hydroxyethyl)imidazolidin-2-one for 1D-1. LCMS (ESI): calculated for C₃₄H₃₀Cl₂N₁₀O₄; [M+H]+: 712.2, 714.2. found: 712.2, 714.2. ¹H NMR (500 Mhz, DMSO-d6) δ 8.11 (s, 2H), 7.97 (d, J=1.9 Hz, 2H), 7.71 (dd, J=7.9, 1.7 Hz, 2H), 7.48 (t, J=7.6 Hz, 2H), 7.32 (dd, J=7.6, 1.7 Hz, 2H), 7.25 (d, J=1.9 Hz, 2H), 6.31 (s, 2H), 3.98 (t, J=5.6 Hz, 4H), 3.45 (dd, J=9.0, 6.7 Hz, 4H), 3.38 (t, J=5.4 Hz, 4H), 3.21 (dd, J=8.9, 6.8 Hz, 4H).

### Example 20. Preparation of 6,6′-(2,2′-dichloro-[1,1′-biphenyl]-3,3′-diyl)bis(3-(2-hydroxyethyl)pyrrolo[2,1-f][1,2,4]triazin-4(3H)-one) (Compound 20)

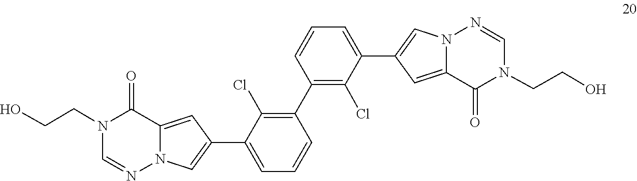 [SMILES: O=c1c2cc(-c3cccc(-c4cccc(-c5cc6c(=O)n(CCO)cnn6c5)c4Cl)c3Cl)cn2ncn1CCO]

Following the procedure of Compound 1, the title Compound 20 was obtained by substituting 2-bromoethan-1-ol for 1D. LCMS (ESI): calculated for C₂₈H₂₂Cl₂N₆O₄; [M+H]+: 577.1, 579.1. found: 577.1, 579.1. 1H NMR (500 Mhz, DMSO-d6) δ 8.09-7.98 (m, 3H), 7.91 (d, J=4.0 Hz, 1H), 7.70 (d, J=7.7 Hz, 2H), 7.48 (m, 2H), 7.38-7.21 (m, 4H), 3.93 (m, 4H), 3.63 (m, 4H).

### Example 21. Preparation of 6,6′-(2,2′-dichloro-[1,1′-biphenyl]-3,3′-diyl)bis(3-(2-(1H-pyrazol-1-yl)ethyl)pyrrolo[2,1-f][1,2,4]triazin-4(3H)-one) (Compound 21)

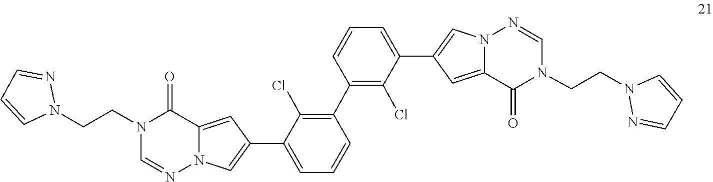 [SMILES: O=c1c2cc(-c3cccc(-c4cccc(-c5cc6c(=O)n(CCn7cccn7)cnn6c5)c4Cl)c3Cl)cn2ncn1CCn1cccn1]

Following the procedure of Compound 1, the title Compound 21 was obtained by substituting 2-(1H-pyrazol-1-yl)ethan-1-ol for 1D-1. LCMS (ESI): calculated for C₃₄H₂₆Cl₂N₁₀O₂; [M+H]+: 677.2, 679.2. found: 677.2, 679.2. ¹H NMR (500 Mhz, DMSO-d6) δ 7.96 (d, J=1.9 Hz, 2H), 7.74 (d, J=2.2 Hz, 2H), 7.70 (dd, J=7.7, 1.7 Hz, 2H), 7.52-7.46 (m, 4H), 7.43 (s, 2H), 7.32 (dd, J=7.6, 1.6 Hz, 2H), 7.28 (d, J=1.9 Hz, 2H), 6.26-6.22 (m, 2H), 4.46 (t, J=5.7 Hz, 4H), 4.28 (t, J=5.7 Hz, 4H).

### Example 22. Preparation of 6,6′-(2,2′-dichloro-[1,1′-biphenyl]-3,3′-diyl)bis(3-(2-(1H-1,2,4-triazol-1-yl)eth yl)pyrrolo[2,1-f][1,2,4]triazin-4(3H)-one) (Compound 22)

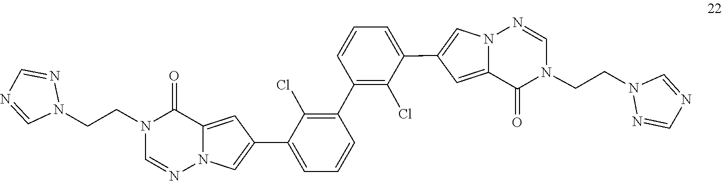 [SMILES: O=c1c2cc(-c3cccc(-c4cccc(-c5cc6c(=O)n(CCn7cncn7)cnn6c5)c4Cl)c3Cl)cn2ncn1CCn1cncn1]

Following the procedure of Compound 1, the title Compound 22 was obtained by substituting 2-(1H-1,2,4-triazol-1-yl)ethan-1-ol for 1D-1. LCMS (ESI): calculated for C₃₂H₂₄Cl₂N₁₂O₂; [M+H]+: 679.2, 681.2. found: 679.2, 681.2. 1H NMR (500 Mhz, DMSO-d6) δ 8.54 (s, 2H), 7.99 (t, J=1.7 Hz, 4H), 7.73-7.66 (m, 4H), 7.48 (t, J=7.6 Hz, 2H), 7.32 (dd, J=7.5, 1.6 Hz, 2H), 7.28 (d, J=1.7 Hz, 2H), 4.56 (d, J=5.8 Hz, 4H), 4.29 (t, J=5.7 Hz, 4H).

### Example 23. Preparation of 2,2′-((2,2′-dichloro-[1,1′-biphenyl]-3,3′-diyl)bis(4-oxopyrrolo[2,1-f][1,2,4]triazine-6,3(4H)-diyl))diacetic acid (Compound 23)

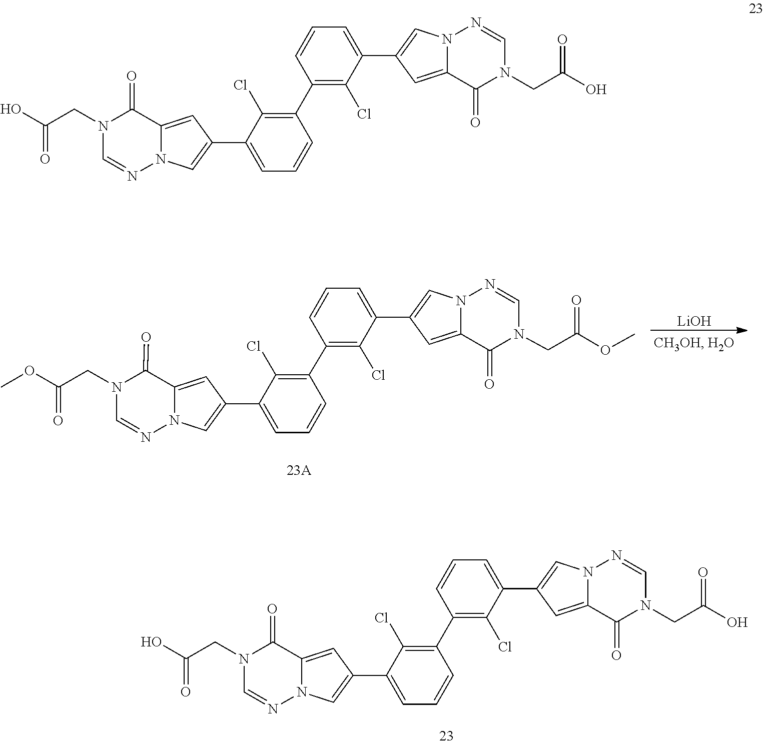

Following the procedure of Compound 1, Compound 23A was obtained by substituting methyl 2-bromoacetate for 1D. Compound 23A (30 mg, 0.05 mmol, 1.0 eq.) and LiOH (4.5 mg, 0.2 mmol, 4 eq) were added into methanol (2.0 mL) and H₂O (0.5 mL), and stirred 1 h. The mixture was purified by prep-HPLC to give compound 23 (1.1 mg, yield: 3.8%). LCMS (ESI): calculated for C₂₈H₁₈Cl₂N₆O₆; [M+H]+: 605.1, 607.1, found: 605.1, 607.1. ¹H NMR (500 Mhz, DMSO-d6) ¹H NMR (500 MHz, DMSO-d6) δ 8.54 (s, 2H), 7.99 (t, J=1.7 Hz, 4H), 7.73-7.66 (m, 4H), 7.48 (t, J=7.6 Hz, 2H), 7.32 (dd, J=7.5, 1.6 Hz, 2H), 7.28 (d, J=1.7 Hz, 2H), 4.56 (d, J=5.8 Hz, 4H)

### Example 24. Preparation of 3,3′-((2,2′-dichloro-[1,1′-biphenyl]-3,3′-diyl)bis(4-oxopyrrolo[2,1-f][1,2,4]triazine-6,3(4H)-diyl))dipropionic acid (Compound 24)

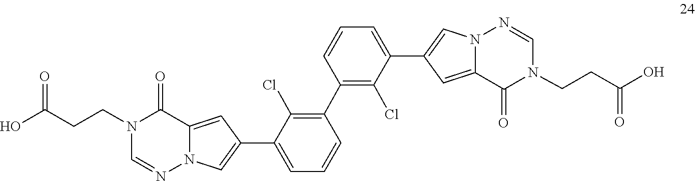 [SMILES: O=C(O)CCn1cnn2cc(-c3cccc(-c4cccc(-c5cc6c(=O)n(CCC(=O)O)cnn6c5)c4Cl)c3Cl)cc2c1=O]

Following the procedure of Compound 23, the title Compound 24 was obtained by methyl 3-bromopropanoate for methyl 2-bromoacetate. LCMS (ESI): calculated for C₃OH₂₂Cl₂N₆O₆; [M+H]+: 633.1, 635.1, found: 633.1, 635.1. ¹H NMR (500 Mhz, DMSO-d6) δ 8.61 (d, J=1.6 Hz, 2H), 8.28 (s, 2H), 7.94 (d, J=1.6 Hz, 2H), 7.68 (m, 4H), 7.61-7.31 (m, 2H), 4.22 (dq, J=25.6, 5.9 Hz, 2H), 2.68 (t, J=5.9 Hz, 2H).

### Example 25. Preparation of 6,6′-(2,2′-dichloro-[1,1′-biphenyl]-3,3′-diyl)bis(3-(2-(2H-1,2,3-triazol-2-yl)eth yl)pyrrolo[2,1-f][1,2,4]triazin-4(3H)-one) (Compound 25)

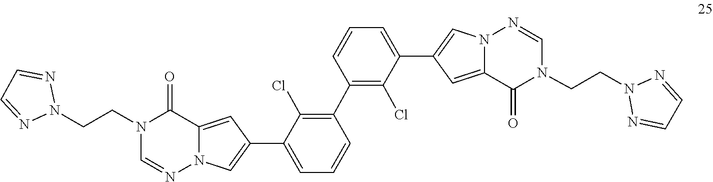 [SMILES: O=c1c2cc(-c3cccc(-c4cccc(-c5cc6c(=O)n(CCn7nccn7)cnn6c5)c4Cl)c3Cl)cn2ncn1CCn1nccn1]

Following the procedure of Compound 1, the title Compound 25 was obtained by substituting 2-(2H-1,2,3-triazol-2-yl)ethan-1-ol for 1D-1. LCMS (ESI): calculated for C₃₂H₂₄Cl₂N₁₂O₂; [M+H]+: 679.1, 681.1, found: 679.1, 681.1. 1H NMR (500 Mhz, DMSO-d6) δ 8.54 (s, 2H), 7.99 (t, J=1.7 Hz, 4H), 7.73-7.66 (m, 4H), 7.48 (t, J=7.6 Hz, 2H), 7.32 (dd, J=7.5, 1.6 Hz, 2H), 7.28 (d, J=1.7 Hz, 2H), 4.56 (d, J=5.8 Hz, 4H), 4.29 (t, J=5.7 Hz, 4H).

### Example 26. Preparation of 6,6′-(2,2′-dichloro-[1,1′-biphenyl]-3,3′-diyl)bis(3-(2-(4-methylpiperazin-1-yl)ethyl)pyrrolo[2,1-f][1,2,4]triazin-4(3H)-one) (Compound 26)

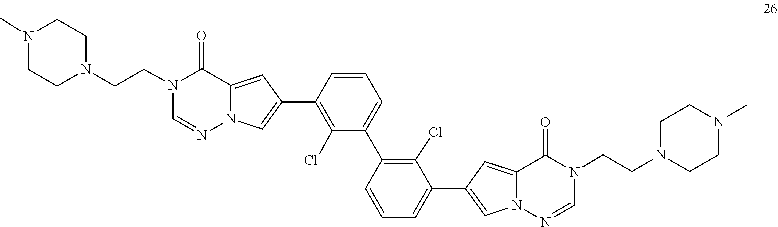

Following the procedure of Compound 1, the title Compound 26 was obtained by substituting 2-(4-methylpiperazin-1-yl)ethan-1-ol for 1D-1. LCMS (ESI): calculated for C₃₈H₄₂Cl₂N₁₀O₂; [M+H]+: 741.3, 743.3, found: 741.3, 743.3. ¹H NMR (500 Mhz, DMSO-d₆) δ 8.41 (d, J=1.6 Hz, 2H), 8.19 (d, J=5.1 Hz, 2H), 8.01 (d, J=1.6 Hz, 2H), 7.68-7.65 (m, 4H), 7.52-7.49 (m, 2H), 4.06 (t, J=6.4 Hz, 1.6 Hz, 4H), 2.64-2.55 (m, 20H), 2.29 (s, 6H).

### Example 27. Preparation of 6,6′-(2,2′-dichloro-[1,1′-biphenyl]-3,3′-diyl)bis(3-((5-oxotetrahydrofuran-2-yl)methyl)pyrrolo[2,1-f][1,2,4]triazin-4(3H)-one) (Compound 27)

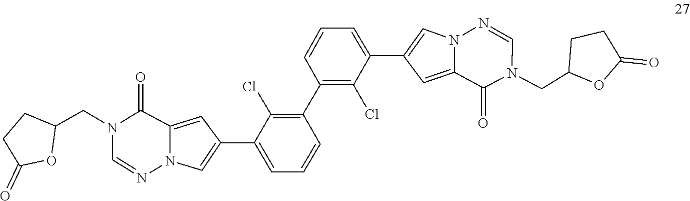 [SMILES: O=C1CCC(Cn2cnn3cc(-c4cccc(-c5cccc(-c6cc7c(=O)n(CC8CCC(=O)O8)cnn7c6)c5Cl)c4Cl)cc3c2=O)O1]

Following the procedure of Compound 1, the title Compound 27 was obtained by substituting 5-(hydroxymethyl)dihydrofuran-2(3H)-one for 1D-1. LCMS (ESI): calculated for C₃₄H₂₆Cl₂N₆O₆; [M+H]+: 685.1, 687.1, found: 685.1, 687.1. ¹H NMR (500 Mhz, DMSO-d₆) δ 8.61 (d, J=1.6 Hz, 2H), 8.18 (d, J=5.1 Hz, 2H), 7.94 (d, J=1.6 Hz, 2H), 7.68 (m, 4H), 7.52 (m, 2H), 4.95 (m, 2H), 4.43 (m, 2H), 4.36 (m, 2H), 2.40-2.13 (m, 6H), 2.13-1.93 (m, 2H).

### Example 28. Preparation of 5,5′-((2,2′-dichloro-[1,1′-biphenyl]-3,3′-diyl)bis(4-oxopyrrolo[2,1-f][1,2,4]triazine-6,3(4H)-diyl))bis(4-hydroxypentanoic acid) (Compound 28)

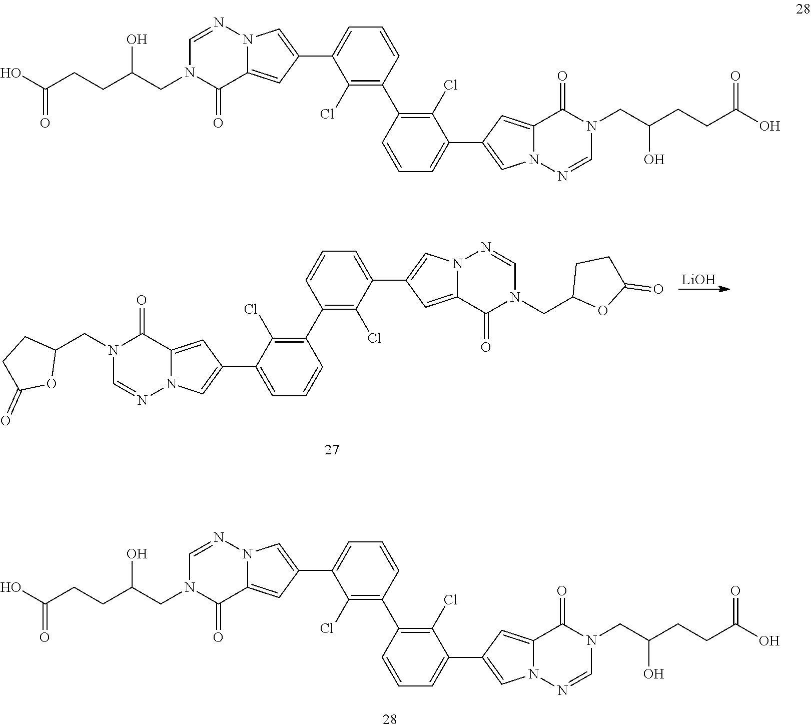

At 25° C., compound 27 (5.0 mg, 7.3 umol, 1.0 eq.) and LiOH (0.7 mg, 30 umol, 4.0 eq.) was dissolved into MeOH/H₂O (2 mL/0.5 mL), and stirred 1 h.

The reaction solution was purified by preparative liquid phase chromatography to give compound 28 (3.0 mg, yield: 57%). LCMS (ESI): calculated for C₃₄H₃₀Cl₂N₆O₈; [M+H]+: 721.1, 723.1, found: 721.1, 723.1. ¹H NMR (500 MHz, DMSO-d6) δ 8.60 (d, J=1.6 Hz, 2H), 8.18 (d, J=5.1 Hz, 2H), 7.94 (d, J=1.6 Hz, 2H), 7.68-7.65 (m, 4H), 7.52-7.50 (m, 2H), 4.96-6.92 (m, 2H), 4.43-4.42 (m, 2H), 4.36-4.33 (m, 2H), 3.90 (s, 2H) 2.40-2.13 (m, 6H), 2.13-1.93 (m, 2H).

### Example 29. Preparation of 7,7′-(2,2′-dichloro-[1,1′-biphenyl]-3,3′-diyl)bis(3-methyl-2-(((S)-5-oxopyrrolidin-2-yl)methyl)pyrrolo[1,2-a]pyrazin-1(2H)-one) (Compound 29)

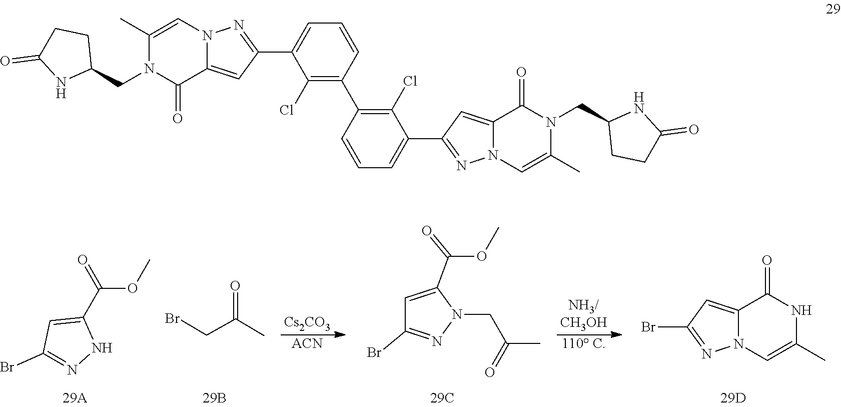

To a stirred mixture of Compound 29A (2.0 g, 9.8 mmol, 1.0 eq) in ACN (20 mL) were added Cs₂CO₃ (6.4 g, 19.6 mmol, 2.0 eq.) and Compound 29B (2.0 g, 14.7 mmol, 1.5 eq.) at 0° C. The resulting mixture was stirred for 1 h at 25° C. The reaction was quenched with water and extracted with EtOAc (50 mL×3). The combined organic layers were washed with brine (100 mL), dried over anhydrous sodium sulfate. After filtration, the filtrate was concentrated under reduced pressure. The residue was purified by silica gel column chromatography, eluted with PE/EtOAc (3:1) to afford Compound 29C (1.4 g, yield: 54.7%). LCMS (ESI): calculated for C₈H₉BrN₂O₃; [M+H]+: 261.1, 263.1, found: 261.1, 263.1.

A mixture of the Compound 29C (1.4 g, 5.36 mmol, 1.0 eq) in NH₃/MeOH (7.0 M, 30 mL) was stirred in a sealed tube at 110° C. for 2 h. After concentration, the residue was purified by flash column chromatography eluting with DCM/MeOH (10:1) to afford Compound 29D (1.0 g, yield: 81.9%). LCMS (ESI): calculated for C₇H₆BrN₃O; [M+H]+: 228.1, 230.1, found: 228.1, 230.1.

Following the procedure of Compound 1, the title Compound 29 was obtained by substituting 29D for 1B. LCMS (ESI): calculated for C₃₆H₃₂Cl₂N₈O₄; [M+H]+: 711.2, 713.2, found: 711.2, 713.2. ¹H NMR (500 MHz, DMSO-d6) δ 7.87 (d, J=2.5 Hz, 2H), 7.81 (s, 2H), 7.78 (s, 2H), 7.56 (t, J=10.0 Hz, 2H), 7.47 (d, J=10.0 Hz, 2H), 7.35 (s, 2H), 4.06-3.98 (m, 4H), 3.94-3.88 (m, 2H), 2.37-2.32 (m, 2H), 2.32 (s, 6H), 2.19-2.14 (m, 2H), 2.09-2.05 (m, 2H), 1.81-1.75 (m, 2H).

### Example 30. Preparation of 7,7′-(2,2′-dichloro-[1,1′-biphenyl]-3,3′-diyl)bis(3-methyl-2-(((S)-5-oxopyrrolidin-2-yl)methyl)pyrrolo[1,2-a]pyrazin-1(2H)-one) (Compound 30)

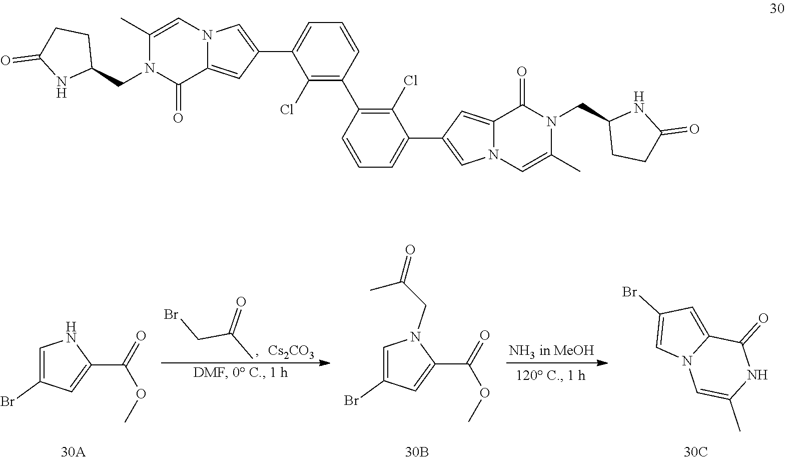

Following the procedure of Compound 29, the title Compound 30C was obtained by substituting methyl 4-bromo-1H-pyrrole-2-carboxylate for 29A, LCMS (ESI): calculated for C₈H₇BrN₂O; [M+H]+: 226.0, 228.0, found: 226.0, 228.0.

Following the procedure of Compound 1, the title Compound 30 was obtained by substituting 30C for 1B. LCMS (ESI): calculated for C₃₈H₃₄Cl₂N₆O₄; [M+H]+: 709.2, 711.2, found: 709.2, 711.2. ¹H NMR (500 Mhz, DMSO-d6) δ 7.87 (s, 2H), 7.79 (s, 2H), 7.71-7.65 (m, 2H), 7.51-7.42 (m, 4H), 7.33-7.27 (m, 2H), 6.87 (d, J=5.9 Hz, 2H), 4.02-3.94 (m, 3H), 3.76-3.69 (m, 3H), 2.28 (s, 6H), 2.23-2.14 (m, 2H), 2.13-2.02 (m, 4H), 1.81-1.72 (m, 2H).

### Example 31. Preparation of 7,7′-(2,2′-dichloro-[1,1′-biphenyl]-3,3′-diyl)bis(3-cyclopropyl-2-(((S)-5-oxopyrrolidin-2-yl)methyl)pyrrolo[1,2-a]pyrazin-1(2H)-one) (Compound 31)

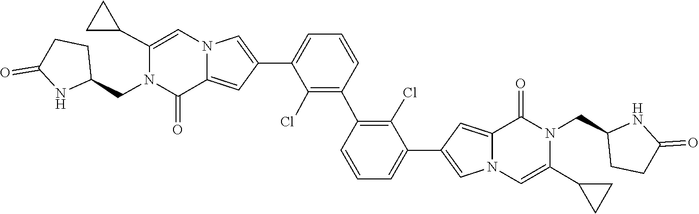 [SMILES: O=C1CC[C@@H](Cn2c(C3CC3)cn3cc(-c4cccc(-c5cccc(-c6cc7c(=O)n(C[C@@H]8CCC(=O)N8)c(C8CC8)cn7c6)c5Cl)c4Cl)cc3c2=O)N1]

Following the procedure of Compound 30, the title Compound 31 was obtained by substituting 2-bromo-1-cyclopropylethan-1-one for 1-bromopropan-2-one. LCMS (ESI): calculated for C₄₂H₃₈Cl₂N₆O₄; [M+H]+: 761.2, 763.3, found: 761.2, 763.3. ¹H NMR (500 Mhz, DMSO-d₆) δ 7.83 (s, 2H), 7.76 (s, 2H), 7.66-7.64 (m, 2H), 7.51-7.48 (m, 4H), 7.29-7.28 (m, 2H), 7.19 (s, 2H), 4.15-4.14 (m, 4H), 4.06 (s, 2H), 2.32-2.30 (m, 2H), 2.16-2.10 (m, 4H), 1.86-1.81 (m, 4H), 0.96-0.94 (m, 4H), 0.82-0.81 (m, 2H), 0.65-0.63 (m, 2H).

### Example 32. Preparation of 2,2′-(2,2′-dichloro-[1,1′-biphenyl]-3,3′-diyl)bis(6-cyclopropyl-5-(((S)-5-oxopyrrolidin-2-yl)methyl)pyrazolo[1,5-a]pyrazin-4(5H)-one) (Compound 32)

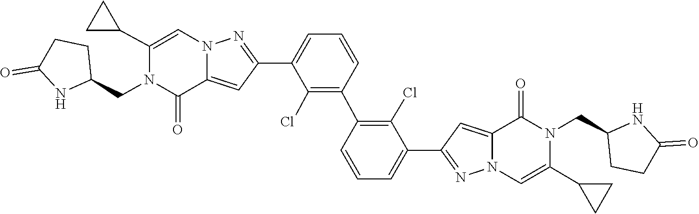 [SMILES: O=C1CC[C@@H](Cn2c(C3CC3)cn3nc(-c4cccc(-c5cccc(-c6cc7c(=O)n(C[C@@H]8CCC(=O)N8)c(C8CC8)cn7n6)c5Cl)c4Cl)cc3c2=O)N1]

Following the procedure of Compound 29, the title Compound 32 was obtained by substituting 2-bromo-1-cyclopropylethan-1-one for 1-bromopropan-2-one. LCMS (ESI): calculated for C₄₀H₃₆Cl₂N₈O₄; [M+H]+: 763.2, 765.2, found: 763.2, 765.2. ¹H NMR (500 Mhz, DMSO-d₆) δ 7.87 (d, J=2.5 Hz, 2H), 7.81 (s, 2H), 7.78 (s, 2H), 7.56 (t, J=10.0 Hz, 2H), 7.47 (d, J=10.0 Hz, 2H), 7.35 (s, 2H), 4.06-3.98 (m, 4H), 3.94-3.88 (m, 2H), 2.35-2.33 (m, 2H), 2.16-2.10 (m, 4H), 1.88-1.85 (m, 4H), 0.97-0.93 (m, 4H), 0.83-0.82 (m, 2H), 0.65-0.61 (m, 2H).

### Example 33. Preparation of 2,2′-(2,2′-dichloro-[1,1′-biphenyl]-3,3′-diyl)bis(6-ethyl-5-(((S)-5-oxopyrrolidin-2-yl)methyl)pyrazolo[1,5-a]pyrazin-4(5H)-one) (Compound 33)

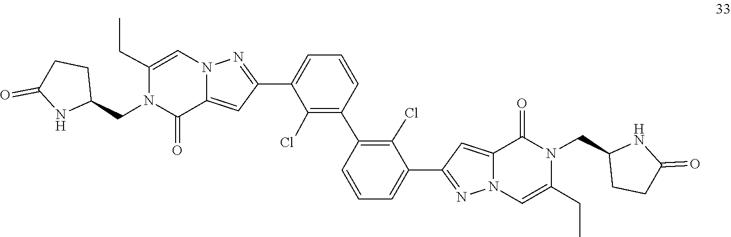 [SMILES: CCc1cn2nc(-c3cccc(-c4cccc(-c5cc6c(=O)n(C[C@@H]7CCC(=O)N7)c(CC)cn6n5)c4Cl)c3Cl)cc2c(=O)n1C[C@@H]1CCC(=O)N1]

Following the procedure of Compound 29, the title Compound 33 was obtained by substituting 1-bromobutan-2-one for 1-bromopropan-2-one. LCMS (ESI): calculated for C₃₈H₃₆Cl₂N₈O₄; [M+H]+: 739.2, 741.2, found: 739.2, 741.2. ¹H NMR (500 Mhz, DMSO-d₆) δ 7.86 (d, J=2.5 Hz, 2H), 7.82 (s, 2H), 7.78 (s, 2H), 7.57 (t, J=10.0 Hz, 2H), 7.46 (d, J=10.0 Hz, 2H), 7.36 (s, 2H), 4.05-3.99 (m, 4H), 3.95-3.90 (m, 2H), 2.35-2.32 (m, 2H), 2.29-2.31 (m, 4H), 2.19-2.14 (m, 2H), 2.12-2.10 (m, 6H), 2.09-2.05 (m, 2H), 1.81-1.75 (m, 2H).

### Example 34. Preparation of 6,6′-(2,2′-dichloro-[1,1′-biphenyl]-3,3′-diyl)bis(2-cyclopropyl-3-(((S)-5-oxopyrrolidin-2-yl)methyl)pyrrolo[2,1-f][1,2,4]triazin-4(3H)-one) (Compound 34)

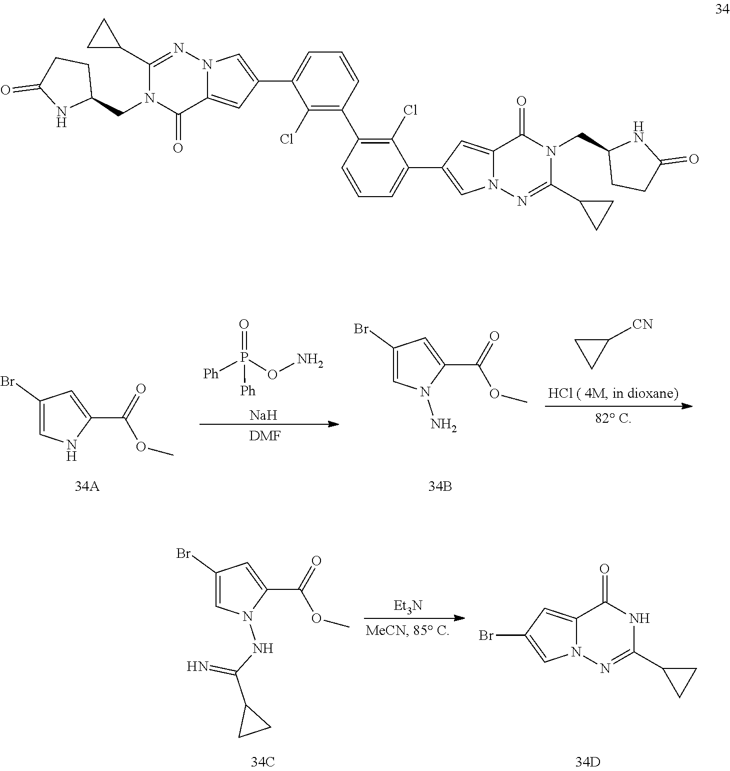

To a solution of 34 A (4.73 g, 23.18 mmol, 1.0 eq) in DMF (100 mL) was added NaH (1.11 g, 60% in oil, 27.9 mmol, 1.2 eq) at 0° C. and stirred for 10 min. Then (aminooxy)diphenylphosphine oxide (6.5 g, 27.9 mmol, 1.2 eq) was added to the mixture at 0° C., the reaction was stirred for 15 h at 25° C. The mixture was poured into water (100 mL) and extracted with EtOAc (50 mL×2). The leyers was concentrated under reduce pressure. The residue was purified by silica gel column chromatogram (PE:EtOAc=1:0-10:1) to afford 38B, 4.99 g. yield: 98.8%. LCMS (ESI): calculated for C₆H₇BrN₂O₂; [M+H]+: 219.04, 221.03 found: 219.35, 221.38.

To a solution of 34B (1.2 g, 5.50 mmol, 1 eq) in cyclopropanecarbonitrile (1.53 g, 22.83 mmol, 4.15 eq.) was added HCl (4.0 M in dioxane, 3.5 mL, 16.5 mmol, 3 eq) at 25° C. and the mixture was stirred for 15 h at 82° C. The reaction mixture was concentrated under reduce pressure to afford 34C (1.56 g. yield: 100%). LCMS (ESI): calculated for C₁₀H₁₂BrN₃O₂; [M+H]+: 285.0, 287.0, found: 285.0, 287.0.

To a solution of 34C (1.56 g, 5.47 mmol, 1.0 eq) in MeCN (15 mL) was added Et₃N (2.0 mL) at 25° C. and the mixture was stirred for 7 h at 85° C. The mixture was concentrated under reduce pressure. The residue was purified by silica gel column chromatogram (PE:EtOAc=5:1-2:1) to afford 34D (1.28 g, yield: 92%). LCMS (ESI): calculated for C₉H₈BrN₃O; [M+H]+: 254.09, 256.13 found: 254.00, 256.02.

Following the procedure of Compound 1, the title Compound 34 was obtained by substituting 34D for 1B. LCMS (ESI): calculated for C₃₈H₃₈Cl₂N₆O₈; [M+H]*: 830.72, found: 830.41. ¹H NMR (500 MHz, DMSO-d6) δ 8.23 (s, 2H), 7.85 (s, 2H), 7.75-7.68 (m, 2H), 7.52 (t, J=7.6 Hz, 2H), 7.39 (s, 2H), 7.33 (d, J=7.4 Hz, 2H), 4.12-4.05 (m, 2H), 3.94-3.89 (m, 6H), 2.27-2.22 (m, 2H), 2.16-2.08 (m, 4H), 1.97-2.94 (M, 8H), 1.79-1.77 (m, 2H).

### Example 35. Preparation of 6,6′-(2,2′-dichloro-[1,1′-biphenyl]-3,3′-diyl)bis(2-methyl-3-(((S)-5-oxopyrrolidin-2-yl)methyl)pyrrolo[2,1-f][1,2,4]triazin-4(3H)-one) (Compound 35)

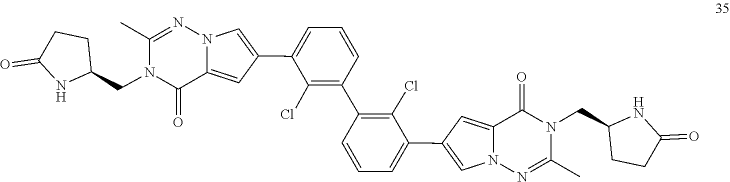 [SMILES: Cc1nn2cc(-c3cccc(-c4cccc(-c5cc6c(=O)n(C[C@@H]7CCC(=O)N7)c(C)nn6c5)c4Cl)c3Cl)cc2c(=O)n1C[C@@H]1CCC(=O)N1]

Following the procedure of Compound 34, the title Compound 35 was obtained by substituting acetonitrile for cyclopropanecarbonitrile. LCMS (ESI): calculated for C₃₆H₃₂Cl₂N₈O₄; [M+H]*: 711.2, 713.2, found: 711.2, 713.2. ¹H NMR (500 Mhz, DMSO-d₆): 8.12 (s, 2H), 8.03 (d, J=1.2 Hz, 2H), 7.85 (s, 2H), 7.72 (d, J=6.4 Hz, 2H), 7.48 (t, J=6.4 Hz, 2H), 7.25 (d, J=1.6 Hz, 2H), 3.94-3.88 (m, 4H), 3.02 (s, 6H), 2.27-2.21 (m, 2H), 2.15-2.08 (m, 4H), 1.77-1.72 (m, 2H).

### Example 36. Preparation of 2,2′-(2,2′-dichloro-[1,1′-biphenyl]-3,3′-diyl)bis(6-methyl-5-(((S)-pyrrolidin-2-yl)methyl)pyrazolo[1,5-a]pyrazin-4(5H)-one) dihydrochloride (Compound 36)

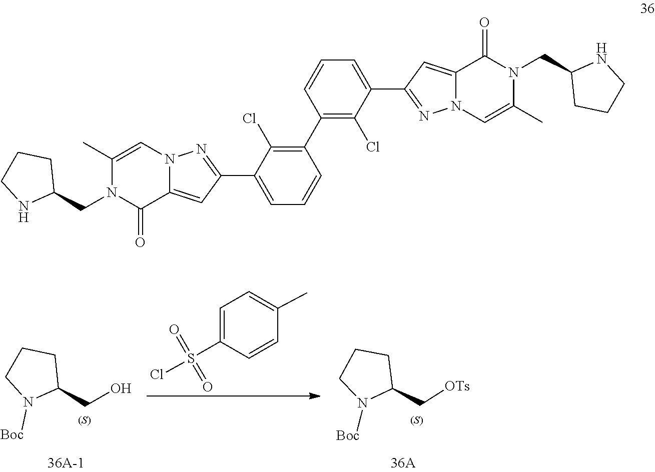

To a stirred mixture of Compound 36A-1 (3.0 g, 14.9 mmol, 1.0 eq.) in DCM (30 mL) were added trimethylamine (3.01 g, 29.9 mmol, 2 eq.), DMAP (0.18 g, 1.5 mmol, 0.1 eq) and 4-methylbenzenesulfonyl chloride (3.70 g, 19.4 mmol, 1.30 eq) at 0° C. The resulting mixture was stirred for 16 h at 25° C. the reaction was quenched with water (50 mL) and extracted with DCM (50 mL×3). The combined organic layers were washed with brine (20 mL), dried over anhydrous sodium sulfate. After filtration, the filtrate was concentrated under reduced pressure. This resulted in Compound 36A (5.0 g. yield: 94.5%). LCMS (ESI): calculated for C₁₇H₂₅NO₅S; [M+H]+: 356.1, found: 356.1.

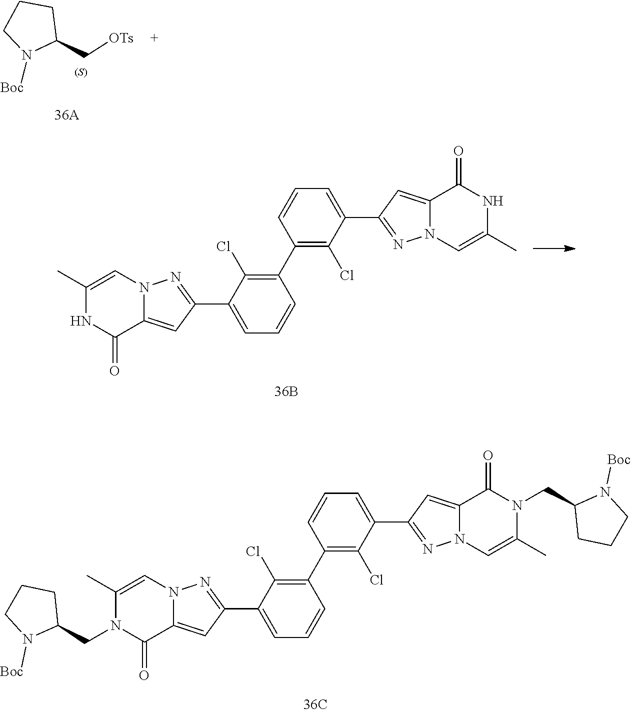

Following the procedure of Compound 1, the title Compound 36C was obtained by substituting 29D for 1B, 36A for 1D.

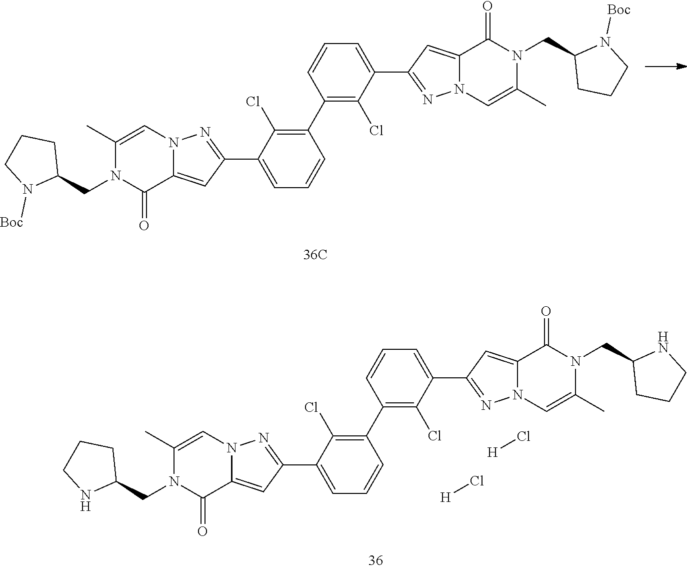

To a stirred mixture of Compound 36C (30 mg, 0.03 mmol, 1.0 eq) in DCM (10 mL) was added HCl in dioxane (4.0 M, 3.0 mL) at 25° C. The resulting mixture was stirred for 3 h at 25° C. and concentrated under reduced pressure. The residue was dissolved in ACN/H₂O (1:4) (8.0 mL) and lyophilized to give Compound 36 (24.6 mg, yield: 96.1%). LCMS (ESI): calculated for C₃H₃₅Cl₄N₈O₂; [M+H]+: 683.3, 685.3, found: 683.3, 685.3. ¹H NMR (500 MHz, DMSO-d₆) δ 7.90-7.84 (m, 4H), 7.60-7.54 (m, 2H), 7.49-7.44 (m, 2H), 7.40-7.36 (m, 2H), 4.45-4.35 (m, 2H), 4.29-4.21 (m, 2H), 3.74-3.64 (m, 2H), 3.52-3.43 (m, 4H), 3.13-3.02 (i, 2H), 2.39 (s, 6H), 2.19-2.09 (m, 2H), 2.06-1.93 (m, 2H), 1.92-1.86 (i, 2H), 1.80-1.70 (m, 2H).

### Example 37. Preparation of 2,2′-(2,2′-dichloro-[1,1′-biphenyl]-3,3′-diyl)bis(6-methyl-5-(((R)-pyrrolidin-2-yl)methyl)pyrazolo[1,5-a]pyrazin-4(5H)-one) dihydrochloride (Compound 37)

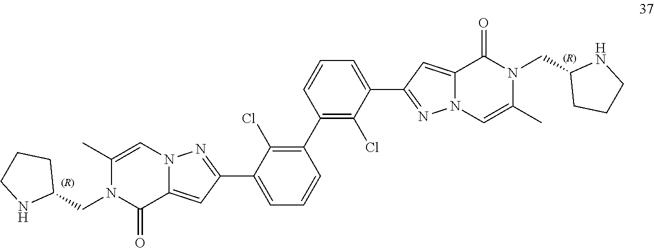

Following the procedure of Compound 36, the title Compound 37 was obtained by substituting tert-butyl (R)-2-(hydroxymethyl)pyrrolidine-1-carboxylate for 36A-1. LCMS (ESI): calculated for C₃₆H₃₈Cl₄N₈O₂; [M+H]+: 683.3, 685.3, found: 683.3, 685.3. ¹H NMR (500 MHz, DMSO-d₆) δ 7.90-7.84 (m, 4H), 7.61-7.54 (m, 2H), 7.49-7.43 (m, 2H), 7.40-7.36 (m, 2H), 4.45-4.35 (m, 2H), 4.29-4.21 (m, 2H), 3.74-3.64 (m, 2H), 3.52-3.43 (m, 4H), 3.12-3.02 (m, 2H), 2.39 (s, 6H), 2.19-2.09 (m, 2H), 2.06-1.93 (m, 2H), 1.92-1.87 (m, 2H), 1.80-1.71 (m, 2H).

### Example 38. Preparation of 7,7′-(2,2′-dichloro-[1,1′-biphenyl]-3,3′-diyl)bis(3-methyl-2-(((S)-pyrrolidin-2-yl)methyl)pyrrolo[1,2-a]pyrazin-1(2H)-one) (Compound 38)

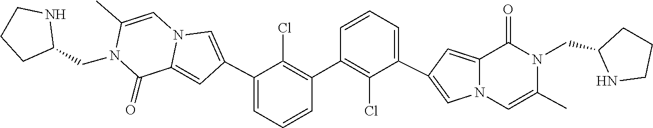 [SMILES: Cc1cn2cc(-c3cccc(-c4cccc(-c5cc6c(=O)n(C[C@@H]7CCCN7)c(C)cn6c5)c4Cl)c3Cl)cc2c(=O)n1C[C@@H]1CCCN1]

Following the procedure of Compound 29 and Compound 36, the title Compound 38 was obtained by substituting methyl 4-bromo-1H-pyrrole-2-carboxylate for 29A. LCMS (ESI): calculated for C₃₆H₃₈Cl₄N₈O₂; [M+H]+: 681.3, 683.3, found: 681.3, 683.3. ¹H NMR (500 MHz, DMSO-d₆) δ 8.13 (s, 2H), 7.91-7.86 (m, 4H), 7.62-7.55 (m, 2H), 7.49-7.44 (m, 2H), 7.42-7.37 (m, 2H), 4.45-4.35 (m, 2H), 4.29-4.23 (m, 2H), 3.74-3.66 (m, 2H), 3.52-3.47 (m, 4H), 3.13-3.02 (m, 2H), 2.32 (s, 6H), 2.17-2.08 (m, 2H), 2.06-1.95 (m, 2H), 1.93-1.89 (m, 2H), 1.80-1.74 (m, 2H).

### Example 39. Preparation of 7,7′-(2,2′-dichloro-[1,1′-biphenyl]-3,3′-diyl)bis(3-methyl-2-(((R)-pyrrolidin-2-yl)methyl)pyrrolo[1,2-a]pyrazin-1(2H)-one) (Compound 39)

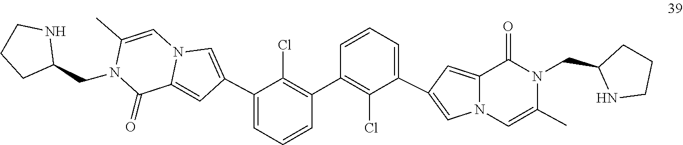 [SMILES: Cc1cn2cc(-c3cccc(-c4cccc(-c5cc6c(=O)n(C[C@H]7CCCN7)c(C)cn6c5)c4Cl)c3Cl)cc2c(=O)n1C[C@H]1CCCN1]

Following the procedure of Compound 38, the title Compound 38 was obtained by substituting tert-butyl (R)-2-(hydroxymethyl)pyrrolidine-1-carboxylate for 36A-1. LCMS (ESI): calculated for C₃₆H₃₈Cl₄N₈O₂; [M+H]+: 681.3, 683.3, found: 681.3, 683.3. ¹H NMR (500 MHz, DMSO-d₆) δ 8.13 (s, 2H), 7.91-7.86 (m, 4H), 7.62-7.55 (m, 2H), 7.49-7.44 (m, 2H), 7.42-7.37 (m, 2H), 4.45-4.35 (m, 2H), 4.29-4.23 (m, 2H), 3.74-3.66 (m, 2H), 3.52-3.47 (m, 4H), 3.13-3.02 (m, 2H), 2.32 (s, 6H), 2.17-2.08 (m, 2H), 2.06-1.95 (m, 2H), 1.93-1.89 (m, 2H), 1.80-1.74 (m, 2H).

### Example 40. Preparation of 7,7′-(2,2′-dichloro-[1,1′-biphenyl]-3,3′-diyl)bis(3-methyl-2-(((R)-pyrrolidin-2-yl)methyl)pyrrolo[1,2-a]pyrazin-1(2H)-one) (Compound 40)

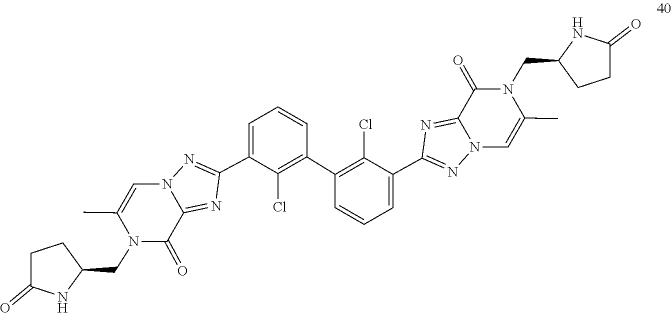 [SMILES: Cc1cn2nc(-c3cccc(-c4cccc(-c5nc6c(=O)n(C[C@@H]7CCC(=O)N7)c(C)cn6n5)c4Cl)c3Cl)nc2c(=O)n1C[C@@H]1CCC(=O)N1]

Following the procedure of Compound 29, the title Compound 40 was obtained by substituting methyl 5-bromo-1H-1,2,4-triazole-3-carboxylate for 29A. LCMS (ESI): calculated for C₃₄H₃₀Cl₂N₁₀O₄; [M+H]+: 713.2, 715.2, found: 713.2, 715.2. ¹H NMR (500 Mhz, DMSO-d6) δ 7.78 (s, 2H), 7.72-7.64 (m, 2H), 7.51-7.43 (m, 4H), 7.32-7.27 (m, 2H), 6.89 (d, J=5.9 Hz, 2H), 4.02-3.94 (m, 3H), 3.76-3.69 (m, 3H), 2.29 (s, 6H), 2.23-2.14 (m, 2H), 2.13-2.02 (m, 4H), 1.81-1.72 (m, 2H).

### Example 41. Preparation of (3S,3'S)-1,1′-(((2,2′-dichloro-[1,1′-biphenyl]-3,3′-diyl)bis(5-methyl-4-oxo-4,5-dihydropyrazolo[1,5-a]pyrazine-2,6-diyl))bis(methylene))bis(pyrrolidine-3-carboxylic acid) (Compound 41)

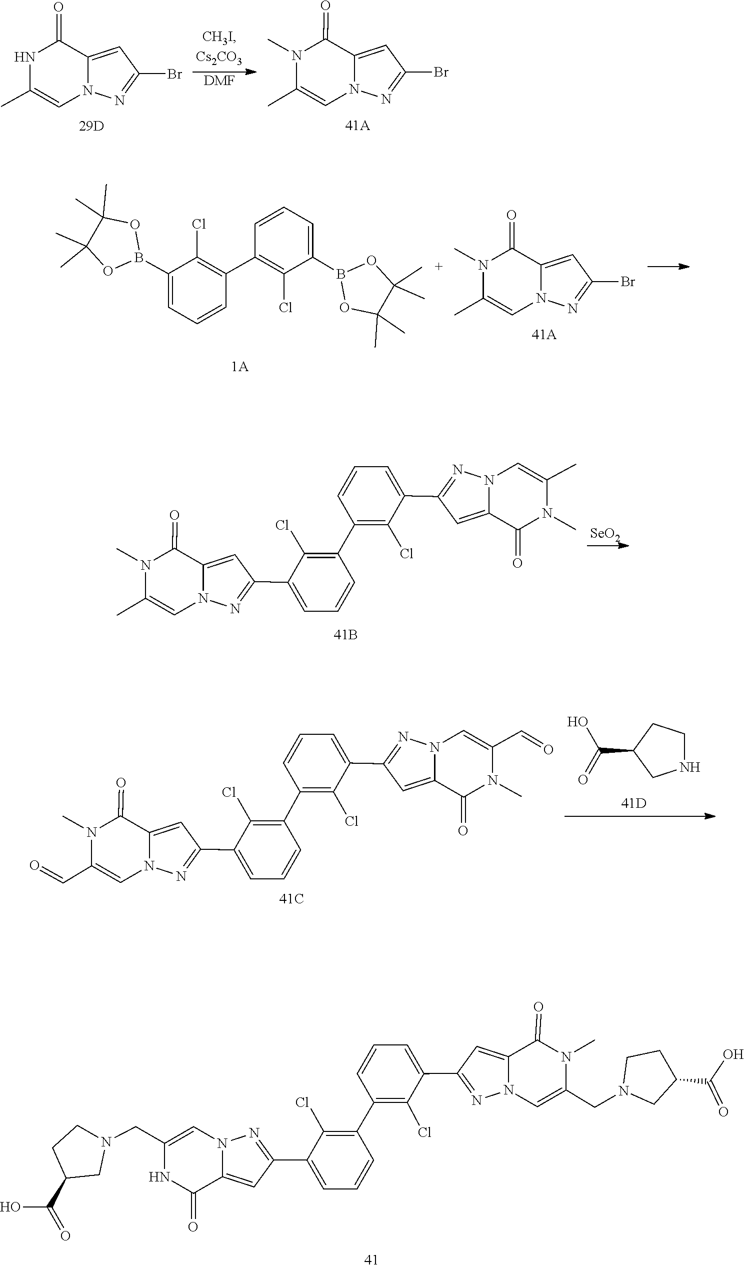

Compound 29D (1.0 g, 4.4 mmol, 1 eq.) and Cs₂CO₃ (2.14 g, 6.6 mmol, 1.5 eq.) were dissolved into DMF (10 mL). 0° C., CH₃I (0.75 g, 5.3 mmol, 1.2 eq.) was added to the reaction mixture, and stirred 1 h. Water (20 mL) was added to precipitate to get compound 41A (0.95 g, yield: 89.5%). LCMS (ESI): calculated for C₈H₈BrN₃O; [M+H]+: 242.0, 244.0, found: 242.0, 244.0.

Under N₂ atmosphere, Compound 1A (180 mg, 0.37 mmol, 1 eq.), Compound 41A (201 mg, 0.81 mmol, 2.2 eq.), Cs₂CO₃ (618 mg, 1.85 mmol, 5.0 eq.), Pd(dppf)Cl₂·DCM (62 mg, 0.07 mmol, 0.20 eq.), was dissolved into DMF (5.0 mL) and H₂O (1.0 mL). The reaction was carried out at 90° C. for 2 h. After cooling, 30 mL water were added and resulting mixture was filtered, the filter cake was washed with EtOAc (3 mL×3) to give compound 41B (180 mg, yield: 90.1%). LCMS (ESI): calculated for C₂₈H₂₂Cl₂N₆O₂; [M+H]+: 545.1, 546.1, found: 545.1, 546.1.

Compound 41B (100 mg, 0.19 mmol, 1.0 eq.), selenium dioxide (404 mg, 1.89 mmol, H₂O eq.) was dissolved into dioxane (35 mL). The reaction was carried out at 90° C. for 24 h. After cooling, 150 mL water and 100 mL DCM were added for extraction, and the organic phase was dried over sodium sulfite and concentrated to afford residue as compound 41C (80 mg, yield: 76.2%). LCMS (ESI): calculated for C₂₈H₁₈Cl₂N₆O₄; [M+H]+: 573.1, 575.1, found: 573.1, 575.1.

Compound 41C (40 mg, 0.07 mmol, 1 eq.), Compound 41D (80 mg, 0.7 mmol, 10 eq.) and titanium tetraisopropanolate (187 mg, 0.7 mmol, 10 eq.) was dissolved into THF (10 mL). The reaction was carried out at 60° C. for 2 h. After cooling to 25° C., NaBH₄ (26 mg, 0.7 mmol, 10 eq.) were added and the reaction was stirred at 25° C. After 2 h, 2 mL MeOH were added and resulting mixture was filtered, the filter cake was washed with MeOH (1 mL). The filtrate is collected and purify by prep-HPLC to give compound 41 (17.6 mg, yield: 32.6%). LCMS (ESI): calculated for C₃₈H₃₆Cl₂N₈O₆; [M+H]+: 771.1, 773.1, found: 771.1, 773.1. ¹H NMR (500 MHz, DMSO-d₆) δ 8.10 (s, 2H), 7.93-7.85 (m, 2H), 7.59-7.56 (m, 2H), 7.52-7.47 (m, 2H), 7.45 (s, 2H), 3.56-3.47 (m, 4H), 3.41 (s, 6H), 3.25-3.16 (m, 4H), 3.14-3.06 (m, 4H), 3.05-2.98 (m, 2H), 2.26-2.17 (m, 2H), 2.14-2.06 (m, 2H).

### Example 42. Preparation of 2,2′-(2,2′-dichloro-[1,1′-biphenyl]-3,3′-diyl)bis(6-(((2-hydroxyethyl)amino)m ethyl)-5-methylpyrazolo[1,5-a]pyrazin-4(5H)-one) (Compound 42)

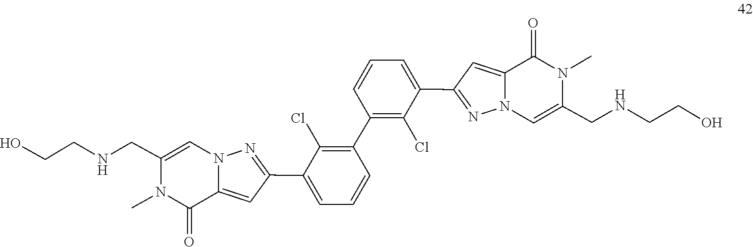 [SMILES: Cn1c(CNCCO)cn2nc(-c3cccc(-c4cccc(-c5cc6c(=O)n(C)c(CNCCO)cn6n5)c4Cl)c3Cl)cc2c1=O]

Following the procedure of Compound 41, the title Compound 42 was obtained by substituting 2-aminoethan-1-ol for 41D. LCMS (ESI): calculated for C₃₂H₃₂Cl₂N₈O₄; [M+H]+: 663.2, 665.2, found: 663.2, 665.2. ¹H NMR (500 MHz, DMSO-d₆) δ 8.10 (s, 2H), 7.92-7.87 (m, 2H), 7.62-7.55 (m, 2H), 7.51-7.46 (m, 2H), 7.45 (s, 2H), 5.33 (s, 2H), 4.35 (s, 4H), 3.73-3.70 (m, 4H), 3.60-3.55 (m, 2H), 3.54 (s, 6H), 3.24-3.09 (m, 4H).

### Example 43. Preparation of (3S,3'S)-1,1′-(((2,2′-dichloro-[1,1′-biphenyl]-3,3′-diyl)bis(2-methyl-1-oxo-1,2-dihydropyrrolo[1,2-a]pyrazine-7,3-diyl))bis(methylene))bis(pyrrolidine-3-carboxylic acid) (Compound 43)

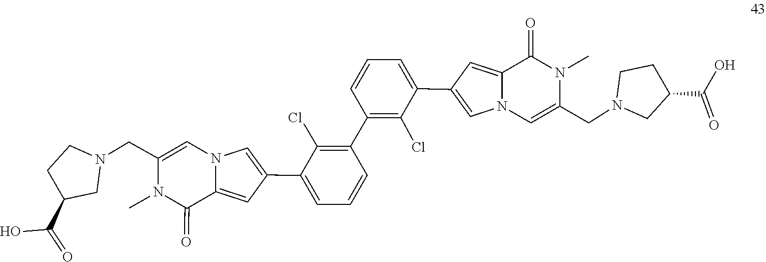 [SMILES: Cn1c(CN2CC[C@H](C(=O)O)C2)cn2cc(-c3cccc(-c4cccc(-c5cc6c(=O)n(C)c(CN7CC[C@H](C(=O)O)C7)cn6c5)c4Cl)c3Cl)cc2c1=O]

Following the procedure of Compound 41, the title Compound 43 was obtained by 30C for 29D. LCMS (ESI): calculated for C₄₀H₃₈Cl₂N₆O₆; [M+H]+: 769.2, 771.2, found: 769.2, 771.2. ¹H NMR (500 Mhz, DMSO-d₆) δ 7.90 (s, 2H), 7.69-7.68 (m, 4H), 7.49 (t, J=6 Hz, 2H), 7.37-7.32 (m, 4H), 3.72-3.52 (m, 16H), 3.44 (s, 6H).

### Example 44. Preparation of N,N′-(((((2,2′-dichloro-[1,1′-biphenyl]-3,3′-diyl)bis(2-methyl-1-oxo-1,2-dihydropyrrolo[1,2-a]pyrazine-7,3-diyl))bis(methylene))bis(azanediyl))bis(ethane-2,1-diyl))diacetamide (Compound 44)

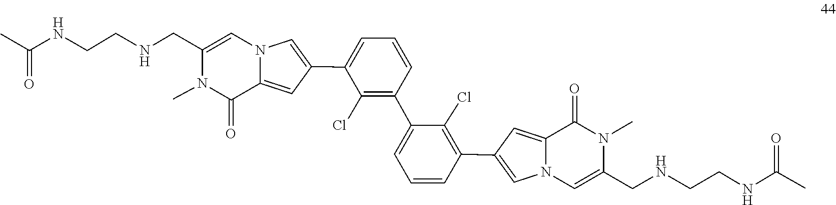 [SMILES: CC(=O)NCCNCc1cn2cc(-c3cccc(-c4cccc(-c5cc6c(=O)n(C)c(CNCCNC(C)=O)cn6c5)c4Cl)c3Cl)cc2c(=O)n1C]

Following the procedure of Compound 43, the title Compound 44 was obtained by substituting N-(2-aminoethyl)acetamide for (S)-pyrrolidine-3-carboxylic acid. LCMS (ESI): calculated for C₃₈H₄₀Cl₂N₈O₄; [M+H]+: 743.3, 745.3, found: 743.3, 745.3. ¹H NMR (500 MHz, DMSO-d6) δ 8.59 (s, 2H), 8.15 (t, J=5.7 Hz, 2H), 7.77 (d, J=1.8 Hz, 2H), 7.64 (dd, J=7.8, 1.7 Hz, 2H), 7.45 (t, J=7.6 Hz, 2H), 7.33 (s, 2H), 7.28 (dd, J=7.5, 1.7 Hz, 2H), 7.23 (d, J=1.7 Hz, 2H), 4.11 (s, 6H), 3.32-3.30 (m, 4H), 3.24-3.10 (m, 4H), 3.07-2.99 (m, 4H), 2.01 (s, 6H).

### Example 45. Preparation of 7,7′-(2,2′-dichloro-[1,1′-biphenyl]-3,3′-diyl)bis(3-(((2-hydroxyethyl)amino)m ethyl)-2-methylpyrrolo[1,2-a]pyrazin-1(2H)-one) (Compound 45)

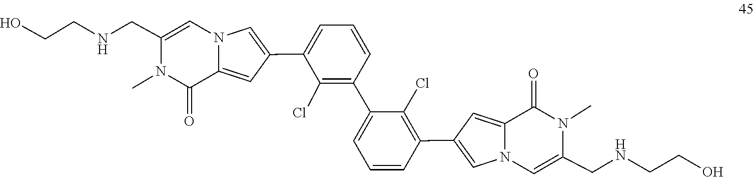 [SMILES: Cn1c(CNCCO)cn2cc(-c3cccc(-c4cccc(-c5cc6c(=O)n(C)c(CNCCO)cn6c5)c4Cl)c3Cl)cc2c1=O]

Following the procedure of Compound 43, the title Compound 45 was obtained by substituting 2-aminoethan-1-ol for (S)-pyrrolidine-3-carboxylic acid. LCMS (ESI): calculated for C34H₃₄Cl₂N₆O₄; [M+H]+: 661.2, 663.2, found: 661.2, 663.2. ¹H NMR (500 MHz, DMSO-d6) δ 8.59 (s, 2H), 8.14 (t, J=5.7 Hz, 2H), 7.76 (d, J=1.8 Hz, 2H), 7.63 (dd, J=7.8, 1.7 Hz, 2H), 7.42 (t, J=7.6 Hz, 2H), 7.34 (s, 2H), 7.26 (dd, J=7.5, 1.7 Hz, 2H), 4.09 (s, 6H), 3.62 (m, 2H), 3.35-3.31 (m, 4H), 3.23-3.18 (m, 4H), 3.06-2.97 (m, 4H).

### Example 46. Preparation of 7,7′-(2,2′-dichloro-[1,1′-biphenyl]-3,3′-diyl)bis(3-(((S)-3-hydroxypyrrolidin-1-yl)methyl)-2-methylpyrrolo[1,2-a]pyrazin-1(2H)-one) (Compound 46)

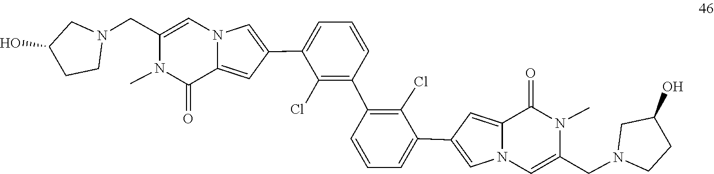 [SMILES: Cn1c(CN2CC[C@H](O)C2)cn2cc(-c3cccc(-c4cccc(-c5cc6c(=O)n(C)c(CN7CC[C@H](O)C7)cn6c5)c4Cl)c3Cl)cc2c1=O]

Following the procedure of Compound 43, the title Compound 46 was obtained by substituting (S)-pyrrolidin-3-ol for (S)-pyrrolidine-3-carboxylic acid. LCMS (ESI): calculated for C₃₈H₃₈Cl₂N₆O₄; [M+H]+: 713.2, 715.2, found: 713.2, 715.2. ¹H NMR (500 Mhz, DMSO-d₆) δ 7.90 (s, 2H), 7.71-7.70 (m, 4H), 7.47 (t, J=6 Hz, 2H), 7.32-7.26 (m, 4H), 3.70-3.52 (m, 16H), 3.42 (s, 6H).

### Example 47. Preparation of (3S,3'S)-1,1′-(((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(pyrrolidine-3-carboxylic acid) (Compound 47)

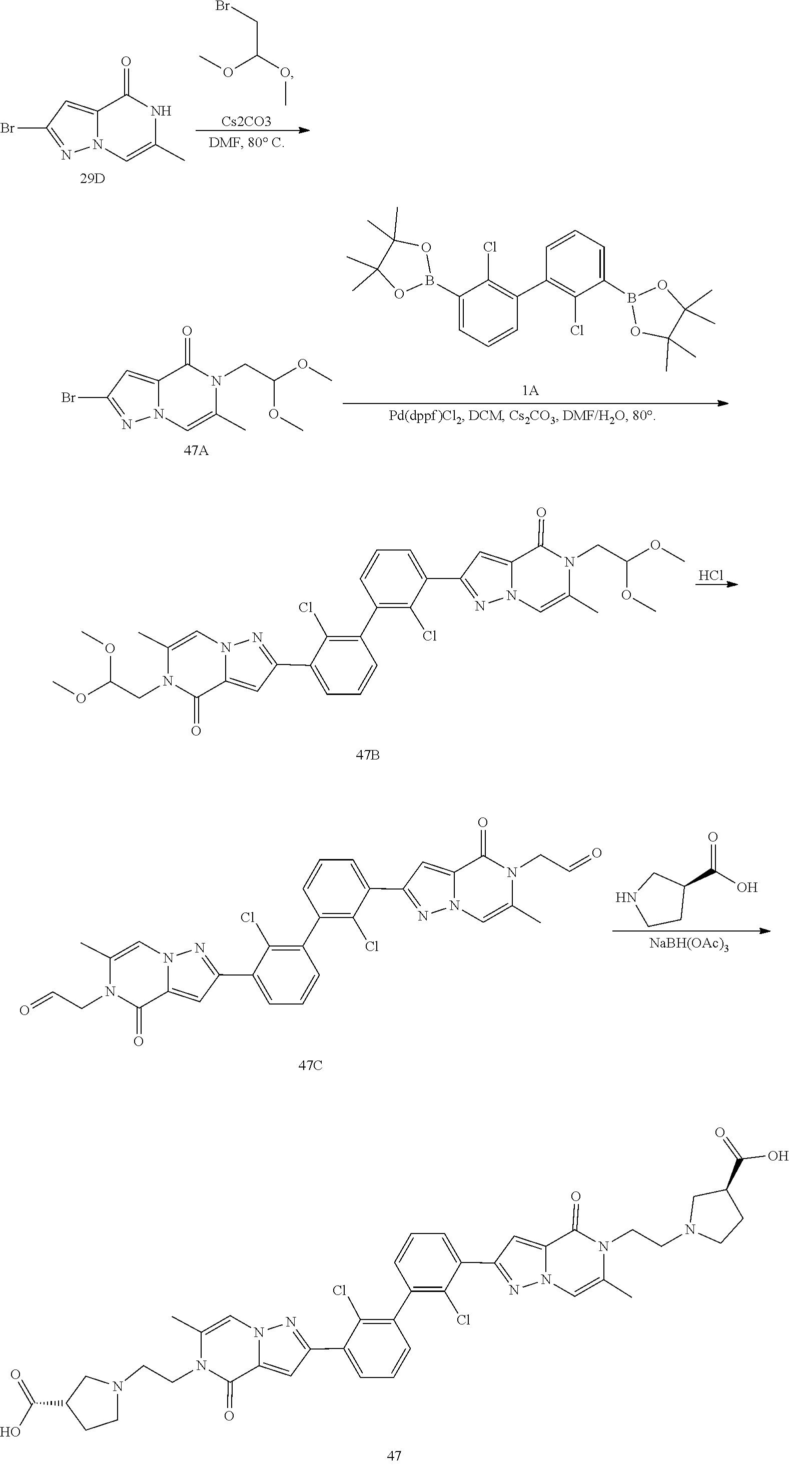

To a stirred mixture of Compound 29D (150 mg, 0.65 mmol, 1 eq) in DMF (2.0 mL) were added Cs₂CO₃ (536 mg, 1.64 mmol, 2.5 eq.) and 2-bromo-1,1-dimethoxyethane (221 mg, 1.32 mmol, 2 eq.) at 0° C. The resulting mixture was stirred for 16 h at 80° C. After cooling to 25° C., the reaction was quenched with water (10 mL) and extracted with EtOAc (10 mL×3). The combined organic layers were washed with brine (20 mL), dried over anhydrous sodium sulfate. After filtration, the filtrate was concentrated under reduced pressure. The residue was purified by silica gel column chromatography, eluted with PE/EtOAc (1:2) to afford Compound 47A, 95 mg. yield: 45.7%. LCMS (ESI): calculated for C₁₁H₁₄BrN₃O₃; [M+H]+: 316.1, 318.1, found: 284.1, 286.1.

To a stirred mixture of Compound 47A (95 mg, 0.30 mmol, 2.2 eq.) in DMF (5.00 mL) were added Compound 1A (65 mg, 0.14 mmol, 1 eq.), Pd(dppf)Cl₂-DCM (22 mg, 0.03 mmol, 0.2 eq.) and Cs₂CO₃ (223 mg, 0.70 mmol, eq.) in H₂O (1.0 mL) at 25° C. The resulting mixture was stirred for 2 h at 80° C. The mixture was allowed to cool down to 25° C. and quenched with water. The resulting mixture was extracted with EtOAc (30 mL×3). The combined organic layers were washed with brine (50 mL), dried over anhydrous sodium sulfate. After filtration, the filtrate was concentrated under reduced pressure. The residue was purified by silica gel column chromatography, eluted with PE/EtOAc (1:2) to afford Compound 47B (80 mg. yield: 84.4%). LCMS (ESI): calculated for C₃₄H₃₄Cl₂N₆O₆; [M+H]+: 693.2, 695.2, found: 693.2, 695.2.

To a stirred mixture of Compound 47B (65 mg, 0.09 mmol, 1 eq.) in HCl in 1,4-dioxane (1.0 M, 3.00 mL) were added H₂O (3.00 mL) at 25° C. The resulting mixture was stirred for 2 h at 85° C. The mixture was allowed to cool down to 25° C. and quenched with water. The resulting mixture was extracted with DCM (30 mL×3). The combined organic layers were washed with saturated sodium bicarbonate aqueous solution (50 mL), dried over anhydrous sodium sulfate. After filtration, the filtrate was concentrated under reduced pressure to give Compound 47C, 50 mg. yield: 89.2%. LCMS (ESI): calculated for C₃₀H₂₂Cl₂N₆O₄; [M+H]+: 601.2, 603.2, found: 601.2, 603.2.

To a stirred mixture of Compound 47C (30 mg, 0.05 mmol, 1.0 eq.) in DCM (10 mL) were added (S)-pyrrolidine-3-carboxylic acid (56 mg, 0.50 mmol, 10 eq.) and a drop of HOAc at 25° C. After stirred for 0.5 h at 25° C., sodium triacetoxyborohydride (211 mg, 1 mmol, 20 eq.) was added. The resulting mixture was stirred for 16 h at 25° C. The reaction mixture was concentrated, and the residue was dissolved in MeOH and purified by preparative liquid phase chromatography to give Compound 47 (16.6 mg, yield: 41.5%). LCMS (ESI): calculated for C₄₀H₄₀Cl₂N₈O₆; [M+H]+: 799.2, 801.2, found: 799.2, 801.2. 1H NMR (500 MHz, DMSO-d6) δ 7.87-7.81 (m, 2H), 7.78 (s, 2H), 7.73-7.68 (m, 2H), 7.59-7.52 (m, 2H), 7.47-7.43 (m, 2H), 3.92-3.72 (m, 4H), 2.93-2.83 (m, 4H), 2.81-2.64 (m, 8H), 2.64-2.56 (m, 2H), 2.30 (s, 6H), 1.97-1.87 (m, 2H), 1.83-1.74 (m, 2H).

### Example 48. Preparation of 7,7′-(2,2′-dichloro-[1,1′-biphenyl]-3,3′-diyl)bis(2-(2-((S)-3-hydroxypyrrolidin-1-yl)ethyl)-3-methylpyrrolo[1,2-a]pyrazin-1(2H)-one) (Compound 48)

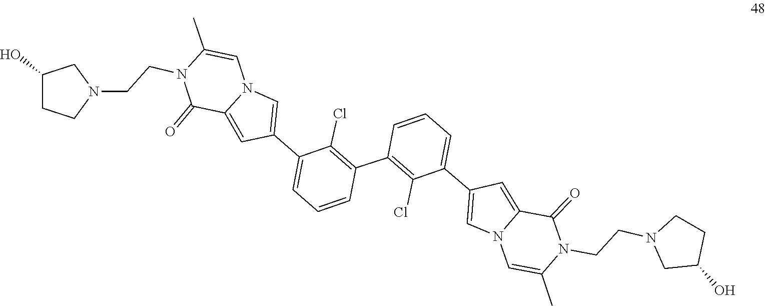 [SMILES: Cc1cn2cc(-c3cccc(-c4cccc(-c5cc6c(=O)n(CCN7CC[C@H](O)C7)c(C)cn6c5)c4Cl)c3Cl)cc2c(=O)n1CCN1CC[C@H](O)C1]

Following the procedure of Compound 47, the title Compound 48 was obtained by substituting 30C for 29D and (S)-pyrrolidin-3-ol for (S)-pyrrolidine-3-carboxylic acid. LCMS (ESI): calculated for C₄₀H₄₂Cl₂N₆O₄; [M+H]+: 741.7, 743.7, found: 741.7, 743.7. ¹H NMR (500 MHz, DMSO-d6) δ 7.81 (s, 2H), 7.70-7.63 (m, 2H), 7.48 (t, J=7.6 Hz, 2H), 7.36 (s, 2H), 7.29 (d, J=7.4 Hz, 2H), 7.24 (d, J=1.7 Hz, 2H), 5.54 (s, 2H), 4.50-4.41 (m, 2H), 4.30-4.19 (m, 4H), 3.84-3.46 (m, 8H), 3.22-3.07 (m, 4H), 2.28 (s, 6H), 2.04-1.93 (m, 2H), 1.90-1.78 (m, 2H).

### Example 49. Preparation of 7,7′-(2,2′-dichloro-[1,1′-biphenyl]-3,3′-diyl)bis(3-methyl-2-(2-((((S)-5-oxopyrrolidin-2-yl)methyl)amino)ethyl)pyrrolo[1,2-a]pyrazin-1(2H)-one) (Compound 49)

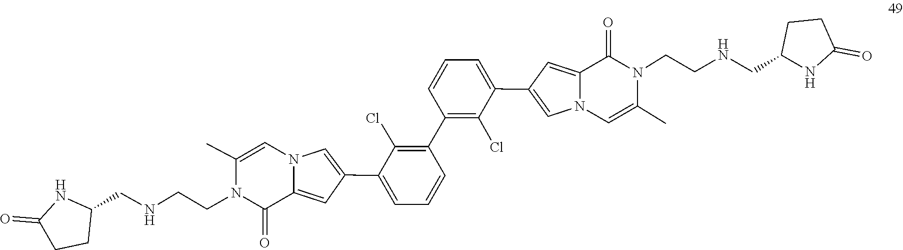 [SMILES: Cc1cn2cc(-c3cccc(-c4cccc(-c5cc6c(=O)n(CCNC[C@@H]7CCC(=O)N7)c(C)cn6c5)c4Cl)c3Cl)cc2c(=O)n1CCNC[C@@H]1CCC(=O)N1]

Following the procedure of Compound 48, the title Compound 49 was obtained by substituting (S)-5-(aminomethyl)pyrrolidin-2-one for (S)-pyrrolidin-3-ol. LCMS (ESI): calculated for C₄₂H₄₄Cl₂N₈O₄; [M+H]+: 795.3, 797.3, found: 795.3, 797.3. 1H NMR (500 MHz, DMSO-d6) δ 8.66 (s, 2H), 7.82 (d, J=1.8 Hz, 2H), 7.67 (d, J=9.2 Hz, 4H), 7.48 (t, J=7.6 Hz, 2H), 7.37 (s, 2H), 7.29 (dd, J=7.5, 1.7 Hz, 2H), 7.24 (d, J=1.7 Hz, 2H), 4.25-4.16 (m, 4H), 3.87-3.80 (m, 2H), 3.27-3.22 (m, 4H), 3.12-3.02 (m, 4H), 2.28 (s, 6H), 2.22-2.14 (m, 6H), 1.80-1.75 (m, 2H).

### Example 50. Preparation of (2S,2'S,4S,4'S)-1,1′-(((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(4-hydroxypyrrolidine-2-carboxylic acid) (Compound 50)

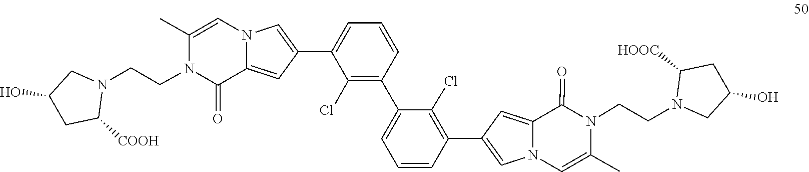 [SMILES: Cc1cn2cc(-c3cccc(-c4cccc(-c5cc6c(=O)n(CCN7C[C@@H](O)C[C@H]7C(=O)O)c(C)cn6c5)c4Cl)c3Cl)cc2c(=O)n1CCN1C[C@@H](O)C[C@H]1C(=O)O]

Following the procedure of Compound 48, the title Compound 50 was obtained by substituting (2S,4S)-4-hydroxypyrrolidine-2-carboxylic acid for (S)-pyrrolidin-3-ol. LCMS (ESI): calculated for C₄₂H₄₂Cl₂N₆O₈; [M+H]+: 829.7, 831.7, found: 829.7, 831.7. ¹H NMR (500 MHz, DMSO-d6) δ 7.81 (d, J=1.8 Hz, 2H), 7.65 (dd, J=7.8, 1.7 Hz, 2H), 7.46 (t, J=7.6 Hz, 2H), 7.34 (s, 2H), 7.27 (dd, J=7.7, 1.7 Hz, 2H), 7.20 (d, J=1.7 Hz, 2H), 5.50 (s, 2H), 4.37-4.33 (m, 2H), 4.21-4.09 (m, 6H), 3.73-3.68 (m, 2H), 3.23-3.17 (m, 2H), 2.26 (s, 6H), 2.19-2.12 (m, 4H).

### Example 51. Preparation of 7,7′-(2,2′-dichloro-[1,1′-biphenyl]-3,3′-diyl)bis(2-(2-((2-hydroxyethyl)amino) ethyl)-3-methylpyrrolo[1,2-a]pyrazin-1(2H)-one) (Compound 51)

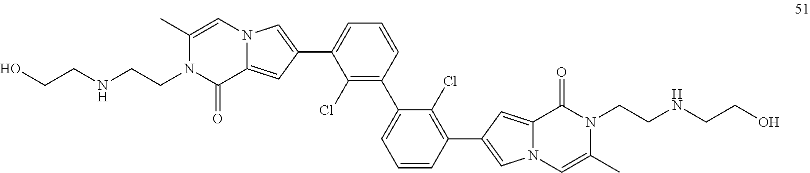 [SMILES: Cc1cn2cc(-c3cccc(-c4cccc(-c5cc6c(=O)n(CCNCCO)c(C)cn6c5)c4Cl)c3Cl)cc2c(=O)n1CCNCCO]

Following the procedure of Compound 48, the title Compound 51 was obtained by substituting 2-aminoethan-1-ol for (S)-pyrrolidin-3-ol. LCMS (ESI): calculated for C₃₆H₃₈Cl₂N₆O₄; [M+H]+: 689.2, 691.2, found: 689.2, 691.2. ¹H NMR (500 MHz, DMSO-d6) δ 8.61 (s, 4H), 7.81 (d, J=1.8 Hz, 2H), 7.67 (dd, J=7.9, 1.6 Hz, 2H), 7.48 (t, J=7.7 Hz, 2H), 7.36 (s, 2H), 7.29 (dd, J=7.6, 1.7 Hz, 2H), 7.23 (d, J=1.7 Hz, 2H), 5.31 (s, 2H), 4.22 (t, J=6.7 Hz, 4H), 3.65 (t, J=6.7 Hz, 4H), 3.26-3.18 (m, 4H), 3.09-3.01 (m, 4H), 2.26 (s, 6H).

### Example 52. Preparation of 2,2′-(2,2′-dichloro-[1,1′-biphenyl]-3,3′-diyl)bis(5-(2-((2-hydroxyethyl)amino) ethyl)-6-methylpyrazolo[1,5-a]pyrazin-4(5H)-one) (Compound 52)

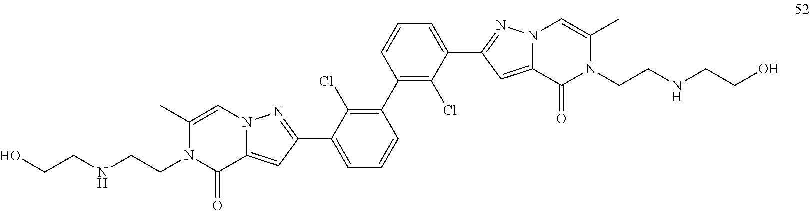 [SMILES: Cc1cn2nc(-c3cccc(-c4cccc(-c5cc6c(=O)n(CCNCCO)c(C)cn6n5)c4Cl)c3Cl)cc2c(=O)n1CCNCCO]

Following the procedure of Compound 47, the title Compound 52 was obtained by substituting 2-aminoethan-1-ol for (S)-pyrrolidine-3-carboxylic acid. LCMS (ESI): calculated for C₃₄H₃₆Cl₂N₈O₄; [M+H]+: 691.2, 693.2, found: 691.2, 693.2. ¹H NMR (500 MHz, DMSO-d₆) δ 7.86-7.83 (m, 2H), 7.78 (s, 2H), 7.72-7.69 (m, 2H), 7.58-7.53 (m, 2H), 7.46-7.44 (m, 2H), 3.85-3.71 (m, 8H), 3.31-3.25 (m, 2H), 2.94-2.86 (m, 6H), 2.80-2.74 (m, 4H), 2.30 (s, 6H).

### Example 53. Preparation of (3S,3'S)-1,1′-(((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(pyrrolidine-3-carbo xylic acid) (Compound 53)

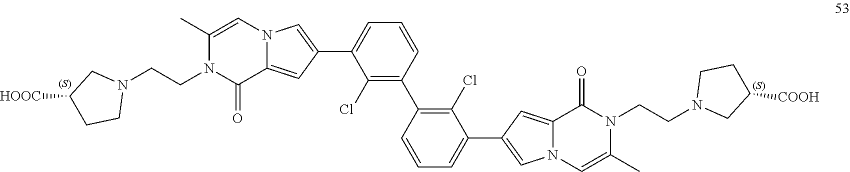

Following the procedure of Compound 48, the title Compound 53 was obtained by substituting (S)-pyrrolidine-3-carboxylic acid for (S)-pyrrolidin-3-ol. LCMS (ESI): calculated for C₄₂H₄₂Cl₂N₆O₆; [M+H]+: 797.2, 799.2, found: 797.2, 799.2. ¹H NMR (500 Mhz, DMSO-d₆) δ 7.81 (s, 2H), 7.67 (d, J=6 Hz, 2H), 7.48 (t, J=6 Hz, 2H), 7.35 (s, 2H), 7.28 (d, J=6 Hz, 2H), 7.23 (s, 2H), 3.92-3.82 (m, 4H), 2.93-2.85 (m, 4H), 2.80-2.65 (m, 8H), 2.64-2.55 (m, 2H), 2.29 (s, 6H), 1.97-1.89 (m, 2H), 1.84-1.76 (m, 2H).

### Example 54. Preparation of N,N′-(((((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(azanediyl))bis(ethane-2,1-diyl))diacetamide (Compound 54)

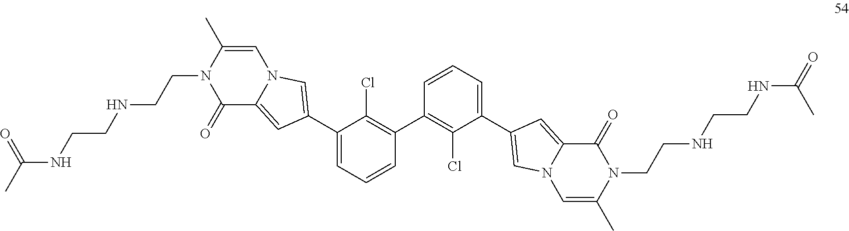 [SMILES: CC(=O)NCCNCCn1c(C)cn2cc(-c3cccc(-c4cccc(-c5cc6c(=O)n(CCNCCNC(C)=O)c(C)cn6c5)c4Cl)c3Cl)cc2c1=O]

Following the procedure of Compound 48, the title Compound 54 was obtained by substituting N-(2-aminoethyl)acetamide for (S)-pyrrolidin-3-ol. LCMS (ESI): calculated for C₄₀H₄Cl₂N₈O₄; [M+H]+: 771.3, 773.3, found: 771.3, 773.3. ¹H NMR (500 MHz, DMSO-d6) δ 8.61 (s, 2H), 8.13 (t, J=5.7 Hz, 2H), 7.81 (d, J=1.8 Hz, 2H), 7.67 (dd, J=7.8, 1.7 Hz, 2H), 7.48 (t, J=7.6 Hz, 2H), 7.36 (s, 2H), 7.29 (dd, J=7.5, 1.7 Hz, 2H), 7.24 (d, J=1.7 Hz, 2H), 4.25-4.13 (m, 4H), 3.32 (q, J=6.1 Hz, 4H), 3.27-3.19 (m, 4H), 3.07-2.99 (m, 4H), 2.27 (s, 6H), 1.85 (s, 6H).

### Example 55. Preparation of 7,7′-(2,2′-dichloro-[1,1′-biphenyl]-3,3′-diyl)bis(3-methyl-2-(2-((pyrrolidin-2-ylmethyl)amino)ethyl)pyrrolo[1,2-a]pyrazin-1(2H)-one) (Compound 55)

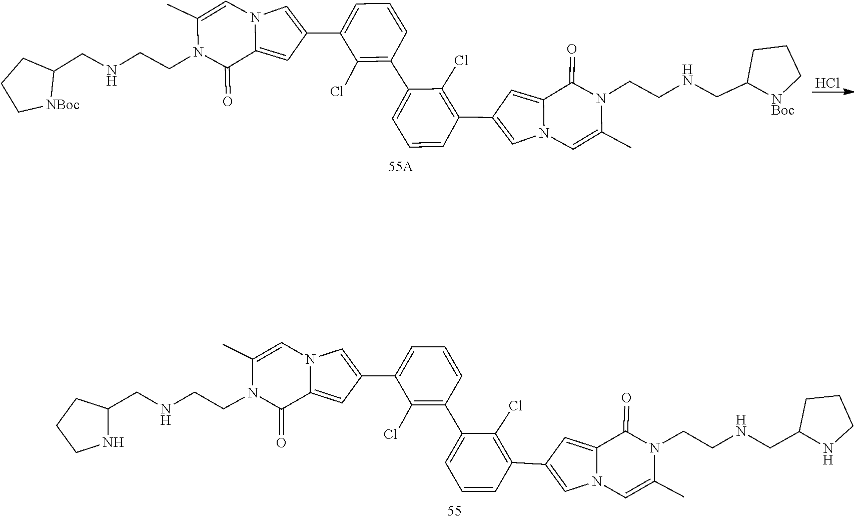 [SMILES: Cc1cn2cc(-c3cccc(-c4cccc(-c5cc6c(=O)n(CCNCC7CCCN7C(=O)OC(C)(C)C)c(C)cn6c5)c4Cl)c3Cl)cc2c(=O)n1CCNCC1CCCN1C(=O)OC(C)(C)C ; Cl ; Cc1cn2cc(-c3cccc(-c4cccc(-c5cc6c(=O)n(CCNCC7CCCN7)c(C)cn6c5)c4Cl)c3Cl)cc2c(=O)n1CCNCC1CCCN1]

Following the procedure of Compound 48, the title Compound 55A was obtained by substituting tert-butyl 2-(aminomethyl)pyrrolidine-1-carboxylate for (S)-pyrrolidin-3-ol.

To a stirred solution of 55A (20 mg, 0.021 mmol, 1 eq.) in MeOH (1.0 mL) was added HCl in dioxane (4.0 M, 1.0 mL) and stirred for 4 h at 25° C. The reaction mixture was concentrated and purify by prep-HPLC to give compound 55 (7.9 mg, yield: 51%). LCMS (ESI): calculated for C₄₂H₄₈Cl₂N₈O₂·4HCl; [M+H]+: 767.6, 769.6, found: 767.6, 769.6, ¹H NMR (500 MHz, DMSO-d6) δ 9.28 (s, 4H), 9.10 (s, 4H), 7.82 (s, 2H), 7.68-7.66 (m, 2H), 7.47 (t, J=7.5 Hz, 2H), 7.37 (s, 2H), 7.29 (d, J=5.0 Hz, 2H), 7.24 (d, J=5.0 Hz, 2H), 4.24-4.18 (m, 4H), 3.83-3.75 (m, 4H), 3.33-3.25 (m, 8H), 2.65-2.60 (m, 2H), 2.29 (s, 6H), 2.16-2.08 (m, 2H), 2.00-1.93 (m, 2H), 1.90-1.83 (m, 2H), 1.73-1.65 (m, 2H).

### Example 56. Preparation of 4,4′-((((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(azanediyl))bis(3-hydroxybutanoic acid) (Compound 56)

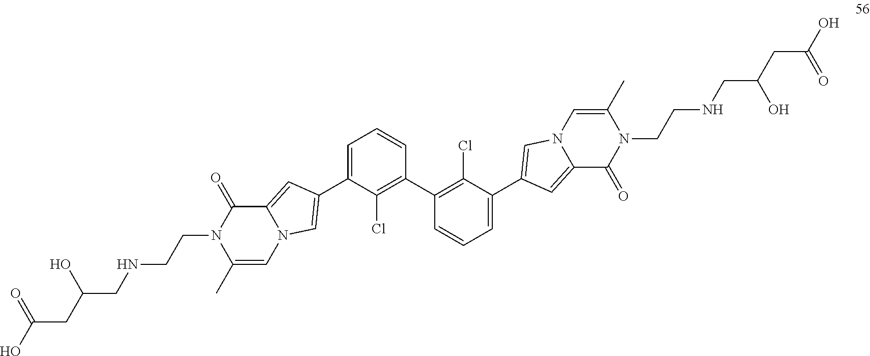 [SMILES: Cc1cn2cc(-c3cccc(-c4cccc(-c5cc6c(=O)n(CCNCC(O)CC(=O)O)c(C)cn6c5)c4Cl)c3Cl)cc2c(=O)n1CCNCC(O)CC(=O)O]

Following the procedure of Compound 48, the title Compound 56 was obtained by substituting 4-amino-3-hydroxybutanoic acid for (S)-pyrrolidin-3-ol. LCMS (ESI): calculated for C₄₀H₄₂Cl₂N₆O₈; [M+H]+: 805.2, 807.2, found: 805.2, 807.2. ¹H NMR (500 MHz, DMSO-d6) δ 8.43 (s, 2H), 7.81 (dd, J=4.9, 1.7 Hz, 2H), 7.67 (dd, J=7.8, 1.8 Hz, 2H), 7.48 (t, J=7.7 Hz, 2H), 7.36 (d, J=6.8 Hz, 2H), 7.29 (d, J=7.6 Hz, 2H), 7.23 (dd, J=4.9, 1.6 Hz, 2H), 5.66 (s, 2H), 3.62 (t, J=4.2 Hz, 2H), 3.27-3.16 (m, 4H), 3.09 (d, J=12.0 Hz, 4H), 2.99-2.88 (m, 4H), 2.43 (dd, J=23.3, 6.3 Hz, 4H), 2.26 (s, 6H).

### Example 57. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-hydroxypropanoic acid) (Compound 57)

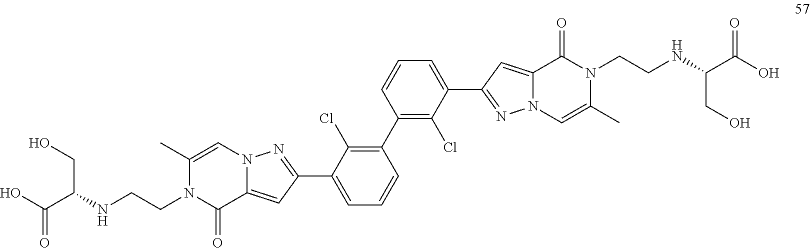 [SMILES: Cc1cn2nc(-c3cccc(-c4cccc(-c5cc6c(=O)n(CCN[C@@H](CO)C(=O)O)c(C)cn6n5)c4Cl)c3Cl)cc2c(=O)n1CCN[C@@H](CO)C(=O)O]

Following the procedure of Compound 47, the title Compound 57 was obtained by substituting L-serine for (S)-pyrrolidine-3-carboxylic acid. LCMS (ESI): calculated for C₃₆H₃₆Cl₂N₈O₈; [M+H]+: 779.2, 781.2, found: 779.2, 781.2. ¹H NMR (500 MHz, DMSO-d₆): δ 7.87-7.81 (m, 2H), 7.78 (s, 2H), 7.73-7.68 (m, 2H), 7.59-7.52 (m, 2H), 7.47-7.43 (m, 2H), 4.99-4.92 (m, 2H), 3.95-3.80 (m, 6H), 3.70-3.58 (m, 4H), 3.50-3.41 (m, 2H), 2.99-2.87 (m, 4H), 2.30 (s, 6H).

### Example 58. Preparation of (2S,2'S)-2,2′-((((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(azanediyl))bis(4-hydroxybutanoic acid) (Compound 58)

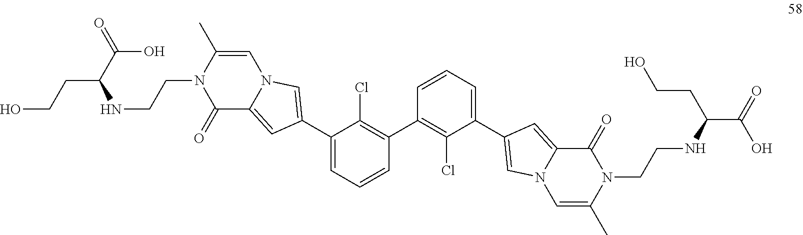 [SMILES: Cc1cn2cc(-c3cccc(-c4cccc(-c5cc6c(=O)n(CCN[C@@H](CCO)C(=O)O)c(C)cn6c5)c4Cl)c3Cl)cc2c(=O)n1CCN[C@@H](CCO)C(=O)O]

Following the procedure of Compound 48, the title Compound 58 was obtained by substituting L-homoserine for (S)-pyrrolidine-3-carboxylic acid. LCMS (ESI): calculated for C₄₀H₄₂Cl₂N₆O₈; [M+H]+: 805.2, 807.2, found: 805.2, 807.2. ¹H NMR (500 MHz, DMSO-d6) δ 8.35 (s, 2H), 7.80 (s, 2H), 7.73-7.65 (m, 2H), 7.47 (t, J=7.6 Hz, 2H), 7.35 (s, 2H), 7.28 (d, J=7.4 Hz, 2H), 7.23 (d, J=1.7 Hz, 2H), 5.56 (s, 2H), 4.54-4.44 (m, 4H), 4.34-4.23 (m, 4H), 4.08-3.94 (m, 4H), 3.55-3.30 (m, 2H), 2.28 (s, 6H), 1.88-1.76 (m, 2H).

### Example 59. Preparation of (2S,2'S,4R,4′R)-1,1′-(((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(4-hydroxypyrrolidine-2-carboxylic acid) (Compound 59)

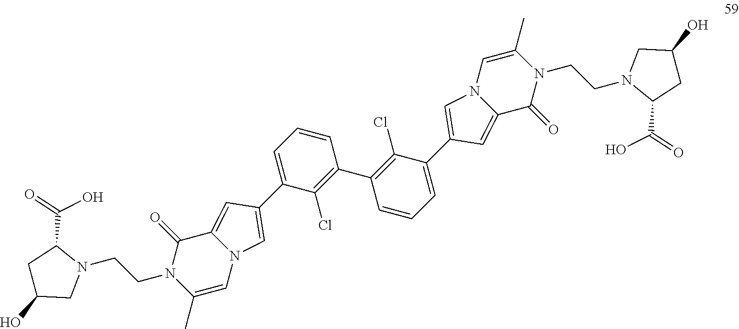 [SMILES: Cc1cn2cc(-c3cccc(-c4cccc(-c5cc6c(=O)n(CCN7C[C@@H](O)C[C@@H]7C(=O)O)c(C)cn6c5)c4Cl)c3Cl)cc2c(=O)n1CCN1C[C@@H](O)C[C@@H]1C(=O)O]

Following the procedure of Compound 48, the title Compound 59 was obtained by substituting (2R,4S)-4-hydroxypyrrolidine-2-carboxylic acid for (S)-pyrrolidine-3-carboxylic acid. LCMS (ESI): calculated for C₄₂H₄₂Cl₂N₆O₈; [M+H]+: 829.2, 831.2, found: 829.2, 831.2. ¹H NMR (500 MHz, DMSO-d6) δ 8.35 (s, 2H), 7.80 (s, 2H), 7.73-7.65 (m, 2H), 7.47 (t, J=7.6 Hz, 2H), 7.35 (s, 2H), 7.28 (d, J=7.4 Hz, 2H), 7.23 (d, J=1.7 Hz, 2H), 5.56 (s, 2H), 4.54-4.44 (m, 4H), 4.34-4.23 (m, 4H), 4.08-3.94 (m, 4H), 3.55-3.30 (m, 2H), 2.28 (s, 6H), 1.88-1.76 (m, 2H).

### Example 60. Preparation of (2S,2'S,3R,3′R)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl)) bis(3-hydroxybutanoic acid) (Compound 60)

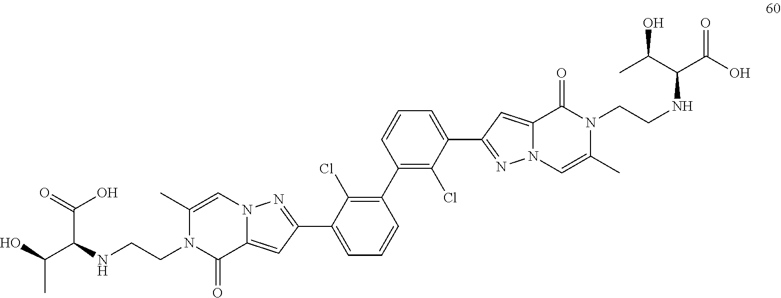 [SMILES: Cc1cn2nc(-c3cccc(-c4cccc(-c5cc6c(=O)n(CCN[C@H](C(=O)O)[C@@H](C)O)c(C)cn6n5)c4Cl)c3Cl)cc2c(=O)n1CCN[C@H](C(=O)O)[C@@H](C)O]

Following the procedure of Compound 47, the title Compound 60 was obtained by substituting L-threonine for (S)-pyrrolidine-3-carboxylic acid. LCMS (ESI): calculated for C₃₈H₄₀Cl₂N₈O₈; [M+H]⁺: 807.2, 809.2, found: 807.2, 809.2. ¹H NMR (500 MHz, DMSO-d₆): δ 7.91-7.86 (m, 2H), 7.79 (s, 2H), 7.59-7.52 (m, 2H), 7.48-7.42 (m, 2H), 7.35 (s, 2H), 4.14-4.03 (m, 4H), 3.82-3.72 (m, 4H), 3.07-2.97 (m, 4H), 2.82-2.74 (m, 4H), 2.38 (s, 6H), 1.23 (s, 6H).

### Example 61. Preparation of (2R,2′R)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-hydroxypropanoic acid) (Compound 61)

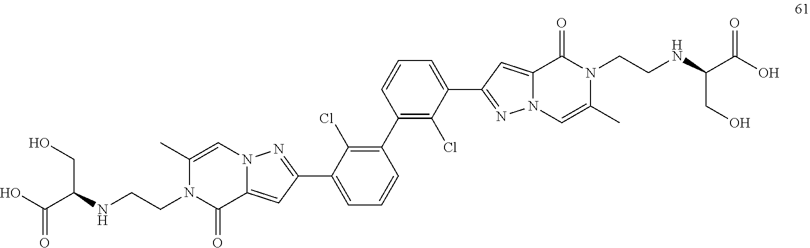 [SMILES: Cc1cn2nc(-c3cccc(-c4cccc(-c5cc6c(=O)n(CCN[C@H](CO)C(=O)O)c(C)cn6n5)c4Cl)c3Cl)cc2c(=O)n1CCN[C@H](CO)C(=O)O]

Following the procedure of Compound 47, the title Compound 61 was obtained by substituting D-serine for (S)-pyrrolidine-3-carboxylic acid. LCMS (ESI): calculated for C₃₆H₃₆Cl₂N₈O₈; [M+H]*: 779.2, 781.2, found: 779.2, 781.2. ¹H NMR (500 MHz, DMSO-d₆): δ 7.87-7.81 (m, 2H), 7.78 (s, 2H), 7.73-7.68 (m, 2H), 7.59-7.52 (m, 2H), 7.47-7.43 (m, 2H), 4.99-4.92 (m, 2H), 3.95-3.80 (m, 6H), 3.70-3.58 (m, 4H), 3.50-3.41 (m, 2H), 2.99-2.87 (m, 4H), 2.30 (s, 6H).

### Example 62. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(4-hydroxybutanoic acid) (Compound 62)

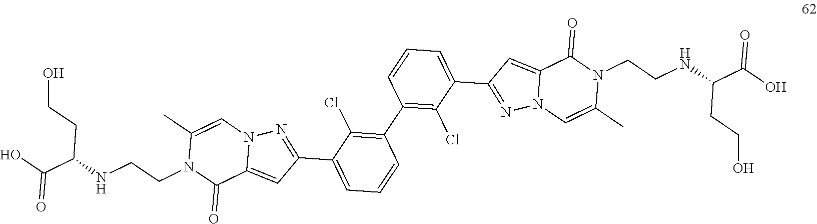 [SMILES: Cc1cn2nc(-c3cccc(-c4cccc(-c5cc6c(=O)n(CCN[C@@H](CCO)C(=O)O)c(C)cn6n5)c4Cl)c3Cl)cc2c(=O)n1CCN[C@@H](CCO)C(=O)O]

Following the procedure of Compound 47, the title Compound 62 was obtained by substituting L-homoserine for (S)-pyrrolidine-3-carboxylic acid. LCMS (ESI): calculated for C₃₈H₄₀Cl₂N₈O₈; [M+H]+: 807.2, 809.2, found: 807.2, 809.2. ¹H NMR (500 MHz, DMSO-d₆): δ 8.36 (s, 2H), 7.88 (d, J=7.7 Hz, 2H), 7.78 (s, 2H), 7.58-7.51 (m, 2H), 7.44 (d, J=7.5 Hz, 2H), 7.35 (s, 2H), 5.38-5.25 (m, 2H), 4.12-4.06 (m, 4H), 3.50-3.46 (m, 6H), 3.17-3.13 (m, 2H), 2.99-2.93 (m, 2H), 2.85-2.77 (m, 2H), 2.37 (s, 6H), 2.04-1.94 (m, 4H).

### Example 63. Preparation of (2S,2'S)-2,2′-((((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(azanediyl))bis(3-hydroxypropanoic acid) (Compound 63)

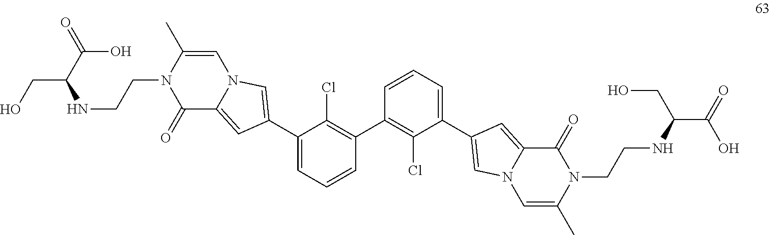 [SMILES: Cc1cn2cc(-c3cccc(-c4cccc(-c5cc6c(=O)n(CCN[C@@H](CO)C(=O)O)c(C)cn6c5)c4Cl)c3Cl)cc2c(=O)n1CCN[C@@H](CO)C(=O)O]

Following the procedure of Compound 48, the title Compound 63 was obtained by substituting L-serine for (S)-pyrrolidin-3-ol. LCMS (ESI): calculated for C₃₈H₃₈Cl₂N₆O₈; [M+H]*: 777.2, 779.2, found: 777.2, 779.2. ¹H NMR (500 MHz, DMSO-d6) δ 8.23 (s, 2H), 7.82 (s, 2H), 7.72-7.64 (m, 2H), 7.49 (t, J=7.6 Hz, 2H), 7.37 (s, 2H), 7.30 (d, J=7.4 Hz, 2H), 7.25 (d, J=1.7 Hz, 2H), 5.58 (s, 2H), 4.50-4.41 (m, 4H), 4.30-4.19 (m, 4H), 4.01-3.87 (m, 4H), 3.58-3.33 (m, 2H), 2.29 (s, 6H).

### Example 64. Preparation of (2S,2'S)-2,2′-((((2,2′-dichloro-[1,1′-biphenyl]-3,3′-diyl)bis(2-methyl-4-oxopyrrolo[2,1-f][1,2,4]triazine-6,3(4H)-diyl))bis(ethane-2,1-diyl))bis(azanediyl))bis(3-hydroxypropanoic acid) (Compound 64)

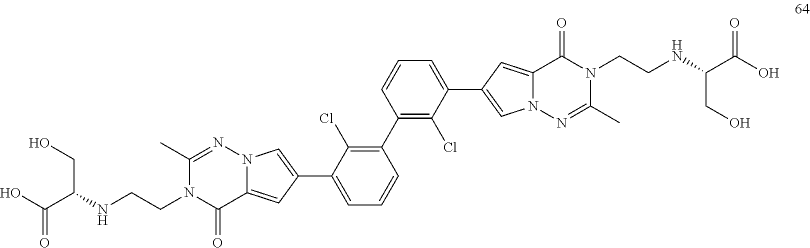 [SMILES: Cc1nn2cc(-c3cccc(-c4cccc(-c5cc6c(=O)n(CCN[C@@H](CO)C(=O)O)c(C)nn6c5)c4Cl)c3Cl)cc2c(=O)n1CCN[C@@H](CO)C(=O)O]

Following the procedure of Compound 34, the core structure 6-bromo-2-methylpyrrolo[2,1-f][1,2,4]triazin-4(3H)-one was obtained by substituting acetonitrile for cyclopropanecarbonitrile. Following the procedure of Compound 47, the title compound 64 was obtained by substituting 6-bromo-2-methylpyrrolo[2,1-f][1,2,4]triazin-4(3H)-one for 29D and L-serine for (S)-pyrrolidine-3-carboxylic acid. LCMS (ESI): calculated for C₃₆H₃₆Cl₂N₈O₈; [M+H]+: 779.2, 781.2, found: 779.2, 781.2. ¹H NMR (500 MHz, DMSO-d6) δ 7.88 (s, 2H), 7.83 (s, 2H), 7.54-7.53 (m, 2H), 7.45-7.43 (m, 2H), 7.36 (s, 2H), 4.02 (s, 4H), 3.78 (s, 2H), 3.70 (s, 2H), 2.99-2.96 (m, 4H), 2.37 (s, 6H), 2.03-2.01 (m, 5H), 1.23 (s, 4H).

### Example 65. Preparation of (2S,2'S)-2,2′-((((2,2′-dichloro-[1,1′-biphenyl]-3,3′-diyl)bis(2-ethyl-4-oxopyrrolo[2,1-f][1,2,4]triazine-6,3(4H)-diyl))bis(ethane-2,1-diyl))bis(azanediyl))bis(3-hydroxypropanoic acid) (Compound 65)

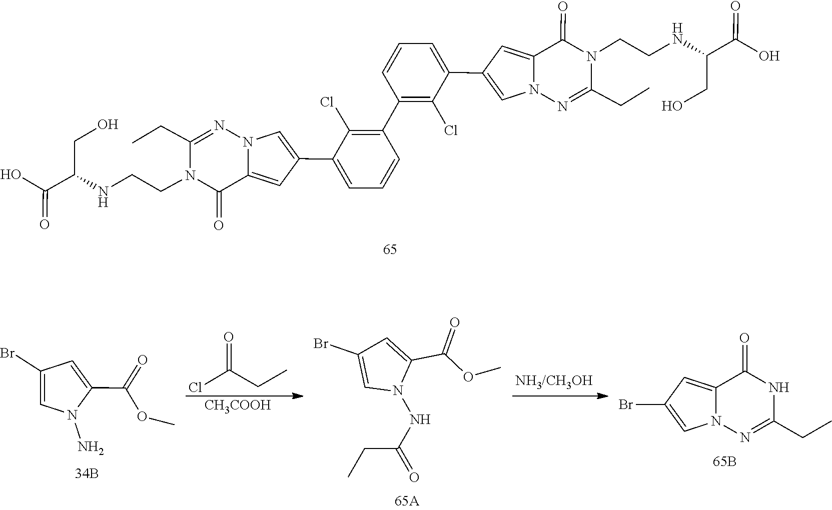

The compound 34B (1.0 g, 4.6 mmol, 1.0 eq.) was dissolved into HOAc (10 mL) at 0° C., propionyl chloride (634 mg, 6.85 mmol, 1.5 eq.) was added to the mixture reaction, and stirred 4 h. The mixture reaction was concentrated and purified by silica gel column chromatography, eluted with PE/EtOAc (3:1) to give compound 65A (0.80 g, yield: 63.7%). LCMS (ESI): calculated for C₉H₁₁BrN₂O₃; [M+H]⁺: 275.0, 277.0, found: 275.0, 277.0.

A mixture of the compound 65A (0.80 g, 2.91 mmol) in NH₃/CH₃OH (7.0 M, 20 mL) was stirred in a sealed tube at 110° C. for 2 h. After concentration, the residue was purified by flash column chromatography eluting with DCM/MeOH (10:1) to afford Compound 65B (0.43 g, yield: 61.1%). LCMS (ESI): calculated for C₈H₈BrN₃O; [M+H]+: 251.0, 253.0, found: 251.0, 253.0.

Following the procedure of Compound 47, the title compound 65 was obtained by substituting 65B for 29D and L-serine for (S)-pyrrolidine-3-carboxylic acid. LCMS (ESI): calculated for C₃₈H₄₀Cl₂N₈O₈; [M+H]⁺: 807.2, 809.2, found: 807.2, 809.2. ¹H NMR (500 MHz, DMSO-d6) δ 7.89 (s, 2H), 7.83 (s, 2H), 7.53-7.52 (m, 2H), 7.46-7.44 (m, 2H), 7.37 (s, 2H), 4.02 (s, 4H), 3.78 (s, 2H), 3.70 (s, 2H), 2.99-2.94 (m, 4H), 2.34-2.33 (m, 4H), 2.05-2.03 (m, 5H), 1.96-1.94 (m, 6H), 1.23 (s, 4H).

### Example 66. Preparation of (2S,2'S)-2,2′-((((2,2′-dichloro-[1,1′-biphenyl]-3,3′-diyl)bis(6-ethyl-4-oxopyrazolo[1,5-a]pyrazine-2,5(4H)-diyl))bis(methylene))bis(azanediyl))bis(3-hydroxy propanoic acid) (Compound 66)

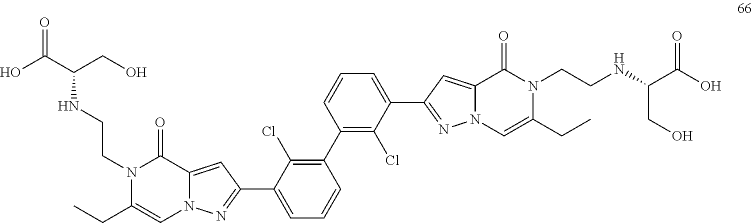 [SMILES: CCc1cn2nc(-c3cccc(-c4cccc(-c5cc6c(=O)n(CCN[C@@H](CO)C(=O)O)c(CC)cn6n5)c4Cl)c3Cl)cc2c(=O)n1CCN[C@@H](CO)C(=O)O]

Following the procedure of Compound 29, the core structure 2-bromo-6-ethylpyrazolo[1,5-a]pyrazin-4(5H)-one was obtained by substituting 1-bromobutan-2-one for 1-bromopropan-2-one.

Following the procedure of Compound 47, the title compound 66 was obtained by substituting 2-bromo-6-ethylpyrazolo[1,5-a]pyrazin-4(5H)-one for 29D and L-serine for (S)-pyrrolidine-3-carboxylic acid. LCMS (ESI): calculated for C₃₆H₃₆Cl₂N₈O₈; [M+H]+: 779.2, 781.2, found: 779.2, 781.2. ¹H NMR (500 MHz, DMSO-d₆) δ 7.85-7.81 (m, 2H), 7.77 (s, 2H), 7.72-7.69 (m, 2H), 7.58-7.53 (m, 2H), 7.47-7.44 (m, 2H), 4.99-4.93 (m, 2H), 3.91-3.82 (m, 4H), 3.71-3.59 (m, 4H), 3.51-3.42 (m, 2H), 2.99-2.87 (m, 4H), 2.37-2.35 (m, 4H), 1.97-1.91 (m, 6H).

### Example 67. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-aminopropanoic acid) (Compound 67)

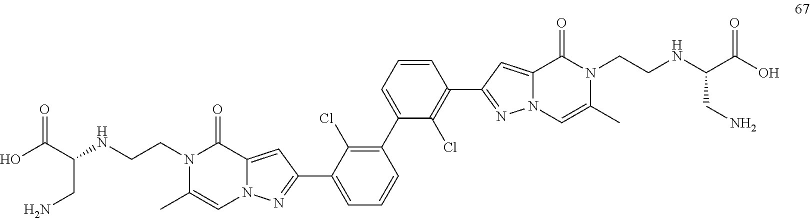 [SMILES: Cc1cn2nc(-c3cccc(-c4cccc(-c5cc6c(=O)n(CCN[C@H](CN)C(=O)O)c(C)cn6n5)c4Cl)c3Cl)cc2c(=O)n1CCN[C@@H](CN)C(=O)O]

Following the procedure of Compound 55, the title Compound 67 was obtained by substituting (S)-2-amino-3-((tert-butoxycarbonyl)amino)propanoic acid for tert-butyl 2-(aminomethyl)pyrrolidine-1-carboxylate. LCMS (ESI): calculated for C₃₆H₃₈Cl₂N₁₀O₆; [M+H]⁺: 777.2, 779.2, found: 777.2, 779.2, 581. ¹H NMR (500 MHz, DMSO-d₆): δ 7.86-7.83 (m, 2H), 7.79 (s, 2H), 7.71-7.69 (m, 2H), 7.57-7.53 (m, 2H), 7.46-7.44 (m, 2H), 4.32-4.28 (m, 2H), 3.93-3.80 (m, 4H), 3.48-3.45 (m, 2H), 3.15 (t, J=10.0 Hz, 4H) 3.00-2.90 (m, 8H), 2.31 (s, 6H).

### Example 68. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-acetamidopropanoic acid) (Compound 68)

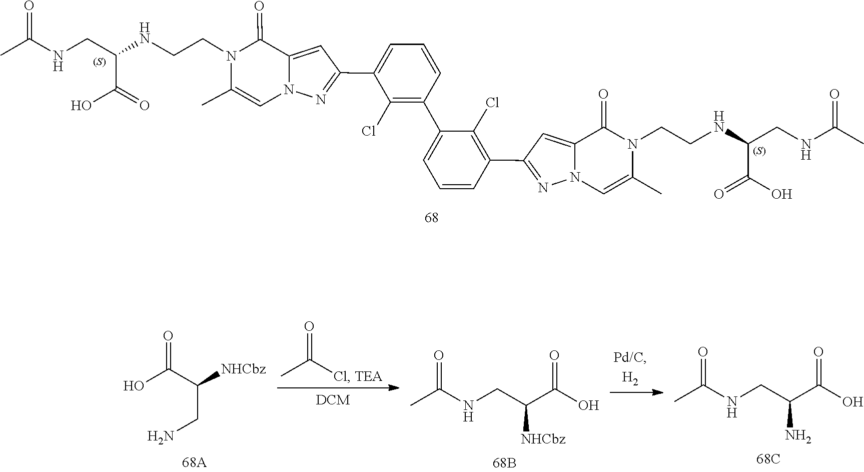

The compound 68A (1.0 g, 4.2 mmol, 1 eq.) and trimethylamine (850 mg, 8.4 mmol, 2 eq.) was dissolved into DCM (10 mL) at 0° C. Acetyl chloride (395 mg, 5.04 mmol, 1.2 eq.) was dropped into the mixture reaction, and stirred 1 h. Water (10 mL) was added and extracted. Organic layer was concentrated to get compound 68B (0.82 g, yield: 69.7%). LCMS (ESI): calculated for C₁₃H₁₆N₂O₅; [M+H]⁺: 281.1, found: 281.1.

Under H₂ atmosphere at 25° C., the compound 68B (0.50 g, 1.78 mmol, 1 eq.) and Pd/C (10% wet., 50 mg) were added into CH₃OH (10 mL), and stirred 5 h. The mixture reaction was filtered and concentrated to get compound 68C (250 mg, yield: 95.9%). LCMS (ESI): calculated for C₅H₁₀N₂O₃; [M+H]⁺: 147.1, found: 147.1.

Following the procedure of Compound 47, the title compound 68 was obtained by substituting compound 68C for (S)-pyrrolidine-3-carboxylic acid. LCMS (ESI): calculated for C₄₀H₄₂Cl₂N₁₀O₈; [M+H]⁺: 861.2, 863.2, found: 861.2, 863.2. ¹H NMR (500 MHz, DMSO-d₆): δ 7.85-7.82 (m, 2H), 7.78 (s, 2H), 7.72-7.69 (m, 2H), 7.58-7.54 (m, 2H), 7.50-7.47 (t, J=7.5 Hz, 2H), 7.46-7.44 (m, 2H), 4.64-4.60 (m, 2H), 3.93-3.78 (m, 6H), 3.36-3.32 (m, 4H), 2.94-2.90 (m, 4H), 2.30 (s, 6H), 1.95 (s, 6H).

### Example 69. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-(dimethylamino)propanoic acid) (Compound 69)

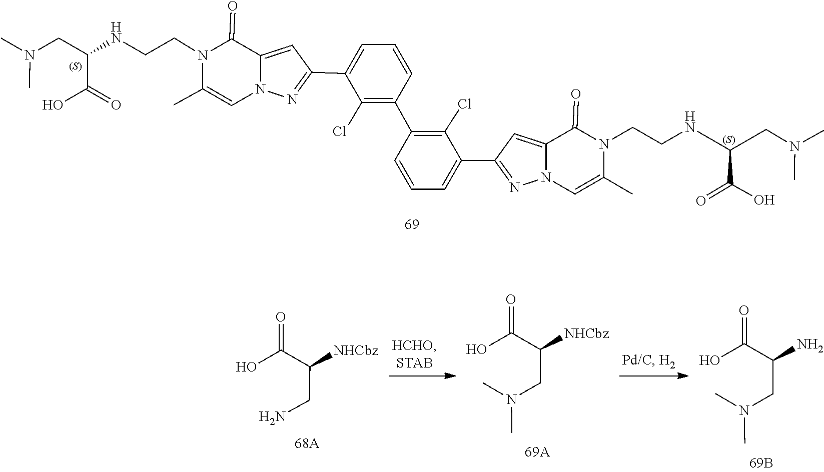

At 25° C., the compound 68A (1.0 g, 4.2 mmol, 1.0 eq.) and HCHO (37% in water, 4.0 mL) were added into CH₃OH (10 mL) and stirred 30 min. Sodium triacetoxyborohyride (4.45 g, 21 mmol, 5 eq.) was added into the mixture reaction, and stirred for 2 h. The mixture reaction was concentrated and purified by prep-HPLC to give compound 69A (0.90 g, yield: 80.5%). LCMS (ESI): calculated for C₁₃H₁₈N₂O₄; [M+H]*: 266.1, found: 266.1.

Under H₂ atmosphere at 25° C., the compound 69A (0.90 g, 3.4 mmol, 1 eq.) and Pd/C (10% wet., 90 mg) were added into CH₃OH (10 mL), and stirred for 5 h. The mixture reaction was filtered and concentrated to get compound 69B (430 mg, yield: 96.3%). LCMS (ESI): calculated for C₅H₁₂N₂O₂; [M+H]⁺: 132.1, found: 132.1.

Following the procedure of Compound 47, the title compound 69 was obtained by substituting compound 69B for (S)-pyrrolidine-3-carboxylic acid. LCMS (ESI): calculated for C₄₀H₄₆Cl₂N₁₀O₆; [M+H]⁺: 833.3, 835.3, found: 833.3, 835.3. ¹H NMR (500 MHz, DMSO-d6): δ 7.86-7.82 (m, 2H), 7.79 (s, 2H), 7.73-7.69 (m, 2H), 7.58-7.55 (m, 2H), 7.50-7.47 (t, J=7.5 Hz, 2H), 7.46-7.43 (m, 2H), 4.64-4.61 (m, 2H), 3.94-3.78 (m, 6H), 3.36-3.33 (m, 4H), 2.94-2.90 (m, 4H), 2.37 (s, 12H), 2.30 (s, 6H).

### Example 70. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(propane-3,1-diyl))bis(azanediyl))bis(3-hydroxypropanoic acid) (Compound 70)

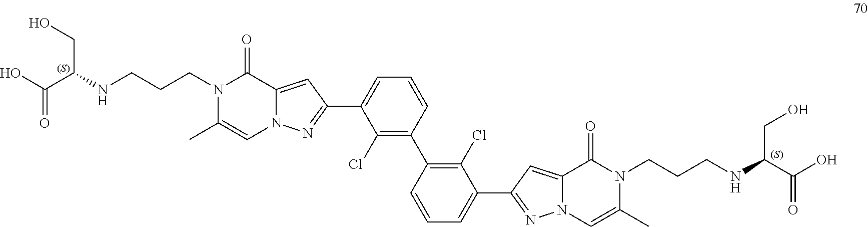

Following the procedure of Compound 47, the title compound 70 was obtained by substituting 3-bromo-1,1-dimethoxypropane for 2-bromo-1,1-dimethoxyethane and L-serine for (S)-pyrrolidine-3-carboxylic acid. LCMS (ESI): calculated for C₃₈H₄₀Cl₂N₈O₈; [M+H]⁺: 807.2, 809.2, found: 807.2, 809.2. ¹H NMR (500 MHz, DMSO-d₆): ¹H NMR (500 Mhz, DMSO-d₆) δ 7.88 (s, 2H), 7.83 (s, 2H), 7.54-7.53 (m, 2H), 7.44-7.43 (m, 2H), 7.36 (s, 2H), 4.02 (s, 4H), 3.78 (s, 2H), 3.70 (s, 2H), 2.99-2.97 (m, 4H), 2.37 (s, 6H), 2.02-2.00 (m, 5H), 1.23 (s, 4H).

### Example 71. Preparation of (2S,2'S)-2,2′-((((2,2′-dichloro-[1,1′-biphenyl]-3,3′-diyl)bis(5-methyl-4-oxo-4,5-dihydropyrazolo[1,5-a]pyrazine-2,6-diyl))bis(ethane-2,1-diyl))bis(azanediyl))bis(3-hydroxypropanoic acid) (Compound 71)

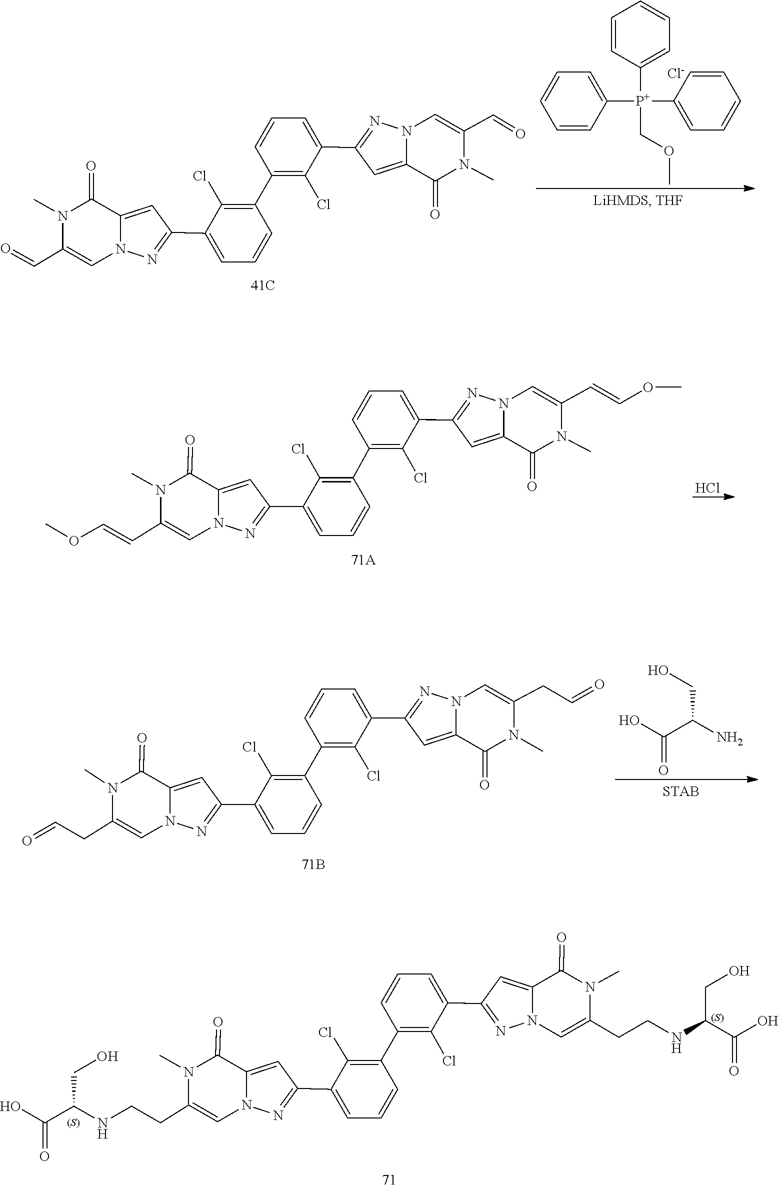

To a mixture of (methoxymethyl)triphenylphosphonium chloride (215 mg, 0.63 mmol, 1.2 eq.) in dry THF (10 mL) was added LiHMDS (1.6 M, 0.39 mL, 1.5 eq.) and stirred for 30 min at 0° C. Compound 41C (300 mg, 0.52 mmol, 1.0 eq.) was added and stirred for 1 h at 25° C. The reaction was quenched with water (10 mL) and extracted with EtOAc (10 mL×3). The combined organic layers were washed with brine (20 mL), dried over anhydrous sodium sulfate. After filtration, the filtrate was concentrated to and purified by silica gel column chromatography, eluted with PE/EtOAc (1:2) to afford Compound 71A (180 mg, yield: 54.7%). LCMS (ESI): calculated for C₃₂H₂₆Cl₂N₆O₄; [M+H]*: 628.1, 630.1, found: 628.1, 630.1.

The mixture of Compound 71A (0.18 g, 0.397 mmol,) in HCl/MeOH (1.0 M, 20 mL) was stirred for 1 h at 25° C., the mixture was concentrated under reduced pressure to give Compound 71B (130 mg, yield: 54.7%). LCMS (ESI): calculated for C₃₀H₂₂Cl₂N₆O₄; [M+H]⁺: 601.1, 603.1, found: 601.1, 603.1.

Following the procedure of Compound 47, the title Compound 71 was obtained by substituting compound 71B for compound 47C and L-serine for (S)-pyrrolidine-3-carboxylic acid. LCMS (ESI): calculated for C₃₆H₃₆Cl₂N₈O₈; [M+H]⁺: 779.2, 781.2, found: 779.2, 781.2. ¹H NMR (500 Mhz, DMSO-d6) δ 7.84-7.82 (m, 2H), 7.70-7.69 (m, 2H), 7.66 (s, 2H), 7.62 (t, J=1.0 Hz, 2H), 7.56-7.50 (m, 2H), 4.97-4.79 (m, 2H), 3.92 (t, J=5.6 Hz, 2H), 3.78-3.54 (m, 4H), 3.53-3.45 (m, 2H), 3.39 (s, 6H), 2.98-2.85 (m, 4H), 2.85-2.66 (m, 4H).

### Example 72. Preparation of N,N′-(((((2,2′-dichloro-[1,1′-biphenyl]-3,3′-diyl)bis(1-oxo-1,2-dihydropyrrolo[1,2-a]pyrazine-7,3-diyl))bis(methylene))bis(azanediyl))bis(ethane-2,1-diyl))diacetamide (Compound 72)

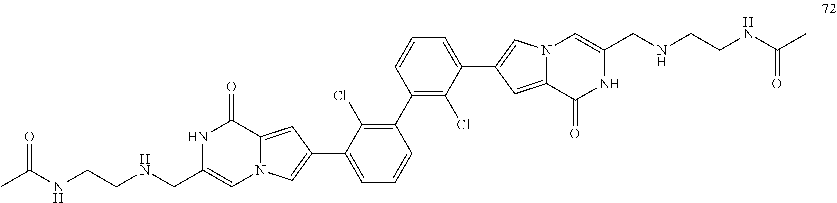 [SMILES: CC(=O)NCCNCc1cn2cc(-c3cccc(-c4cccc(-c5cc6c(=O)[nH]c(CNCCNC(C)=O)cn6c5)c4Cl)c3Cl)cc2c(=O)[nH]1]

Following the procedure of Compound 41, the title Compound 72 was obtained by substituting 7-bromo-3-methylpyrrolo[1,2-a]pyrazin-1(2H)-one for 41A and substituting N-(2-aminoethyl)acetamide for (S)-pyrrolidine-3-carboxylic acid. LCMS (ESI): calculated for C₃₆H₃₆Cl₂N₈O₄; [M+H]⁺: 715.2, 717.2, found: 715.2, 717.2. 1H NMR (500 Mhz, DMSO-d6) δ: 10.52 (s, 2H), 8.14 (t, J=4.8, 2H), 7.99 (s, 2H), 7.72-7.70 (m, 2H), 7.55 (s, 2H), 7.49 (t, J=6.4 Hz, 2H), 7.33-7.30 (m, 4H), 4.02 (s, 4H), 3.74 (s, 11H), 3.36-3.32 (m, 4H), 3.04 (s, 4H), 1.85 (s, 6H).

### Example 73. Preparation of N,N′-(((((2,2′-dichloro-[1,1′-biphenyl]-3,3′-diyl)bis(1-oxo-2-(((S)-5-oxopyrrolidin-2-yl)methyl)-1,2-dihydropyrrolo[1,2-a]pyrazine-7,3-diyl))bis(methylene)) bis(azanediyl))bis(ethane-2,1-diyl))diacetamide (Compound 73)

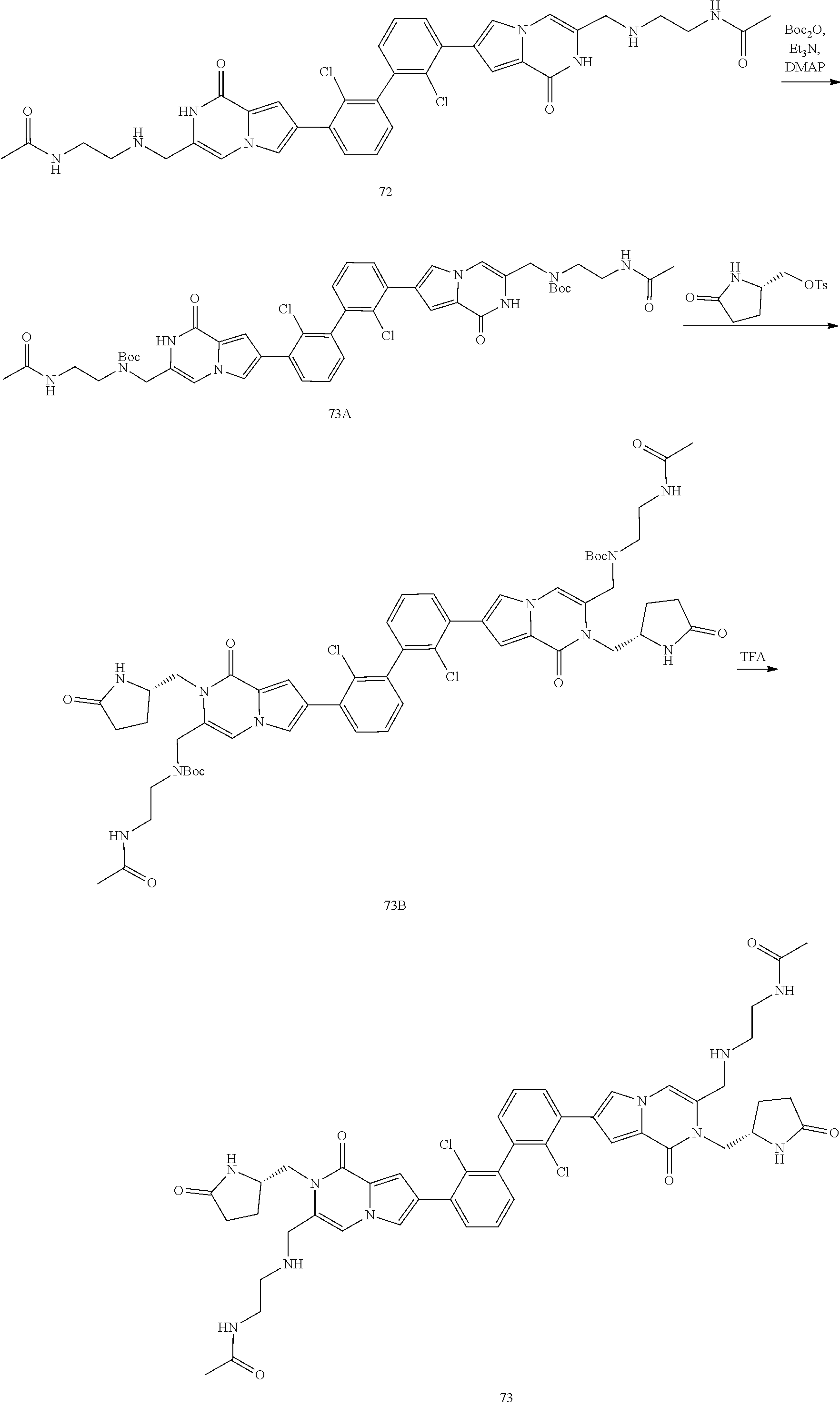

Compound 72 (200 mg, 0.28 mml, 10 eq.), Et₃ (14 mg, 1.4 mmol, 5 eq.), Boc₂O (306 mg, 1.4 mm 5 eq.) and 4-Dimethylaminopyridine (3.4 mg, 0.028 mmol, 0.1 eq) were added into DCM (5 mL), and stirred for 2 h. The mixture reaction was added water (10 mL) and extracted with DCM (10 mL×3), then the organic lay was concentrated to get compound 73A (280 mg) without purification to next step. LCMS (ESI): calculated for C₄₆H₅₂Cl₂N₈O₈; [M+H]⁺: 915.3, 917.3, found: 915.3, 917.3.

Compound 73A (280 mg, 0.31 mmol, 1.0 eq.), Cs₂CO₃ (398 mg, 1.22 mmol, 4 eq.) and compound 1D (247 mg, 0.92 mmol, 3.0 mmol) were added into DMF (3.0 mL), and stirred 1 h at 65° C. The reaction was filtered and the filtrate was purified by prep-HPLC to give compound 73B (25 mg, 21.5%). LCMS (ESI): calculated for C₅₆H₆₆Cl₂N₁₀O₁₀; [M+H]⁺: 1109.4, 1111.4, found: 1109.4, 1111.4.

Compound 73B (25 mg, 22.5 umol) was added into the trifluoroacetic acid (3 mL), and stirred 1 h at 25° C. The reaction was purified by prep-HPLC to give compound 73 (10 mg, yield: 48.8%). LCMS (ESI): calculated for C₄₆H₅₀Cl₂N₁₀O₆; [M+H]⁺: 909.3, 911.3, found: 909.3, 911.3. ¹H NMR (500 MHz, DMSO-d6) δ 8.59 (s, 2H), 8.15 (t, J=5.7 Hz, 2H), 7.77 (d, J=1.8 Hz, 2H), 7.64 (dd, J=7.8, 1.7 Hz, 2H), 7.45 (t, J=7.6 Hz, 2H), 7.33 (s, 2H), 7.28 (dd, J=7.5, 1.7 Hz, 2H), 7.23 (d, J=1.7 Hz, 2H), 4.02-3.94 (m, 3H), 3.76-3.69 (m, 3H), 3.32-3.30 (m, 4H), 3.24-3.1 (m, 4H), 3.15-3.13 (m, 2H), 3.07-2.99 (m, 4H), 2.23-2.14 (m, 2H), 2.13-2.04 (m, 4H), 2.01 (s, 6H), 1.81-1.72 (m, 2H).

### Example 74. Preparation of 7,7′-(2,2′-dichloro-[1,1′-biphenyl]-3,3′-diyl)bis(2-methyl-3-(((((S)-5-oxopyrrolidin-2-yl)methyl)amino)methyl)pyrrolo[1,2-a]pyrazin-1(2H)-one) (Compound 74)

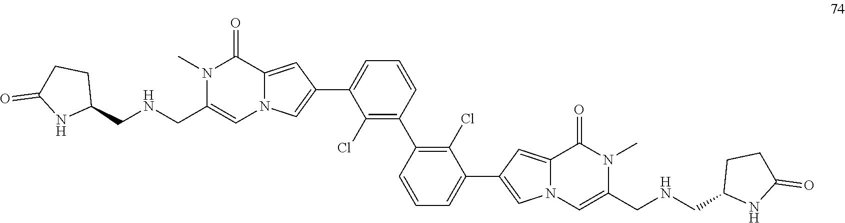 [SMILES: Cn1c(CNC[C@@H]2CCC(=O)N2)cn2cc(-c3cccc(-c4cccc(-c5cc6c(=O)n(C)c(CNC[C@@H]7CCC(=O)N7)cn6c5)c4Cl)c3Cl)cc2c1=O]

Following the procedure of Compound 43, the title Compound 74 was obtained by substituting (S)-5-(aminomethyl)pyrrolidin-2-one for (S)-pyrrolidine-3-carboxylic acid. LCMS (ESI): calculated for C₄₀H₄₀Cl₂N₈O₄; [M+H]⁺: 767.2, 769.2, found: 767.2, 769.2. ¹H NMR (500 Mhz, DMSO-d₆) δ: 7.95 (s, 2H), 7.73-7.71 (m, 4H), 7.60 (s, 2H), 7.50 (t, J=6 Hz, 2H), 7.31-7.28 (m, 4H), 4.27 (s, 4H), 3.88 (s, 4H), 3.47 (s, 6H), 3.17 (s, 2H), 2.23-2.14 (m, 8H), 1.82-1.80 (m, 2H).

### Example 75. Preparation of, 6′-(2,2′-dichloro-[1,1′-biphenyl]-3,3′-diyl)bis(2-(((S)-pyrrolidin-2-yl)meth yl)pyrrolo[2,1-f][1,2,4]triazin-4(3H)-one) (Compound 75)

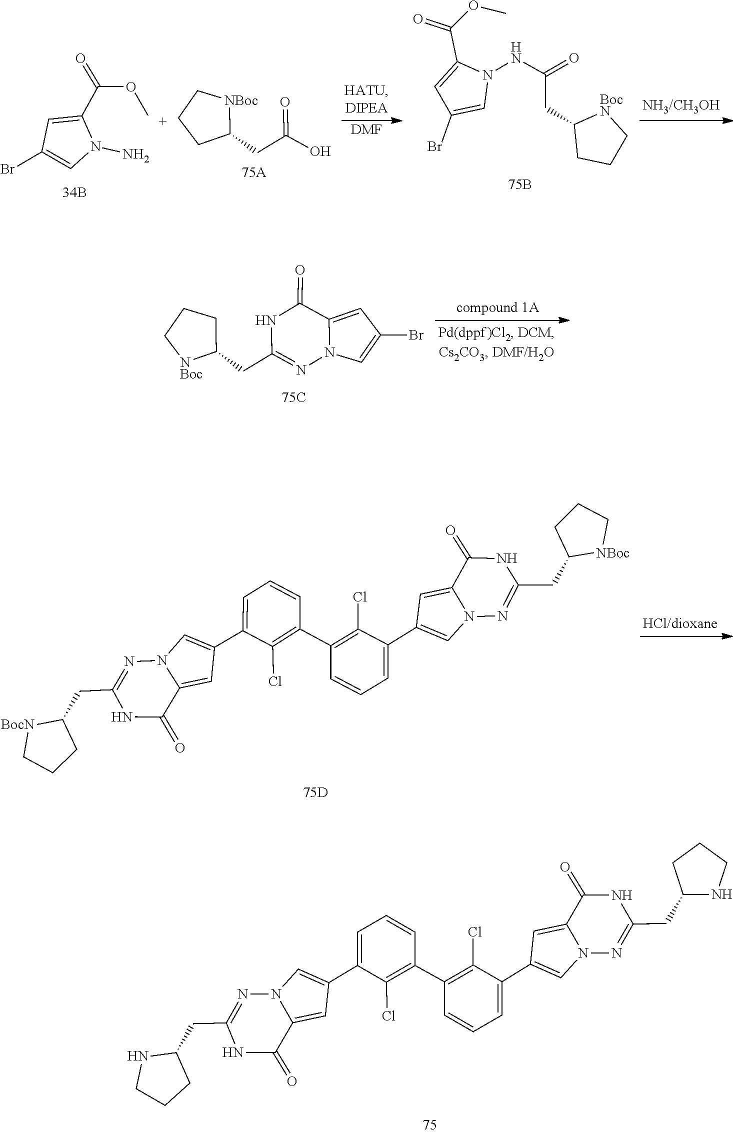

Compound 75A (534 mg, 2.28 mmol, 1.02 eq), DIPEA (500 mg, 4.56 mmol, 2.0 eq) and HATU (1.12 g, 2.74 mmol, 1.2 eq) were added into DMF (5 mL), and stirred for 15 min at 25° C. Compound 34B (500 mg, 2.28 mmol, 1.0 eq) was added into the mixture reaction, and stirred for overnight at 25° C. Water (20 mL) was added into the mixture reaction, then that was extracted with DCM (20 mL×3). The combined organics were dried over Na₂SO₄, filtered, concentrated and purified by silica gel column chromatography, eluted with PE: EtOAc (1:1) to obtain 75B (355 mg, yield: 35.4%). LCMS (ESI): calculated for C₁₇H₂₄BrN₃O₅; [M+H]+: 430.3, 432.3, found: 430.3, 432.3.

A mixture of the Compound 75B (335 mg, 0.778 mmol, 1 eq) in NH₃/MeOH (7.0 M, 15 mL) was stirred in a sealed tube at 110° C. for 8 h. After concentration, the residue was purified by flash column chromatography eluting with PE/EtOAc (1:1) to afford Compound 75C (278 mg, yield: 86.9%). LCMS (ESI): calculated for C₁H₂₁BrN₄03; [M+H]+: 397.2, 399.2, found: 397.2, 399.2, 297.2, 299.2.

Under N₂, Compound 1A (54 mg, 0.114 mmol, 1 eq.), Compound 75C (100 mg, 0.25 mmol, 2.2 eq.), Cs₂CO₃ (200 mg, 1.25 mmol, 5 eq.), Pd(dppf)Cl₂-DCM (30 mg, 0.07 mmol, 0.25 eq), was dissolved into DMF (5.0 mL) and H₂O (1.0 mL). The reaction was carried out at 90° C. for 3 h. After cooling, 30 mL water were added and resulting mixture was filtered, the filter cake was washed with ethyl acetate (3 mL×3) to give compound 75D (21 mg, yield: 22.1%). LCMS (ESI): calculated for C₄₄H₄₈Cl₂N₈O₈; [M+H]+: 855.8, 857.8 found: 855.8, 857.8.

To a solution of 75D (54 mg, 0.025 mmol, 1 eq.) was added MeOH and HCl in dioxane/(4.0M, 2.0 mL) at 25° C. The mixture was stirred for 4 h at 25° C. The mixture reaction was concentrated and purify by prep-HPLC to give compound 75 (8.3 mg, yield: 50.2%). LCMS (ESI): calculated for C34H₃₂Cl₂N₈O₂·2HCl; [M+H]+: 655.5, 657.5, found: 655.5, 657.5, ¹H NMR (500 MHz, DMSO-d6): δ 11.95 (s, 2H), 9.38 (s, 2H), 9.18 (s, 2H), 7.96 (s, 2H), 7.71 (d, J=2.5 Hz, 2H), 7.46 (d, J=5.0 Hz, 2H), 7.31 (d, J=2.5 Hz, 2H), 7.24 (s, 2H), 3.90 (t, J=5.0 Hz, 2H), 3.34-3.30 (m, 4H), 3.19-3.17 (m, 2H), 3.08-3.03 (m, 2H), 2.24-2.17 (m, 2H), 2.00-1.97 (m, 2H), 1.91-1.86 (m, 2H), 1.75-1.66 (m, 2H).

### Example 76. Preparation of 6,6′-(2,2′-dichloro-[1,1′-biphenyl]-3,3′-diyl)bis(3-methyl-2-(((S)-pyrrolidin-2-yl)methyl)pyrrolo[2,1-f][1,2,4]triazin-4(3H)-one) (Compound 76)

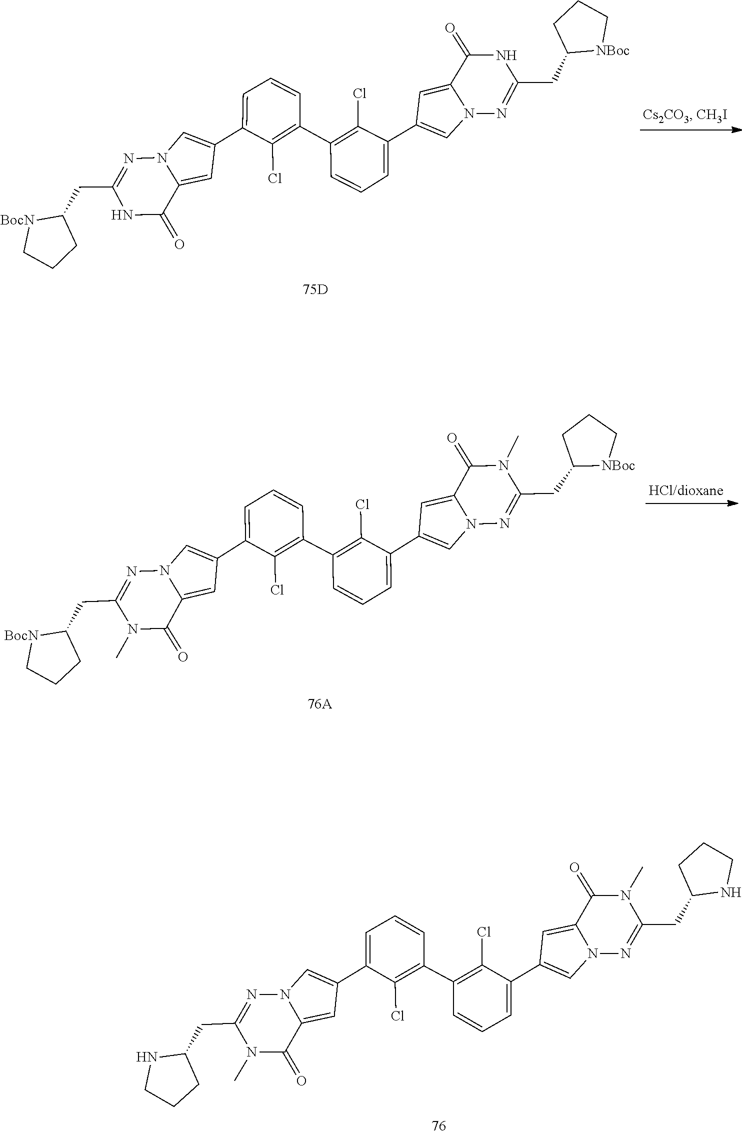 [SMILES: CC(C)(C)OC(=O)N1CCC[C@H]1Cc1nn2cc(-c3cccc(-c4cccc(-c5cc6c(=O)[nH]c(C[C@@H]7CCCN7C(=O)OC(C)(C)C)nn6c5)c4Cl)c3Cl)cc2c(=O)[nH]1 ; Cn1c(C[C@@H]2CCCN2C(=O)OC(C)(C)C)nn2cc(-c3cccc(-c4cccc(-c5cc6c(=O)n(C)c(C[C@@H]7CCCN7C(=O)OC(C)(C)C)nn6c5)c4Cl)c3Cl)cc2c1=O ; Cn1c(C[C@@H]2CCCN2)nn2cc(-c3cccc(-c4cccc(-c5cc6c(=O)n(C)c(C[C@@H]7CCCN7)nn6c5)c4Cl)c3Cl)cc2c1=O]

To a solution of Compound 75D (50 mg, 0.05 mmol, 1.0 eq.) into DMF (2.5 mL) was added Cs₂CO₃ (85 mg, 0.25 mmol, 5.0 eq.) and stirred for 10 min at 25° C., then CH₃I was added (48 mg, 0.30 mmol, 6.0 eq). The reaction mixture was stirred for 1 h. The reaction mixture was added into water (20 mL), The solution was extracted with DCM (20 mL×3) and the combined organics were dried over Na₂SO₄, filtered, concentrated, and purified by silica gel column chromatography, eluted with PE/EtOAc (1:1) to afford Compound 76A (30 mg, yield: 58.4%). LCMS (ESI): calculated for C₄₆H₅₂Cl₂N₈O₆; [M+H]+: 883.8, 885.8, found: 883.8, 885.8.

To a solution of Compound 76A (30 mg, 0.034 mmol, 1.0 eq.) was added MeOH (2 mL) and HCl in dioxane (4.0 M, 2.0 mL) at 25° C. The mixture was stirred for 4 h at 25° C. The mixture reaction was concentrated and purify by prep-HPLC to give compound 76 (10 mg, yield: 43.1%). LCMS (ESI): calculated for C₃₆H₃₆Cl₂N₈O₂; [M+H]+: 682.2, 684.2, found: 682.2, 684.2, ¹H NMR (500 MHz, DMSO-d6) δ 9.35 (s, 2H), 9.14 (s, 2H), 8.00 (d, J=5.0 Hz, 2H), 7.73 (d, J=2.5 Hz, 2H), 7.50 (d, J=7.5 Hz, 2H), 7.33 (d, J=10.0 Hz, 2H), 7.26 (s, 2H), 4.03-3.99 (m, 2H), 3.43 (s, 6H), 3.34-3.30 (m, 4H), 3.23-3.17 (m, 4H), 2.27-2.20 (m, 2H), 2.03-1.97 (m, 2H), 1.94-1.86 (m, 2H), 1.79-1.71 (m, 2H).

### Example 77. Preparation of diethyl 2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))(2S,2'S)-bis(3-hydroxypropanoate) (Compound 77)

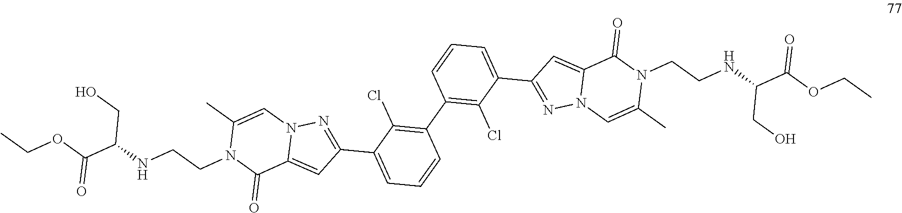 [SMILES: CCOC(=O)[C@H](CO)NCCn1c(C)cn2nc(-c3cccc(-c4cccc(-c5cc6c(=O)n(CCN[C@@H](CO)C(=O)OCC)c(C)cn6n5)c4Cl)c3Cl)cc2c1=O]

Following the procedure of Compound 47, the title Compound 77 was obtained by substituting ethyl L-serinate for (S)-pyrrolidine-3-carboxylic acid. LCMS (ESI): calculated for C₄₀H₄₄Cl₂N₈O₈; [M+H]+: 835.2, 837.2, found: 835.2, 837.2. ¹H NMR (500 MHz, DMSO-d₆) δ 7.90-7.83 (m, 4H), 7.60-7.53 (m, 2H), 7.48-7.43 (m, 2H), 7.38 (s, 2H), 4.40-4.19 (m, 8H), 3.95-3.85 (m, 3H), 3.58-3.33 (m, 11H), 2.36 (s, 6H), 1.23-1.25 (m, 6H).

### Example 78. Preparation of diethyl 1,1′-(((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))(3S,3'S)-bis(pyrrolidine-3-carboxylate) (Compound 78)

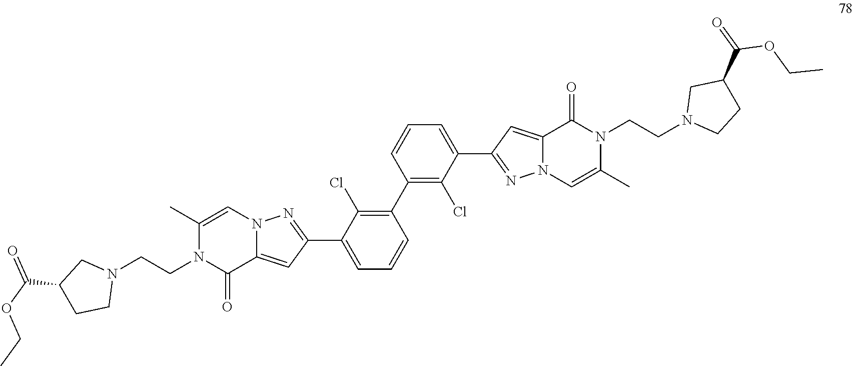 [SMILES: CCOC(=O)[C@H]1CCN(CCn2c(C)cn3nc(-c4cccc(-c5cccc(-c6cc7c(=O)n(CCN8CC[C@H](C(=O)OCC)C8)c(C)cn7n6)c5Cl)c4Cl)cc3c2=O)C1]

Following the procedure of Compound 47, the title Compound 78 was obtained by substituting ethyl (S)-pyrrolidine-3-carboxylate for (S)-pyrrolidine-3-carboxylic acid. LCMS (ESI): calculated for C₄₄H₄₈Cl₂N₈O₆; [M+H]+: 855.3, 857.3, found: 855.3, 857.3. ¹H NMR (500 MHz, DMSO-d₆) δ 7.86-7.82 (m, 2H), 7.78 (s, 2H), 7.72-7.69 (m, 2H), 7.56 (dd, J=8.6, 7.7 Hz, 2H), 7.46-7.44 (m, 2H), 4.21-4.08 (m, 4H), 3.93-3.73 (m, 4H), 2.94-2.85 (m, 4H), 2.82-2.69 (m, 6H), 2.69-2.64 (m, 2H), 2.63-2.57 (m, 2H), 2.30 (s, 6H), 2.00-1.90 (m, 2H), 1.85-1.75 (m, 2H), 1.28-1.21 (m, 6H).

### Example 79. Preparation of 1,1′-(((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(piperidine-2-carboxylic acid) (Compound 79)

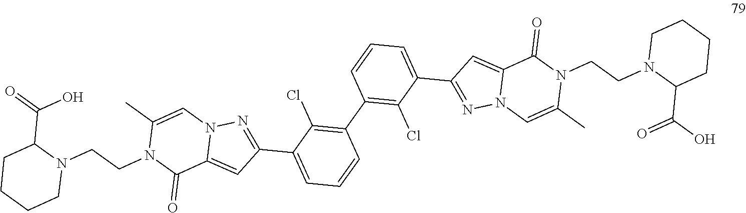 [SMILES: Cc1cn2nc(-c3cccc(-c4cccc(-c5cc6c(=O)n(CCN7CCCCC7C(=O)O)c(C)cn6n5)c4Cl)c3Cl)cc2c(=O)n1CCN1CCCCC1C(=O)O]

Following the procedure of Compound 47, the title Compound 79 was obtained by substituting piperidine-2-carboxylic acid for (S)-pyrrolidine-3-carboxylic acid. LCMS (ESI): calculated for C₄₂H₄₄Cl₂N₈O₆; [M+H]+: 827.3, 829.3, found: 827.3, 829.3. ¹H NMR (500 MHz, DMSO-d₆) δ 7.88-7.85 (m, 2H), 7.84 (s, 2H), 7.74-7.71 (m, 2H), 7.59-7.55 (m, 2H), 7.47-7.46 (m, 2H), 3.97-3.91 (m, 2H), 3.87-3.81 (m, 2H), 3.31-3.25 (m, 2H), 2.98-2.90 (m, 2H), 2.87-2.75 (m, 4H), 2.68-2.60 (m, 2H), 2.29 (s, 6H), 1.89-1.80 (m, 4H), 1.67-1.50 (m, 8H).

### Example 80. Preparation of (2S,2'S)-2,2′-((((2,2′-dichloro-[1,1′-biphenyl]-3,3′-diyl)bis(5-methyl-4-oxo-4,5-dihydropyrazolo[1,5-a]pyrazine-2,6-diyl))bis(methylene))bis(azanediyl))bis(3-hydroxypropanoic acid) (Compound 80)

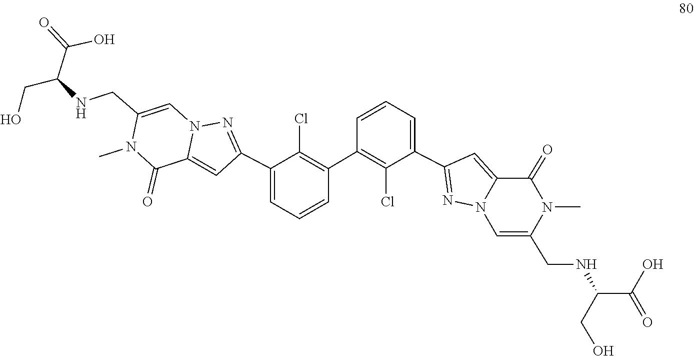 [SMILES: Cn1c(CN[C@@H](CO)C(=O)O)cn2nc(-c3cccc(-c4cccc(-c5cc6c(=O)n(C)c(CN[C@@H](CO)C(=O)O)cn6n5)c4Cl)c3Cl)cc2c1=O]

Following the procedure of Compound 41, the title Compound 80 was obtained by substituting L-serine for (S)-pyrrolidine-3-carboxylic acid. LCMS (ESI): calculated for C₃₄H₃₂Cl₂N₈O₈; [M+H]*: 751.58, found: 751.15. ¹H NMR (500 MHz, DMSO-d6) δ 7.83 (s, 2H), 7.73-7.66 (m, 2H), 7.51 (t, J=7.6 Hz, 2H), 7.38 (s, 2H), 7.31 (d, J=7.4 Hz, 2H), 7.26 (d, J=1.7 Hz, 2H), 5.56 (s, 2H), 4.53-4.42 (m, 4H), 3.92 (s, 6H), 3.53 (s, 4H), 3.49-3.34 (m, 2H).

### Example 81. Preparation of 2,2′-(2,2′-dichloro-[1,1′-biphenyl]-3,3′-diyl)bis(6-methyl-5-(((S)-pyrrolidin-3-yl)methyl)pyrazolo[1,5-a]pyrazin-4(5H)-one) (Compound 81)

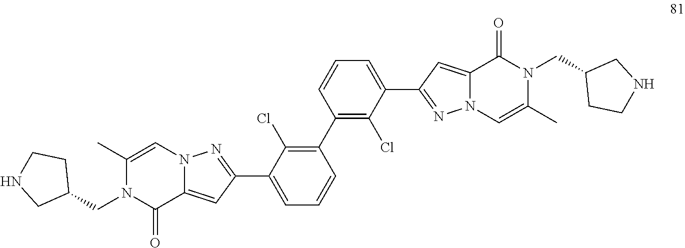 [SMILES: Cc1cn2nc(-c3cccc(-c4cccc(-c5cc6c(=O)n(C[C@H]7CCNC7)c(C)cn6n5)c4Cl)c3Cl)cc2c(=O)n1C[C@H]1CCNC1]

Following the procedure of Compound 36, the title Compound 81 was obtained by substituting tert-butyl (S)-3-(hydroxymethyl)pyrrolidine-1-carboxylate for tert-butyl (S)-2-(hydroxymethyl)pyrrolidine-1-carboxylate (36A-1). LCMS (ESI): calculated for C₃₆H₃₆Cl₂N₈O₂; [M+H]*: 683.2, 685.2, found: 683.2, 685.2. ¹H NMR (500 Mhz, DMSO-d6) δ 7.85 (s, 2H), 7.70-7.64 (m, 2H), 7.49 (t, J=7.6 Hz, 2H), 7.39 (s, 2H), 7.26 (d, J=7.4 Hz, 2H), 7.21 (d, J=1.7 Hz, 2H), 5.54 (s, 2H), 4.50-4.41 (m, 2H), 4.26-4.18 (m, 4H), 3.84-3.46 (m, 4H), 3.12-3.06 (m, 4H), 2.27 (s, 6H), 2.18-2.09 (m, 2H).

### Example 82. Preparation of 3,3′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(oxetane-3-carboxylic acid) (Compound 82)

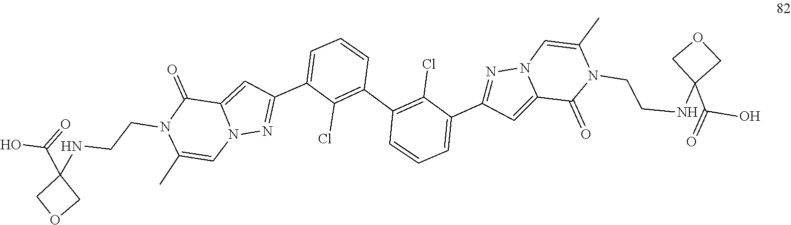 [SMILES: Cc1cn2nc(-c3cccc(-c4cccc(-c5cc6c(=O)n(CCNC7(C(=O)O)COC7)c(C)cn6n5)c4Cl)c3Cl)cc2c(=O)n1CCNC1(C(=O)O)COC1]

Following the procedure of Compound 47, the title Compound 82 was obtained by substituting 3-aminooxetane-3-carboxylic acid for (S)-pyrrolidine-3-carboxylic acid. LCMS (ESI): calculated for C₃₈H₃₆Cl₂N₈O₈; [M+H]⁺: 803.65, found: 803.25. ¹H NMR (500 MHz, DMSO-d₆) δ 7.88 (dd, J=7.8, 1.8 Hz, 2H), 7.83 (s, 2H), 7.56 (q, J=7.3 Hz, 2H), 7.45 (dd, J=7.6, 1.8 Hz, 2H), 7.38 (s, 2H), 4.76-4.71 (s, 4H), 4.60-4.46 (m, 4H), 4.24-4.18 (m, 4H), 3.44 (s, 2H), 3.18-3.14 (m, 4H), 2.40 (s, 6H).

### Example 83. Preparation of 2,2′-(2,2′-dichloro-[1,1′-biphenyl]-3,3′-diyl)bis(6-methyl-5-(2-(piperazin-1-yl) ethyl)pyrazolo[1,5-a]pyrazin-4(5H)-one) (Compound 83)

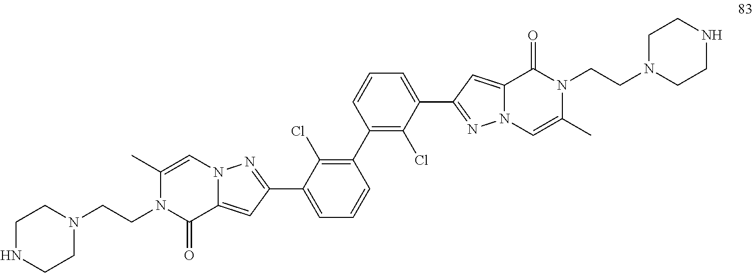 [SMILES: Cc1cn2nc(-c3cccc(-c4cccc(-c5cc6c(=O)n(CCN7CCNCC7)c(C)cn6n5)c4Cl)c3Cl)cc2c(=O)n1CCN1CCNCC1]

Following the procedure of Compound 47, the title Compound 83 was obtained by substituting piperazine for (S)-pyrrolidine-3-carboxylic acid. LCMS (ESI): calculated for C₃₈H₄₂Cl₂N₁₀O₂; [M+H]⁺: 741.3, 743.3, found: 741.3, 743.3. ¹H NMR (500 MHz, DMSO-d₆) δ 7.88-7.85 (m, 2H), 7.84 (s, 2H), 7.74-7.71 (m, 2H), 7.59-7.55 (m, 2H), 7.48-7.45 (m, 2H), 3.86-3.72 (m, 4H), 2.70-2.67 (m, 8H), 2.58-2.54 (m, 12H), 2.34-2.31 (m, 2H), 2.29 (s, 6H).

### Example 84. Preparation of (2S,2'S,5S,5'S)-1,1′-(((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(5-hydroxypiperidine-2-carboxylic acid) (Compound 84)

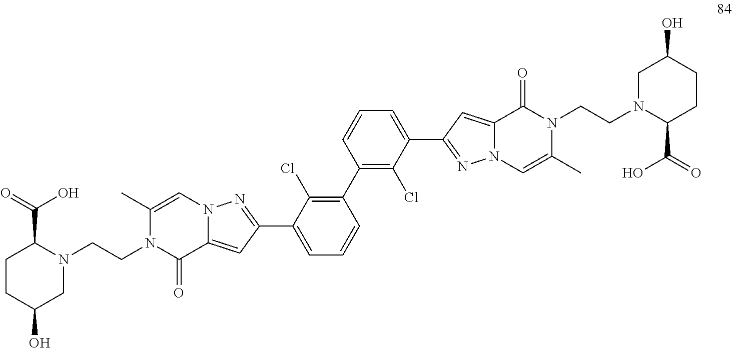 [SMILES: Cc1cn2nc(-c3cccc(-c4cccc(-c5cc6c(=O)n(CCN7C[C@@H](O)CC[C@H]7C(=O)O)c(C)cn6n5)c4Cl)c3Cl)cc2c(=O)n1CCN1C[C@@H](O)CC[C@H]1C(=O)O]

Following the procedure of Compound 47, the title Compound 84 was obtained by substituting (2S,5S)-5-hydroxypiperidine-2-carboxylic acid for (S)-pyrrolidine-3-carboxylic acid. LCMS (ESI): calculated for C₄₂H₄₄Cl₂N₈O₈; [M+H]⁺: 859.3, 861.3, found: 859.3, 861.3. ¹H NMR (500 MHz, DMSO-d₆) δ 7.88-7.85 (m, 2H), 7.84 (s, 2H), 7.74-7.71 (m, 2H), 7.59-7.55 (m, 2H), 7.46 (s, 2H), 4.24-4.18 (m, 2H), 3.97-3.71 (m, 6H), 3.43-3.37 (m, 2H), 3.03-2.96 (m, 2H), 2.92-2.81 (m, 4H), 2.79-2.73 (m, 2H), 2.29 (s, 6H), 1.90-1.63 (m, 8H).

### Example 85. Preparation of 1,1′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-hydroxycyclobutane-1-carboxylic acid (Compound 85)

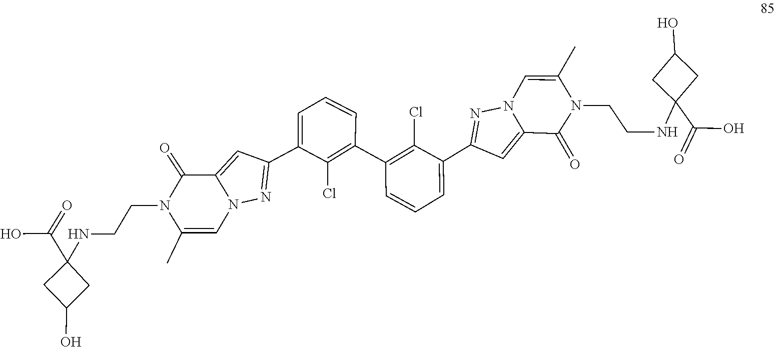 [SMILES: Cc1cn2nc(-c3cccc(-c4cccc(-c5cc6c(=O)n(CCNC7(C(=O)O)CC(O)C7)c(C)cn6n5)c4Cl)c3Cl)cc2c(=O)n1CCNC1(C(=O)O)CC(O)C1]

Following the procedure of Compound 47, the title Compound 85 was obtained by substituting 1-amino-3-hydroxycyclobutane-1-carboxylic acid for (S)-pyrrolidine-3-carboxylic acid. LCMS (ESI): calculated for C₄₀H₄₀Cl₂N₈O₈; [M+H]⁺: 831.71, found: 799.42. ¹H NMR (500 MHz, DMSO-d₆) δ 7.89 (dd, J=7.8, 1.8 Hz, 2H), 7.80 (d, J=12.7 Hz, 2H), 7.56-7.45 (m, 6H), 7.36 (d, J=2.6 Hz, 2H), 4.16 (s, 2H), 4.09-4.02 (m, 4H), 3.35-3.29 (m, 4H), 2.39 (m, 6H), 2.44-2.38 (m, 4H), 2.24-2.20 (m, 4H).

### Example 86. Preparation of 4,4′-(((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(morpholine-3-carboxylic acid) (Compound 86)

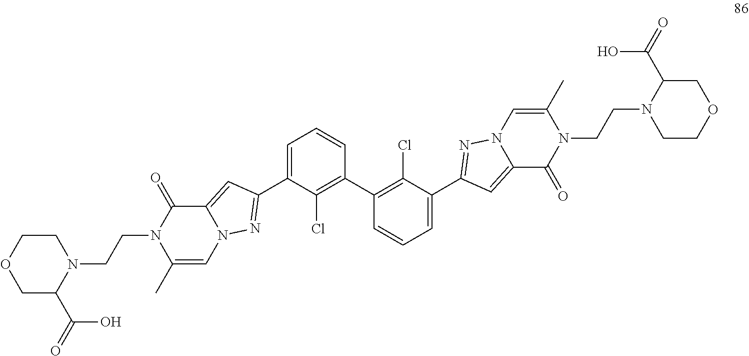 [SMILES: Cc1cn2nc(-c3cccc(-c4cccc(-c5cc6c(=O)n(CCN7CCOCC7C(=O)O)c(C)cn6n5)c4Cl)c3Cl)cc2c(=O)n1CCN1CCOCC1C(=O)O]

Following the procedure of Compound 47, the title Compound 86 was obtained by substituting morpholine-3-carboxylic acid for (S)-pyrrolidine-3-carboxylic acid. LCMS (ESI): calculated for C₄₀H₄₀Cl₂N₈O₈; [M+H]⁺: 830.23, found: 830.20. ¹H NMR (500 MHz, DMSO-d₆) δ 7.89 (d, J=7.8 Hz, 2H), 7.78 (s, 2H), 7.56 (t, J=7.7 Hz, 2H), 7.44 (d, J=7.6 Hz, 2H), 7.35 (s, 2H), 4.09 (t, J=6.5 Hz, 4H), 3.85-3.53 (m, 10H), 3.24-2.76 (m, 8H), 2.40 (s, 6H).

### Example 87. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(propane-1,2-diyl))bis(azanediyl))bis(3-hydroxypropanoic acid) (Compound 87)

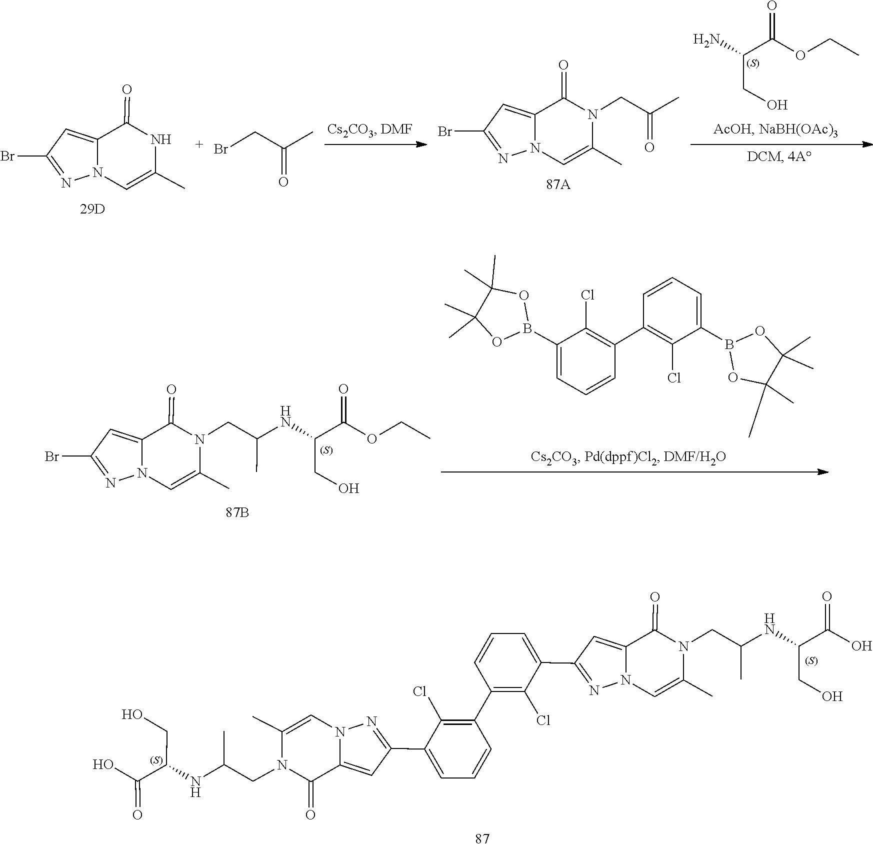

To a stirred mixture of Compound 29D (300 mg, 1.32 mmol, 1.0 eq) in DMF (6 mL) were added Cs₂CO₃ (857 mg, 2.63 mmol, 2.0 eq.) and 1-bromopropan-2-one (288 mg, 2.1 mmol, 1.6 eq.) at 0° C. The resulting mixture was stirred for 1 h at 25° C. The reaction was quenched with water and extracted with EtOAc (50 mL×3). The combined organic layers were washed with brine (100 mL), dried over anhydrous sodium sulfate. After filtration, the filtrate was concentrated under reduced pressure. The residue was purified by silica gel column chromatography, eluted with PE/EtOAc (2:1) to afford Compound 87A, 300 mg, yield: 80.7%. LCMS (ESI): calculated for C₈H₉BrN₂O₃; [M+H]+: 284.11, 286.11, found: 284.11, 286.11.

To a stirred solution of Compound 87A (200 mg, 0.709 mmol, 1.0 eq.) in DCM (10.00 mL) was added ethyl L-serinate (234 mg, 1.76 mmol, 2.5 eq.) and a drop of HOAc at 25° C. After stirred for 0.5 h at 25° C., the sodium triacetoxyborohydride (895 mg, 4.21 mmol, 6.0 eq.) was added. The resulting mixture was stirred for 16 h at 25° C. The reaction mixture was concentrated, and purified by preparative liquid phase chromatography to give Compound 87B (71 mg, yield: 25.1%). LCMS (ESI): calculated for C₁₅H₂₁BrN₄O; [M+H]+: 401.3, 403.3, found: 401.3, 403.3.

Under N2 atmosphere, Compound 87B (70 mg, 0.17 mmol, 2.2 eq.), Compound 1A (37 mg, 0.079 mmol, 1.0 eq.), Cs₂CO₃ (129 mg, 0.40 mmol, 5 eq.), Pd(dppf)Cl₂-DCM (15 mg, 0.05 mmol, 0.30 eq.), were dissolved into DMF (5.0 mL) and H₂O (1.0 mL). The reaction was carried out at 90° C. for 2 h. After cooling, 30 mL water were added and 30 mL EtOAc were added for extraction, and the organic phase was washed with water and concentrated to afford residue. The residue was purified by prep-HPLC to give Compound 87 (6.9 mg, yield: 9%). LCMS (ESI): calculated for C₃₈H₄₀Cl₂N₈O₈; [M+H]+: 807.7, 809.7, found: 807.7, 809.7. ¹H NMR (500 Mhz, DMSO-d6) δ 11.64 (s, 2H), 8.24 (s, 2H), 7.83 (s, 2H), 7.71-7.63 (m, 2H), 7.46 (t, J=7.6 Hz, 2H), 7.35 (s, 2H), 7.30 (d, J=7.4 Hz, 2H), 7.25 (d, J=1.7 Hz, 2H), 5.58 (s, 2H), 4.50-4.41 (m, 4H), 4.30-4.19 (m, 4H), 4.01-3.87 (m, 2H), 3.79-3.67 (m, 6H), 3.54-3.23 (m, 2H), 2.21 (s, 6H).

### Example 88. Preparation of 1,1′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(4-hydroxycyclohexane-1-carboxylic acid) (Compound 88)

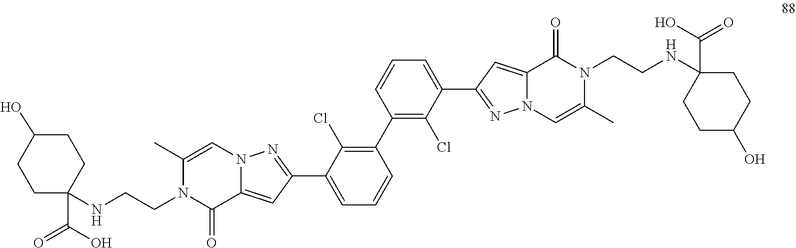 [SMILES: Cc1cn2nc(-c3cccc(-c4cccc(-c5cc6c(=O)n(CCNC7(C(=O)O)CCC(O)CC7)c(C)cn6n5)c4Cl)c3Cl)cc2c(=O)n1CCNC1(C(=O)O)CCC(O)CC1]

Following the procedure of Compound 47, the title Compound 88 was obtained by substituting 1-amino-4-hydroxycyclohexane-1-carboxylic acid for (S)-pyrrolidine-3-carboxylic acid. LCMS (ESI): calculated for C₄₄H₄₈Cl₂N₈O₈; [M+H]⁺: 887.3, 889.3, found: 887.3, 889.3. ¹H NMR (500 MHz, DMSO-d₆) δ 7.88-7.85 (m, 2H), 7.84 (s, 2H), 7.74-7.71 (m, 2H), 7.59-7.54 (m, 2H), 7.46 (s, 2H), 4.25-4.21 (m, 2H), 3.83-3.79 (m, 4H), 3.76-3.70 (m, 2H), 3.50-3.47 (m, 2H), 2.87-2.71 (m, 4H), 2.29 (s, 6H), 1.95-1.85 (m, 4H), 1.82-1.69 (m, 8H), 1.62-1.52 (m, 4H).

### Example 89. Preparation of (2S,2'S,3S,3'S)-1,1′-(((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(3-hydroxypyrrolidine-2-carboxylic acid) (Compound 89)

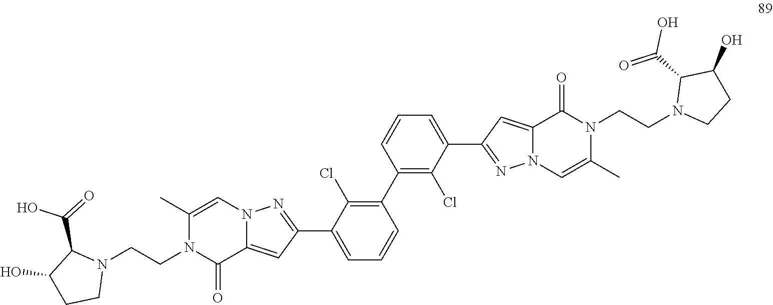 [SMILES: Cc1cn2nc(-c3cccc(-c4cccc(-c5cc6c(=O)n(CCN7CC[C@H](O)[C@H]7C(=O)O)c(C)cn6n5)c4Cl)c3Cl)cc2c(=O)n1CCN1CC[C@H](O)[C@H]1C(=O)O]

Following the procedure of Compound 47, the title Compound 89 was obtained by substituting (2S,3S)-3-hydroxypyrrolidine-2-carboxylic acid for (S)-pyrrolidine-3-carboxylic acid. LCMS (ESI): calculated for C₄₀H₄₀Cl₂N₈O₈; [M+H]⁺: 831.2, 833.2, found: 831.2, 833.2. ¹H NMR (500 MHz, DMSO-d₆) δ 7.88-7.85 (m, 2H), 7.84 (s, 2H), 7.74-7.71 (m, 2H), 7.59-7.54 (m, 2H), 7.46 (s, 2H), 4.97-4.93 (m, 2H), 4.17-4.10 (m, 2H), 3.98-3.77 (m, 4H), 3.30 (s, 2H), 3.04-2.93 (m, 2H), 2.91-2.74 (m, 6H), 2.29 (s, 6H), 1.89-1.72 (m, 4H).

### Example 90. Preparation of 2,2′-(2,2′-dichloro-[1,1′-biphenyl]-3,3′-diyl)bis(5-(2-(((1r,3r)-3-hydroxycyclobutyl)amino)ethyl)-6-methylpyrazolo[1,5-a]pyrazin-4(5H)-one) (Compound 90)

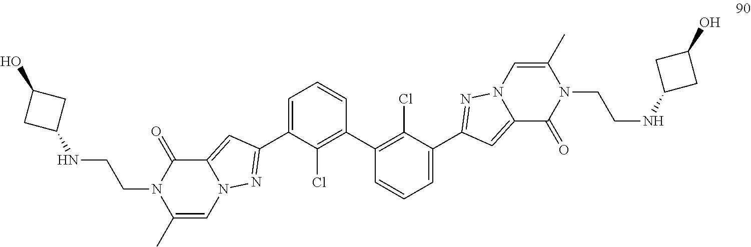 [SMILES: Cc1cn2nc(-c3cccc(-c4cccc(-c5cc6c(=O)n(CCN[C@H]7C[C@H](O)C7)c(C)cn6n5)c4Cl)c3Cl)cc2c(=O)n1CCN[C@H]1C[C@H](O)C1]

Following the procedure of Compound 47, the title Compound 90 was obtained by substituting (1R,3R)-3-aminocyclobutan-1-ol for (S)-pyrrolidine-3-carboxylic acid. LCMS (ESI): calculated for C₃₈H₄₀Cl₂N₈O₄; [M+H]⁺: 743.69, found: 743.53. ¹H NMR (500 MHz, DMSO-d₆) δ 8.83 (s, 4H), 7.93-7.77 (m, 2H), 7.57 (t, J=7.7 Hz, 2H), 7.46 (dd, J=7.6, 1.8 Hz, 2H), 7.39 (s, 2H), 5.29 (s, 2H), 4.33 (m, 2H), 4.24 (m, 4H), 3.82 (d, J=9.2 Hz, 2H), 3.21-3.08 (m, 4H), 2.40-2.32 (m, 4H), 2.38 (d, J=9.3 Hz, 6H), 2.11-1.99 (m, 4H).

### Example 91. Preparation of 2,2′-(2,2′-dichloro-[1,1′-biphenyl]-3,3′-diyl)bis(5-(2-(((1s,3s)-3-hydroxycyclobutyl)amino)ethyl)-6-methylpyrazolo[1,5-a]pyrazin-4(5H)-one) (Compound 91)

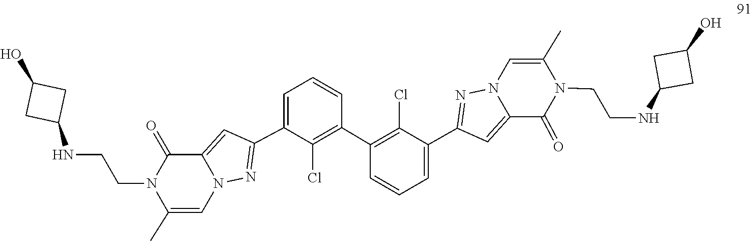 [SMILES: Cc1cn2nc(-c3cccc(-c4cccc(-c5cc6c(=O)n(CCN[C@H]7C[C@@H](O)C7)c(C)cn6n5)c4Cl)c3Cl)cc2c(=O)n1CCN[C@H]1C[C@@H](O)C1]

Following the procedure of Compound 47, the title Compound 91 was obtained by substituting (1S,3S)-3-aminocyclobutan-1-ol for (S)-pyrrolidine-3-carboxylic acid. LCMS (ESI): calculated for C₃₈H₄₀Cl₂N₈O₄; [M+H]⁺: 743.69, found: 743.51. ¹H NMR (500 MHz, DMSO-d₆) δ 8.83 (s, 4H), 7.95-7.76 (m, 2H), 7.57 (t, J=7.7 Hz, 2H), 7.46 (dd, J=7.6, 1.7 Hz, 2H), 7.38 (s, 2H), 5.42 (s, 2H), 4.30 (m, 2H), 4.24 (t, J=6.6 Hz, 4H), 3.91 (p, J=7.3 Hz, 2H), 3.19-3.06 (m, 6.9 Hz, 4H), 2.38-2.30 (m, 4H), 2.36 (s, 6H), 2.09-1.97 (m, 4H).

### Example 92. Preparation of 2,2′-(2,2′-dichloro-[1,1′-biphenyl]-3,3′-diyl)bis(5-(((2S,4R)-4-hydroxypyrrolidin-2-yl)methyl)-6-methylpyrazolo[1,5-a]pyrazin-4(5H)-one) (Compound 92)

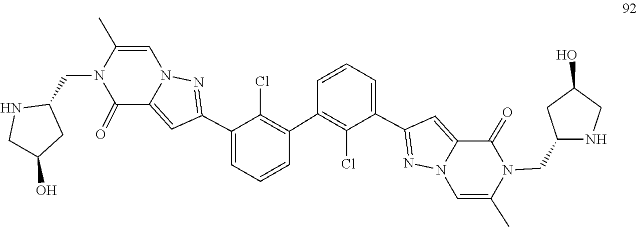 [SMILES: Cc1cn2nc(-c3cccc(-c4cccc(-c5cc6c(=O)n(C[C@@H]7C[C@@H](O)CN7)c(C)cn6n5)c4Cl)c3Cl)cc2c(=O)n1C[C@@H]1C[C@@H](O)CN1]

Following the procedure of Compound 29, the title Compound 92 was obtained by substituting ((2S,4R)-4-hydroxypyrrolidin-2-yl)methyl 4-methylbenzenesulfonate for (S)-(5-oxopyrrolidin-2-yl)methyl 4-methylbenzenesulfonate. LCMS (ESI): calculated for C₃₆H₃₆Cl₂N₈O₄; [M+H]⁺: 715.64, found: 715.24. ¹H NMR (500 MHz, DMSO-d₆) δ 8.19 (m, 2H), 7.89-7.85 (m, 2H), 7.81-7.72 (m, 2H), 7.59-7.51 (m, 2H), 7.46-7.42 (m, 2H), 7.38-7.29 (m, 2H), 4.26 (s, 2H), 4.13-4.01 (m, 2H), 3.95-3.81 (m, 2H), 3.71-3.74 (m, 2H), 3.06-3.01 (m, 2H), 2.74-2.67 (m, 2H), 2.35 (m, 6H), 1.86-1.81 (m, 2H), 1.66-1.60 (m, 2H).

### Example 93. Preparation of 2,2′-(2,2′-dichloro-[1,1′-biphenyl]-3,3′-diyl)bis(5-(2-(2-(2-hydroxyethyl)pyrrolidin-1-yl)ethyl)-6-methylpyrazolo[1,5-a]pyrazin-4(5H)-one) (Compound 93)

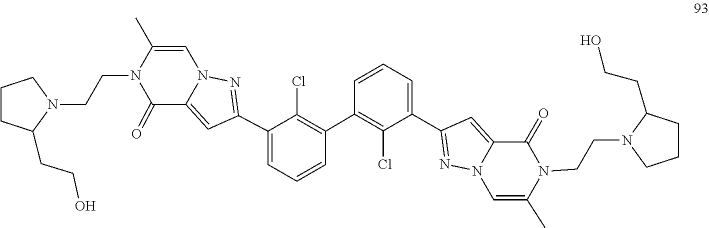 [SMILES: Cc1cn2nc(-c3cccc(-c4cccc(-c5cc6c(=O)n(CCN7CCCC7CCO)c(C)cn6n5)c4Cl)c3Cl)cc2c(=O)n1CCN1CCCC1CCO]

Following the procedure of Compound 47, the title Compound 93 was obtained by substituting 2-(pyrrolidin-2-yl)ethan-1-ol for (S)-pyrrolidine-3-carboxylic acid. LCMS (ESI): calculated for C₄₂H₄₈Cl₂N₈O₄; [M+H]⁺: 799.80, found: 799.12. ¹H NMR (500 MHz, DMSO-d₆) δ 7.89 (dd, J=7.8, 1.8 Hz, 2H), 7.81 (s, 2H), 7.56 (t, J=7.7 Hz, 2H), 7.45 (dd, J=7.8, 1.8 Hz, 2H), 7.36 (s, 2H), 4.12 (s, 4H), 3.51-3.37 (m, 14H), 3.13 (s, 2H), 2.36 (s, 6H), 1.99-1.89 (m, 4H), 1.80-1.72 (m, 4H), 1.43 (s, 4H).

### Example 94. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(propane-2,1-diyl))bis(azanediyl))bis(3-hydroxypropanoic acid) (Compound 94)

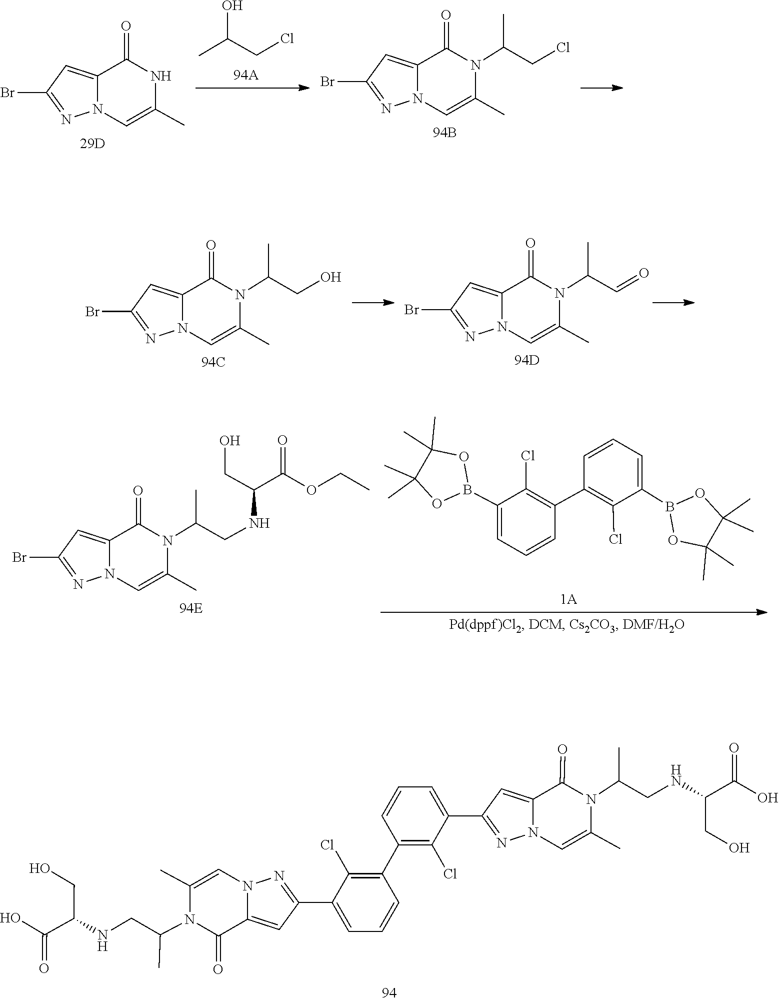

To a stirred mixture of Compound 29D (100 mg, 0.44 mmol, 1 eq) in THF (10 mL) were added Compound 94A (82 mg, 0.88 mmol, 2.0 eq.), PPh₃ (173 mg, 0.66 mmol, 1.5 eq.) and DIAD (133 mg, 0.66 mmol, 1.5 eq.) at 0° C. under N₂. The resulting mixture was stirred for 16 h at 25° C. under N₂. The reaction was quenched with water (10 mL) and extracted with EtOAc (10 mL×3). The combined organic layers were washed with brine (20 mL), dried over anhydrous sodium sulfate. After filtration, the filtrate was concentrated under reduced pressure. The residue was purified by silica gel column chromatography, eluted with PE/EtOAc (1:1) to afford Compound 94B. 80 mg. yield: 60.0%. LCMS (ESI): calculated for C₁₀H₁₁BrClN₃O; [M+H]+: 304.1, 306.1, found: 304.1, 306.1.

To a stirred mixture of Compound 94B (80 mg, 0.26 mmol, 1 eq) in DMF (5.0 mL) were added K₂CO₃ (72 mg, 0.52 mmol, 2.0 eq.) and H₂O (1.0 mL) at 25° C. The resulting mixture was stirred for 2 h at 60° C. After cooling, the reaction was quenched with water (30 mL) and extracted with EtOAc (10 mL×3). The combined organic layers were washed with brine (20 mL), dried over anhydrous sodium sulfate. After filtration, the filtrate was concentrated under reduced pressure to give Compound 94C (65 mg. yield: 86.6%). LCMS (ESI): calculated for C₁₀H₁₂BrN₃O₂; [M+H]+: 286.01, 288.01, found: 286.01, 288.01.

To a stirred mixture of Compound 94C (65 mg, 0.22 mmol, 1.0 eq) in DCM (5.0 mL) was added Dess-Martin periodinate (186 mg, 0.44 mmol, 2.0 eq.) at 25° C. The resulting mixture was stirred for 2 h at 25° C. The reaction was quenched with water (30 mL) and extracted with DCM (10 mL×3). The combined organic layers were washed with brine (20 mL), dried over anhydrous sodium sulfate. After filtration, the filtrate was concentrated under reduced pressure to give Compound 94D (60 mg. yield: 92.7%). LCMS (ESI): calculated for C₁₀H₁₀BrN₃O₂; [M+H]+: 284.01, 286.01, found: 284.01, 286.01.

To a stirred mixture of Compound 94D (60 mg, 0.20 mmol, 1.0 eq) in DMA (5.0 mL) were added ethyl L-serinate hydrochloride (136 mg, 0.8 mmol, 4.0 eq.), DIEA (103 mg, 0.8 mmol, 4.0 eq) and a drop of HOAc at 25° C. After stirred for 0.5 h, sodium triacetoxyborohydride (339 mg, 1.6 mmol, 8.0 eq) was added and the resulting mixture was stirred for 12 h at 25° C. The reaction was quenched with water (30 mL) and extracted with EtOAc (15 mL×3). The combined organic layers were washed with brine (20 mL), dried over anhydrous sodium sulfate. After filtration, the filtrate was concentrated under reduced pressure to give Compound 94E (30 mg. yield: 35.4%). LCMS (ESI): calculated for C₁₅H₂₁BrN₄O₄; [M+H]+: 401.1, 403.1, found: 401.1, 403.1.

To a stirred mixture of Compound 1A (17 mg, 0.04 mmol, 1 eq) in DMF (5.0 mL) were added Compound 94E (30 mg, 0.08 mmol, 2.1 eq.), Pd(dppf)Cl₂DCM (6.0 mg, 0.01 mmol, 0.20 eq), Cs₂CO₃ (58 mg, 0.20 mmol, 5.0 eq.) and H₂O (1.0 mL) at 25° C. The resulting mixture was stirred for 2 h at 80° C. under N₂. After cooling to 25° C., aq. NaOH (2.0 M, 1.0 mL) was added, and the resulting mixture was stirred for 1 h at 60° C. After cooling to 25° C., the resulting mixture was filtered. The filtrate is collected and purify by prep-HPLC to give compound 94 (5.0 mg, yield: 17%). LCMS (ESI): calculated for C₃₈H₄₀Cl₂N₈O₈; [M+H]+: 807.2, 809.2, found: 807.2, 809.2. ¹H NMR (500 MHz, DMSO-d₆) δ 7.88-7.85 (m, 2H), 7.84 (s, 2H), 7.74-7.71 (m, 2H), 7.59-7.55 (m, 2H), 7.46-7.44 (m, 2H), 4.95-4.81 (m, 4H), 4.48-4.36 (m, 2H), 3.67-3.39 (m, 6H), 2.97-2.83 (m, 4H), 2.28 (s, 6H), 1.32 (s, 6H).

### Example 95. Preparation of (2S,2'S)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-hydroxy-2-methylpropanoic acid) (Compound 95)

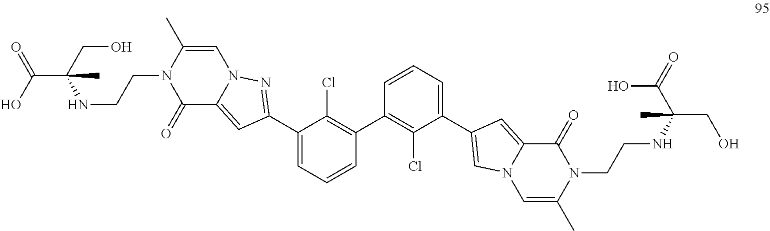 [SMILES: Cc1cn2cc(-c3cccc(-c4cccc(-c5cc6c(=O)n(CCN[C@@](C)(CO)C(=O)O)c(C)cn6n5)c4Cl)c3Cl)cc2c(=O)n1CCN[C@@](C)(CO)C(=O)O]

Following the procedure of Compound 47, the title Compound 95 was obtained by substituting (S)-2-amino-3-hydroxy-2-methylpropanoic acid for (S)-pyrrolidine-3-carboxylic acid. LCMS (ESI): calculated for C₃₈H₄₀Cl₂N₈O₈; [M+H]⁺: 807.69, found: 807.01. ¹H NMR (500 MHz, DMSO-d₆) δ 8.06 (s, 2H), 7.90-7.82 (m, 4H), 7.57 (t, J=7.7 Hz, 2H), 7.46 (dd, J=7.6, 1.7 Hz, 2H), 7.38 (s, 2H), 4.33-4.26 (t, J=7.6 Hz, 4H), 3.89 (s, 2H), 3.77-7.73 (m, 4H), 3.62-3.55 (m, 4H), 3.17 (d, J=7.8 Hz, 6H), 2.38 (s, 6H). 1.35 (s, 6H).

### Example 96. Preparation of (2R,2′R)-2,2′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-hydroxy-2-methylpropanoic acid) (Compound 96)

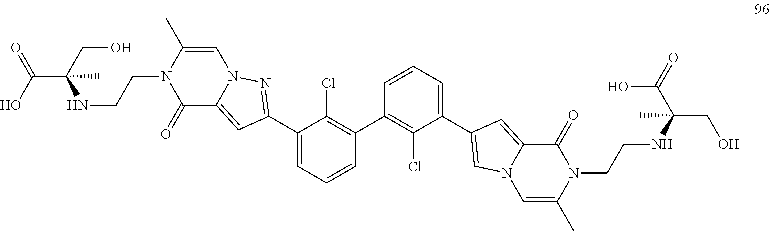 [SMILES: Cc1cn2cc(-c3cccc(-c4cccc(-c5cc6c(=O)n(CCN[C@](C)(CO)C(=O)O)c(C)cn6n5)c4Cl)c3Cl)cc2c(=O)n1CCN[C@](C)(CO)C(=O)O]

Following the procedure of Compound 47, the title Compound 96 was obtained by substituting (R)-2-amino-3-hydroxy-2-methylpropanoic acid for (S)-pyrrolidine-3-carboxylic acid. LCMS (ESI): calculated for C₃₈H₄₀Cl₂N₈O₈; [M+H]⁺: 807.69, found: 807.01. ¹H NMR (500 MHz, DMSO-d₆) δ 8.06 (s, 2H), 7.90-7.82 (m, 4H), 7.57 (t, J=7.7 Hz, 2H), 7.46 (dd, J=7.6, 1.7 Hz, 2H), 7.38 (s, 2H), 4.33-4.26 (t, J=7.6 Hz, 4H), 3.89 (s, 2H), 3.77-7.73 (m, 4H), 3.62-3.55 (m, 4H), 3.17 (d, J=7.8 Hz, 6H), 2.38 (s, 6H). 1.35 (s, 6H).

### Example 97. Preparation of (1s,1's,3s,3's)-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(cyclobutane-3,1-diyl) diacetate (Compound 97)

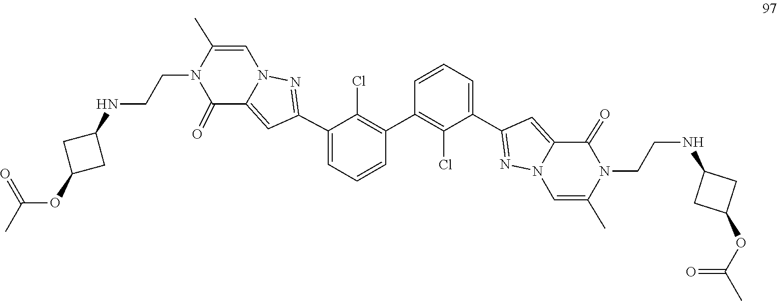 [SMILES: CC(=O)O[C@H]1C[C@@H](NCCn2c(C)cn3nc(-c4cccc(-c5cccc(-c6cc7c(=O)n(CCN[C@H]8C[C@@H](OC(C)=O)C8)c(C)cn7n6)c5Cl)c4Cl)cc3c2=O)C1]

Following the procedure of Compound 47, the title Compound 97 was obtained by substituting (1S,3S)-3-aminocyclobutyl acetate hydrochloride for (S)-pyrrolidine-3-carboxylic acid. LCMS (ESI): calculated for C₄₂H₄₆Cl₄N₈O₆; [M+H]⁺: 900.68, found: 828.12. ¹H NMR (500 MHz, DMSO-d₆) δ 8.81 (s, 4H), 7.93-7.74 (m, 2H), 7.55 (t, J=7.7 Hz, 2H), 7.44 (dd, J=7.6, 1.7 Hz, 2H), 7.36 (s, 2H), 5.40 (s, 2H), 4.28 (m, 2H), 4.22 (t, J=6.6 Hz, 4H), 3.90 (p, J=7.3 Hz, 2H), 3.18-3.05 (m, 6.9 Hz, 4H), 2.36-2.29 (m, 4H), 2.35 (s, 6H), 2.06-1.94 (m, 4H), 2.02 (s, 6H).

### Example 98. Preparation of ((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(ethane-2,1-diyl) diacetate (Compound 98)

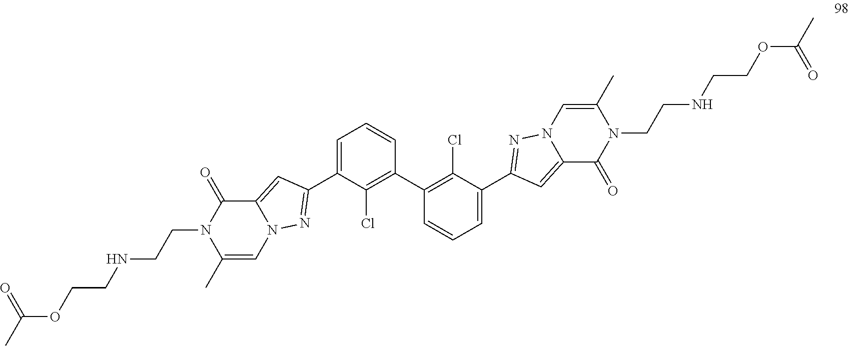 [SMILES: CC(=O)OCCNCCn1c(C)cn2nc(-c3cccc(-c4cccc(-c5cc6c(=O)n(CCNCCOC(C)=O)c(C)cn6n5)c4Cl)c3Cl)cc2c1=O]

Following the procedure of Compound 47, the title Compound 98 was obtained by substituting 2-aminoethyl acetate hydrochloride for (S)-pyrrolidine-3-carboxylic acid. LCMS (ESI): calculated for C₃₈H₄₀Cl₂N₈O₆; [M+H]⁺: 775.2, 777.2, found: 775.2, 777.2. ¹H NMR (500 MHz, DMSO-d₆): δ 7.88-7.85 (m, 2H), 7.84 (s, 2H), 7.75-7.71 (m, 2H), 7.59-7.54 (m, 2H), 7.48-7.46 (m, 2H), 4.14-4.08 (m, 4H), 3.83-3.76 (m, 4H), 3.07-3.01 (m, 4H), 2.86-2.81 (m, 2H), 2.76-2.72 (m, 4H), 2.29 (s, 6H), 2.02 (s, 6H).

### Example 99. Preparation of diethyl 1,1′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-hydroxycyclobutane-1-carboxylate) (Compound 99)

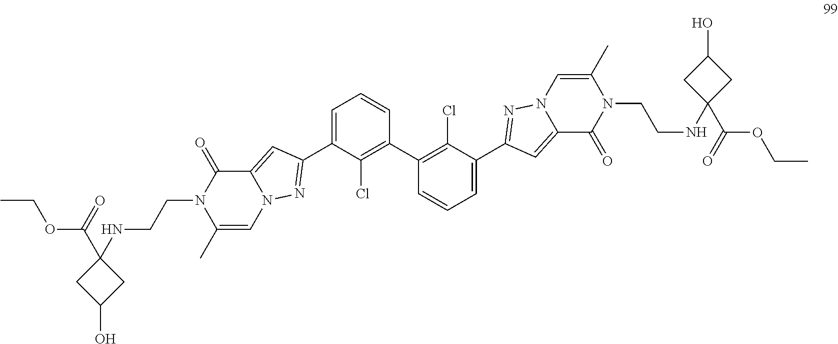 [SMILES: CCOC(=O)C1(NCCn2c(C)cn3nc(-c4cccc(-c5cccc(-c6cc7c(=O)n(CCNC8(C(=O)OCC)CC(O)C8)c(C)cn7n6)c5Cl)c4Cl)cc3c2=O)CC(O)C1]

Following the procedure of Compound 47, the title Compound 99 was obtained by substituting ethyl 1-amino-3-hydroxycyclobutane-1-carboxylate for (S)-pyrrolidine-3-carboxylic acid. LCMS (ESI): calculated for C₄₄H₄₈Cl₂N₈O₈; [M+H]⁺: 887.3, 889.3, found: 887.3, 889.3. ¹H NMR (500 MHz, DMSO-d₆) δ 7.89 (dd, J=7.8, 1.8 Hz, 2H), 7.81 (d, J=12.7 Hz, 2H), 7.56-7.44 (m, 6H), 7.36 (d, J=2.6 Hz, 2H), 4.17 (s, 2H), 4.12 (m, 4H), 4.08-4.02 (m, 4H), 3.34-3.29 (m, 4H), 2.39 (m, 6H), 2.44-2.38 (m, 4H), 2.24-2.20 (m, 4H), 1.24 (d, J=10 Hz, 6H).

### Example 100. Preparation of diethyl 2,2′-((((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))bis(azanediyl))(2S,2'S)-bis(3-hydroxypropanoate) (Compound 100)

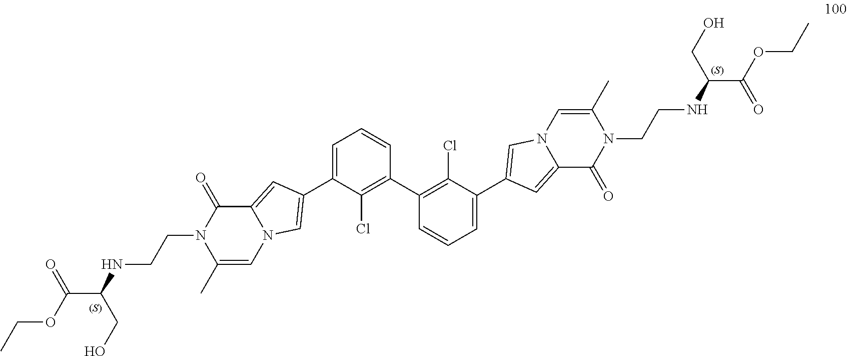

Following the procedure of Example 48, the title Compound 100 was obtained by substituting ethyl L-serinate for (S)-pyrrolidin-3-ol. LCMS (ESI): calculated for C₄₂H₄₆Cl₂N₆O₈; [M+H]⁺: 833.3, 835.3 found: 833.3, 835.3. ¹H NMR (500 MHz, DMSO-d6) δ 7.76 (d, J=1.8 Hz, 2H), 7.67 (dd, J=7.8, 1.7 Hz, 2H), 7.46 (t, J=7.7 Hz, 2H), 7.28 (ddd, J=5.0, 3.7, 1.7 Hz, 4H), 7.18 (d, J=1.7 Hz, 2H), 4.83 (s, 2H), 4.07 (q, J=7.1 Hz, 4H), 3.99-3.91 (m, 4H), 3.58-3.52 (m, 4H), 2.82 (dt, J=11.7, 6.9 Hz, 2H), 2.70-2.63 (m, 2H), 2.52 (d, J=1.9 Hz, 2H), 2.27 (s, 6H), 1.16 (d, J=7.2 Hz, 6H).

### Example 101. Preparation of (1r,1′r,3r,3′r)-1,1′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))bis(3-hydroxycyclobutane-1-carboxylic acid) (Compound 101)

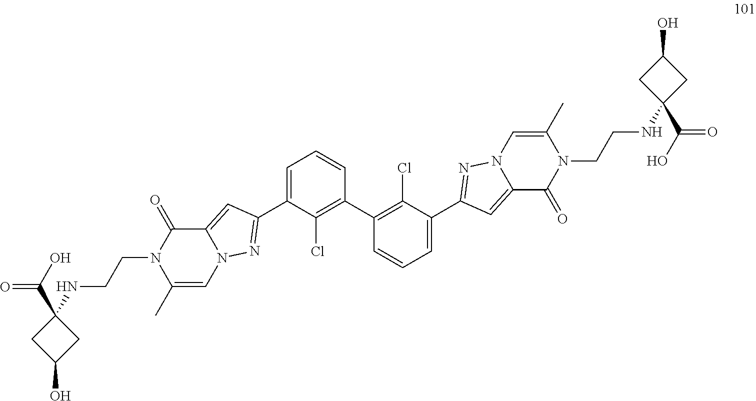 [SMILES: Cc1cn2nc(-c3cccc(-c4cccc(-c5cc6c(=O)n(CCN[C@]7(C(=O)O)C[C@@H](O)C7)c(C)cn6n5)c4Cl)c3Cl)cc2c(=O)n1CCN[C@]1(C(=O)O)C[C@@H](O)C1]

Following the procedure of Example 47, the title Compound 101 was obtained by substituting (1R,3R)-1-amino-3-hydroxycyclobutane-1-carboxylic acid for (S)-pyrrolidine-3-carboxylic acid. LCMS (ESI): calculated for C₄₀H₄₀Cl₂N₈O₈; [M+H]+: 831.2, 833.2, found: 831.2, 833.2. ¹H NMR (500 MHz, DMSO-d₆) δ 7.89-7.85 (m, 2H), 7.84 (s, 2H), 7.75-7.71 (m, 2H), 7.59-7.54 (m, 2H), 7.48-7.46 (m, 2H), 5.02-4.98 (m, 2H), 4.80-4.77 (m, 2H), 3.92-3.84 (m, 2H), 3.83-3.78 (m, 4H), 2.87-2.73 (m, 4H), 2.68-2.62 (m, 4H), 2.29 (s, 6H), 2.24-2.19 (m, 4H).

### Example 102. Preparation of (1S,1'S,3S,3'S)-1,1′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl)) bis(3-hydroxycyclobutane-1-carboxylic acid) (Compound 102)

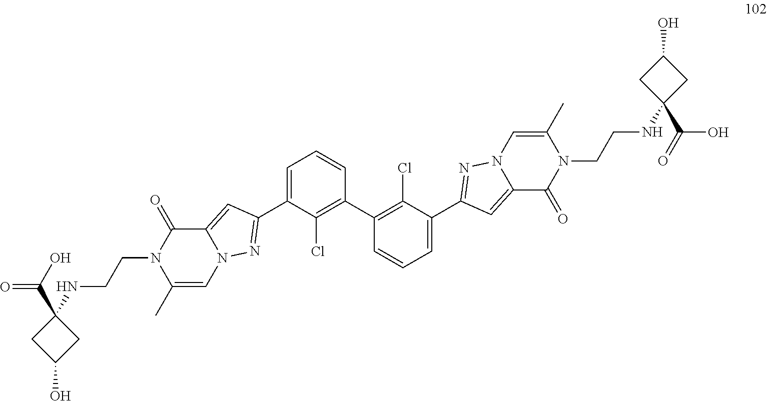 [SMILES: Cc1cn2nc(-c3cccc(-c4cccc(-c5cc6c(=O)n(CCN[C@]7(C(=O)O)C[C@H](O)C7)c(C)cn6n5)c4Cl)c3Cl)cc2c(=O)n1CCN[C@]1(C(=O)O)C[C@H](O)C1]

Following the procedure of Example 47, the title Compound 102 was obtained by substituting (1S,3S)-1-amino-3-hydroxycyclobutane-1-carboxylic acid for (S)-pyrrolidine-3-carboxylic acid. LCMS (ESI): calculated for C₄₀H₄₀Cl₂N₈O₈; [M+H]+: 831.2, 833.2, found: 831.2, 833.2. ¹H NMR (500 MHz, DMSO-d₆) δ 7.89-7.85 (m, 2H), 7.84 (s, 2H), 7.75-7.71 (m, 2H), 7.59-7.54 (m, 2H), 7.48-7.46 (m, 2H), 5.02-4.98 (m, 2H), 4.80-4.77 (m, 2H), 3.92-3.84 (m, 2H), 3.83-3.78 (m, 4H), 2.87-2.73 (m, 4H), 2.68-2.62 (m, 4H), 2.29 (s, 6H), 2.24-2.19 (m, 4H).

### Example 103. Preparation of diethyl 1,1′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))(1R,1′R,3R,3′R)-bis(3-hydroxycyclobutane-1-carboxylate) (Compound 103)

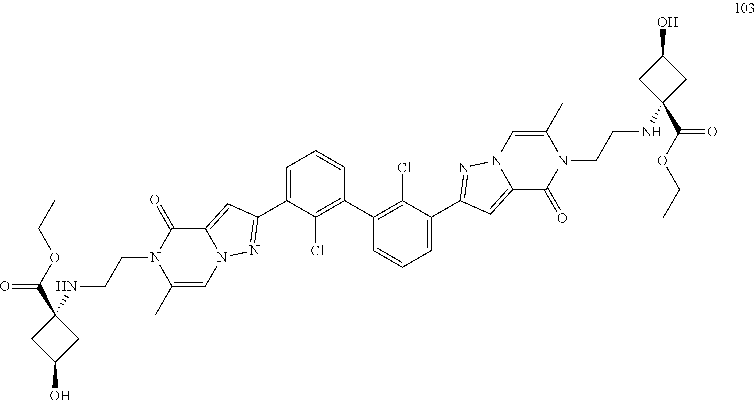 [SMILES: CCOC(=O)[C@]1(NCCn2c(C)cn3nc(-c4cccc(-c5cccc(-c6cc7c(=O)n(CCN[C@]8(C(=O)OCC)C[C@@H](O)C8)c(C)cn7n6)c5Cl)c4Cl)cc3c2=O)C[C@H](O)C1]

Following the procedure of Example 47, the title Compound 103 was obtained by substituting ethyl (1R,3R)-1-amino-3-hydroxycyclobutane-1-carboxylate for (S)-pyrrolidine-3-carboxylic acid. LCMS (ESI): calculated for C₄₄H₄₈Cl₂N₈O₈; [M+H]+: 887.3, 889.3, found: 887.3, 889.3. ¹H NMR (500 MHz, DMSO-d6) δ 7.95-7.88 (m, 2H), 7.77 (s, 2H), 7.60-7.54 (m, 2H), 7.49-7.42 (m, 2H), 7.37 (s, 2H), 5.12-5.05 (m, 2H), 4.12-4.02 (m, 6H), 4.01-3.94 (m, 4H), 2.75-2.61 (m, 6H), 2.41 (s, 6H), 2.10-1.98 (m, 8H), 1.19-1.13 (m, 6H).

### Example 104. Preparation of diethyl 1,1′-((((2,2′-dichloro-[1,1′-biphenyl]-3,3′-diyl)bis(6-methyl-4-oxopyrazolo[1,5-a]pyrazine-2,5(4H)-diyl))bis(ethane-2,1-diyl))bis(azanediyl))(1S,1'S,3S,3'S)-bis(3-hydroxycyclobutane-1-carboxylate) (Compound 104)

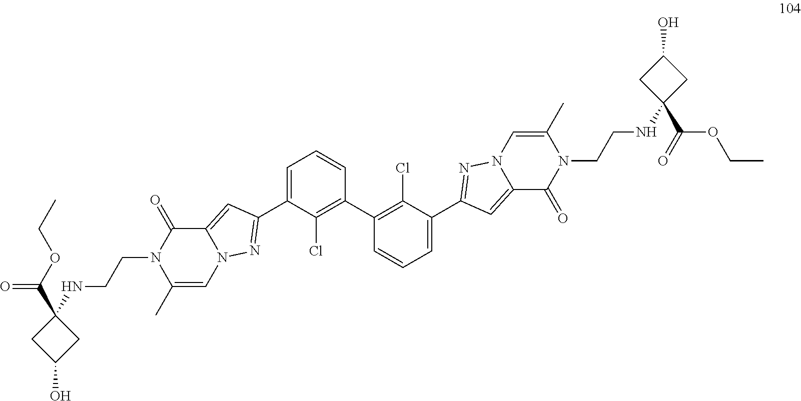 [SMILES: CCOC(=O)[C@]1(NCCn2c(C)cn3nc(-c4cccc(-c5cccc(-c6cc7c(=O)n(CCN[C@]8(C(=O)OCC)C[C@H](O)C8)c(C)cn7n6)c5Cl)c4Cl)cc3c2=O)C[C@@H](O)C1]

Following the procedure of Example 47, the title Compound 104 was obtained by substituting ethyl (1S,3S)-1-amino-3-hydroxycyclobutane-1-carboxylate for (S)-pyrrolidine-3-carboxylic acid. LCMS (ESI): calculated for C₄₄H₄₅Cl₂N₈O₈; [M+H]+: 887.3, 889.3, found: 887.3, 889.3. ¹H NMR (500 MHz, DMSO-d6) δ 7.95-7.88 (m, 2H), 7.77 (s, 2H), 7.60-7.54 (m, 2H), 7.49-7.42 (m, 2H), 7.37 (s, 2H), 5.12-5.05 (m, 2H), 4.12-4.02 (m, 6H), 4.01-3.94 (m, 4H), 2.75-2.61 (m, 6H), 2.41 (s, 6H), 2.10-1.98 (m, 8H), 1.19-1.13 (m, 6H).

### Example 105. Preparation of diethyl 1,1′-(((2,2′-dichloro-[1,1′-biphenyl]-3,3′-diyl)bis(3-methyl-1-oxopyrrolo[1,2-a]pyrazine-7,2(1H)-diyl))bis(ethane-2,1-diyl))(3S,3'S)-bis(pyrrolidine-3-carboxylate) (Compound 105)

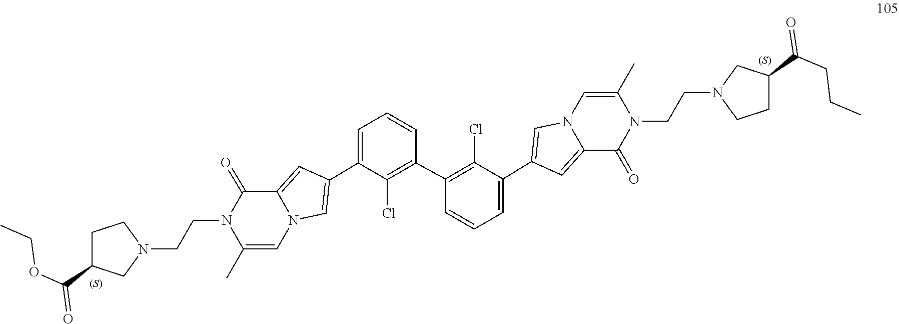

Following the procedure of Example 48, the title Compound 105 was obtained by substituting ethyl (S)-pyrrolidine-3-carboxylate for (S)-pyrrolidin-3-ol. LCMS (ESI): calculated for C₄₆H₅₀Cl₂N₆O₆; [M+H]⁺: 853.84, found: 853.43. ¹H NMR (500 MHz, DMSO-d6) δ 7.77 (d, J=1.8 Hz, 2H), 7.67 (dd, J=7.8, 1.7 Hz, 2H), 7.47 (t, J=7.6 Hz, 2H), 7.32-7.26 (m, 4H), 7.18 (d, J=1.7 Hz, 2H), 4.07 (q, J=7.1 Hz, 4H), 3.98 (t, J=7.1 Hz, 4H), 3.04-2.95 (m, 2H), 2.81 (t, J=8.8 Hz, 2H), 2.69 (dd, J=9.2, 6.1 Hz, 2H), 2.65-2.55 (m, 8H), 2.27 (s, 6H), 1.99-1.90 (m, 4H), 1.18 (t, J=7.1 Hz, 6H).

### Example 106. Preparation of diethyl 1,1′-(((2,2′-dichloro-[1,1′-biphenyl]-3,3′-diyl)bis(2-methyl-1-oxo-1,2-dihydro pyrrolo[1,2-a]pyrazine-7,3-diyl))bis(methylene))(3S,3'S)-bis(pyrrolidine-3-carboxylate) (Compound 106)

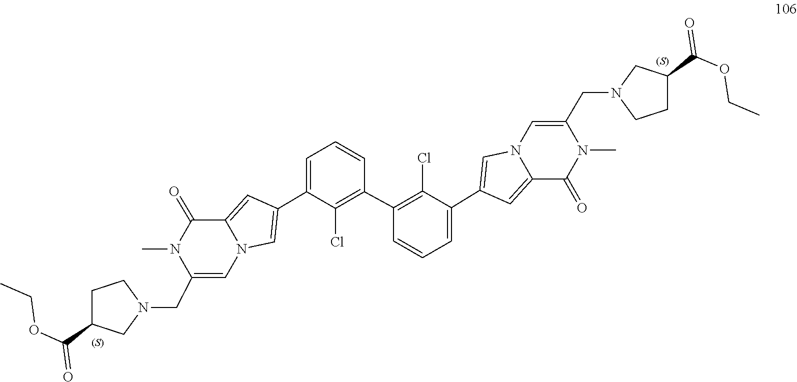

Following the procedure of Example 43, the title Compound 106 was obtained by substituting ethyl (S)-pyrrolidine-3-carboxylate for (S)-pyrrolidine-3-carboxylic acid. LCMS (ESI): calculated for C₄₄H₄₆Cl₂N₆O₆; [M+H]⁺: 825.79, found: 825.51. ¹H NMR (500 MHz, DMSO-d6) δ 7.81 (s, 2H), 7.71-7.65 (m, 2H), 7.50 (t, J=7.6 Hz, 2H), 7.38 (s, 2H), 7.31 (d, J=7.4 Hz, 2H), 7.26 (d, J=1.7 Hz, 2H), 4.05 (q, J=7.1 Hz, 4H), 3.94 (s, 6H), 3.55 (s, 4H), 3.21-3.08 (m, 4H), 3.04-2.93 (m, 4H), 2.63-2.55 (m, 2H), 2.04-1.94 (m, 2H), 1.90-1.77 (m, 2H), 1.14 (t, J=7.1 Hz, 6H).

### BIOLOGICAL EXAMPLES

### Example 107. PD-1/PD-L1 Homogeneous Time-Resolved Fluorescence (HTRF) Binding Assay

The assays were conducted in a standard black 384-well polystyrene plate with a final volume of 20 μL. Inhibitors were first serially diluted in DMSO and then added to the plate wells before the addition of other reaction components. The final concentration of DMSO in the assay was 1%. The assays were carried out at 25° C. in the PBS buffer (pH 7.4) with 0.05% Tween-20 and 0.1% BSA. Recombinant human PD-L1 protein (19-238) with a Histag at the C-terminus was purchased from AcroBiosystems (PD1-H5229). Recombinant human PD-1 protein (25-167) with Fe tag at the C-terminus was also purchased from AcroBiosystems (PD1-H5257). PD-L1 and PD-1 proteins were diluted in the assay buffer and 10 μL was added to the plate well. Plates were centrifuged and proteins were preincubated with inhibitors for 40 minutes. The incubation was followed by the addition of 10 μL of HTRF detection buffer supplemented with Europium cryptate-labeled anti-human IgG (PerkinElmer-AD0212) specific for Fe and anti-His antibody conjugated to SureLight®—Allophycocyanin (APC, PerkinElmer-AD0059H). After centrifugation, the plate was incubated at 25° C. for 60 min. before reading on a PHERAstar FS plate reader (665 nm/620 nm ratio). Final concentrations in the assay were −3 nM PD1, 10 nM PD-L1, 1 nM europium anti-human IgG and 20 nM anti-His-Allophycocyanin. IC₅₀ determination was performed by fitting the curve of percent control activity versus the log of the inhibitor concentration.

TABLE 2
PD-1/PD-L1 HTRF activity

| Compound | HTRF |
|---|---|
| ID | IC₅₀ |
| 1 | A |
| 2 | B |
| 3 | A |
| 4 | C |
| 5 | B |
| 6 | C |
| 7 | A |
| 8 | B |
| 9 | C |
| 10 | C |
| 11 | C |
| 12 | B |
| 13 | C |
| 14 | B |
| 15 | A |
| 16 | B |
| 17 | A |
| 18 | B |
| 19 | C |
| 20 | C |
| 21 | C |
| 22 | B |
| 23 | C |
| 24 | C |
| 25 | B |
| 26 | C |
| 27 | C |
| 28 | C |
| 29 | A |
| 30 | A |
| 31 | B |
| 32 | B |
| 33 | B |
| 34 | B |
| 35 | A |
| 36 | A |
| 37 | B |
| 38 | B |
| 39 | B |
| 40 | A |
| 41 | B |
| 42 | A |
| 43 | B |
| 44 | B |
| 45 | A |
| 46 | B |
| 47 | A |
| 48 | B |
| 49 | A |
| 50 | B |
| 51 | A |
| 52 | A |
| 53 | A |
| 54 | A |
| 55 | C |
| 56 | C |
| 57 | A |
| 58 | B |
| 59 | C |
| 60 | B |
| 61 | B |
| 62 | B |
| 63 | A |
| 64 | B |
| 65 | B |
| 66 | A |
| 67 | C |
| 68 | B |
| 69 | C |
| 70 | B |
| 71 | A |
| 72 | B |
| 73 | B |
| 74 | B |
| 75 | B |
| 76 | B |
| 79 | B |
| 80 | B |
| 81 | B |
| 82 | C |
| 83 | C |
| 84 | C |
| 85 | B |
| 86 | C |
| 87 | C |
| 88 | B |
| 89 | B |
| 90 | A |
| 91 | A |
| 92 | A |
| 93 | B |
| 94 | B |
| 95 | B |
| 96 | B |
| 101 | A |
| 102 | A |

A: activity <10 nM;
B: 10 nM ≤ activity <100 nM;
C: Activity ≥100 nM

### Example 108. PD-L1 Dimerization

Compounds were tested in biochemical protein-protein interaction assays to determine if they can specifically dimerize the extracellular domains of PD-L1.
(1) Diluted cpd 1:3 in succession in DMSO for each column for 10 samples
(2) Transferred 0.2 μL cpds solution in each row to 384 assay plate using Echo, each column containing 2 replicates
(3) Added 20 μL prepared mixture include PD-L1-Eu and PD-L1-A2 solution to assay plate, centrifuged at 1000 rpm for 1 min.
(4) Incubated at 25° C. for 120 mA.
(5) Read fluorescence signal on Envision 2104 plate reader.
(6) Read the Ratio (665 nm/615 nm) signal on Envision.
(7) Analyzed the raw data using the equation (V. Data analysis)

TABLE 3
Dimerization EC50

| Compound | Dimerization |
|---|---|
| ID | EC₅₀ |
| 36 | A |
| 43 | B |
| 47 | B |
| 49 | C |
| 51 | B |
| 52 | A |
| 57 | B |
| 63 | B |
| 76 | A |
| 80 | C |
| 85 | B |
| 90 | A |
| 91 | A |
| 95 | C |
| 96 | C |
| 101 | A |

A: EC50 <50 nM;
B: 50 nM ≤ EC50 <100 nM;
C: EC50 ≥100 nM

### Example 109. PDL1 Jurkat-NFAT Reporter Assay

### Preparation of Hep3B-OS8-hPDL1

1. Hep3B-OS8-hPDL1 cells were cultured in 1640 medium supplemented with 10% fetal bovine serum, 1% penicillin and streptomycin, in which 100 μg/mL G418 and Hygromycin B were also added.

2. Cells were re-suspended with RPMI 1640 medium containing 10% FBS and the cell density was adjusted to 1.25×10⁵ cells/mL.

3. Cells were seeded into 96-well flat bottom plate (1.25×10⁴ cells/100 μL/well).

b. Compounds solutions preparation

4. Removed the medium from pre-plated Hep3B-OS8-PDL1 cells. Washed once with 200 μL Assay Medium.

5. Prepared the Compounds dilutions in RPMI 1640 medium with 10% FBS according to the Layout.

6. Added compounds into each well in a volume of 50 μL in 9 concentrations (3, 1, 0.3, 0.1, 0.03, 0.01, 0.003, 0.001 and 0.0003 PM)}. Keytruda will be included as positive control at the concentration of 5 μg/mL.

7. Incubated for 20-30 min at 37° C., 5% CO₂.

c. Preparation of Jurkat-NFAT-PD1

8. Jurkat-NFAT-PD1 cells were cultured in 1640 medium supplemented with 10% fetal bovine serum, 1% penicillin and streptomycin, in which 1000 μg/mL Hygromycin B and 0.3 sg/mL puromycin were also added.

9. On the second day of assay, the cells were re-suspended with RPMI 1640 medium containing 10% FBS and the cell density was adjusted to 2.5×10⁵ cells/mL.

10. Cells were seeded into 96-well flat bottom plate (1.25×10⁴ cells/50 μL/well).

11. Incubated the assay plate in a humidified 37° C., 5% CO₂ incubator for 6 hours.

12. Equilibrated cultured cells at room temperature for 5-10 mins.

13. Added equal volume (100 μL/well) of ONE-Glo™ Luciferase Assay System to each well, waited at least 3 mins to allow complete cell lysis and measure in a luminometer.

TABLE 4

| Compound | Jurkat-NFAT |
|---|---|
| ID | EC₅₀ |
| 1 | A |
| 74 | B |

A: EC50 <500 nM;
B: EC50 ≥500 nM

### Example 110. Hep3B-OS8-hPDL1 and T Cells Co-Culture Assay

### Tumor Preparation

1. Hep3B-OS8-hPDL1 cells were cultured in 1640 medium supplemented with 10% fetal bovine serum, 1% penicillin and streptomycin, in which 100 μg/mL G418 and Hygromycin B were also added.

2. Hep3B-OS8-hPDL1 cells were harvested and treated with 10 μg/mL mitomycin C at 37° C. for 1.5 h, and the cells were then washed thoroughly with PBS for four times.

3. Cells were re-suspended with RPMI 1640 medium containing 10% FBS and the cell density was adjusted to 5×10⁵ cells/mL.

4. Cells were seeded into 96-well flat bottom plate (2.5×10⁴ cells/50 μL/well).

b. CD3+ T cells isolation (30 mL blood)

5. The human blood sample from an individual donor was diluted by the same volume of sterile PBS, for instance add 25 mL sterile PBS into 25 mL fresh whole blood and mix sufficiently by gentle shake.

6. Transferred 15 mL Lymphoprep medium into a new 50 mL centrifuge tube.

7. Added the diluted blood sample as soft as possible onto the surface of the Ficoll medium to make sure there is a clear dividing line between the two liquids, and the volume ratio between Ficoll and diluted blood (30 mL) is 1:2.

8. Moved the tube gently to centrifuge, at 1000×g, 25 min, 20° C., with the acceleration (5) and Min deceleration (0) settings during the centrifugation.

9. Four interfaces in all could be observed after the centrifugation, which are layers of plasma, mononuclear cells, the Ficoll medium and RBCs from top to bottom, and move the tube as gently as possible so as keep the four interfaces separated. Absorbed and transferred the second layer of mononuclear cells into another new sterile centrifuge tube carefully, and absorbed certain volume of plasma instead of the Ficoll medium if inevitable.

10. Added sterile PBS of the three times the volume of PBMCs in the tube with PBMC.

11. Washed the cells twice with 5-10 mL PBS before cell counting with cytometer. Centrifuge at 350×g, 10 min, 20° C. With the acceleration (5) and deceleration (5) settings during the centrifugation.

12. Resuspended the cells with recommended medium and adjust the density of the PBMCs to the final concentration of 5×10⁷ cells/mL.

13. Isolated CD3+ T cells with EasySep™ Human T Cell Isolation Kit (STEMCELL Technologies #17951), and the cells were seeded into 96-well flat bottom plate (5×10⁴ cells/100 μL/well). c. Compounds solutions preparation

14. Prepared the Compounds dilutions in RPMI 1640 medium with 10% FBS according to the Layout. 15. Added compounds into each well in a volume of 50 μL. {3 compounds (GLC01-258, GLC01-269, GLC01-465) in 7 concentrations (0.03, 0.1, 0.3, 1, 3, 10 and 30 μM) and 6 compounds (GLC01-411, GLC01-292, GLC01-445, GLC01-475, GLC01-470 and GLC01-468) in the same concentration (1 μM)}.

16. Keytruda was included as positive control, the concentration was 5 μg/mL. 17. Incubated for 72 hours at 37° C., 5% CO₂.

18. Collected supernatant by centrifugation and measure IFN-γ by ELISA.

TABLE 5
Hep3B-OS8-hPDL1 and T cells co-culture assay activity

| Compound | Tumor and T |
|---|---|
| ID | Cell EC₅₀ |
| 36 | A |
| 47 | C |
| 51 | B |
| 52 | B |
| 53 | C |
| 57 | A |
| 63 | C |
| 76 | B |
| 85 | A |
| 89 | A |

A: EC50 <50 nM;
B: 50 nM ≤ EC50 <100 nM;
C: EC50 ≥100 nM

### Example 111. Mouse PK Study

(1) The compounds were weighed and dissolved in the vehicle of 1 mg/mL 5% solutol in saline, shaken well, and sonicated to form a colorless clear solution. The solution was given orally at a dose of 10 mg/kg to a group of 3 mice after an overnight fast.

(2) Blood was collected from the submandibular vein, and heparin sodium was used for anticoagulation. The blood was placed on ice after collection and plasma was separated by centrifugation within 1 hour (centrifugation conditions: 8000 rpm, 6 minutes, 2-8° C.). Blood sampling time points were 0.083, 0.25, 0.5, 1, 2, 4, 8, and 24 hours.

(3) The samples were stored in a −20′° C. freezer. The plasma sample (40 μL) was added with 160 μL of ice-cold acetonitrile containing internal standard, vortexed for 3 minutes, and centrifuged at 11,000 rpm for 5 minutes. 100 μL of the supernatant was added to 100 μL of water, and 5 μL of the supernatant was injected in LC/MS/MS instrument to detect the compound (if the compound was an ester, acid was detected). The results are shown in Table 6.

TABLE 6

| Compound | | Tₘₐₓ | | T_{1/2} |
|---|---|---|---|---|
| ID | Cₘₐₓ | (h) | AUCₗₐₛₜ | (h) |
| 1 | C | 1 | F | 2.23 |
| 77 | A | 0.33 | D | 2.7 |
| 78 | B | 0.833 | D | 4.31 |
| 97 | C | 0.333 | F | 2.68 |
| 99 | B | 2.17 | E | 4.23 |
| 100 | A | 0.833 | D | 5.3 |
| 105 | C | 1 | F | 2.8 |

A: Cmax ≥1000 ng/mL;
B: 100 ng/mL ≤ Cmax <1000 ng/mL;
C: Cmax <100 ng/mL
D: AUCₗₐₛₜ ≥2000 ng*h/mL;
E: 500 ng*h/mL ≤ SAUCₗₐₛₜ <2000 ng*h/mL;
F: AUCₗₐₛₜ <500 ng*h/mL

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives there intended, unless the context specifically indicates the contrary.

1. A compound of Formula (I): 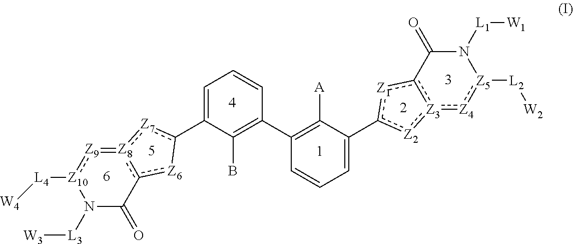
or a pharmaceutically acceptable salt, stereoisomer, mixture of stereoisomers, solvate, or tautomer thereof, wherein:
each of A and B is independently selected from the group consisting of halogen, cyano, —N₃, alkyl and substituted alkyl, amine, alkylamine, and alkoxy;
Z₁ is ═N—, —N(R¹)—, ═C(R²)—, or —S—;
Z₂ is ═N—, —N(R¹)—, ═C(R²)—, or —S—;
Z₆ is ═N—, —N(R¹)—, ═C(R²)—, or —S—;
Z₇ is ═N—, —N(R¹)—, ═C(R²)—, or —S—;
each R¹ is independently —H, alkyl, cycloalkyl, substituted alkyl, alkenyl, alkynyl, or aryl; each R² is independently halogen, cyano, cycloalkyl, substituted alkyl, alkenyl, alkynyl, or aryl;
Z₃ is —N═, —N(R³)—, ═C(R⁴)—, or —(R⁵R⁶)—;
Z₄ is —N═, —N(R³)—, ═C(R⁴)—, or —C(R⁵R⁶)—;
Z₅ is —N═, —N(R³)—, ═C(R⁴)—, or —C(R⁵R⁶)—;
Z₈ is —N═, —N(R³)—, ═C(R⁴)—, or —C(R⁵R⁶)—;
Z₉ is —N═, —N(R³)—, ═C(R⁴)—, or —C(R⁵R⁶);
Z₁₀ is —N═, —N(R³)—, ═C(R⁴)—, or —C(R⁵R⁶)—;
each R³ is independently —H, alkyl, cycloalkyl, substituted alkyl, alkenyl, alkynyl, or aryl;
each of R⁴, R⁵ and R⁶ is independently halogen, cyano, cycloalkyl, substituted alkyl, alkenyl, alkynyl, or aryl;
each of L₁ and L₃ is independently an alkyl, substituted alkyl or hetereoatom chain, containing m atoms between Ring 3 and W₁, and between Ring 6 and W₃, wherein m=0, 1, 2, 3, 4, 5 or 6; when m is 0, W₁ or W₃ is directly linked to the corresponding nitrogen in Ring 3 or Ring 6, respectively;
each of W₁ and W₃ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid;
each of L₂ and L₄ is independently an alkyl, substituted alkyl or hetereoatom chain, containing m atoms between Ring 3 and W₂, and between Ring 6 and W₄, wherein m=0, 1, 2, 3, 4, 5 or 6; when m is 0, W₂ or W₄ is directly linked to the corresponding nitrogen in Ring 3 or Ring 6, respectively;
each of W₂ and W₄ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

2. The compound of claim 1,
wherein each of A and B is independently —Cl or methyl;
Z₁ is ═N—, —N(R¹)—, ═C(R²)—, or —S—;
Z₂ is ═N—, —N(R¹)—, ═C(R²)—, or —S—;
Z₆ is ═N—, —N(R¹)—, ═C(R²)—, or —S—;
Z₇ is ═N—, —N(R¹)—, ═C(R²), or —S—;
each R¹ is independently —H, alkyl, cycloalkyl, or substituted alkyl; each R² is independently —H, —F, or methyl;
Z₃ is —N═, —N(R³)—, ═C(R⁴)—, or —C(R⁵R⁶)—;
Z₄ is —N═, —N(R³)—, ═C(R⁴)—, or —C(R⁵R⁶)—;
Z₅ is —N═, —N(R³)—, ═C(R⁴)—, or —C(R⁵R⁶)—;
Z₈ is —N═, —N(R³)—, ═C(R⁴)—, or —C(R⁵R⁶)—;
Z₉ is —N═, —N(R³)—, ═C(R⁴)—, or —C(R⁵R⁶)—;
Z₁₀ is —N═, —N(R³)—, ═C(R⁴)—, or —C(R⁵R⁶)—;
each R³ is independently —H, alkyl, cycloalkyl, or substituted alkyl;
each of R⁴, R⁵ and R⁶ is independently halogen, cyano, cycloalkyl, or substituted alkyl;
each of L₁ and L₃ is independently an alkyl, substituted alkyl or hetereoatom chain, containing m atoms between Ring 3 and W₁, and between Ring 6 and W₃, wherein m=0, 1, 2, 3, 4, 5 or 6; when m is 0, W₁ or W₃ is directly linked to the corresponding nitrogen in Ring 3 or Ring 6, respectively;
each of W₁ and W₃ is independently hydrogen, a three member ring or substituted three member ring, a four member ring or substituted four member ring, a four member heterocyclic ring or substituted four member heterocyclic ring, a five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid;
each of L₂ and L₄ is independently an alkyl, substituted alkyl or hetereoatom chain, containing m atoms between Ring 3 and W₂, and between Ring 6 and W₄, wherein m=0, 1, 2, 3, 4, 5 or 6; when m is 0, W₂ or W₄ is directly linked to the corresponding nitrogen in Ring 3 or Ring 6, respectively-,
each of W₂ and W₄ is independently hydrogen, a three member ring, four member ring, five member heterocyclic ring or substituted five member heterocyclic ring, a six member heterocyclic ring or substituted six member heterocyclic ring, a carboxylalkyl group or substituted carboxylalkyl group, a cyanoalkyl group or substituted cyanoalkyl group, an aminoalkyl group or substituted aminoalkyl group, a hydroxyalkyl group or substituted hydroxyalkyl group, an amino acid, an ester of amino acid, an amide of amino acid, a unnatural amino acid, an ester of unnatural amino acid or an amide of unnatural amino acid.

3. The compound of claim 1, wherein Ring 2 and Ring 3, and/or Ring 5 and Ring 6 of the compound are independently selected from the group consisting of:

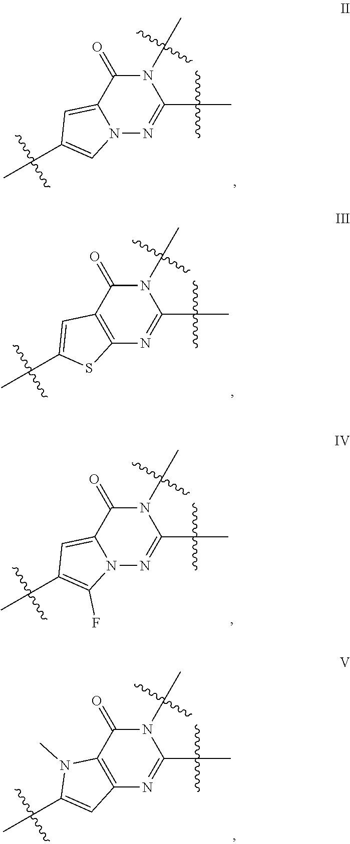 [SMILES: *c1cc2c(=O)n(*)c(*)nn2c1 ; *c1cc2c(=O)n(*)c(*)nc2s1 ; *c1cc2c(=O)n(*)c(*)nn2c1F ; *c1nc2cc(*)n(C)c2c(=O)n1*]

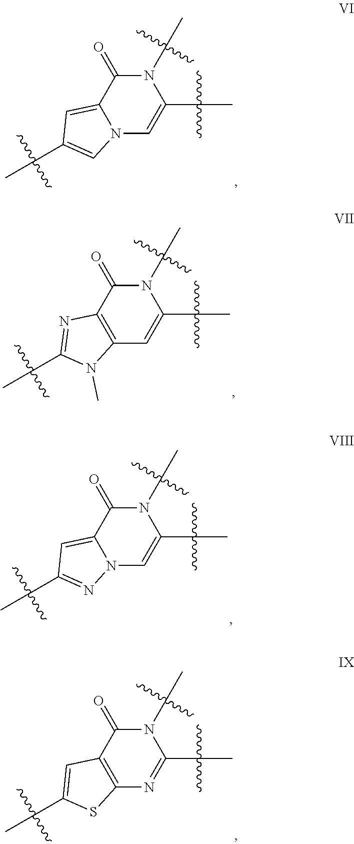 [SMILES: *c1cc2c(=O)n(*)c(*)cn2c1 ; *c1cc2c(nc(*)n2C)c(=O)n1* ; *c1cc2c(=O)n(*)c(*)cn2n1 ; *c1cc2c(=O)n(*)c(*)nc2s1]

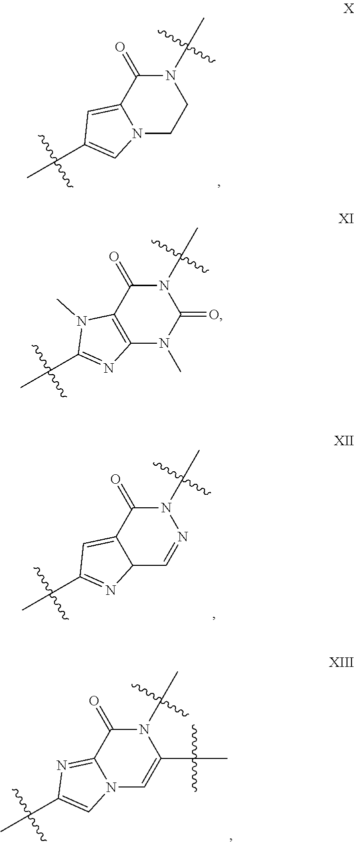 [SMILES: *c1cc2n(c1)CCN(*)C2=O ; *c1nc2c(c(=O)n(*)c(=O)n2C)n1C ; *C1=NC2C=NN(*)C(=O)C2=C1 ; *c1cn2cc(*)n(*)c(=O)c2n1]

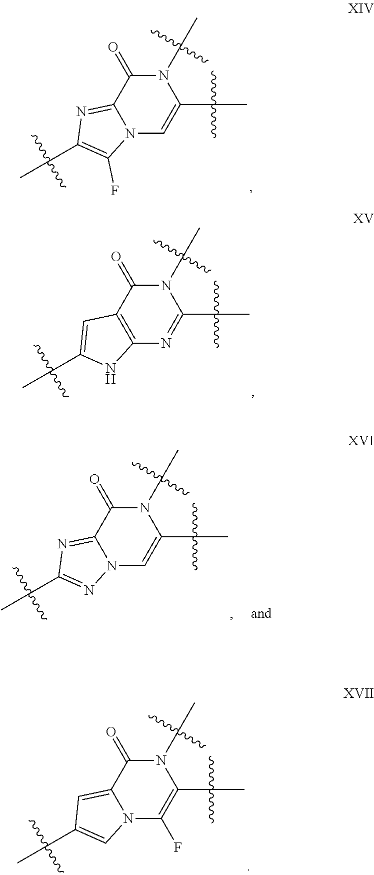 [SMILES: *c1nc2c(=O)n(*)c(*)cn2c1F ; *c1cc2c(=O)n(*)c(*)nc2[nH]1 ; *c1nc2c(=O)n(*)c(*)cn2n1 ; *c1cc2c(=O)n(*)c(*)c(F)n2c1]

4. The compound of claim 1, wherein Ring 2 and Ring 3, and/or Ring 5 and Ring 6 of the compound are independently selected from the group consisting of

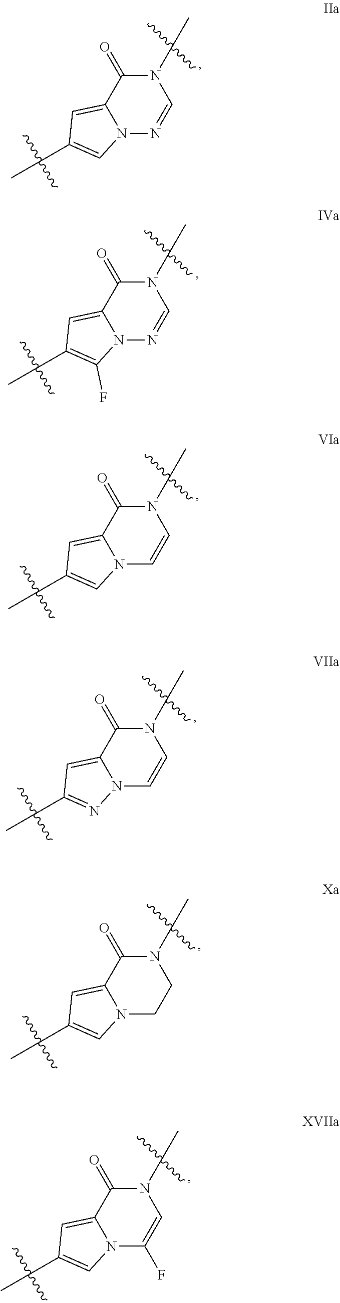 [SMILES: *c1cc2c(=O)n(*)cnn2c1 ; *c1cc2c(=O)n(*)cnn2c1F ; *c1cc2c(=O)n(*)ccn2c1 ; *c1cc2c(=O)n(*)ccn2n1 ; *c1cc2n(c1)CCN(*)C2=O ; *c1cc2c(=O)n(*)cc(F)n2c1]

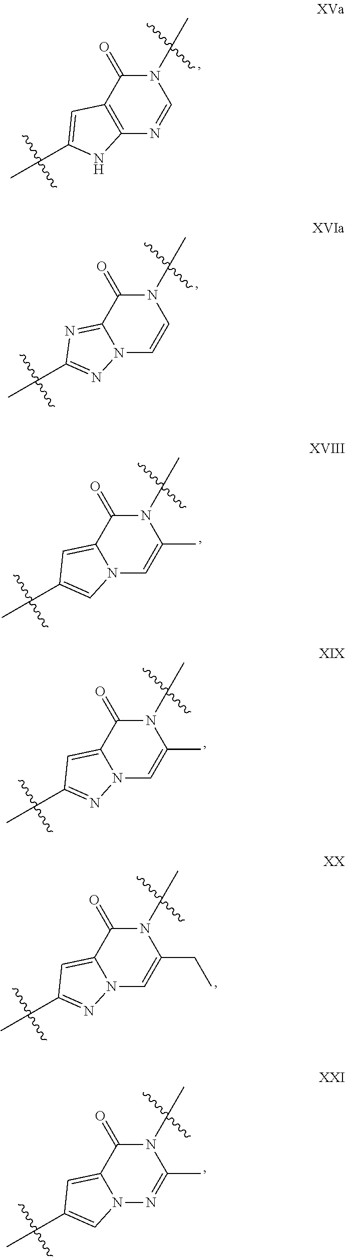 [SMILES: *c1cc2c(=O)n(*)cnc2[nH]1 ; *c1nc2c(=O)n(*)ccn2n1 ; *c1cc2c(=O)n(*)c(C)cn2c1 ; *c1cc2c(=O)n(*)c(C)cn2n1 ; *c1cc2c(=O)n(*)c(CC)cn2n1 ; *c1cc2c(=O)n(*)c(C)nn2c1]

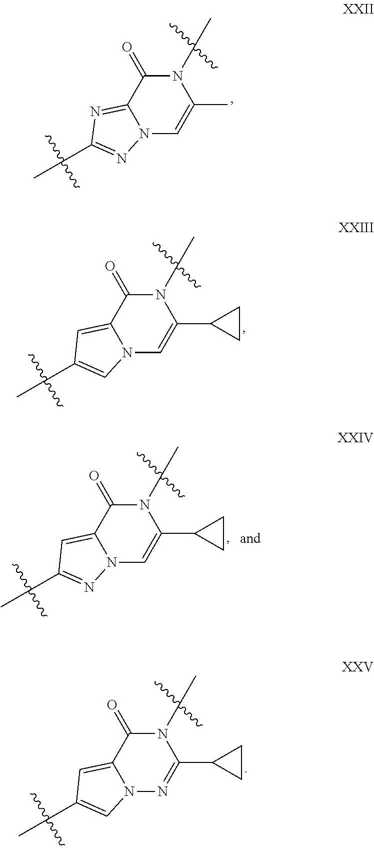 [SMILES: *c1nc2c(=O)n(*)c(C)cn2n1 ; *c1cc2c(=O)n(*)c(C3CC3)cn2c1 ; *c1cc2c(=O)n(*)c(C3CC3)cn2n1 ; *c1cc2c(=O)n(*)c(C3CC3)nn2c1]

5. The compound of claim 1, wherein each of L₁, L₂, L₃ and L₄ is independently a C₁-C₃ alkyl.

6. The compound of claim 1, wherein
each of W₁, W₂, W₃ and W₄ is independently selected from the group consisting of:

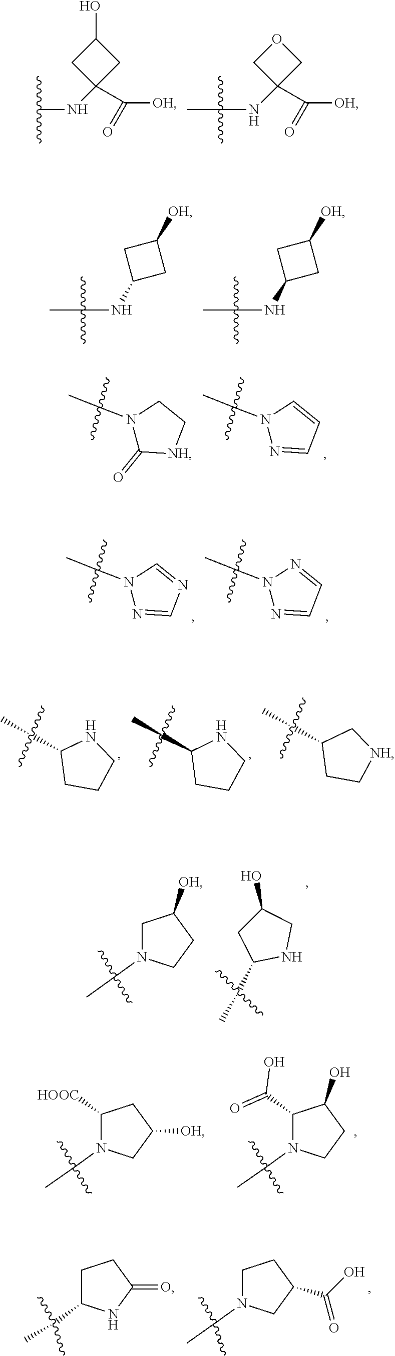

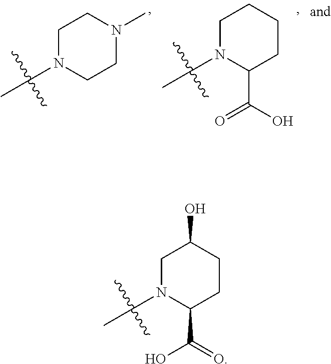

7. The compound of claim 1, wherein one or more of W₁, W₂, W₃ and W₄ have a general formula of: 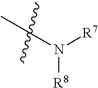
wherein each of R⁷ and R⁸ is independently —H, alkyl, or substituted alkyl.

8. The compound of claim 7, wherein R⁷ is one of 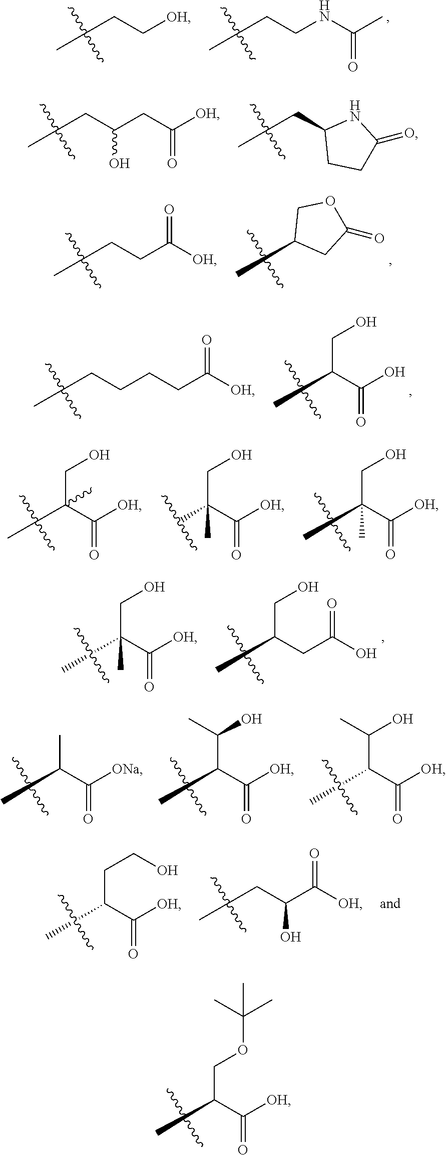
and wherein R⁸ is independently —H, alkyl, or substituted alkyl.

9. The compound of claim 7, wherein R⁷ is an ester of one of 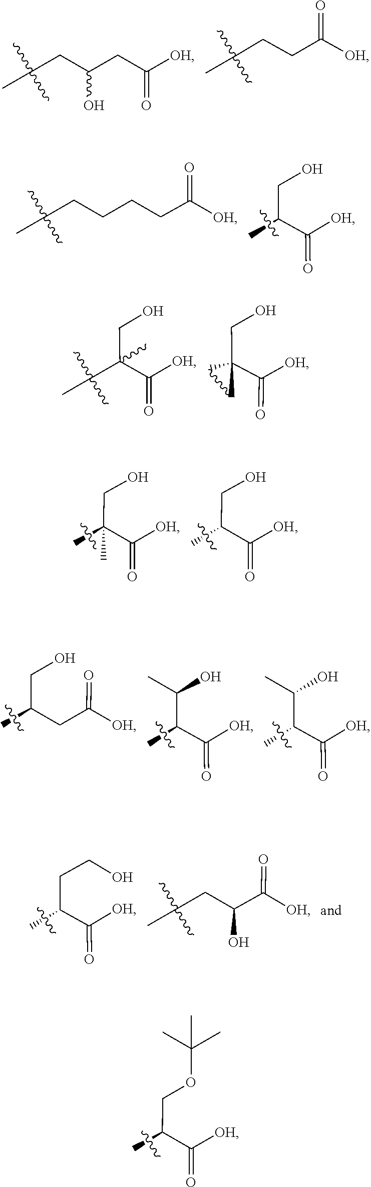
and wherein R⁸ is independently —H, alkyl, or substituted alkyl.

10. The compound of claim 1, wherein the compound is selected from the compounds listed in Table 1.

11. The compound of claim 1, wherein the compound is selected from the group consisting of

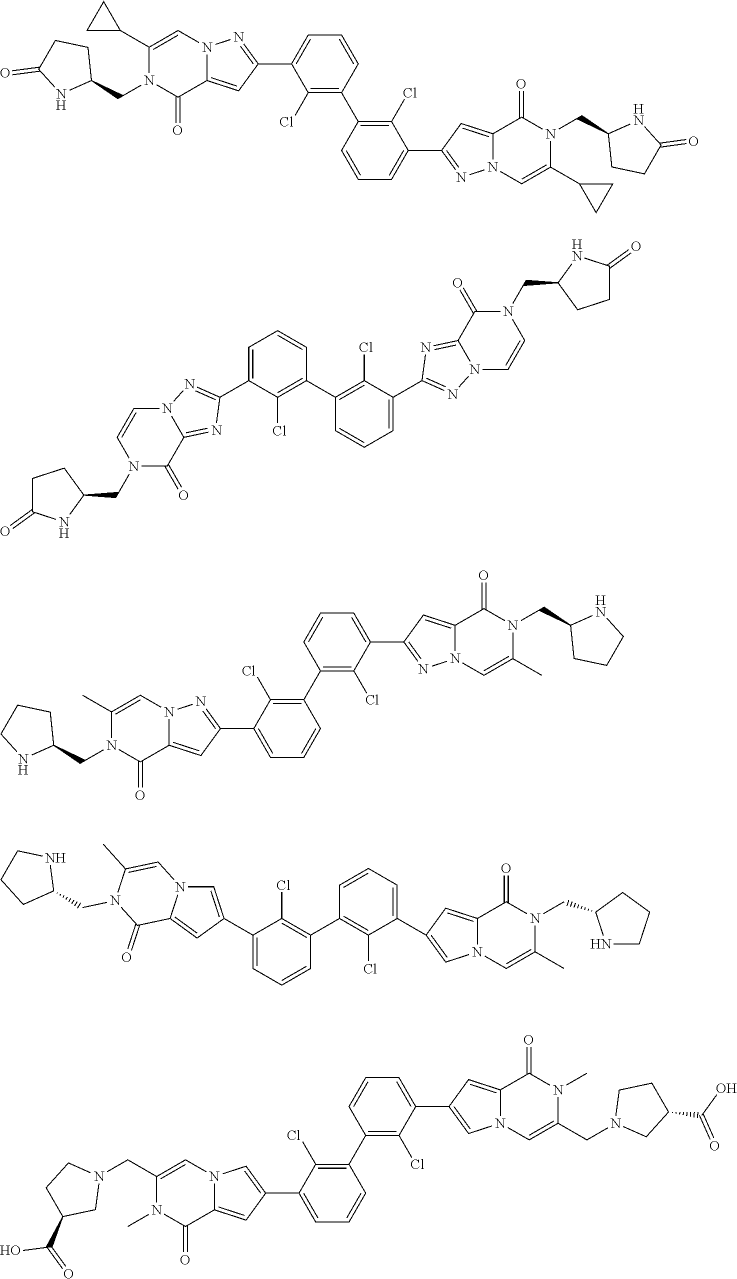

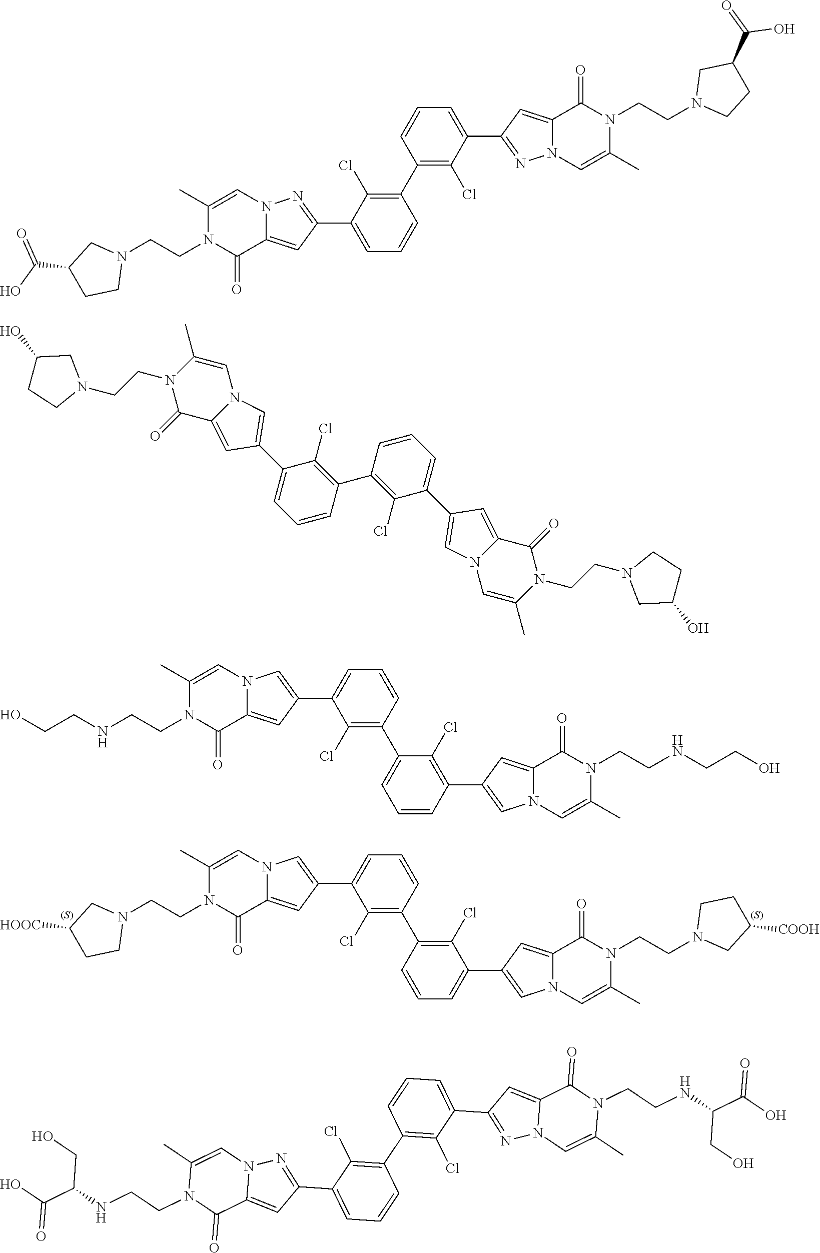

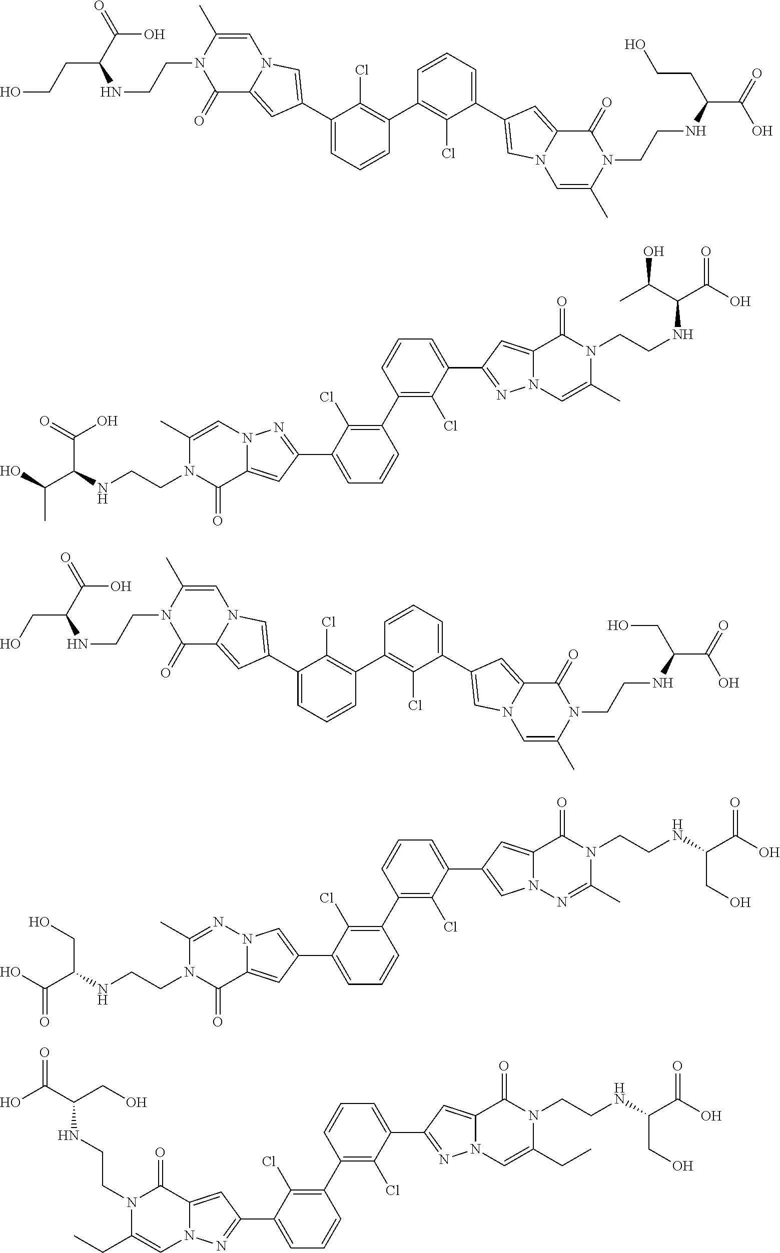

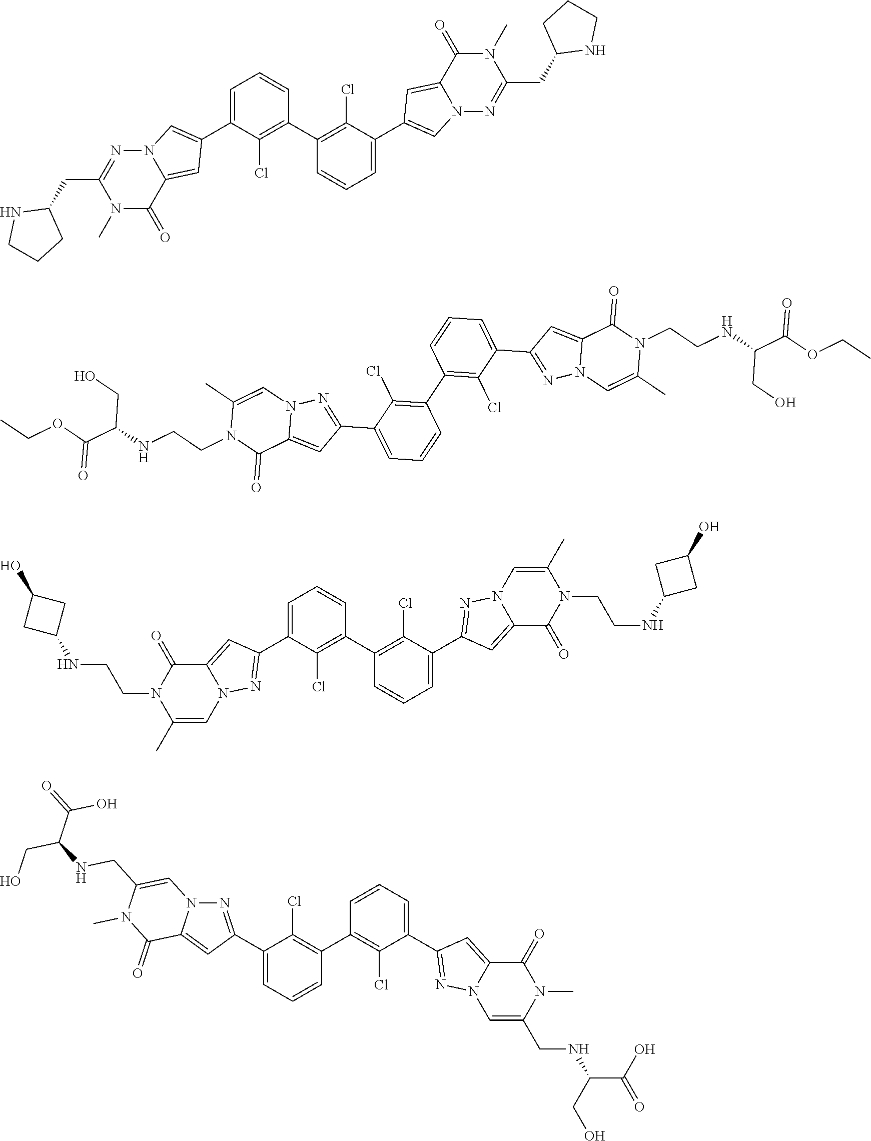 [SMILES: Cn1c(C[C@@H]2CCCN2)nn2cc(-c3cccc(-c4cccc(-c5cc6c(=O)n(C)c(C[C@@H]7CCCN7)nn6c5)c4Cl)c3Cl)cc2c1=O ; CCOC(=O)[C@H](CO)NCCn1c(C)cn2nc(-c3cccc(-c4cccc(-c5cc6c(=O)n(CCN[C@@H](CO)C(=O)OCC)c(C)cn6n5)c4Cl)c3Cl)cc2c1=O ; Cc1cn2nc(-c3cccc(-c4cccc(-c5cc6c(=O)n(CCN[C@H]7C[C@H](O)C7)c(C)cn6n5)c4Cl)c3Cl)cc2c(=O)n1CCN[C@H]1C[C@H](O)C1 ; Cn1c(CN[C@@H](CO)C(=O)O)cn2nc(-c3cccc(-c4cccc(-c5cc6c(=O)n(C)c(CN[C@@H](CO)C(=O)O)cn6n5)c4Cl)c3Cl)cc2c1=O]

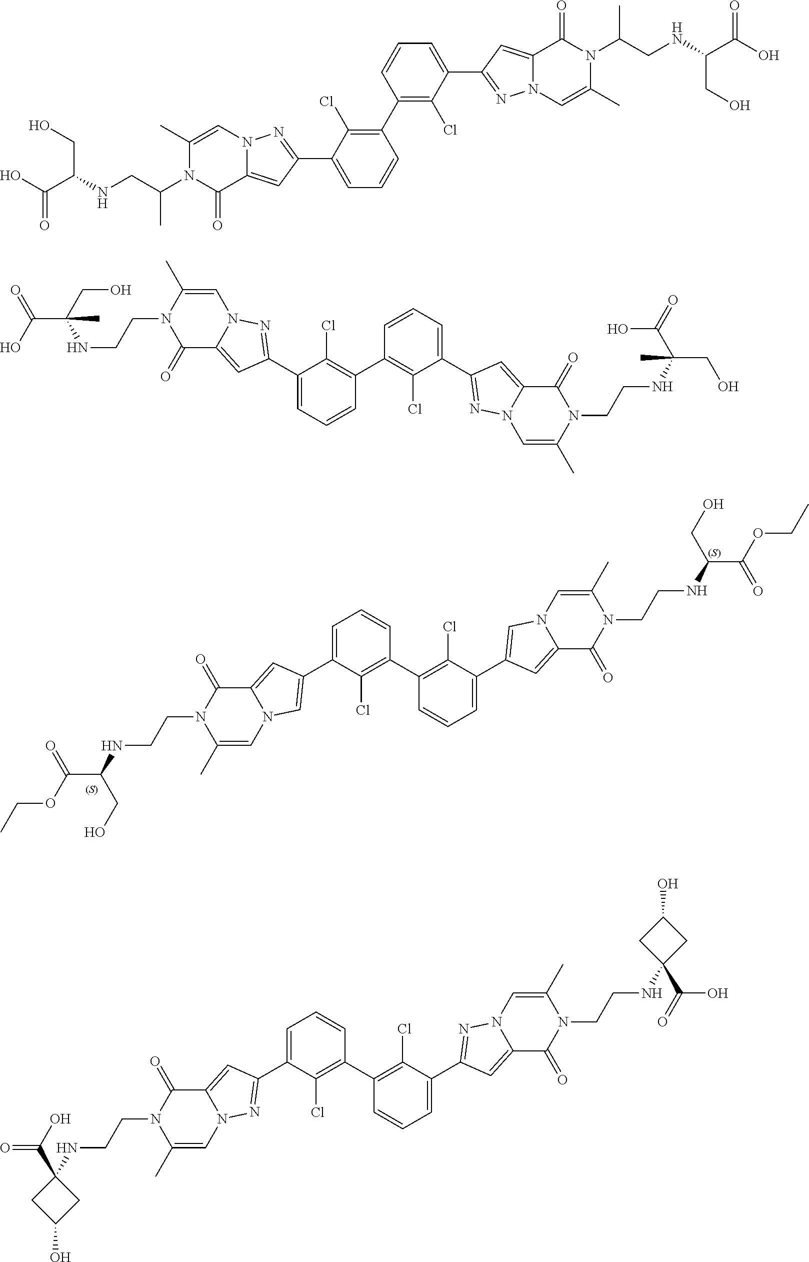

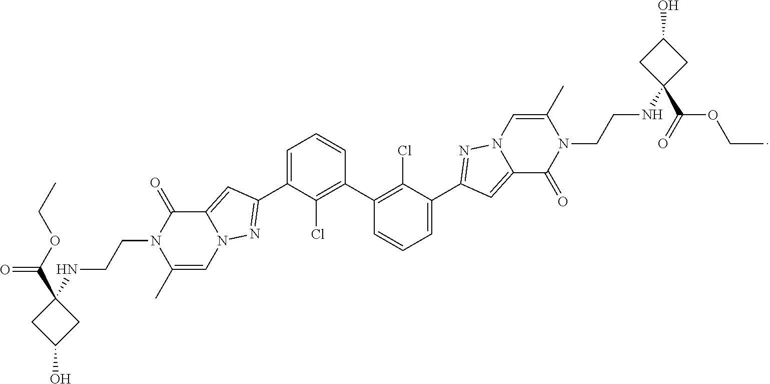 [SMILES: CCOC(=O)[C@]1(NCCn2c(C)cn3nc(-c4cccc(-c5cccc(-c6cc7c(=O)n(CCN[C@]8(C(=O)OCC)C[C@H](O)C8)c(C)cn7n6)c5Cl)c4Cl)cc3c2=O)C[C@@H](O)C1]

12. The compound of claim 1, wherein the compound has the formula of 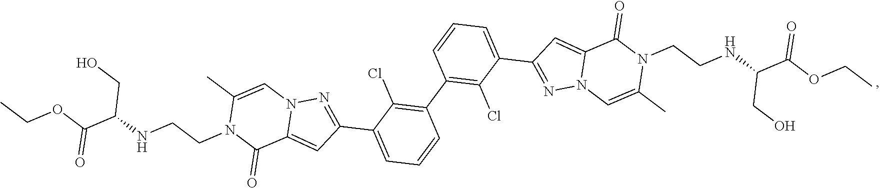 [SMILES: CCOC(=O)[C@H](CO)NCCn1c(C)cn2nc(-c3cccc(-c4cccc(-c5cc6c(=O)n(CCN[C@@H](CO)C(=O)OCC)c(C)cn6n5)c4Cl)c3Cl)cc2c1=O]
or is a pharmaceutically acceptable salt, stereoisomer, mixture of stereoisomers, deuterium substituted analog, or tautomer thereof.

13. The compound of claim 1, wherein the compound has the formula of 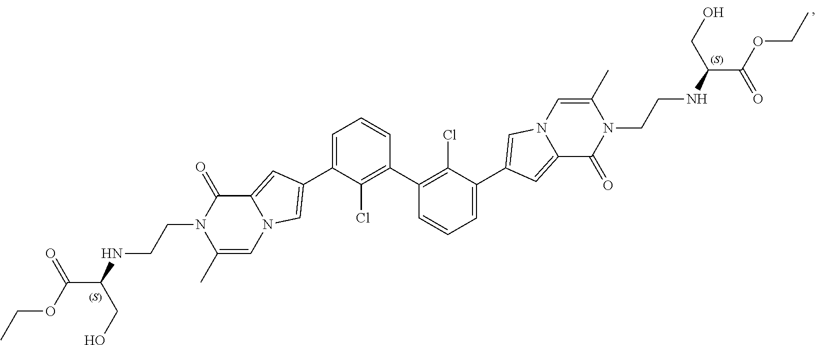
or is a pharmaceutically acceptable salt, stereoisomer, mixture of stereoisomers, deuterium substituted analog, or tautomer thereof.

14. The compound of claim 1, wherein the compound has the formula of 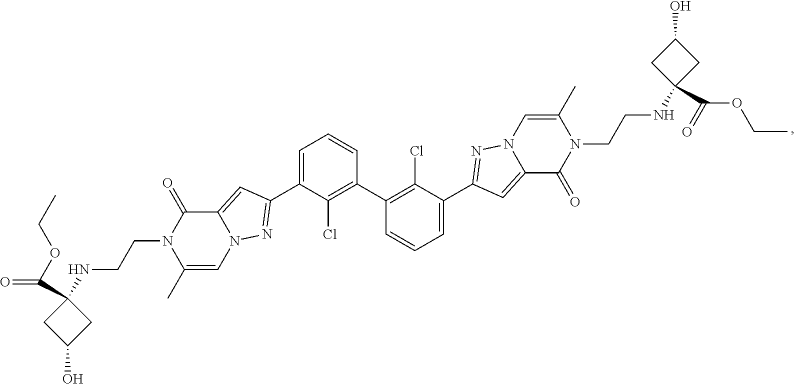 [SMILES: CCOC(=O)[C@]1(NCCn2c(C)cn3nc(-c4cccc(-c5cccc(-c6cc7c(=O)n(CCN[C@]8(C(=O)OCC)C[C@H](O)C8)c(C)cn7n6)c5Cl)c4Cl)cc3c2=O)C[C@@H](O)C1]
or is a pharmaceutically acceptable salt, stereoisomer, mixture of stereoisomers, deuterium substituted analog, or tautomer thereof.

15. A pharmaceutical composition, comprising:
the compound of claim 1; and
a pharmaceutically acceptable carrier.

16. A method for treating a disease associated with PD-L1 in a subject, comprising the step of:
administering to a subject in need of such treatment an effective amount of the pharmaceutical composition of claim 15.

17. The method of claim 16, wherein the disease is cancer.

18. The method of claim 16, wherein the disease is an immune-related disease.

19. The method of claim 16, wherein the disease is an infectious disease.

20. The method of claim 19, wherein the infectious disease is hepatitis B virus infection.
